(19) United States
(12) Patent Application Publication
(10) Pub. No.: US 2018/0296663 A1
(43) Pub. Date: Oct. 18, 2018

(54) VACCINE COMPOSITION

(71) Applicant: CureVac AG, Tübingen (DE)

(72) Inventors: Madeleine HIPP, Burladingen (DE); Regina HEIDENREICH, Tübingen (DE); Mariola FOTIN-MLECZEK, Sindelfingen (DE); Patrick BAUMHOF, Dusslingen (DE); Johannes LUTZ, Tübingen (DE); Aleksandra KOWALCZYK, Stuttgart (DE)

(21) Appl. No.: 15/736,699

(22) PCT Filed: Jun. 17, 2016
(86) PCT No.: PCT/EP2016/064093

§ 371 Date: Dec. 14, 2017

(30) Foreign Application Priority Data
Jun. 17, 2015 (EP) PCT/EP2015/001226

(51) Int. Cl. A61K 39/145; A61K 39/205; A61K 39/39

(52) U.S. Cl. CPC A61K 39/145; A61K 2039/53; A61K 39/39; A61K 39/205

(57) ABSTRACT

A composition or combination comprising at least a first immunogenic component and at least a second adjuvant component, wherein the first immunogenic component comprises at least one nucleic acid molecule encoding at least one epitope of at least one antigen, and wherein the second adjuvant component comprises at least one immune potentiator compound and/or at least one delivery system compound.

The present invention relates to a combination or composition comprising at least a first immunogenic component and at least a second adjuvant component. The combination or composition may be used as pharmaceutical composition especially for the treatment or prophylaxis of an infectious disease or an allergy or an autoimmune disease or a cancer or tumor disease.

Vaccines are one of the most economic and influential public health measures and have contributed greatly to decrease the mortality due to infectious diseases and other diseases. Despite their success for diseases like polio, tetanus or small pox, there is still a medical need to produce vaccines for other diseases like HIV where no vaccines are yet available, or to replace existing vaccines with advanced vaccines that show increased efficacy or improved product characteristics. Several problems in vaccine development have proved difficult to solve: Vaccines are often inefficient for the very young and the very old; many vaccines need to be given several times, and the protection they confer wanes over time, requiring booster administrations. As generally accepted, many vaccines would be enabled or improved if they could elicit a stronger and more durable immune response. Therefore, development of vaccines is still ongoing.

Besides traditional vaccines it is known to use gene therapeutic approaches for providing vaccines. Nucleic acid based vaccines differ from traditional vaccines due to the in situ antigen production and the ease with which they may be produced. Various methods have been developed for introducing DNA into cells, such as calcium phosphate transfection, polybrene transfection, protoplast fusion, electroporation, microinjection and lipofection. DNA viruses may likewise be used as a DNA vehicle achieving a very high transfection rate. Nevertheless, the use of DNA entails the risk of the DNA being inserted into an intact gene of the host cell's genome by e.g. recombination. In this case the affected gene may be mutated and inactivated or may give rise to misinformation. The use of RNA as a gene therapeutic agent or vaccine is substantially safer, because RNA does not involve the risk of being integrated into the genome.

Thus, RNA expression systems have considerable advantages over DNA expression systems in gene therapy and especially in genetic vaccination. Therefore, especially mRNA-based vaccines are a promising vaccine platform and are a powerful tool to express vaccination antigens. These vaccines use messenger RNAs (mRNAs) encoding the vaccination antigen of choice, which are injected into the patient and are taken up by local somatic and immune cells. Once inside the cytosol the mRNAs are translated and the vaccination antigens are produced as proteins or peptides, which induce an immune response. The magnitude, duration and character of the immune response depend on the immunostimulatory context, in which the antigens are presented. To elicit strong immune responses, the antigens should be presented to the immune system in a pro-inflammatory context.

Compared to inactivated or live-attenuated pathogens nucleic acid based vaccines are less immunogenic when administered alone. One approach to improve the immune response is the addition of an adjuvant, e.g. aluminium salts or oil-in-water emulsions (Mbow M. L. et al. (2010), Current Opinion in Immunology 22: 1-6).

Accordingly, the development of new efficient and safe pharmaceutical compositions that include adjuvants for vaccination purposes which support induction and maintenance of an adaptive immune response by initiating or boosting a parallel innate immune response represents a main challenging problem.

Adjuvants are usually defined as compounds that can increase and/or modulate the intrinsic immunogenicity of an antigen. To reduce negative side effects, new vaccines have a more defined composition that often leads to lower immunogenicity compared with previous whole-cell or virus-based vaccines. Adjuvants are therefore required to assist new vaccines to induce potent and persistent immune responses, with the additional benefit that less antigen and fewer injections are needed. Now it is clear that the adaptive immune response mainly depends on the level and specificity of the initial danger signals perceived by innate immune cells following infection or vaccination (Guy, B. (2007), Nat Rev Microbiol 5(7): 505-17.).

Unfortunately, only a few licensed adjuvants are available so far. Most prominent is Alum, which is known to be safe, but also represents a very weak adjuvant. Many further adjuvants have been developed, e.g. including the administration of pathogens, CpG-nucleotides, etc. Most of these new or “established” adjuvants, however, still do not satisfy the above requirements, since many new and emerging problems have to be considered and solved.

These problems inter alia include new and re-emerging infectious diseases, repeated administrations, threat of pandemic flu, etc.

Furthermore, the new vaccine targets are usually more difficult to develop and—due to their specifically tailored immune responses—require more potent adjuvants to enable success. Moreover, there are still a significant number of important pathogens for which we do not even have effective vaccines at present. This represents a very challenging future target. To enable vaccine development against such targets, more potent pharmaceutical compositions that include adjuvants and such targets will be necessary. Therefore, the new adjuvants in such compositions will need to offer advantages, including more heterologous antibody responses, covering pathogen diversity, induction of potent functional antibody responses, ensuring pathogen killing or neutralization and induction of more effective T cell responses, for direct and indirect pathogen killing, particularly the induction of cytotoxic T cells which are part of a Th1 immune response. In addition, adjuvants may be necessary to achieve more pragmatic effects, including antigen dose reduction and overcoming antigen competition in combination vaccines. Moreover, against the background of an aging population, which is increasingly susceptible to infectious diseases, new adjuvants will be necessary to overcome the natural deterioration of the immune response with age (O'Hagan, D. T. and E. De Gregorio (2009), Drug Discov Today 14(11-12): 541-51).

The review of O'Hagan (2009; supra) summarizes some reasons for the urgent need of new effective adjuvants e.g. the requirement of a lower antigen dose in vaccines, the necessity to increase the breadth of an immune response and the heterologous activity, to enable complex combination vaccines, and to overcome antigenic competition, to overcome limited immune response in some groups of the population, such as the elderly, the young children, and infants, patients with chronic diseases and the immunocompromised, to increase effector T cell response and antibody titers, to induce protective responses more rapidly and also to extend the duration of response by enhancing memory B and T cell responses.

Summarizing the above, new efficient and safe pharmaceutical compositions that include immunostimulating agents or adjuvants are required, which are preferably efficient in inducing an innate immune response, particularly in inducing the anti-viral cytokine IFN-alpha; and which are also efficient in supporting an adaptive immune response; safe, i.e. not associated with any long-term effects; which are well tolerated; which are available via a simple synthetic pathway; which exhibit low cost storage conditions (particularly feasible lyophilisation); which require simple and inexpensive components; which are biodegradable; which are compatible with many different kinds of vaccine antigens; which are capable of codelivery of antigen and immune potentiator, etc.

As already explained above adjuvants or immunostimulating agents usually act via their capability to induce an innate immune response. The innate immune system forms the dominant system of host defense in most organisms and comprises barriers such as humoral and chemical barriers including, e.g., inflammation, the complement system and cellular barriers. The innate immune system is typically based on a small number of receptors, called pattern recognition receptors. They recognize conserved molecular patterns that distinguish foreign organisms, like viruses, bacteria, fungi and parasites, from cells of the host. A new class of vaccine adjuvants target signal pathways which relate to pattern recognition receptors (PRRs). Pathogen-associated molecular patterns (PAMPs) are recognized by the immune system by means of these PRRs, which trigger the production of proinflammatory cytokines and immune activation.

Especially five families of PRRs have been shown to initiate proinflammatory signaling pathways:
Toll-like receptors (TLRs),
NOD-like receptors (NLRs),
RIG-I-like receptors (RLRs),
C-type lectin receptors (CLRs) and
Cytosolic dsDNA sensors (CDSs).

PAMPs include viral nucleic acids, components of bacterial and fungal walls, flagellar proteins, and more. The first family of PRRs studied in detail was the TLR-family. TLRs are transmembrane proteins which recognize ligands of the extracellular milieu or of the lumen of endosomes. Following ligand-binding they transduce the signal via cytoplasmic adaptor proteins which leads to triggering of a host-defense response and entailing production of antimicrobial peptides, proinflammatory chemokines and cytokines, antiviral cytokines, etc. (see e.g. Meylan, E., J. Tschopp, et al. (2006), Nature 442(7098): 39-44). Further relevant components of the immune system include e.g cytoplasmic RNA and DNA sensors, and Type I interferons. Therefore, the immunostimulating agents or adjuvants are defined herein preferably as inducers of an innate immune response, which activate PRRs. Hereby, a cascade of signals is elicited, which e.g. may result in the release of cytokines (e.g. IFN-alpha) supporting the innate immune response. Accordingly, it is preferably a feature of an immunostimulating agent or adjuvant to bind to such receptors and activate such PRRs. Ideally, such an agent or adjuvant additionally supports the adaptive immune response by e.g. shifting the immune response such that the preferred class of Th cells is activated. Depending on the disease or disorder to be treated a shift to a Th1-based immune response may be preferred or, in other cases, a shift to a Th2 immune response may be preferred.

As an example, among the above developed new adjuvants, some nucleic acids like CpG DNA oligonucleotides or isRNA (immunostimulating RNA) turned out to be promising candidates for new immunostimulating agents or adjuvants, as they allow the therapeutic or prophylactic induction of an innate immune response. Comprehensibly, such nucleic acid based adjuvants usually have to be delivered effectively to the site of action to allow induction of an effective innate immune response without unnecessary loss of adjuvant activity and, in some cases, without the necessity to increase the administered volume above systemically tolerated levels.

One approach to solve this issue may be the transfection of cells which are part of the innate immune system (e.g. dendritic cells, plasmacytoid dendritic cells (pDCs)) with immunostimulatory nucleic acids, which are ligands of PRRs, (e.g. TLRs), and thus may lead to immunostimulation by the nucleic acid ligand. Further approaches may be the direct transfection of nucleic acid based adjuvants. All of these approaches, however, are typically impaired by inefficient delivery of the nucleic acid and consequently diminished adjuvant activity, in particular when administered locally.

However, one main disadvantage of such nucleic acid based adjuvant approaches until today is their limited ability to cross the plasma membrane of mammalian cells, resulting in poor cellular access and inadequate therapeutic efficacy. Until today this hurdle represents a major challenge for nucleic acid transfection based applications, e.g. biomedical developments and accordingly the commercial success of many biopharmaceuticals (see e.g. Foerg, C. and Merkle, H. P. (2008), J Pharm Sci 97, 144-62).

Transfection of nucleic acids or genes into cells or tissues has been investigated up to date in the context of in vitro transfection purposes and in the context of gene therapeutic approaches. However, no adjuvants are available so far which are based on such gene delivery techniques which are efficient and safe, in particular no licensed adjuvants. This is presumably due to the complex requirements of adjuvants in general in combination with stability issues to be solved in the case of nucleic acid based adjuvants. Nevertheless, transfection of nucleic acids or genes into cells or tissues for eliciting an (innate and/or adaptive) immune response appears to provide a promising approach to provide new adjuvants.

However, many of these approaches utilize transfection of nucleic acids or genes into cells or tissues without the purpose to induce an innate immune response. There are even some gene therapeutic therapies, which have to strictly avoid induction of an innate immune response. Even in the rare cases, where vaccination is carried out to induce an adaptive antigen-specific immune response using administration of nucleic acids, e.g. in tumor vaccinations using DNA or mRNA encoded antigens, induction of an adaptive immune response is typically carried out as an active immunization against the encoded antigen but not as an accompanying adjuvant therapy and thus may require additional administration of a separate adjuvant to induce an innate immune response.

Even if many transfection methods are known in the art, transfer or insertion of nucleic acids or genes into an individual's cells still represents a major challenge today and is not yet solved satisfactorily. To address this complex issue a variety of methods were developed in the last decade. These include transfection by calcium phosphate, cationic lipids, cationic polymers, and liposomes. Further methods for transfection are electroporation and viral transduction.

However, as known to a skilled person, systems for transfer or insertion of nucleic acids or genes have to fulfil several requirements for in vivo applications which include efficient nucleic acid delivery into an individual's cells with high functionality, protection of the nucleic acid against ubiquitously occurring nucleases, release of the nucleic acid in the cell, no safety concerns, feasible manufacturing in a commercially acceptable form amenable to scale-up and storage stability under low cost conditions (e.g feasible lyophilisation). These requirements are to be added to the complex requirements of an adjuvant particularly if it is in the form of a nucleic acid as outlined above.

Some successful strategies for the transfer or insertion of nucleic acids or genes available today rely on the use of viral vectors, such as adenoviruses, adeno-associated viruses, retroviruses, and herpes viruses. Viral vectors are able to mediate gene transfer with high efficiency and the possibility of long-term gene expression. However, the acute immune response (“cytokine storm”), immunogenicity, and insertion mutagenesis uncovered in gene therapy clinical trials have raised serious safety concerns about some commonly used viral vectors.

Another solution to the problem of transfer or insertion of nucleic acids or genes may be found in the use of non-viral vectors. Although non-viral vectors are not as efficient as viral vectors, many non-viral vectors have been developed to provide a safer alternative. Methods of non-viral nucleic acid delivery have been explored using physical (carrier-free nucleic acid delivery) and chemical approaches (synthetic vector-based nucleic acid delivery). Physical approaches usually include needle injection, electroporation, gene gun, ultrasound, and hydrodynamic delivery, employ a physical force that permeates the cell membrane and facilitates intracellular gene transfer. The chemical approaches typically use synthetic or naturally occurring compounds (e.g. cationic lipids, cationic polymers, lipid-polymer hybrid systems) as carriers to deliver the nucleic acid into the cells. Although significant progress has been made in the basic science and applications of various nonviral nucleic acid delivery systems, the majority of non-viral approaches are still much less efficient than viral vectors, especially for in vivo gene delivery (see e.g. Gao, X., Kim, K. & Liu, D., AAPS J9, E92-104 (2007)).

Such transfection agents as defined above typically have been used successfully solely in in vitro reactions. For application of nucleic acids in vivo, however, further requirements have to be fulfilled. For example, complexes between nucleic acids and transfection agents have to be stable in physiological salt solutions with respect to agglomerisation. Furthermore, such complexes typically must not interact with parts of the complement system of the host and thus must not be immunogenic itself as the carrier itself shall not induce an adaptive immune response in the individual. Additionally, the complex shall protect the nucleic acid from early extracellular degradation by ubiquitously occurring nucleases.

One more promising approach utilizes cationic polymers. Cationic polymers turned out to be efficient in transfection of nucleic acids, as they can tightly complex and condense a negatively charged nucleic acid. Thus, a number of cationic polymers have been explored as carriers for in vitro and in vivo gene delivery. These include polyethylenimine (PEI), polyamidoamine and polypropylamine dendrimers, polyallylamine, cationic dextran, chitosan, cationic proteins and cationic peptides. Although most cationic polymers share the function of condensing DNA into small particles and facilitate cellular uptake via endocytosis through charge-charge interaction with anionic sites on cell surfaces, their transfection activity and toxicity differs dramatically.

Also in the context of mRNA-based vaccines several approaches use already adjuvants to improve the immunogenicity of mRNA-based vaccines (for review Schlake T. et al. (2012), RNA Biol November 9(11)). Examples are lipopolysaccharide (LPS), polyinosinic:polycytidylic acid (polyl:C), protamine-complexed mRNA (Fotin-Mleczek M. et al. (2010), J Immunother 34: 1-15; Diken M. et al. (2011), Gene Ther 18: 702-8; WO2010/037539 A1) or recombinant proteins like GM-CSF (Carralot J. P. et al. (2004), Cellular and Molecular Life Sciences 61(18): 2418-24), Flt-3 ligand (Kreiter S. et al. (2011), Cancer Research 71(19): 6132-42), or CD40 ligand (CD40L). Moreover the use of a complex of nucleic acids with disulfide-crosslinked cationic components leads to increased immune responses as described in WO2012/013326 A1.

The immunostimulatory effect of RNA complexed to short cationic peptides was demonstrated by Fotin-Mleczek et al. (WO2009/030481). These formulations appear to efficiently induce the cytokine production in immunocompetent cells. In the above context, cationic polymers exhibit better transfection efficiency with rising molecular weight. However, a rising molecular weight also leads to a rising toxicity of the cationic polymer. In this above context, (high molecular weight) PEI is perhaps the most active and most studied polymer for transfection of nucleic acids, in particular for gene delivery purposes. Unfortunately, it exhibits the same drawback due to its non-biodegradable nature and toxicity. Furthermore, even though polyplexes formed by high molecular weight polymers exhibit improved stability under physiological conditions, data have indicated that such polymers can hinder vector unpacking. To overcome this negative impact, Read et al. (see Read, M. L. et al., J Gene Med. 5, 232-245 (2003); and Read, M. L. et al., Nucleic Acids Res 33, e86 (2005)) developed a new type of synthetic vector based on a linear reducible polycation (RPC) prepared by oxidative polycondensation of the peptide Cys-Lys₁₀-Cys. This peptide Cys-Lys₁₀-Cys can be cleaved by the intracellular environment to facilitate release of nucleic acids. In this context, Read et al. (2003, supra) could show that polyplexes formed by these RPCs are destabilised by reducing conditions enabling efficient release of DNA and mRNA. However, examining the transfection efficiency in vitro Read et al. (2003, supra) also observed that N/P (nitrogen to phosphor atoms) ratios of 2 were unsatisfying and higher N/P ratios were necessary to improve transfection efficiency. Additionally, Read et al. (2003, supra) observed that chloroquine or the cationic lipid DOTAP was additionally necessary to enhance transfection efficiency to adequate levels. As a consequence, Read et al. (2005, supra) included histidine residues into the RPCs which have a known endosomal buffering capacity and showed that such histidine-rich RPCs can be cleaved by the intracellular reducing environment. This approach enabled efficient cytoplasmic delivery of a broad range of nucleic acids, including plasmid DNA, mRNA and siRNA molecules without the requirement for the endosomolytic agent chloroquine. For in vivo application Read et al. (2005, supra) proposed modifications with the hydrophilic polymer poly[N-(2hydroxy-propyl)methacrylamide]. Unfortunately, they could not prevent aggregation of polyplexes and binding of polycationic complexes to serum proteins. Furthermore, strong cationic charged complexes are formed (positive zeta potential) when complexing the nucleic acid due to the large excess of cationic polymer, which is characterized by the high N/P ratio. Accordingly, such complexes are only of limited use in vivo due to their strong tendency of salt induced agglomeration and interactions with serum contents (opsonization). Additionally, these (positively charged) complexes may excite complement activation, when used for purposes of gene therapy. It has also turned out that these positively charged RPC based complexes showed poor translation of the nucleic acid cargo subsequent to local administration into the dermis.

Thus, in vivo application of nucleic acids appears to be still one of the most challenging problems because plasma proteins with anionic charges may non-specifically bind to positively charged complexes and rapidly remove them e.g. via the reticulo-endothelial system. Opsonization and activation of the complement system by cationic complexes are additional physiological phenomena that can participate in lowering the efficacy of in vivo administered cationic complexes. This particularly applies to administration of nucleic acid-based drugs, e.g. the transfection of nucleic acids into cells or tissues, particularly if the expression of an encoded protein or peptide or transcription of an RNA of the transfected nucleic acid is intended.

Mucosal immune responses are pivotal for the protection against many pathogens that infect the body via the gastrointestinal or respiratory tract. Unfortunately, mucosal immunity is difficult to achieve by vaccinations that are not given via the mucosal route (e.g. oral, intranasal, intrapulmonary vaccination). Non-mucosal delivery of vaccines yields only limited mucosal immunity and hence only limited protection against pathogens that infect the gastrointestinal or pulmonary tract.

In summary, depending on sequence, formulation and application route, nucleic acid based vaccines and especially mRNA based vaccines might exhibit only limited immunostimulatory capacities, which leads to reduced humoral and cellular immune responses. The prior art does not provide feasible means or methods, which, on the one hand side, allow to establish efficient and safe adjuvants for vaccination purposes, and which, on the other hand side, are furthermore suited for in vivo delivery of nucleic acids, in particular for compacting and stabilizing a nucleic acid for the purposes of nucleic acid transfection in vivo without exhibiting the negative side effects as discussed above. More precisely, no means or methods are known in the prior art in the above context, which are, on the one hand side, stable enough to carry a nucleic acid cargo to the target before they are metabolically cleaved, and which, on the other hand side, can be cleared from the tissue before they can accumulate and reach toxic levels.

Therefore, there is still a need to improve the immunogenicity and the immune response of nucleic acid based vaccines and especially of mRNA based vaccines. It is the object of the present invention to provide improved compositions and methods, which address the above mentioned problems. The object underlying the present invention is solved by the subject matter of the present invention, preferably by the subject matter of the claims. Particularly, the object underlying the present invention is solved according to a first aspect by a composition and by a pharmaceutical composition as defined in the claims. According to further aspects of the invention the object is solved by a kit and a vaccine and by a method of treatment or prophylaxis as defined in the claims.

For the sake of clarity and readability the following scientific background information and definitions are provided. Any technical features disclosed thereby can be part of each and every embodiment of the invention. Additional definitions and explanations can be provided in the context of this disclosure.

Immune system: The immune system may protect organisms from infection. If a pathogen breaks through a physical barrier of an organism and enters this organism, the innate immune system provides an immediate, but non-specific response. If pathogens evade this innate response, vertebrates possess a second layer of protection, the adaptive immune system. Here, the immune system adapts its response during an infection to improve its recognition of the pathogen. This improved response is then retained after the pathogen has been eliminated, in the form of an immunological memory, and allows the adaptive immune system to mount faster and stronger attacks each time this pathogen is encountered. According to this, the immune system comprises the innate and the adaptive immune system. Each of these two parts contains so called humoral and cellular components.

Immune response: An immune response may typically either be a specific reaction of the adaptive immune system to a particular antigen (so called specific or adaptive immune response) or an unspecific reaction of the innate immune system (so called unspecific or innate immune response).

Adaptive immune system: The adaptive immune system is composed of highly specialized, systemic cells and processes that eliminate or prevent pathogenic growth. The adaptive immune response provides the vertebrate immune system with the ability to recognize and remember specific pathogens (to generate immunity), and to mount stronger attacks each time the pathogen is encountered. The system is highly adaptable because of somatic hypermutation (a process of increased frequency of somatic mutations), and V(D)J recombination (an irreversible genetic recombination of antigen receptor gene segments). This mechanism allows a small number of genes to generate a vast number of different antigen receptors, which are then uniquely expressed on each individual lymphocyte. Because the gene rearrangement leads to an irreversible change in the DNA of each cell, all of the progeny (offspring) of that cell will then inherit genes encoding the same receptor specificity, including the Memory B cells and Memory T cells that are the keys to long-lived specific immunity. Immune network theory is a theory of how the adaptive immune system works, that is based on interactions between the variable regions of the receptors of T cells, B cells and of molecules made by T cells and B cells that have variable regions.

Adaptive immune response: The adaptive immune response is typically understood to be antigen-specific. Antigen specificity allows for the generation of responses that are tailored to specific antigens, pathogens or pathogen-infected cells. The ability to mount these tailored responses is maintained in the body by “memory cells”. Should a pathogen infect the body more than once, these specific memory cells are used to quickly eliminate it. In this context, the first step of an adaptive immune response is the activation of naïve antigen-specific T cells or different immune cells able to induce an antigen-specific immune response by antigen-presenting cells. This occurs in the lymphoid tissues and organs through which naïve T cells are constantly passing. Cell types that can serve as antigen-presenting cells are inter alia dendritic cells, macrophages, and B cells. Each of these cells has a distinct function in eliciting immune responses. Dendritic cells take up antigens by phagocytosis and macropinocytosis and are stimulated by contact with e.g. a foreign antigen to migrate to the local lymphoid tissue, where they differentiate into mature dendritic cells. Macrophages ingest particulate antigens such as bacteria and are induced by infectious agents or other appropriate stimuli to express MHC molecules. The unique ability of B cells to bind and internalize soluble protein antigens via their receptors may also be important to induce T cells. Presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which do not recognize and bind antigen directly, but instead recognize short peptide fragments e.g. of pathogen-derived protein antigens, which are bound to MHC molecules on the surfaces of other cells.

Cellular immunity/cellular immune response: Cellular immunity relates typically to the activation of macrophages, natural killer cells (NK), antigen-specific cytotoxic T-lymphocytes, and the release of various cytokines in response to an antigen. In a more general way, cellular immunity is not related to antibodies but to the activation of cells of the immune system. A cellular immune response is characterized e.g. by activating antigen-specific cytotoxic T-lymphocytes that are able to induce apoptosis in body cells displaying epitopes of an antigen on their surface, such as virus-infected cells, cells with intracellular bacteria, and cancer cells displaying tumor antigens; activating macrophages and natural killer cells, enabling them to destroy pathogens; and stimulating cells to secrete a variety of cytokines that influence the function of other cells involved in adaptive immune responses and innate immune responses.

Humoral immunity/humoral immune response: Humoral immunity refers typically to antibody production and the accessory processes that may accompany it. A humoral immune response may be typically characterized, e.g., by Th2 activation and cytokine production, germinal center formation and isotype switching, affinity maturation and memory cell generation. Humoral immunity also typically may refer to the effector functions of antibodies, which include pathogen and toxin neutralization, classical complement activation, and opsonin promotion of phagocytosis and pathogen elimination.

Innate immune system: The innate immune system, also known as non-specific immune system, comprises the cells and mechanisms that defend the host from infection by other organisms in a non-specific manner. This means that the cells of the innate system recognize and respond to pathogens in a generic way, but unlike the adaptive immune system, it does not confer long-lasting or protective immunity to the host. The innate immune system may be e.g. activated by ligands of pathogen-associated molecular patterns (PAMP) receptors, e.g. Toll-like receptors (TLRs) or other auxiliary substances such as lipopolysaccharides, TNF-alpha, CD40 ligand, or cytokines, monokines, lymphokines, interleukins or chemokines, immunostimulatory nucleic acids, immunostimulatory RNA (isRNA), CpG-DNA, antibacterial agents, or anti-viral agents. Typically a response of the innate immune system includes recruiting immune cells to sites of infection, through the production of chemical factors, including specialized chemical mediators, called cytokines; activation of the complement cascade; identification and removal of foreign substances present in organs, tissues, the blood and lymph, by specialized white blood cells; activation of the adaptive immune system through a process known as antigen presentation; and/or acting as a physical and chemical barrier to infectious agents.

Adjuvant/adjuvant component: An adjuvant or an adjuvant component in the broadest sense is typically a (e.g. pharmacological or immunological) agent or composition that may modify, e.g. enhance, the efficacy of other agents, such as a drug or vaccine. Conventionally the term refers in the context of the invention to a compound or composition that serves as a carrier or auxiliary substance for immunogens and/or other pharmaceutically active compounds. It is to be interpreted in a broad sense and refers to a broad spectrum of substances that are able to increase the immunogenicity of antigens incorporated into or co-administered with an adjuvant in question. In the context of the present invention an adjuvant will preferably enhance the specific immunogenic effect of the active agents of the present invention. Typically, “adjuvant” or “adjuvant component” has the same meaning and can be used mutually. Adjuvants may be divided, e.g., into immuno potentiators, antigenic delivery systems or even combinations thereof. The term “adjuvant” is typically understood not to comprise agents which confer immunity by themselves. An adjuvant assists the immune system unspecifically to enhance the antigen-specific immune response by e.g. promoting presentation of an antigen to the immune system or induction of an unspecific innate immune response. Furthermore, an adjuvant may preferably e.g. modulate the antigen-specific immune response by e.g. shifting the dominating Th2-based antigen specific response to a more Th1-based antigen specific response or vice versa and/or by inducing of mucosal immune responses and/or increased IgA titers. Accordingly, an adjuvant may favourably modulate cytokine expression/secretion, antigen presentation, type of immune response etc.

Advantages of adjuvants include the enhancement of the immunogenicity of antigens, modification of the nature of the immune response, the reduction of the antigen amount needed for a successful immunization, the reduction of the frequency of booster immunizations needed and an improved immune response in elderly and immunocompromised vaccinees. These may be co-administered by any route, e.g., intramusculary, subcutaneous, IV or intradermal injections.

Antigen: The term “antigen” refers typically to a substance which may be recognized by the immune system and may be capable of triggering an antigen-specific immune response, e.g. by formation of antibodies or antigen-specific T-cells as part of an adaptive immune response. An antigen may be a protein or peptide. In this context, the first step of an adaptive immune response is the activation of naïve antigen-specific T cells by antigen-presenting cells. This occurs in the lymphoid tissues and organs through which naïve T cells are constantly passing. The three cell types that can serve as antigen-presenting cells are dendritic cells, macrophages, and B cells. Each of these cells has a distinct function in eliciting immune responses. Tissue dendritic cells take up antigens by phagocytosis and macropinocytosis and are stimulated by infection to migrate to the local lymphoid tissue, where they differentiate into mature dendritic cells. Macrophages ingest particulate antigens such as bacteria and are induced by infectious agents to express MHC class II molecules. The unique ability of B cells to bind and internalize soluble protein antigens via their receptors may be important to induce T cells. By presenting the antigen on MHC molecules leads to activation of T cells which induces their proliferation and differentiation into armed effector T cells. The most important function of effector T cells is the killing of infected cells by CD8+ cytotoxic T cells and the activation of macrophages by Th1 cells which together make up cell-mediated immunity, and the activation of B cells by both Th2 and Th1 cells to produce different classes of antibody, thus driving the humoral immune response. T cells recognize an antigen by their T cell receptors which does not recognize and bind antigen directly, but instead recognize short peptide fragments e.g. of pathogens' protein antigens, which are bound to MHC molecules on the surfaces of other cells.

T cells: T cells fall into two major classes that have different effector functions. The two classes are distinguished by the expression of the cell-surface proteins CD4 and CD8. These two types of T cells differ in the class of MHC molecule that they recognize. There are two classes of MHC molecules—MHC class I and MHC class II molecules—which differ in their structure and expression pattern on tissues of the body. CD4+ T cells bind to a MHC class II molecule and CD8+ T cells to a MHC class I molecule. MHC class I and MHC class II molecules have distinct distributions among cells that reflect the different effector functions of the T cells that recognize them. MHC class I molecules present peptides of cytosolic and nuclear origin e.g. from pathogens, commonly viruses, to CD8+ T cells, which differentiate into cytotoxic T cells that are specialized to kill any cell that they specifically recognize. Almost all cells express MHC class I molecules, although the level of constitutive expression varies from one cell type to the next. But not only pathogenic peptides from viruses are presented by MHC class I molecules, also self-antigens like tumor antigens are presented by them. MHC class I molecules bind peptides from proteins degraded in the cytosol and transported in the endoplasmic reticulum. The CD8+ T cells that recognize MHC class I:peptide complexes at the surface of infected cells are specialized to kill any cells displaying foreign peptides and so rid the body of cells infected with viruses and other cytosolic pathogens. The main function of CD4+ T cells (CD4+ helper T cells) that recognize MHC class II molecules is to activate other effector cells of the immune system. Thus MHC class II molecules are normally found on B lymphocytes, dendritic cells, and macrophages, cells that participate in immune responses, but not on other tissue cells. Macrophages, for example, are activated to kill the intravesicular pathogens they harbour, and B cells to secrete immunoglobulins against foreign molecules. MHC class II molecules are prevented from binding to peptides in the endoplasmic reticulum and thus MHC class II molecules bind peptides from proteins which are degraded in endosomes. They can capture peptides from pathogens that have entered the vesicular system of macrophages, or from antigens internalized by immature dendritic cells or the immunoglobulin receptors of B cells. Pathogens that accumulate in large numbers inside macrophage and dendritic cell vesicles tend to stimulate the differentiation of Th1 cells, whereas extracellular antigens tend to stimulate the production of Th2 cells. Th1 cells activate the microbicidal properties of macrophages and induce B cells to make IgG antibodies that are very effective of opsonising extracellular pathogens for ingestion by phagocytic cells, whereas Th2 cells initiate the humoral response by activating naïve B cells to secrete IgM, and induce the production of weakly opsonising antibodies such as IgG1 and IgG3 (mouse) and IgG2 and IgG4 (human) as well as IgA and IgE (mouse and human).

Epitope (also called “antigen determinant”): T cell epitopes may comprise fragments preferably having a length of about 6 to about 20 or even more amino acids, e.g. fragments as processed and presented by MHC class I molecules, preferably having a length of about 8 to about 10 amino acids, e.g. 8, 9, or 10, (or even 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T cells in form of a complex consisting of the peptide fragment and an MHC molecule. B cell epitopes are typically fragments located on the outer surface of (native) protein or peptide antigens.

Vaccine: A vaccine is typically understood to be a prophylactic or therapeutic material providing at least one antigen or antigenic function. The antigen or antigenic function may stimulate the body's adaptive immune system to provide an adaptive immune response.

Antigen-providing mRNA: An antigen-providing mRNA may typically be an mRNA, having at least one open reading frame that can be translated by a cell or an organism provided with that mRNA. The product of this translation is a peptide or protein that may act as an antigen, preferably as an immunogen. The product may also be a fusion protein composed of more than one immunogen, e.g. a fusion protein that consist of two or more epitopes, peptides or proteins, wherein the epitopes, peptides or proteins may be linked by linker sequences.

5′-CAP-Structure: A 5′-CAP is typically a modified nucleotide, particularly a guanine nucleotide, added to the 5′ end of an mRNA molecule. Preferably, the 5′-CAP is added using a 5′-5′-triphosphate linkage (also named m7GpppN). Further examples of 5′-CAP structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-CAP structures may be used in the context of the present invention to modify the mRNA sequence of the inventive composition. Further modified 5′-CAP structures which may be used in the context of the present invention are CAP1 (methylation of the ribose of the adjacent nucleotide of m7GpppN), CAP2 (methylation of the ribose of the 2^{nd} nucleotide downstream of the m7GpppN), CAP3 (methylation of the ribose of the 3^{rd} nucleotide downstream of the m7GpppN), CAP4 (methylation of the ribose of the 4^{th} nucleotide downstream of the m7GpppN), ARCA (anti-reverse CAP analogue), modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

Fragments of proteins: “Fragments” of proteins or peptides in the context of the present invention may, typically, comprise a sequence of a protein or peptide as defined herein, which is, with regard to its amino acid sequence (or its encoded nucleic acid molecule), N-terminally and/or C-terminally truncated compared to the amino acid sequence of the original (native) protein (or its encoded nucleic acid molecule). Such truncation may thus occur either on the amino acid level or correspondingly on the nucleic acid level. A sequence identity with respect to such a fragment as defined herein may therefore preferably refer to the entire protein or peptide as defined herein or to the entire (coding) nucleic acid molecule of such a protein or peptide. For example such fragment may have a length of about 6 to about 20 or even more amino acids, e.g. fragments as processed and presented by MHC class I molecules, preferably having a length of about 8 to about 10 amino acids, e.g. 8, 9, or 10, (or even 6, 7, 11, or 12 amino acids), or fragments as processed and presented by MHC class II molecules, preferably having a length of about 13 or more amino acids, e.g. 13, 14, 15, 16, 17, 18, 19, 20 or even more amino acids, wherein these fragments may be selected from any part of the amino acid sequence. These fragments are typically recognized by T-cells in form of a complex consisting of the peptide fragment and an MHC molecule, i.e. the fragments are typically not recognized in their native form. Fragments of proteins or peptides may comprise at least one epitope of those proteins or peptides. Furthermore also domains of a protein, like the extracellular domain, the intracellular domain or the transmembrane domain and shortened or truncated versions of a protein may be understood to comprise a fragment of a protein.

Variants of proteins: “Variants” of proteins or peptides as defined in the context of the present invention may be generated, having an amino acid sequence which differs from the original sequence in one or more mutation(s), such as one or more substituted, inserted and/or deleted amino acid(s). Preferably, these fragments and/or variants have the same biological function or specific activity compared to the full-length native protein, e.g. its specific antigenic property. “Variants” of proteins or peptides as defined in the context of the present invention may comprise conservative amino acid substitution(s) compared to their native, i.e. non-mutated physiological, sequence. Those amino acid sequences as well as their encoding nucleotide sequences in particular fall under the term variants as defined herein. Substitutions in which amino acids, which originate from the same class, are exchanged for one another are called conservative substitutions. In particular, these are amino acids having aliphatic side chains, positively or negatively charged side chains, aromatic groups in the side chains or amino acids, the side chains of which can enter into hydrogen bridges, e.g. side chains which have a hydroxyl function. This means that e.g. an amino acid having a polar side chain is replaced by another amino acid having a likewise polar side chain, or, for example, an amino acid characterized by a hydrophobic side chain is substituted by another amino acid having a likewise hydrophobic side chain (e.g. serine (threonine) by threonine (serine) or leucine (isoleucine) by isoleucine (leucine)). Insertions and substitutions are possible, in particular, at those sequence positions which cause no modification to the three-dimensional structure or do not affect the binding region. Modifications to a three-dimensional structure by insertion(s) or deletion(s) can easily be determined e.g. using CD spectra (circular dichroism spectra) (Urry, 1985, Absorption, Circular Dichroism and ORD of Polypeptides, in: Modern Physical Methods in Biochemistry, Neuberger et al. (ed.), Elsevier, Amsterdam). A “variant” of a protein or peptide may have at least 70%, 75%, 80%, 85%, 90%, 95%, 98% or 99% amino acid identity over a stretch of 10, 20, 30, 50, 75 or 100 amino acids of such protein or peptide. Furthermore, variants of proteins or peptides as defined herein, which may be encoded by a nucleic acid molecule, may also comprise those sequences, wherein nucleotides of the encoding nucleic acid sequence are exchanged according to the degeneration of the genetic code, without leading to an alteration of the respective amino acid sequence of the protein or peptide, i.e. the amino acid sequence or at least part thereof may not differ from the original sequence within the above meaning.

Identity of a sequence: In order to determine the percentage to which two sequences are identical, e.g. nucleic acid sequences or amino acid sequences as defined herein, preferably the amino acid sequences encoded by a nucleic acid sequence of the polymeric carrier as defined herein or the amino acid sequences themselves, the sequences can be aligned in order to be subsequently compared to one another. Therefore, e.g. a position of a first sequence may be compared with the corresponding position of the second sequence. If a position in the first sequence is occupied by the same component (residue) as is the case at a position in the second sequence, the two sequences are identical at this position. If this is not the case, the sequences differ at this position. If insertions occur in the second sequence in comparison to the first sequence, gaps can be inserted into the first sequence to allow a further alignment. If deletions occur in the second sequence in comparison to the first sequence, gaps can be inserted into the second sequence to allow a further alignment. The percentage to which two sequences are identical is then a function of the number of identical positions divided by the total number of positions including those positions which are only occupied in one sequence. The percentage to which two sequences are identical can be determined using a mathematical algorithm. A preferred, but not limiting, example of a mathematical algorithm which can be used is the algorithm of Karlin et al. (1993), PNAS USA, 90:5873-5877 or Altschul et al. (1997), Nucleic Acids Res., 25:3389-3402. Such an algorithm is integrated in the BLAST program. Sequences which are identical to the sequences of the present invention to a certain extent can be identified by this program.

Derivative of a protein or peptide: A derivative of a peptide or protein is typically understood to be a molecule that is derived from another molecule, such as said peptide or protein. A “derivative” of a peptide or protein also encompasses fusions comprising a peptide or protein used in the present invention. For example, the fusion comprises a label, such as, for example, an epitope, e.g., a FLAG epitope or a V5 epitope. For example, the epitope is a FLAG epitope. Such a tag is useful for, for example, purifying the fusion protein.

Nucleic acid: The term nucleic acid means any DNA or RNA molecule and is used synonymous with polynucleotide. Wherever herein reference is made to a nucleic acid or nucleic acid sequence encoding a particular protein and/or peptide, said nucleic acid or nucleic acid sequence, respectively, preferably also comprises regulatory sequences allowing in a suitable host, e.g. a human being, its expression, i.e. transcription and/or translation of the nucleic acid sequence encoding the particular protein or peptide.

Peptide: A peptide is a polymer of amino acid monomers. Usually the monomers are linked by peptide bonds. The term “peptide” does not limit the length of the polymer chain of amino acids. In some embodiments of the present invention a peptide may for example contain less than 50 monomer units. Longer peptides are also called polypeptides, typically having 50 to 600 monomeric units, more specifically 50 to 300 monomeric units.

Pharmaceutically effective amount: A pharmaceutically effective amount in the context of the invention is typically understood to be an amount that is sufficient to induce an immune response.

Protein: A protein typically consists of one or more peptides and/or polypeptides folded into 3-dimensional form, facilitating a biological function.

Poly(C) sequence: A poly(C) sequence is typically a long sequence of cytosine nucleotides, typically about 10 to about 200 cytosine nucleotides, preferably about 10 to about 100 cytosine nucleotides, more preferably about 10 to about 70 cytosine nucleotides or even more, preferably about 20 to about 50, or even about 20 to about 30 cytosine nucleotides. A poly(C) sequence may preferably be located 3′ of the coding region comprised by a nucleic acid.

Poly(A) tail: A poly(A) tail also called “3′-poly(A) tail” is typically a long sequence of adenosine nucleotides of up to about 400 adenosine nucleotides, e.g. from about 25 to about 400, preferably from about 50 to about 400, more preferably from about 50 to about 300, even more preferably from about 50 to about 250, most preferably from about 60 to about 250 adenosine nucleotides, added to the 3′ end of a RNA.

Stabilized nucleic acid: A stabilized nucleic acid, typically, exhibits a modification increasing resistance to in vivo degradation (e.g. degradation by an exo- or endo-nuclease) and/or ex vivo degradation (e.g. by the manufacturing process prior to vaccine administration, e.g. in the course of the preparation of the vaccine solution to be administered). Stabilization of RNA can, e.g., be achieved by providing a 5′-CAP-Structure, a poly(A) tail, or any other UTR-modification. It can also be achieved by backbone-modification or modification of the G/C-content of the nucleic acid. Various other methods are known in the art and conceivable in the context of the invention.

Carrier/polymeric carrier: A carrier in the context of the invention may typically be a compound that facilitates transport and/or complexation of another compound. Said carrier may form a complex with said other compound. A polymeric carrier is a carrier that is formed of a polymer.

Cationic component: The term “cationic component” typically refers to a charged molecule, which is positively charged (cation) at a pH value of typically about 1 to 9, preferably of a pH value of or below 9 (e.g. 5 to 9), of or below 8 (e.g. 5 to 8), of or below 7 (e.g. 5 to 7), most preferably at physiological pH values, e.g. about 7.3 to 7.4. Accordingly, a cationic peptide, protein or polymer according to the present invention is positively charged under physiological conditions, particularly under physiological salt conditions of the cell in vivo. A cationic peptide or protein preferably contains a larger number of cationic amino acids, e.g. a larger number of Arg, His, Lys or Orn than other amino acid residues (in particular more cationic amino acids than anionic amino acid residues like Asp or Glu) or contains blocks predominantly formed by cationic amino acid residues. The definition “cationic” may also refer to “polycationic” components.

3′-untranslated region (3′-UTR): A 3′-UTR is typically the part of an mRNA which is located between the protein coding region (i.e. the open reading frame) and the poly(A) sequence of the mRNA. A 3′-UTR of the mRNA is not translated into an amino acid sequence. The 3′-UTR sequence is generally encoded by the gene which is transcribed into the respective mRNA during the gene expression process. The genomic sequence is first transcribed into pre-mature mRNA, which comprises optional introns. The pre-mature mRNA is then further processed into mature mRNA in a maturation process. This maturation process comprises the steps of 5′-capping, splicing the pre-mature mRNA to excise optional introns and modifications of the 3′-end, such as polyadenylation of the 3′-end of the pre-mature mRNA and optional endo- or exonuclease cleavages etc. In the context of the present invention, a 3′-UTR corresponds to the sequence of a mature mRNA which is located 3′ to the stop codon of the protein coding region, preferably immediately 3′ to the stop codon of the protein coding region, and which extends to the 5′-side of the poly(A) sequence, preferably to the nucleotide immediately 5′ to the poly(A) sequence. The term “corresponds to” means that the 3′-UTR sequence may be an RNA sequence, such as in the mRNA sequence used for defining the 3′-UTR sequence, or a DNA sequence which corresponds to such RNA sequence. In the context of the present invention, the term “a 3′-UTR of a gene”, such as “a 3′-UTR of an albumin gene”, is the sequence which corresponds to the 3′-UTR of the mature mRNA derived from this gene, i.e. the mRNA obtained by transcription of the gene and maturation of the pre-mature mRNA. The term “3′-UTR of a gene” encompasses the DNA sequence and the RNA sequence of the 3′-UTR.

5′-untranslated region (5′-UTR): A 5′-UTR is typically understood to be a particular section of messenger RNA (mRNA). It is located 5′ of the open reading frame of the mRNA. Typically, the 5′-UTR starts with the transcriptional start site and ends one nucleotide before the start codon of the open reading frame. The 5′-UTR may comprise elements for controlling gene expression, also called regulatory elements. Such regulatory elements may be, for example, ribosomal binding sites or a 5′-Terminal Oligopyrimidine Tract. The 5′-UTR may be posttranscriptionally modified, for example by addition of a 5′-CAP. In the context of the present invention, a 5′UTR corresponds to the sequence of a mature mRNA which is located between the 5′-CAP and the start codon. Preferably, the 5′-UTR corresponds to the sequence which extends from a nucleotide located 3′ to the 5′-CAP, preferably from the nucleotide located immediately 3′ to the 5′-CAP, to a nucleotide located 5′ to the start codon of the protein coding region, preferably to the nucleotide located immediately 5′ to the start codon of the protein coding region. The nucleotide located immediately 3′ to the 5′-CAP of a mature mRNA typically corresponds to the transcriptional start site. The term “corresponds to” means that the 5′-UTR sequence may be an RNA sequence, such as in the mRNA sequence used for defining the 5′-UTR sequence, or a DNA sequence which corresponds to such RNA sequence. In the context of the present invention, the term “a 5′-UTR of a gene”, such as “a 5′-UTR of a TOP gene”, is the sequence which corresponds to the 5′-UTR of the mature mRNA derived from this gene, i.e. the mRNA obtained by transcription of the gene and maturation of the pre-mature mRNA. The term “5′-UTR of a gene” encompasses the DNA sequence and the RNA sequence of the 5′-UTR.

5′ Terminal Oliqopyrimidine Tract (TOP): The 5′ terminal oligopyrimidine tract (TOP) is typically a stretch of pyrimidine nucleotides located at the 5′ terminal region of a nucleic acid molecule, such as the 5′ terminal region of certain mRNA molecules or the 5′ terminal region of a functional entity, e.g. the transcribed region, of certain genes. The sequence starts with a cytidine, which usually corresponds to the transcriptional start site, and is followed by a stretch of usually about 3 to 30 pyrimidine nucleotides. For example, the TOP may comprise 3, 4, 5, 6, 7, 8, 9, 10, 11, 12, 13, 14, 15, 16, 17, 18, 19, 20, 21, 22, 23, 24, 25, 26, 27, 28, 29, 30 or even more nucleotides. The pyrimidine stretch and thus the 5′ TOP ends one nucleotide 5′ to the first purine nucleotide located downstream of the TOP. mRNA that contains a 5′ terminal oligopyrimidine tract is often referred to as TOP mRNA. Accordingly, genes that provide such messenger RNAs are referred to as TOP genes. TOP sequences have, for example, been found in genes and mRNAs encoding peptide elongation factors and ribosomal proteins.

TOP motif: In the context of the present invention, a TOP motif is a nucleic acid sequence which corresponds to a 5′ TOP as defined above. Thus, a TOP motif in the context of the present invention is preferably a stretch of pyrimidine nucleotides having a length of 3-30 nucleotides. Preferably, the TOP-motif consists of at least 3 pyrimidine nucleotides, preferably at least 4 pyrimidine nucleotides, preferably at least 5 pyrimidine nucleotides, more preferably at least 6 nucleotides, more preferably at least 7 nucleotides, most preferably at least 8 pyrimidine nucleotides, wherein the stretch of pyrimidine nucleotides preferably starts at its 5′ end with a cytosine nucleotide. In TOP genes and TOP mRNAs, the TOP-motif preferably starts at its 5′ end with the transcriptional start site and ends one nucleotide 5′ to the first purine residue in said gene or mRNA. A TOP motif in the sense of the present invention is preferably located at the 5′end of a sequence which represents a 5′-UTR or at the 5′ end of a sequence which codes for a 5′-UTR. Thus, preferably, a stretch of 3 or more pyrimidine nucleotides is called “TOP motif” in the sense of the present invention if this stretch is located at the 5′ end of a respective sequence, such as the mRNA, the 5′-UTR element of the mRNA, or the nucleic acid sequence which is derived from the 5′-UTR of a TOP gene as described herein. In other words, a stretch of 3 or more pyrimidine nucleotides which is not located at the 5′-end of a 5′-UTR or a 5′-UTR element but anywhere within a 5′-UTR or a 5′-UTR element is preferably not referred to as “TOP motif”.

TOP gene: TOP genes are typically characterised by the presence of a 5′ terminal oligopyrimidine tract. Furthermore, most TOP genes are characterized by a growth-associated translational regulation. However, also TOP genes with a tissue specific translational regulation are known. As defined above, the 5′-UTR of a TOP gene corresponds to the sequence of a 5′-UTR of a mature mRNA derived from a TOP gene, which preferably extends from the nucleotide located 3′ to the 5′-CAP to the nucleotide located 5′ to the start codon. A 5′-UTR of a TOP gene typically does not comprise any start codons, preferably no upstream AUGs (uAUGs) or upstream open reading frames (uORFs). Therein, upstream AUGs and upstream open reading frames are typically understood to be AUGs and open reading frames that occur 5′ of the start codon (AUG) of the open reading frame that should be translated. The 5′-UTRs of TOP genes are generally rather short. The lengths of 5′-UTRs of TOP genes may vary between 20 nucleotides up to 500 nucleotides, and are typically less than about 200 nucleotides, preferably less than about 150 nucleotides, more preferably less than about 100 nucleotides. Exemplary 5′-UTRs of TOP genes in the sense of the present invention are the nucleic acid sequences extending from the nucleotide at position 5 to the nucleotide located immediately 5′ to the start codon (e.g. the ATG) in the sequences according to SEQ ID Nos. 1-1363, SEQ ID NO. 1395, SEQ ID NO. 1421 and SEQ ID NO. 1422 of the international patent application WO2013/143700 or homologs or variants thereof, whose disclosure is incorporated herewith by reference. In this context a particularly preferred fragment of a 5′UTR of a TOP gene is a 5′-UTR of a TOP gene lacking the 5′ TOP motif. The term ‘5′UTR of a TOP gene’ preferably refers to the 5′-UTR of a naturally occurring TOP gene.

Fragment of a nucleic acid sequence, particularly an mRNA: A fragment of a nucleic acid sequence consists of a continuous stretch of nucleotides corresponding to a continuous stretch of nucleotides in the full-length nucleic acid sequence which is the basis for the nucleic acid sequence of the fragment, which represents at least 20%, preferably at least 30%, more preferably at least 40%, more preferably at least 50%, even more preferably at least 60%, even more preferably at least 70%, even more preferably at least 80%, and most preferably at least 90% of the full-length nucleic acid sequence. Such a fragment, in the sense of the present invention, is preferably a functional fragment of the full-length nucleic acid sequence.

Variant of a nucleic acid sequence, particularly an mRNA: A variant of a nucleic acid sequence refers to a variant of nucleic acid sequences which forms the basis of a nucleic acid sequence. For example, a variant nucleic acid sequence may exhibit one or more nucleotide deletions, insertions, additions and/or substitutions compared to the nucleic acid sequence from which the variant is derived. Preferably, a variant of a nucleic acid sequence is at least 40%, preferably at least 50%, more preferably at least 60%, more preferably at least 70%, even more preferably at least 80%, even more preferably at least 90%, most preferably at least 95% identical to the nucleic acid sequence the variant is derived from. Preferably, the variant is a functional variant. A “variant” of a nucleic acid sequence may have at least 70%, 75%, 80%, 85%, 90%, 95%, 98% or 99% nucleotide identity over a stretch of 10, 20, 30, 50, 75 or 100 nucleotide of such nucleic acid sequence.

The present invention provides a combination of a first component and a second component, wherein the first component is typically an immunogenic component, preferably as described herein, and wherein the second component is typically an adjuvant component, preferably as defined herein.

Preferably, the first (immunogenic) component as described herein comprises at least one nucleic acid molecule encoding at least one antigen, or a fragment or variant thereof. More preferably, the first (immunogenic) component comprises at least one nucleic acid molecule encoding at least one epitope derived from an antigen as described herein, or a fragment or variant thereof.

According to the invention, it is further preferred that the second (adjuvant) component of the combination according to the invention comprises at least one adjuvant compound, wherein the at least one adjuvant compound is preferably an immune potentiator compound as described herein and/or a delivery system compound as described herein. More preferably, the second (adjuvant) component of the combination according to the invention comprises at least one immune potentiator compound, preferably as described herein, and/or at least one delivery system compound, preferably as described herein. Even more preferably, the second (adjuvant) component comprises an adjuvant compound as described herein, which enhances the immune response against an antigen. Most preferably, the second (adjuvant) component comprises an adjuvant compound that unspecifically enhances the immune response against an antigen, preferably as described herein. In certain embodiments, the immune potentiator compound may also function as a delivery system compound, preferably as described herein.

According to a preferred embodiment, the second (adjuvant) component of the combination according to the invention comprises—in addition or alternatively to the at least one immune potentiator compound described herein—at least one delivery system compound, preferably as described herein. In the context of the present invention, a delivery system compound is preferably a compound enhancing delivery of the first (immunogenic) component, preferably the at least one nucleic acid molecule encoding an antigen or an epitope derived from an antigen, to the immune system of a subject upon administration of the combination according to the invention to the subject. More preferably, the delivery system compound enhances delivery of the first (immunogenic) component, preferably the at least one nucleic acid molecule encoding an antigen or an epitope derived from an antigen, to antigen presenting cells of a subject. In certain embodiments, the delivery system compound may also function as an immune potentiator compound, preferably as described herein.

The inventors surprisingly found that the combination of a first (immunogenic) component and a second (adjuvant) component as described herein is suitable for enhancing the immune response against an antigen. In particular, it was found that the immunogenicity of a first (immunogenic) component can be significantly increased by combined administration to a subject of the first (immunogenic) component with a second (adjuvant) component, preferably as defined herein. In this context, it is noted that the combination according to the invention may be provided in the form of one formulation/composition. Alternatively, the combination of the present invention may comprise separate formulations, which are preferably administered to a subject concurrently or in a time-staggered manner, preferably as described herein. The disclosure provided herein relating to the “combination” according to the invention (and uses thereof) preferably applies to the “composition” according to the invention (and uses thereof) and vice versa.

In embodiments, where the first (immunogenic) component and the second (adjuvant) component of the combination according to the invention are not comprised in the same composition, but formulated separately, it is preferred that the first (immunogenic) component and the second (adjuvant) component are administered to a subject concurrently, wherein the term ‘concurrently’ as used in this context preferably comprises two events that take place within the same 5 minutes. Alternatively, the second (adjuvant) component of the combination according to the invention, if provided as a separate formulation, is preferably administered to a subject within 24 hours, more preferably within 23, 22, 21, 20, 19, 18, 17, 16, 15, 14, 13, 12, 11, 10, 9, 8, 7, 6, 5, 4, 3 or 2 hours, after the administration to the same subject of the first (immunogenic) component of the combination according to the invention, or vice versa. According to a preferred embodiment, the second (adjuvant) component of the combination according to the invention, if provided as separate formulation, is preferably administered to a subject within 60 minutes, more preferably within 50, 40, 30, 20, 10 or 5 minutes, most preferably within 10 minutes, after administration to the same subject of the first (immunogenic) component, or vice versa.

The composition according to the invention comprises at least a first immunogenic component and at least a second adjuvant component. The first immunogenic component comprises at least one nucleic acid molecule encoding at least one epitope of at least one antigen. The second adjuvant component comprises at least one immune potentiator compound and/or at least one delivery system compound. An immune potentiator in the sense of the present invention is a component which more or less directly engages with the immune system thereby increasing and/or modulating responses to antigens. An immune potentiator compound may also be defined as a more or less general immunostimulant. A delivery system compound in the sense of the present invention is a component which presents vaccine antigens to the immune system in an optimized manner thereby increasing and/or modulating the immune response to the antigen. A delivery system compound may also provide controlled release and/or depot delivery of the antigen respectively the nucleic acid molecule encoding the antigen.

The second (adjuvant) component typically comprises at least one adjuvant compound, preferably an immune potentiator compound and/or a delivery system compound, wherein the at least one adjuvant compound, preferably the immune potentiator compound or the delivery system compound, is preferably selected from the group consisting of vitamin compounds, polymeric carrier cargo complexes, preferably as described herein, emulsion- or surfactant-based compounds, nucleotide- or nucleoside based compounds, protein- or peptide-based compounds, hydrocarbon-based or carbohydrate-based compounds, lipid-based compounds, polymeric compounds, preferably polymeric microparticle compounds, cytokine or hormone compounds, toxin compounds, vehicle compounds, mineral salt compounds, immune stimulating complexes (ISCOM), and virus-like particles.

According to a preferred embodiment, the second (adjuvant) component comprises at least one adjuvant compound, preferably an immune potentiator compound and/or a delivery system compound, wherein the at least one adjuvant compound, preferably the immune potentiator compound or the delivery system compound, is a mineral salt compound. In the context of the present invention, a mineral salt compound is preferably an aluminium compound or a calcium compound. More preferably, the mineral salt compound as used herein is an aluminium phosphate compound or a calcium phosphate compound, more preferably an aluminium phosphate salt or a calcium phosphate salt, most preferably an aluminium phosphate salt, such as Adju-Phos. In a particularly preferred embodiment, the second (adjuvant) component comprises at least one adjuvant compound, preferably an immune potentiator compound and/or a delivery system compound, wherein the at least one adjuvant compound, preferably the immune potentiator compound or the delivery system compound, is an aluminium compound, more preferably an aluminium phosphate compound or an aluminium hydroxide compound, even more preferably an aluminium phosphate compound, most preferably an aluminium phosphate salt.

Preferably the second adjuvant component of the inventive composition comprises at least one vitamin compound as immune potentiator compound. In a preferred embodiment the vitamin compound is a vitamin A compound and/or vitamin A derivative compound, preferably a retinoid compound. It has been shown by the inventors that a composition comprising these compounds is particularly effective when used for vaccination. Surprisingly a vitamin compound, especially a vitamin A or vitamin A derivative compound, used as adjuvant is able to effectively improve the immunogenicity of the first immunogenic component of the inventive composition. It was surprisingly found that such a vitamin compound resulted in increased antigen-specific IgA titers. By administration of the inventive composition it is possible to increase and/or modulate the immune response which is provoked by the immunogenic compound. The inventive composition may be used generally to provide improved vaccines, especially mRNA based vaccines, for a broad range of indications.

In a preferred embodiment of the inventive composition the vitamin compound is selected from the list consisting of: retinoic acid, preferably all-trans retinoic acid (ATRA), retinyl palmitate, retinol ester, retinol, retinal, tretinoin, Retin-A, isotretinoin, alitretinoin, etretinate, acitretin, tazarotene, bexarotene and Adapalene (polyaromatic retinoid). Especially preferred is all-trans retinoic acid. Moreover it is especially preferred to use depot variants of vitamin A or vitamin A derivatives.

Especially the use of a vitamin A compound or a vitamin A derivative compound for the adjuvant component of the inventive composition allows enhancement of the generation of mucosal immune responses even after non-mucosal immunization. The administration of a vitamin compound in combination with an mRNA based vaccine is able to modulate the peripheral lymphoid tissues to allow the efficient generation of mucosal immune responses after non-mucosal immunization (e.g. intradermal, intramuscular, or subcutaneous immunization) with mRNA based vaccines. All-trans retinoic acid (ATRA) is particularly preferred for the purposes of the invention. ATRA is the major metabolic derivative of vitamin A and has already a proven record of safety in the clinical treatment of e.g. acne (Berger R. et al. (2007), Clin Ther 29: 1086-1097).

It has been described by Hammerschmidt S. I. et al that the application of vitamin A or vitamin A derivatives like retinoic acid has been used to induce mucosal immunity in combination with protein antigens (J Clin Invest (2011) August, 121(8): 3051-61), wherein retinoic acid induces homing of protective T and B cells to the gut after subcutaneous immunization in mice. Tan X. et al. described that retinoic acid as a vaccine adjuvant enhances CD8+ T cell response and mucosal protection from viral challenge (J Virol (2011) August, 85(16): 8316-27). Now it has been shown by the inventors that a vitamin compound and especially a vitamin A compound or a vitamin A derivative compound is particularly effective as adjuvant in combination with an mRNA based vaccine.

The first immunogenic component and the second adjuvant component, preferably comprising the vitamin compound, may be administered in different ways. For example it is possible to combine an intramuscular vaccination with an mRNA based vaccine (first immunogenic component of the composition) and a subcutaneous application of the second adjuvant component, especially the vitamin compound.

In a further preferred embodiment of the inventive composition the vitamin compound is a vitamin E compound and/or a vitamin C compound and/or a vitamin D compound, preferably selected from the list consisting of: tocopherol, mixture of Squalene plus Tween 80 plus α-tocopherol (AS03), vitamin D3, and 25-dihydroxycholecalciferol (Calcitrol), wherein these vitamin compounds may be administered as single adjuvant component or, especially preferred, they may be combined with a vitamin A or vitamin A derivative or with other adjuvant compounds.

It is preferred to combine the vitamin compound with a further adjuvant component. It is especially preferred to combine the vitamin compound with a further adjuvant component comprising a polymeric carrier cargo complex. The polymeric carrier cargo complex comprises as a carrier a complex of at least one cationic and/or oligocationic and/or polycationic component and as a cargo at least one nucleic acid molecule. Preferably the cationic and/or oligocationic and/or polycationic component is at least one disulfide-crosslinked cationic component. By the combination of these adjuvant components it is possible to enhance the immunostimulatory effect of the inventive composition. In a preferred embodiment of the polymeric carrier cargo complex the cationic and/or oligocationic and/or polycationic component comprises cationic peptides, wherein preferably the cationic peptides are selected from peptides according to formula (I)

(Arg)ₗ;(Lys)ₘ;(His)ₙ;(Orn)ₒ;(Xaa)ₓ,

wherein
l+m+n+o+x=3-100, and
l, m, n or o=independently of each other is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, 81-90 and 91-100, provided that the overall content of Arg, Lys, His and Orn represents at least 10% of all amino acids of the cationic peptide; and Xaa is any amino acid selected from native (=naturally occurring) or non-native amino acids except of Arg, Lys, His or Orn; and
x=any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, 81-90, provided, that the overall content of Xaa does not exceed 90% of all amino acids of the cationic peptide,
or are selected from peptides according to subformula (Ia)

{(Arg)ₗ;(Lys)ₘ;(His)ₙ;(Orn)ₒ;(Xaa′)ₓ(Cys)_{y}}
or from peptides according to subformula (Ib)

Cys₁{(Arg)ₗ;(Lys)ₘ;(His)ₙ;(Orn)ₒ;(Xaa)ₓ}Cys₂
wherein (Arg)ₗ; (Lys)ₘ; (His)ₙ; (Orn)ₙ; and x are as defined above; Xaa′ is any amino acid selected from native (=naturally occurring) or non-native amino acids except of Arg, Lys, His, Orn; or Cys and y is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, and 81-90, provided that the overall content of Arg (Arginine), Lys (Lysine), His (Histidine) and Orn (Ornithine) represents at least 10% of all amino acids of the oligopeptide and wherein Cys₁ and Cys₂ are Cysteines proximal to, or terminal to (Arg)ₗ;(Lys)ₘ;(His)ₙ;(Orn)ₒ;(Xaa)ₓ.)

The disulfide-bonds of the cationic and/or oligocationic and/or polycationic component are preferably formed by cysteine residues contained in the cationic peptides. In preferred embodiments the cysteine residue is located proximal to the terminal ends of the cationic peptides, preferably at both of the terminal ends or at only one of the terminal ends of the cationic peptides. Preferably the cationic and/or oligocationic and/or polycationic component comprises an arginine-rich peptide, preferably the peptide CysArg₁₂Cys according to SEQ ID NO. 7 and/or the peptide CysArg₁₂ according to SEQ ID NO. 8.

The nucleic acid molecule of the polymeric carrier cargo complex is preferably an RNA molecule, preferably a guanosine-rich and uracil-rich RNA molecule. In preferred embodiments the cargo nucleic acid molecule is an immunostimulatory nucleic acid molecule, preferably an immunostimulatory RNA molecule (isRNA), more preferably a non-coding immunostimulatory nucleic acid molecule, more preferably a nucleic acid molecule according to SEQ ID NO. 2 (see also FIG. 2). The nitrogen/phosphate ratio (N/P ratio) of the polymeric carrier cargo complex may be adjusted to a certain value thereby increasing the efficiency of this adjuvant component. Preferably the cationic and/or oligocationic and/or polycationic component of the polymeric carrier and the cargo nucleic acid molecule comprised in said polymeric carrier cargo complex are provided in a N/P ratio in the range of 0.1-20, or in the range of 0.1-5, or in the range of 0.1-1, or in the range of 0.5-0.9.

An immunostimulatory RNA (isRNA) in the context of the invention may typically be a RNA that is able to induce an innate immune response itself. It usually does not have an open reading frame and thus does not provide a peptide-antigen or immunogen but elicits an innate immune response e.g. by binding to a specific kind of Toll-like-receptor (TLR) or other suitable receptors. However, of course also mRNAs having an open reading frame and coding for a peptide/protein (e.g. an antigenic function) may induce an innate immune response.

Further details of the polymeric carrier cargo complex will be apparent from the following explanations. In this context the cationic components, which form the basis for the polymeric carrier of the polymeric carrier cargo complex preferably by disulfide-crosslinkage, are typically selected from any suitable cationic or oligocationic or polycationic peptide, protein or polymer suitable for this purpose, particular any cationic or oligocationic or polycationic peptide, protein or polymer capable to complex a nucleic acid as defined according to the present invention, and thereby preferably condensing the nucleic acid. The cationic or oligocationic or polycationic peptide, protein or polymer, is preferably a linear molecule, however, branched cationic or oligocationic or polycationic peptides, proteins or polymers may also be used.

Each cationic or polycationic protein, peptide or polymer of the polymeric carrier contains preferably at least one —SH moiety, most preferably at least one cysteine residue or any further chemical group exhibiting an —SH moiety, capable to form a disulfide linkage upon condensation with at least one further cationic or oligocationic or polycationic protein, peptide or polymer as cationic component of the polymeric carrier as mentioned herein.

Each cationic or oligocationic or polycationic protein, peptide or polymer or any further component of the polymeric carrier is preferably linked to its neighbouring component(s) (cationic proteins, peptides, polymers or other components) via disulfide-crosslinking. Preferably, the disulfide-crosslinking is a reversible disulfide bond (—S—S—) between at least one cationic or polycationic protein, peptide or polymer and at least one further cationic or polycationic protein, peptide or polymer or other component of the polymeric carrier. The disulfide-crosslinking is typically formed by condensation of —SH-moieties of the components of the polymeric carrier particularly of the cationic components. Such an —SH-moiety may be part of the structure of the cationic or polycationic protein, peptide or polymer or any further component of the polymeric carrier prior to disulfide-crosslinking or may be added prior to disulfide-crosslinking by a modification as defined below. In this context, the sulphurs adjacent to one component of the polymeric carrier, necessary for providing a disulfide bond, may be provided by the component itself, e.g. by a —SH moiety as defined herein or may be provided by modifying the component accordingly to exhibit a —SH moiety. These —SH-moieties are typically provided by each of the component, e.g. via a cysteine or any further (modified) amino acid or compound of the component, which carries a —SH moiety. In the case that the cationic component or any further component of the polymeric carrier is a peptide or protein it is preferred that the —SH moiety is provided by at least one cysteine residue. Alternatively, the component of the polymeric carrier may be modified accordingly with a —SH moiety, preferably via a chemical reaction with a compound carrying a —SH moiety, such that each of the components of the polymeric carrier carries at least one such —SH moiety. Such a compound carrying a —SH moiety may be e.g. an (additional) cysteine or any further (modified) amino acid or compound of the component of the polymeric carrier, which carries a —SH moiety. Such a compound may also be any non-amino compound or moiety, which contains or allows to introduce a —SH moiety into the component as defined herein. Such non-amino compounds may be attached to the component of the polymeric carrier according to the present invention via chemical reactions or binding of compounds, e.g. by binding of a 3-thio propionic acid or 2-iminothiolane (Traut's reagent), by amide formation (e.g. carboxylic acids, sulphonic acids, amines, etc.), by Michael addition (e.g maleinimide moieties, α,β unsatured carbonyls, etc.), by click chemistry (e.g. azides or alkines), by alkene/alkine methatesis (e.g. alkenes or alkines), imine or hydrozone formation (aldehydes or ketons, hydrazins, hydroxylamins, amines), complexation reactions (avidin, biotin, protein G) or components which allow Sₙ-type substitution reactions (e.g halogenalkans, thiols, alcohols, amines, hydrazines, hydrazides, sulphonic acid esters, oxyphosphonium salts) or other chemical moieties which can be utilized in the attachment of further components. In some cases the —SH moiety may be masked by protecting groups during chemical attachment to the component. Such protecting groups are known in the art and may be removed after chemical coupling. In each case, the —SH moiety, e.g. of a cysteine or of any further (modified) amino acid or compound, may be present at the terminal ends or internally at any position of the component of the polymeric carrier. As defined herein, each of the components of the polymeric carrier typically exhibits at least one —SH— moiety, but may also contain two, three, four, five, or even more —SH-moieties. Additionally to binding of cationic components a —SH moiety may be used to attach further components of the polymeric carrier as defined herein, particularly an amino acid component, e.g. antigen epitopes, antigens, antibodies, cell penetrating peptides (e.g. TAT), ligands, etc.

According to one first alternative, at least one cationic (or oligocationic or polycationic) component of the polymeric carrier may be selected from cationic or oligocationic or polycationic peptides or proteins. Such peptides or proteins preferably exhibit a length of about 3 to 100 amino acids, preferably a length of about 3 to 50 amino acids, more preferably a length of about 3 to 25 amino acids, e.g. a length of about 3 to 10; 5 to 20; 5 to 15; 8 to 15, 16 or 17; 10 to 15, 16, 17, 18, 19, or 20; or 15 to 25 amino acids. Alternatively or additionally, such peptides or proteins may exhibit a molecular weight of about 0.1 kDa to about 100 kDa, including a molecular weight of about 0.5 kDa to about 100 kDa, preferably of about 10 kDa to about 50 kDa, even more preferably of about 10 kDa to about 30 kDa.

In the specific case that the cationic component of the polymeric carrier comprises a cationic or oligocationic or polycationic peptide or protein, the cationic properties of the peptide or protein or of the entire polymeric carrier, if the polymeric carrier is entirely composed of cationic or oligocationic or polycationic peptides or proteins, may be determined upon its content of cationic amino acids. Preferably, the content of cationic amino acids in the cationic or oligocationic or polycationic peptide or protein and/or the polymeric carrier is at least 10%, 20%, or 30%, preferably at least 40%, more preferably at least 50%, 60% or 70%, but also preferably at least 80%, 90%, or even 95%, 96%, 97%, 98%, 99% or 100%, most preferably at least 30%, 40%, 50%, 60%, 70%, 80%, 90%, 95%, 96%, 97%, 98%, 99% or 100%, or may be in the range of about 10% to 90%, more preferably in the range of about 15% to 75%, even more preferably in the range of about 20% to 50%, e.g. 20, 30, 40 or 50%, or in a range formed by any two of the afore mentioned values, provided, that the content of all amino acids, e.g. cationic, lipophilic, hydrophilic, aromatic and further amino acids, in the cationic or oligocationic or polycationic peptide or protein, or in the entire polymeric carrier, if the polymeric carrier is entirely composed of cationic or oligocationic or polycationic peptides or proteins, is 100%.

In this context, cationic amino acids are preferably the naturally occurring amino acids Arg (Arginine), Lys (Lysine), His (Histidine), and Orn (Ornithin). However, in a broader sense any non-natural amino acid carrying a cationic charge on its side chain may also be envisaged to carry out the invention. Preferably, however, are those cationic amino acids, the side chains of which are positively charged under physiological pH conditions. In a more preferred embodiment, these amino acids are Arg, Lys, and Orn.

Preferably, such cationic or oligocationic or polycationic peptides or proteins of the polymeric carrier, which comprise or are additionally modified to comprise at least one —SH moeity, are selected from, without being restricted thereto, cationic peptides or proteins such as protamine, nucleoline, spermine or spermidine, oligo- or poly-L-lysine (PLL), basic polypeptides, oligo or poly-arginine, cell penetrating peptides (CPPs), chimeric CPPs, such as Transportan, or MPG peptides, HIV-binding peptides, Tat, HIV-1 Tat (HIV), Tat-derived peptides, members of the penetratin family, e.g. Penetratin, Antennapedia-derived peptides (particularly from Drosophila antennapedia), pAntp, pIsl, etc., antimicrobial-derived CPPs e.g. Buforin-2, Bac715-24, SynB, SynB(1), pVEC, hCT-derived peptides, SAP, MAP, KALA, PpTG20, Loligomere, FGF, Lactoferrin, histones, VP22 derived or analog peptides, HSV, VP22 (Herpes simplex), MAP, KALA or protein transduction domains (PTDs), PpT620, prolin-rich peptides, arginine-rich peptides, lysine-rich peptides, Pep-1, L-oligomers, Calcitonin peptide(s), etc.

Alternatively or additionally, such cationic or oligocationic or polycationic peptides or proteins of the polymeric carrier, which comprise or are additionally modified to comprise at least one —SH moeity, are selected from, without being restricted thereto, following cationic peptides having the following sum formula (I):

{(Arg)ₗ;(Lys)ₘ;(His)ₙ;(Orn)ₒ;(Xaa)ₓ};

wherein l+m+n+o+x=3-100, and l, m, n or o independently of each other is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, 81-90 and 91-100 provided that the overall content of Arg (Arginine), Lys (Lysine), His (Histidine) and Orn (Ornithine) represents at least 10% of all amino acids of the oligopeptide; and Xaa is any amino acid selected from native (=naturally occurring) or non-native amino acids except of Arg, Lys, His or Orn; and x is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, 81-90, provided, that the overall content of Xaa does not exceed 90% of all amino acids of the oligopeptide. Any of amino acids Arg, Lys, His, Orn and Xaa may be positioned at any place of the peptide. In this context cationic peptides or proteins in the range of 7-30 amino acids are particular preferred. Even more preferred peptides of this formula are oligoarginines such as e.g. Arg₇, Arg₈, Arg₉, Arg₁₂, His₃Arg₉, Arg₉His₃, His₃Arg₉His₃, His₆Arg₉His₆, His₃Arg₄His₃, His₆Arg₄His₆, TyrSer₂Arg₉Ser₂Tyr, (ArgLysHis)₄, Tyr(ArgLysHis)₂Arg, etc.

According to a further particularly preferred embodiment, cationic or oligocationic or polycationic peptides or proteins of the polymeric carrier, having the empirical sum formula (I) as shown above and which comprise or are additionally modified to comprise at least one —SH moeity, may be preferably selected from, without being restricted thereto, at least one of the following subgroup of formulae. The following formulae (as with empirical formula (I)) do not specify any amino acid order, but are intended to reflect empirical formulae by exclusively specifying the (number of) amino acids as components of the respective peptide. Accordingly, as an example, empirical formula Arg₍₇₋₂₉₎Lys₁ is intended to mean that peptides falling under this formula contain 7 to 29 Arg residues and 1 Lys residue of whatsoever order. If the peptides contain 7 Arg residues and 1 Lys residue, all variants having 7 Arg residues and 1 Lys residue are encompassed. The Lys residue may therefore be positioned anywhere in the e.g. 8 amino acid long sequence composed of 7 Arg and 1 Lys residues.

According to a further particular preferred embodiment, cationic or oligocationic or polycationic peptides or proteins of the polymeric carrier, having the empirical sum formula (I) as shown above and which comprise or are additionally modified to comprise at least one —SH moeity, may be, without being restricted thereto, selected from the subgroup consisting of generic formulas Arg₇ (also termed as R₇), Arg₉ (also termed R₉), Arg₁₂ (also termed as R₁₂).

According to a one further particular preferred embodiment, the cationic or polycationic peptide or protein of the polymeric carrier, when defined according to formula {(Arg)ₗ;(Lys)ₘ;(His)ₙ;(Orn)ₒ;(Xaa)ₓ} (formula (I)) as shown above and which comprise or are additionally modified to comprise at least one —SH moeity, may be, without being restricted thereto, selected from subformula (Ia):

{(Arg)ₗ;(Lys)ₘ;(His)ₙ;(Orn)ₒ;(Xaa′)ₓ(Cys)_{y}} formula (Ia)

wherein (Arg)ₗ;(Lys)ₘ;(His)ₙ;(Orn)ₒ; and x are as defined herein, Xaa′ is any amino acid selected from native (=naturally occurring) or non-native amino acids except of Arg, Lys, His, Orn or Cys and y is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80 and 81-90, provided that the overall content of Arg (Arginine), Lys (Lysine), His (Histidine) and Orn (Ornithine) represents at least 10% of all amino acids of the oligopeptide.

This embodiment may apply to situations, wherein the cationic or oligocationicor polycationic peptide or protein of the polymeric carrier, e.g. when defined according to empirical formula (Arg)ₗ;(Lys)ₘ;(His)ₙ;(Orn)ₒ;(Xaa)ₓ (formula (I)) as shown above, comprises or has been modified with at least one cysteine as —SH moiety in the above meaning such that the cationic or oligocationic or polycationic peptide as cationic component carries at least one cysteine, which is capable to form a disulfide bond with other components of the polymeric carrier.

In a preferred embodiment, the polymeric carrier cargo complex comprises a carrier, which comprises or consists of the peptide CysArg₁₂ (SEQ ID NO:8). Therein, the peptide having the sequence according to SEQ ID NO: 8 is preferably further modified by an amino acid component (AA) as defined herein.

According to another particular preferred embodiment, the cationic or polycationic peptide or protein of the polymeric carrier, when defined according to formula {(Arg)ₗ(Lys)ₘ;(His)ₙ;(Orn)ₒ;(Xaa)ₓ} (formula (I)) as shown above, may be, without being restricted thereto, selected from subformula (Ib):

Cys₁{(Arg)ₗ;(Lys)ₘ;(His)ₙ;(Orn)ₒ;(Xaa)ₓ}Cys₂ (formula Ib)

wherein empirical formula {(Arg)ₗ;(Lys)ₘ;(His)ₙ;(Orn)ₒ;(Xaa)ₓ} (formula (I)) is as defined herein and forms a core of an amino acid sequence according to (semiempirical) formula (I) and wherein Cys₁ and Cys₂ are Cysteines proximal to, or terminal to (Arg)ₗ;(Lys)ₘ;(His)ₙ;(Orn)ₒ;(Xaa)ₓ. Exemplary examples may comprise any of the above sequences flanked by two Cys.

This embodiment may apply to situations, wherein the cationic or oligocationic or polycationic peptide or protein of the polymeric carrier, e.g. when defined according to empirical formula (Arg)ₗ;(Lys)ₘ;(His)ₙ;(Orn)ₒ;(Xaa)ₓ (formula (I)) as shown above, has been modified with at least two cysteines as —SH moieties in the above meaning such that the cationic or polycationic peptide of the polymeric carrier cargo complex as cationic component carries at least two (terminal) cysteines, which are capable to form a disulfide bond with other components of the polymeric carrier.

In a preferred embodiment, the polymeric carrier cargo complex comprises a carrier, which comprises or consists of the peptide CysArg₁₂Cys (SEQ ID NO: 7). Therein, the peptide having the sequence according to SEQ ID NO: 7 is preferably further modified by an amino acid component (AA) as defined herein.

According to a second alternative, at least one cationic (or oligocationic or polycationic) component of the polymeric carrier may be selected from e.g. any (non-peptidic) cationic or oligocationic or polycationic polymer suitable in this context, provided that this (non-peptidic) cationic or polycationic polymer exhibits or is modified to exhibit at least one —SH-moiety, which provide for a disulfide bond linking the cationic or oligocationic or polycationic polymer with another component of the polymeric carrier as defined herein. Thus, likewise as defined herein, the polymeric carrier may comprise the same or different cationic or polycationic polymers.

In the specific case that the cationic component of the polymeric carrier comprises a (non-peptidic) cationic or oligocationic or polycationic polymer, the cationic properties of the (non-peptidic) cationic or oligocationic or polycationic polymer may be determined upon its content of cationic charges when compared to the overall charges of the components of the cationic polymer. Preferably, the content of cationic charges in the cationic polymer at a (physiological) pH as defined herein is at least 10%, 20%, or 30%, preferably at least 40%, more preferably at least 50%, 60% or 70%, but also preferably at least 80%, 90%, or even 95%, 96%, 97%, 98%, 99% or 100%, most preferably at least 30%, 40%, 50%, 60%, 70%, 80%, 90%, 95%, 96%, 97%, 98%, 99% or 100%, or may be in the range of about 10% to 90%, more preferably in the range of about 30% to 100%, even preferably in the range of about 50% to 100%, e.g. 50, 60, 70, 80%, 90% or 100%, or in a range formed by any two of the afore mentioned values, provided, that the content of all charges, e.g. positive and negative charges at a (physiological) pH as defined herein, in the entire cationic polymer is 100%.

Preferably, the (non-peptidic) cationic component of the polymeric carrier represents a cationic or polycationic polymer, typically exhibiting a molecular weight of about 0.1 or 0.5 kDa to about 100 kDa, preferably of about 1 kDa to about 75 kDa, more preferably of about 5 kDa to about 50 kDa, even more preferably of about 5 kDa to about 30 kDa, or a molecular weight of about 10 kDa to about 50 kDa, even more preferably of about 10 kDa to about 30 kDa. Additionally, the (non-peptidic) cationic or polycationic polymer typically exhibits at least one —SH-moiety, which is capable to form a disulfide linkage upon condensation with either other cationic components or other components of the polymeric carrier as defined herein.

In the above context, the (non-peptidic) cationic component of the polymeric carrier may be selected from acrylates, modified acrylates, such as pDMAEMA (poly(dimethylaminoethyl methylacrylate)), chitosanes, aziridines or 2-ethyl-2-oxazoline (forming oligo ethylenimines or modified oligoethylenimines), polymers obtained by reaction of bisacrylates with amines forming oligo beta aminoesters or poly amido amines, or other polymers like polyesters, polycarbonates, etc. Each molecule of these (non-peptidic) cationic or polycationic polymers typically exhibits at least one —SH-moiety, wherein these at least one —SH-moiety may be introduced into the (non-peptidic) cationic or polycationic polymer by chemical modifications, e.g. using imonothiolan, 3-thio propionic acid or introduction of —SH-moieties containing amino acids, such as cysteine or any further (modified) amino acid. Such —SH-moieties are preferably as already defined above.

In the context of the polymeric carrier, the cationic components, which form basis for the polymeric carrier, may be the same or different from each other. It is also particularly preferred that the polymeric carrier of the present invention comprises mixtures of cationic peptides, proteins or polymers and optionally further components as defined herein, which are preferably crosslinked by disulfide bonds as described herein.

In this context, the polymeric carrier cargo complex due to its variable polymeric carrier advantageously allows to combine desired properties of different (short) cationic or oligocationic or polycationic peptides, proteins or polymers or other components. The polymeric carrier, e.g., allows to efficiently compact nucleic acids for the purpose of efficient transfection of nucleic acids, for adjuvant therapy, for the purposes of gene therapy, for gene knock-down or others strategies without loss of activity, particularly exhibiting an efficient transfection of a nucleic acid into different cell lines in vitro but particularly transfection in vivo. The polymeric carrier and thus the polymeric carrier cargo complex is furthermore not toxic to cells, provides for efficient release of its nucleic acid cargo, is stable during lyophilization and is applicable as immunostimulating agent or adjuvant. Preferably, the polymer carrier cargo complex may induce the anti-viral cytokine IFN-alpha.

In particular, the polymeric carrier preferably formed by disulfide-linked cationic components allows considerably to vary its peptide or polymeric content and thus to modulate its biophysical/biochemical properties, particularly the cationic properties of the polymeric carrier, quite easily and fast, e.g. by incorporating as cationic components the same or different cationic peptide(s) or polymer(s) and optionally adding other components into the polymeric carrier. Even though consisting of quite small non-toxic monomer units the polymeric carrier forms a long cationic binding sequence providing a strong condensation of the nucleic acid cargo and complex stability. Under the reducing conditions of the cytosol (e.g. cytosolic GSH), the complex is rapidly degraded into its (cationic) components, which are further degraded (e.g. into oligopeptides). This supports the liberation of the nucleic acid cargo in the cytosol. Due to degradation into small oligopeptides or polymers in the cytosol, no toxicity is observed as known for high-molecular oligopeptides or polymers, e.g. from high-molecular polyarginine.

Accordingly, the polymeric carrier of the inventive composition may comprise different (short) cationic or oligocationic or polycationic peptides, proteins or polymers selected from cationic or oligocationic or polycationic peptides, proteins or (non-peptidic) polymers as defined above, optionally together with further components as defined herein.

Additionally, the polymeric carrier of the polymeric carrier cargo complex as defined above, more preferably at least one of the different (short) cationic or oligocationic or polycationic peptides or (non-peptidic) polymers forming basis for the polymeric carrier, may be, preferably prior to the disulfide-crosslinking, be modified with at least one further component. Alternatively, the polymeric carrier as such may be modified with at least one further component. It may also optionally comprise at least one further component, which typically forms the polymeric carrier disulfide together with the other the (short) cationic or oligocationic or polycationic peptides as defined above via disulfide crosslinking.

To allow modification of a cationic or oligocationic or polycationic peptide or a (non-peptidic) polymer as defined above, each of the components of the polymeric carrier may (preferably already prior to disulfide-crosslinking) also contain at least one further functional moiety, which allows attaching such further components as defined herein. Such functional moieties may be selected from functionalities which allow the attachment of further components, e.g. functionalities as defined herein, e.g. by amide formation (e.g. carboxylic acids, sulphonic acids, amines, etc.), by Michael addition (e.g maleinimide moieties, α,β unsatured carbonyls, etc.), by click chemistry (e.g. azides or alkines), by alkene/alkine methatesis (e.g. alkenes or alkines), imine or hydrozone formation (aldehydes or ketons, hydrazins, hydroxylamins, amines), complexation reactions (avidin, biotin, protein G) or components which allow S-type substitution reactions (e.g halogenalkans, thiols, alcohols, amines, hydrazines, hydrazides, sulphonic acid esters, oxyphosphonium salts) or other chemical moieties which can be utilized in the attachment of further components.

According to a particularly preferred embodiment, the further component, which may be contained in the polymeric carrier or which may be used to modify the different (short) cationic or oligocationic or polycationic peptides or (non-peptidic) polymers forming basis for the polymeric carrier of the polymeric carrier cargo complex is an amino acid component (AA), which may e.g. modify the biophysical/biochemical properties of the polymeric carrier as defined herein. According to the present invention, the amino acid component (AA) comprises a number of amino acids preferably in a range of about 1 to 100, preferably in a range of about 1 to 50, more preferably selected from a number comprising 1, 2, 3, 4, 5, 6, 7, 8, 9, 10, 11, 12, 13, 14 or 15-20, or may be selected from a range formed by any two of the afore mentioned values. In this context the amino acids of amino acid component (AA) can be chosen independently from each other. For example if in the polymeric carrier two or more (AA) components are present they can be the same or can be different from each other.

The amino acid component (AA) may contain or may be flanked (e.g. terminally) by a —SH containing moiety, which allows introducing this component (AA) via a disulfide bond into the polymeric carrier as defined herein. In the specific case that the —SH containing moiety represents a cysteine, the amino acid component (AA) may also be read as -Cys-(AA)-Cys-, -Cys-(AA) or (AA)-Cys, wherein Cys represents Cysteine and provides for the necessary —SH-moiety for a disulfide bond. The —SH containing moiety may be also introduced into amino acid component (AA) using any of modifications or reactions as shown above for the cationic component or any of its components.

Furthermore, the amino acid component (AA) may be provided with two —SH-moieties (or even more), e.g. in a form represented by formula HS-(AA)-SH to allow binding to two functionalities via disulfide bonds, e.g. if the amino acid component (AA) is used as a linker between two further components (e.g. as a linker between two cationic polymers). In this case, one —SH moiety is preferably protected in a first step using a protecting group as known in the art, leading to an amino acid component (AA) of formula HS-(AA)-S-protecting group. Then, the amino acid component (AA) may be bound to a further component of the polymeric carrier, to form a first disulfide bond via the non-protected —SH moiety. The protected—SH-moiety is then typically deprotected and bound to a further free —SH-moiety of a further component of the polymeric carrier to form a second disulfide bond.

Alternatively, the amino acid component (AA) may be provided with other functionalities as already described above for the other components of the polymeric carrier, which allow binding of the amino acid component (AA) to any of components of the polymeric carrier.

Thus, according to the present invention, the amino acid component (AA) may be bound to further components of the polymeric carrier with or without using a disulfide linkage. Binding without using a disulfide linkage may be accomplished by any of the reactions described above, preferably by binding the amino acid component (AA) to the other component of the polymeric carrier using an amid-chemistry as defined herein. If desired or necessary, the other terminus of the amino acid component (AA), e.g. the N- or C-terminus, may be used to couple another component, e.g. a ligand L. For this purpose, the other terminus of the amino acid component (AA) preferably comprises or is modified to comprise a further functionality, e.g. an alkyn-species (see above), which may be used to add the other component via e.g. click-chemistry. If the ligand is bound via an acid-labile bond, the bond is preferably cleaved off in the endosome and the polymeric carrier presents amino acid component (AA) at its surface.

The amino acid component (AA) may occur as a further component of the polymeric carrier as defined above, e.g. as a linker between cationic components e.g. as a linker between one cationic peptide and a further cationic peptide, as a linker between one cationic polymer and a further cationic polymer, as a linker between one cationic peptide and a cationic polymer, all preferably as defined herein, or as an additional component of the polymeric carrier, e.g. by binding the amino acid component (AA) to the polymeric carrier or a component thereof, e.g. via side chains, SH-moieties or via further moieties as defined herein, wherein the amino acid component (AA) is preferably accordingly modified.

According to a further and particularly preferred alternative, the amino acid component (AA), may be used to modify the polymeric carrier, particularly the content of cationic components in the polymeric carrier as defined above.

In this context it is preferable, that the content of cationic components in the polymeric carrier is at least 10%, 20%, or 30%, preferably at least 40%, more preferably at least 50%, 60% or 70%, but also preferably at least 80%, 90%, or even 95%, 96%, 97%, 98%, 99% or 100%, most preferably at least 30%, 40%, 50%, 60%, 70%, 80%, 90%, 95%, 96%, 97%, 98%, 99% or 100%, or may be in the range of about 30% to 100%, more preferably in the range of about 50% to 100%, even preferably in the range of about 70% to 100%, e.g. 70, 80, 90 or 100%, or in a range formed by any two of the afore mentioned values, provided, that the content of all components in the polymeric carrier is 100%.

In the context of the present invention, the amino acid component (AA) may be selected from the following alternatives.

According to a first alternative, the amino acid component (AA) may be an aromatic amino acid component (AA). The incorporation of aromatic amino acids or sequences as amino aromatic acid component (AA) into the polymeric carrier of the present invention enables a different (second) binding of the polymeric carrier to the nucleic acid due to interactions of the aromatic amino acids with the bases of the nucleic acid cargo in contrast to the binding thereof by cationic charged sequences of the polymeric carrier molecule to the phosphate backbone. This interaction may occur e.g. by intercalations or by minor or major groove binding. This kind of interaction is not prone to decompaction by anionic complexing partners (e.g. Heparin, Hyaluronic acids) which are found mainly in the extracellular matrix in vivo and is also less susceptible to salt effects.

For this purpose, the amino acids in the aromatic amino acid component (AA) may be selected from either the same or different aromatic amino acids e.g. selected from Trp, Tyr, or Phe. Additionally, the aromatic amino acid component (AA) may contain or may be flanked by a —SH containing moiety, which allows introducing this component via a disulfide bond as a further part of the polymeric carrier as defined above, e.g. as a linker. Such a —SH containing moiety may be any moiety as defined herein suitable to couple one component as defined herein to a further component as defined herein. As an example, such a —SH containing moiety may be a cysteine. Additionally, the aromatic amino acid component (AA) may contain or represent at least one proline, which may serve as a structure breaker of longer sequences of Trp, Tyr and Phe in the aromatic amino acid component (AA), preferably two, three or more prolines.

According to a second alternative, the amino acid component (AA) may be a hydrophilic (and preferably non-charged polar) amino acid component (AA). The incorporation of hydrophilic (and preferably non-charged polar) amino acids or sequences as amino hydrophilic (and preferably non-charged polar) acid component (AA) into the polymeric carrier of the present invention enables a more flexible binding to the nucleic acid cargo. This leads to a more effective compaction of the nucleic acid cargo and hence to a better protection against nucleases and unwanted decompaction. It also allows provision of a (long) polymeric carrier which exhibits a reduced cationic charge over the entire carrier and in this context to better adjusted binding properties, if desired or necessary.

For this purpose, the amino acids in the hydrophilic (and preferably non-charged polar) amino acid component (AA) may be selected from either the same or different hydrophilic (and preferably non-charged polar) amino acids e.g. selected from Thr, Ser, Asn or Gln. All peptide combinations may also be combined with each other as suitable.

Additionally, the hydrophilic (and preferably non-charged polar) amino acid component (AA) may contain or may be flanked by a —SH containing moiety, which allows introducing this component via a disulfide bond as a further part of generic formula (I) above, e.g. as a linker. Such a —SH containing moiety may be any moiety as defined herein suitable to couple one component as defined herein to a further component as defined herein. As an example, such a —SH containing moiety may be a cysteine.

Additionally, the hydrophilic (and preferably non-charged polar) amino acid component (AA) may contain at least one proline, which may serve as a structure breaker of longer sequences of Ser, Thr and Asn in the hydrophilic (and preferably non-charged polar) amino acid component (AA), preferably two, three or more prolines.

According to a third alternative, the amino acid component (AA) may be a lipohilic amino acid component (AA). The incorporation of lipohilic amino acids or sequences as amino lipohilic acid component (AA) into the polymeric carrier of the present invention enables a stronger compaction of the nucleic acid cargo and/or the polymeric carrier and its nucleic acid cargo when forming a complex. This is particularly due to interactions of one or more polymer strands of the polymeric carrier, particularly of lipophilic sections of lipohilic amino acid component (AA) and the nucleic acid cargo. This interaction will preferably add an additional stability to the complex between the polymeric carrier and its nucleic acid cargo. This stabilization may somehow be compared to a sort of non covalent crosslinking between different polymerstrands. Especially in aqueous environment this interaction is typically strong and provides a significant effect.

For this purpose, the amino acids in the lipophilic amino acid component (AA) may be selected from either the same or different lipophilic amino acids e.g. selected from Leu, Val, Ile, Ala, Met. Additionally, the lipophilic amino acid component (AA) may contain or may be flanked by a —SH containing moiety, which allows introducing this component via a disulfide bond as a further part of the polymeric carrier above, e.g. as a linker. Such a —SH containing moiety may be any moiety as defined herein suitable to couple one component as defined herein to a further component as defined herein. As an example, such a —SH containing moiety may be a cysteine.

Additionally, the lipophilic amino acid component (AA) may contain at least one proline, which may serve as a structure breaker of longer sequences of Leu, Val, Ile, Ala and Met in the lipophilic amino acid component (AA), preferably two, three or more prolines.

Finally, according to a fourth alternative, the amino acid component (AA) may be a weak basic amino acid component (AA). The incorporation of weak basic amino acids or sequences as weak basic amino acid component (AA) into the polymeric carrier of the present invention may serve as a proton sponge and facilitates endosomal escape (also called endosomal release) (proton sponge effect). Incorporation of such a weak basic amino acid component (AA) preferably enhances transfection efficiency.

For this purpose, the amino acids in the weak basic amino acid component (AA) may be selected from either the same or different weak amino acids e.g. selected from histidine or aspartate (aspartic acid). Additionally, the weak basic amino acid component (AA) may contain or may be flanked by a —SH containing moiety, which allows introducing this component via a disulfide bond as a further part of generic formula (I) above, e.g. as a linker. Such a —SH containing moiety may be any moiety as defined herein suitable to couple one component as defined herein to a further component as defined herein. As an example, such a —SH containing moiety may be a cysteine.

Additionally, the weak basic amino acid component (AA) may contain at least one proline, which may serve as a structure breaker of longer sequences of histidine or aspartate (aspartic acid) in the weak basic amino acid component (AA), preferably two, three or more prolines.

According to a fifth alternative, the amino acid component (AA) may be a signal peptide or signal sequence, a localization signal or sequence, a nuclear localization signal or sequence (NLS), an antibody, a cell penetrating peptide, (e.g. TAT), etc. Preferably such an amino acid component (AA) is bound to the polymeric carrier or to another component of the polymeric carrier via a (reversible) disulfide bond. In this context the signal peptide or signal sequence, a localization signal or sequence, a nuclear localization signal or sequence (NLS), an antibody, a cell penetrating peptide, (e.g. TAT), etc.; additionally comprises at least one —SH— moiety. In this context a signal peptide, a localization signal or sequence or a nuclear localization signal or sequence (NLS), may be used to direct the polymeric carrier cargo complex to specific target cells (e.g. hepatocytes or antigen-presenting cells) and preferably allows a translocalization of the polymeric carrier to a specific target, e.g. into the cell, into the nucleus, into the endosomal compartment, sequences for the mitochondrial matrix, localisation sequences for the plasma membrane, localisation sequences for the Golgi apparatus, the nucleus, the cytoplasm and the cytosceleton, etc. Such signal peptide, a localization signal or sequence or a nuclear localization signal may be used for the transport of any of the herein defined nucleic acids, preferably an RNA or a DNA, more preferably an shRNA or a pDNA, e.g. into the nucleus. Without being limited thereto, such a signal peptide, a localization signal or sequence or a nuclear localization signal may comprise, e.g., localisation sequences for the endoplasmic reticulum. Such an additional component may be bound e.g. to a cationic polymer or to any other component of the polymeric carrier as defined herein. Preferably this signal peptide, localization signal or sequence or nuclear localization signal or sequence (NLS), is bound to the polymeric carrier or to another component of the polymeric carrier via a (reversible) disulfide bond. For this purpose the (AA) component additionally comprises at least one —SH moiety as defined herein. The binding to any of components of the polymeric carrier may also be accomplished using an acid-labile bond, preferably via a side chain of any of components of the polymeric carrier, which allows to detach or release the additional component at lower pH-values, e.g. at physiological pH-values as defined herein.

Additionally, according to another alternative, the amino acid component (AA) may be a functional peptide or protein, which may modulate the functionality of the polymeric carrier accordingly. Such functional peptides or proteins as the amino acid component (AA) preferably comprise any peptides or proteins as defined herein, e.g. as defined below as therapeutically active proteins. According to one alternative, such further functional peptides or proteins may comprise so called cell penetrating peptides (CPPs) or cationic peptides for transportation. Particularly preferred are CPPs, which induce a pH-mediated conformational change in the endosome and lead to an improved release of the polymeric carrier (in complex with a nucleic acid) from the endosome by insertion into the lipid layer of the liposome. Such an amino acid component (AA) may also be bound to any component of the polymeric carrier as defined herein. Preferably it is bound to the polymeric carrier or to another component of the polymeric carrier via a (reversible) disulfide bond. For the above purpose, the amino acid component (AA) preferably comprises at least one —SH moiety as defined herein. The binding to any of components of the polymeric carrier may also be accomplished using an SH-moiety or an acid-labile bond, preferably via a side chain of any of components of the polymeric carrier which allows to detach or release the additional component at lower pH-values, e.g. at physiological pH-values as defined herein.

According to a last alternative, the amino acid component (AA) may consist of any peptide or protein which can execute any favorable function in the cell. Particularly preferred are peptides or proteins selected from therapeutically active proteins or peptides, from antigens, e.g. tumor antigens, pathogenic antigens (animal antigens, viral antigens, protozoal antigens, bacterial antigens, allergic antigens), autoimmune antigens, or further antigens, from allergens, from antibodies, from immunostimulatory proteins or peptides, from antigen-specific T-cell receptors, or from any other protein or peptide suitable for a specific (therapeutic) application as defined below for coding nucleic acids. Particularly preferred are peptide epitopes from antigens as defined herein.

The polymeric carrier may comprise at least one of the above mentioned cationic or oligocationic or polycationic peptides, proteins or polymers or further components, e.g. (AA), wherein any of the above alternatives may be combined with each other, and may be formed by polymerizing same in a condensation polymerization reaction via their —SH-moieties.

According to another aspect, the polymeric carrier of the polymeric carrier cargo complex or single components thereof, e.g. of the above mentioned cationic or oligocationic or polycationic peptides, proteins or polymers or further components, e.g. (AA), may be further modified with a ligand, preferably a carbohydrate, more preferably a sugar, even more preferably mannose. Preferably this ligand is bound to the polymeric carrier or to a component of the polymeric carrier via a (reversible) disulfide bond or via Michael addition. In the case that the ligand is bound by a disulfide bond the ligand additionally comprises at least one —SH-moiety. These ligands may be used to direct the polymeric carrier cargo complex to specific target cells (e.g. hepatocytes or antigen-presenting cells). In this context mannose is particular preferred as ligand in the case that dendritic cells are the target especially for vaccination or adjuvant purposes.

According to a further embodiment of the invention, the polymeric carrier cargo complex may comprise (AA) components as defined above which do not comprise —SH moieties. These (AA) components can be added before or during the complexation reaction of the at least one nucleic acid molecule. Thereby, the (AA) component(s) is/are (non-covalently) incorporated into the polymeric carrier cargo complex without inclusion of the (AA) component(s) in the polymeric carrier itself by (covalent) polymerization.

According to one specific embodiment, the entire polymeric carrier cargo complex may be formed by a polymerization or condensation (of at least one) of the above mentioned cationic or oligocationic or polycationic peptides, proteins or polymers or further components, e.g. (AA), preferably via their —SH-moieties in a first step and complexing the first nucleic acid to such a polymeric carrier in a second step. The polymeric carrier may thus contain a number of at least one or even more of the same or different of the above defined cationic or polycationic peptides, proteins or polymers or further components, e.g. (AA), the number preferably determined by the above range.

According to one alternative specific embodiment, the polymeric carrier cargo complex is formed by carrying out the polymerization or condensation of at least one of the above mentioned cationic or oligocationic or polycationic peptides, proteins or polymers or further components, e.g. (AA), preferably via their —SH-moieties simultaneously to complexing the nucleic acid cargo to the (in situ prepared) polymeric carrier. Likewise, the polymeric carrier may thus also here contain a number of at least one or even more of the same or different of the above defined cationic or oligocationic or polycationic peptides, proteins or polymers or further components, e.g. (AA), the number preferably determined by the above range.

The polymeric carrier cargo complex additionally comprises as a cargo at least one first nucleic acid molecule. In the context of the present invention, such a first nucleic acid molecule may be any suitable nucleic acid, selected e.g. from any (single-stranded or double-stranded) DNA, preferably, without being limited thereto, e.g. genomic DNA, single-stranded DNA molecules, double-stranded DNA molecules, coding DNA, DNA primers, DNA probes, immunostimulatory DNA, a (short) DNA oligonucleotide ((short) oligodesoxyribonucleotides), viral DNA, or may be selected e.g. from any PNA (peptide nucleic acid) or may be selected e.g. from any (single-stranded or double-stranded) RNA, preferably, without being limited thereto, a (short) RNA oligonucleotide ((short) oligoribonucleotide), a coding RNA, a messenger RNA (mRNA), a viral RNA, replicons, an immunostimulatory RNA, a small interfering RNA (siRNA), an antisense RNA, a micro RNA, a small nuclear RNA (snRNA), a small-hairpin (sh) RNA or riboswitches, ribozymes or aptamers; etc. The nucleic acid molecule of the polymeric carrier cargo complex may also be a ribosomal RNA (rRNA), a transfer RNA (tRNA), a messenger RNA (mRNA), or a viral RNA (vRNA).

The nucleic acid of the polymeric carrier cargo complex may be a single- or a double-stranded nucleic acid molecule or a partially double-stranded or partially single stranded nucleic acid, which are at least partially self complementary (both of these partially double-stranded or partially single stranded nucleic acid molecules are typically formed by a longer and a shorter single-stranded nucleic acid molecule or by two single stranded nucleic acid molecules, which are about equal in length, wherein one single-stranded nucleic acid molecule is in part complementary to the other single-stranded nucleic acid molecule and both thus form a double-stranded nucleic acid molecule in this region, i.e. a partially double-stranded or partially single stranded nucleic acid molecule. Preferably, the nucleic acid molecule may be a single-stranded nucleic acid molecule. Furthermore, the nucleic acid molecule may be a circular or linear nucleic acid molecule, preferably a linear nucleic acid molecule.

Preferably, the nucleic acid molecule of the polymeric carrier cargo complex is an RNA. More preferably, the nucleic acid molecule of the polymeric carrier cargo complex is a (linear) single-stranded RNA, even more preferably an mRNA or an immunostimulatory RNA (isRNA). In an especially preferred embodiment the nucleic acid molecule of the polymeric carrier cargo complex is a non-coding immunostimulatory RNA according to SEQ ID NO: 2.

Furthermore, the immunostimulatory nucleic acid, as used herein, is preferably selected from an immunostimulatory RNA (isRNA), which preferably elicits an innate immune response. In this context it is particular preferred that the isRNA carries a triphosphate at its 5′-end which is the case for in vitro transcribed RNA. An immunostimulatory RNA may also occur as a short RNA oligonucleotide as defined herein. An immunostimulatory RNA as used herein may furthermore be selected from any class of RNA molecules, found in nature or being prepared synthetically, and which can induce an innate immune response and may support an adaptive immune response induced by an antigen. In this context, an immune response may occur in various ways. A substantial factor for a suitable (adaptive) immune response is the stimulation of different T cell sub-populations. T-lymphocytes are typically divided into two sub-populations, the T-helper 1 (Th1) cells and the T-helper 2 (Th2) cells, with which the immune system is capable of destroying intracellular (Th1) and extracellular (Th2) pathogens (e.g. antigens). The two Th cell populations differ in the pattern of the effector proteins (cytokines) produced by them. Thus, Th1 cells assist the cellular immune response by activation of macrophages and cytotoxic T cells. Th2 cells, on the other hand, promote the humoral immune response by stimulation of B-cells for conversion into plasma cells and by formation of antibodies (e.g. against antigens). The Th1/Th2 ratio is therefore of great importance in the induction and maintenance of an adaptive immune response. In connection with the present invention, the Th1/Th2 ratio of the (adaptive) immune response is preferably shifted in the direction towards the cellular response (Th1 response) and a cellular immune response is thereby induced. According to one example, the innate immune system which may support an adaptive immune response may be activated by ligands of Toll-like receptors (TLRs). TLRs are a family of highly conserved pattern recognition receptor (PRR) polypeptides that recognize pathogen-associated molecular patterns (PAMPs) and play a critical role in innate immunity in mammals. Currently at least thirteen family members, designated TLR1-TLR13 (Toll-like receptors: TLR1, TLR2, TLR3, TLR4, TLR5, TLR6, TLR7, TLR8, TLR9, TLR10, TLR11, TLR12 or TLR13), have been identified. Furthermore, a number of specific TLR ligands have been identified. It was e.g. found that unmethylated bacterial DNA and synthetic analogs thereof (CpG DNA) are ligands for TLR9 (Hemmi H et al. (2000) Nature 408:740-5; Bauer S et al. (2001) Proc Natl. Acad. Sci. USA 98, 9237-42). Furthermore, it has been reported that ligands for certain TLRs include certain nucleic acid molecules and that certain types of RNA are immunostimulatory in a sequence-independent or sequence-dependent manner, wherein these various immunostimulatory RNAs may e.g. stimulate TLR3, TLR7, or TLR8, or intracellular receptors such as RIG-I, MDA-5, etc.

Preferably, an immunostimulatory nucleic acid, preferably an immunostimulatory RNA (isRNA), as used herein, may comprise any RNA sequence known to be immunostimulatory, including, without being limited thereto, RNA sequences representing and/or encoding ligands of TLRs, preferably selected from human family members TLR1-TLR10 or murine family members TLR1-TLR13, more preferably selected from (human) family members TLR1-TLR10, even more preferably from TLR7 and TLR8, ligands for intracellular receptors for RNA (such as RIG-I or MDA-5, etc.) (see e.g. Meylan, E., Tschopp, J. (2006). Toll-like receptors and RNA helicases: two parallel ways to trigger antiviral responses. Mol. Cell 22, 561-569), or any other immunostimulatory RNA sequence. Furthermore, immunostimulatory RNA molecules may include any other RNA capable of eliciting an immune response. Without being limited thereto, such an immunostimulatory RNA may include ribosomal RNA (rRNA), transfer RNA (tRNA), messenger RNA (mRNA), and viral RNA (vRNA). Such an immunostimulatory RNA may comprise a length of 1000 to 5000, of 500 to 5000, of 5 to 5000, or of 5 to 1000, 5 to 500, 5 to 250, of 5 to 100, of 5 to 50 or of 5 to 30 nucleotides.

An immunostimulatory RNA as used herein may furthermore be selected from any class of RNA molecules, found in nature or being prepared synthetically, and which can induce an innate immune response and may support an adaptive immune response induced by an antigen. In this context, an immune response may occur in various ways. A substantial factor for a suitable (adaptive) immune response is the stimulation of different T-cell sub-populations. T-lymphocytes are typically divided into two sub-populations, the T-helper 1 (Th1) cells and the T-helper 2 (Th2) cells, with which the immune system is capable of destroying intracellular (Th1) and extracellular (Th2) pathogens (e.g. antigens). The two Th cell populations differ in the pattern of the effector proteins (cytokines) produced by them. Thus, Th1 cells assist the cellular immune response by activation of macrophages and cytotoxic T-cells. Th2 cells, on the other hand, promote the humoral immune response by stimulation of B-cells for conversion into plasma cells and by formation of antibodies (e.g. against antigens). The Th1/Th2 ratio is therefore of great importance in the induction and maintenance of an adaptive immune response. In connection with the present invention, the Th1/Th2 ratio of the (adaptive) immune response is preferably shifted in the direction towards the cellular response (Th1 response) and a cellular immune response is thereby induced. According to one example, the innate immune system which may support an adaptive immune response, may be activated by ligands of Toll-like receptors (TLRs). TLRs are a family of highly conserved pattern recognition receptor (PRR) polypeptides that recognize pathogen-associated molecular patterns (PAMPs) and play a critical role in innate immunity in mammals. Currently at least thirteen family members, designated TLR1-TLR13 (Toll-like receptors: TLR1, TLR2, TLR3, TLR4, TLR5, TLR6, TLR7, TLR8, TLR9, TLR10, TLR11, TLR12 or TLR13), have been identified. Furthermore, a number of specific TLR ligands have been identified. It was e.g. found that unmethylated bacterial DNA and synthetic analogs thereof (CpG DNA) are ligands for TLR9 (Hemmi H et al. (2000) Nature 408:740-5; Bauer S et al. (2001) Proc Natl Acad Sci USA 98, 9237-42). Furthermore, it has been reported that ligands for certain TLRs include certain nucleic acid molecules and that certain types of RNA are immunostimulatory in a sequence-independent or sequence-dependent manner, wherein these various immunostimulatory RNAs may e.g. stimulate TLR3, TLR7, or TLR8, or intracellular receptors such as RIG-I, MDA-5, etc. E.g. Lipford et al. determined certain G,U-containing oligoribonucleotides as immunostimulatory by acting via TLR7 and TLR8 (see WO 03/086280). The immunostimulatory G,U-containing oligoribonucleotides described by Lipford et al. were believed to be derivable from RNA sources including ribosomal RNA, transfer RNA, messenger RNA, and viral RNA.

According to a particularly preferred embodiment, such immunostimulatory nucleic acid sequences is preferably RNA preferably consisting of or comprising a nucleic acid of the following formula (II) or (Ill):

GₗXₘGₙ, (formula II)p

wherein:
G is guanosine, uridine (uracil) or an analogue of guanosine or uridine (uracil);
X is guanosine, uridine (uracil), adenosine, thymidine, cytidine (cytosine) or an analogue of the above-mentioned nucleotides;
l is an integer from 1 to 40,
   wherein
   when l=1 G is guanosine or an analogue thereof,
   when l>1 at least 50% of the nucleotides are guanosine or an analogue thereof;
m is an integer and is at least 3;
   wherein
   when m=3 X is uridine (uracil) or an analogue thereof,
   when m>3 at least 3 successive uridines (uracils) or analogues of uridine (uracil) occur;
n is an integer from 1 to 40,
   wherein
   when n=1 G is guanosine or an analogue thereof,
   when n>1 at least 50% of the nucleotides are guanosine or an analogue thereof.

CₗXₘCₙ, (formula III)

wherein:
C is cytidine (cytosine), uridine (uracil) or an analogue of cytidine (cytosine) or uridine (uracil);
X is guanosine, uridine (uracil), adenosine, thymidine, cytidine (cytosine) or an analogue of the above-mentioned nucleotides;
l is an integer from 1 to 40,
   wherein
   when l=1 C is cytosine (cytosine) or an analogue thereof,
   when l>1 at least 50% of the nucleotides are cytidine (cytosine) or an analogue thereof;
m is an integer and is at least 3;
   wherein
   when m=3 X is uridine (uracil) or an analogue thereof,
   when m>3 at least 3 successive uridines (uracils) or analogues of uridine (uracil) occur;
n is an integer from 1 to 40,
   wherein
   when n=1 C is cytidine (cytosine) or an analogue thereof,
   when n>1 at least 50% of the nucleotides are cytidine (cytosine) or an analogue thereof.

The nucleic acids of formula (II) or (III), which may be used as the nucleic acid cargo of the polymeric carrier cargo complex may be relatively short nucleic acid molecules with a typical length of approximately from 5 to 100 (but may also be longer than 100 nucleotides for specific embodiments, e.g. up to 200 nucleotides), from 5 to 90 or from 5 to 80 nucleotides, preferably a length of approximately from 5 to 70, more preferably a length of approximately from 8 to 60 and, more preferably a length of approximately from 15 to 60 nucleotides, more preferably from 20 to 60, most preferably from 30 to 60 nucleotides. If the nucleic acid of the inventive nucleic acid cargo complex has a maximum length of e.g. 100 nucleotides, m will typically be <=98. The number of nucleotides G in the nucleic acid of formula (II) is determined by l or n. l and n, independently of one another, are each an integer from 1 to 40, wherein when l or n=1 G is guanosine or an analogue thereof, and when l or n>1 at least 50% of the nucleotides are guanosine or an analogue thereof. A nucleotide adjacent to Xₘ in the nucleic acid of formula (II) according to the invention is preferably not a uracil. Similarly, the number of nucleotides C in the nucleic acid of formula (III) according to the invention is determined by l or n. l and n, independently of one another, are each an integer from 1 to 40, wherein when l or n=1 C is cytosine or an analogue thereof, and when l or n>1 at least 50% of the nucleotides are cytosine or an analogue thereof. A nucleotide adjacent to Xₘ in the nucleic acid of formula (III) according to the invention is preferably not a uracil. Preferably, for formula (II), when l or n>1, at least 60%, 70%, 80%, 90% or even 100% of the nucleotides are guanosine or an analogue thereof, as defined above. The remaining nucleotides to 100% (when guanosine constitutes less than 100% of the nucleotides) in the flanking sequences G₁ and/or Gₙ are uracil or an analogue thereof, as defined hereinbefore. Also preferably, l and n, independently of one another, are each an integer from 2 to 30, more preferably an integer from 2 to 20 and yet more preferably an integer from 2 to 15. The lower limit of l or n can be varied if necessary and is at least 1, preferably at least 2, more preferably at least 3, 4, 5, 6, 7, 8, 9 or 10. This definition applies correspondingly to formula (III).

According to a further particularly preferred embodiment, such immunostimulatory nucleic acid sequences particularly isRNA consist of or comprise a nucleic acid of formula (IV) or (V):

(NᵤGₗXₘGₙNᵥ)ₐ, (formula IV)

wherein:
G is guanosine (guanine), uridine (uracil) or an analogue of guanosine (guanine) or uridine (uracil), preferably guanosine (guanine) or an analogue thereof;
X is guanosine (guanine), uridine (uracil), adenosine (adenine), thymidine (thymine), cytidine (cytosine), or an analogue of these nucleotides (nucleosides), preferably uridine (uracil) or an analogue thereof;
N is a nucleic acid sequence having a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides);
a is an integer from 1 to 20, preferably from 1 to 15, most preferably from 1 to 10;
l is an integer from 1 to 40,
   wherein when l=1, G is guanosine (guanine) or an analogue thereof, when l>1, at least 50% of these nucleotides (nucleosides) are guanosine (guanine) or an analogue thereof;
m is an integer and is at least 3;
   wherein when m=3, X is uridine (uracil) or an analogue thereof, and
      when m>3, at least 3 successive uridines (uracils) or analogues of uridine (uracil) occur;
n is an integer from 1 to 40,
   wherein when n=1, G is guanosine (guanine) or an analogue thereof,
      when n>1, at least 50% of these nucleotides (nucleosides) are guanosine (guanine) or an analogue thereof;
u,v may be independently from each other an integer from 0 to 50, preferably wherein when u=0, v≥1, or
   when v=0, u≥1;
      wherein the nucleic acid molecule of formula (IV) has a length of at least 50 nucleotides, preferably of at least 100 nucleotides, more preferably of at least 150 nucleotides, even more preferably of at least 200 nucleotides and most preferably of at least 250 nucleotides.

(NᵤCₗXₘCₙNᵥ)ₐ, (formula V)

wherein:
C is cytidine (cytosine), uridine (uracil) or an analogue of cytidine (cytosine) or uridine (uracil), preferably cytidine (cytosine) or an analogue thereof;
X is guanosine (guanine), uridine (uracil), adenosine (adenine), thymidine (thymine), cytidine (cytosine) or an analogue of the above-mentioned nucleotides (nucleosides), preferably uridine (uracil) or an analogue thereof;
N is each a nucleic acid sequence having independent from each other a length of about 4 to 50, preferably of about 4 to 40, more preferably of about 4 to 30 or 4 to 20 nucleic acids, each N independently being selected from guanosine (guanine), uridine (uracil), adenosine (adenine), thymidine (thymine), cytidine (cytosine) or an analogue of these nucleotides (nucleosides);
a is an integer from 1 to 20, preferably from 1 to 15, most preferably from 1 to 10;
l is an integer from 1 to 40,
   wherein when l=1, C is cytidine (cytosine) or an analogue thereof,
      when l>1, at least 50% of these nucleotides (nucleosides) are cytidine (cytosine) or an analogue thereof;
m is an integer and is at least 3;
   wherein when m=3, X is uridine (uracil) or an analogue thereof,
      when m>3, at least 3 successive uridines (uracils) or analogues of uridine (uracil) occur;
n is an integer from 1 to 40,
   wherein when n=1, C is cytidine (cytosine) or an analogue thereof,
      when n>1, at least 50% of these nucleotides (nucleosides) are cytidine (cytosine) or an analogue thereof.
u, v may be independently from each other an integer from 0 to 50,
   preferably wherein when u=0, v≥1, or
      when v=0, u≤1;
         wherein the nucleic acid molecule of formula (V) according to the invention has a length of at least 50 nucleotides, preferably of at least 100 nucleotides, more preferably of at least 150 nucleotides, even more preferably of at least 200 nucleotides and most preferably of at least 250 nucleotides.

For formula (V), any of the definitions given above for elements N (i.e. Nᵤ and Nᵥ) and X (Xₘ), particularly the core structure as defined above, as well as for integers a, l, m, n, u and v, similarly apply to elements of formula (V) correspondingly, wherein in formula (V) the core structure is defined by CₗXₘCₙ. The definition of bordering elements Nᵤ and Nᵥ is identical to the definitions given above for Nᵤ and Nᵥ.

According to a very particularly preferred embodiment, the inventive nucleic acid molecule according to formula (IV) may be selected from e.g. any of the following sequences:

(R2025, SEQ ID NO: 2)
GGGAGAAAGCUCAAGCUUAUCCAAGUAGGCUGGUCACCUGUACAACGUA
GCCGGUAUUUUUUUUUUUUUUUUUUUUUUGACCGUCUCAAGGUCCAAGU
UAGUCUGCCUAUAAAGGUGCGGAUCCACAGCUGAUGAAAGACUUGUGCG
GUACGGUUAAUCUCCCCUUUUUUUUUUUUUUUUUUUUUAGUAAAUGCGU
CUACUGAAUCCAGCGAUGAUGCUGGCCCAGAUCUUCGACCACAAGUGCA
UAUAGUAGUCAUCGAGGGUCGCCUUUUUUUUUUUUUUUUUUUUUUUGGC
CCAGUUCUGAGACUUCGCUAGAGACUACAGUUACAGCUGCAGUAGUAAC
CACUGCGGCUAUUGCAGGAAAUCCCGUUCAGGUUUUUUUUUUUUUUUUU
UUUUCCGCUCACUAUGAUUAAGAACCAGGUGGAGUGUCACUGCUCUCGA
GGUCUCACGAGAGCGCUCGAUACAGUCCUUGGAAGAAUCUUUUUUUUUU
UUUUUUUUUUUUGUGCGACGAUCACAGAGAACUUCUAUUCAUGCAGGUC
UGCUCUAG
(SEQ ID NO: 9)
GGGAGAAAGCUCAAGCUUAUCCAAGUAGGCUGGUCACCUGUACAACGUA
GCCGGUAUUUUUUUUUUUUUUUUUUUUUUGACCGUCUCAAGGUCCAAGU
UAGUCUGCCUAUAAAGGUGCGGAUCCACAGCUGAUGAAAGACUUGUGCG
GUACGGUUAAUCUCCCCUUUUUUUUUUUUUUUUUUUUUAGUAAAUGCGU
CUACUGAAUCCAGCGAUGAUGCUGGCCCAGAUCUUCGACCACAAGUGCA
UAUAGUAGUCAUCGAGGGUCGCCUUUUUUUUUUUUUUUUUUUUUUUGGC
CCAGUUCUGAGACUUCGCUAGAGACUACAGUUACAGCUGCAGUAGUAAC
CACUGCGGCUAUUGCAGGAAAUCCCGUUCAGGUUUUUUUUUUUUUUUUU
UUUUCCGCUCACUAUGAUUAAGAACCAGGUGGAGUGUCACUGCUCUCGA
GGUCUCACGAGAGCGCUCGAUACAGUCCUUGGAAGAAUCUUUUUUUUUU
UUUUUUUUUUUUGUGCGACGAUCACAGAGAACUUCUAUUCAUGCAGGUC
UGCUCUA
(SEQ ID NO: 10)
GGGAGAAAGCUCAAGCUUAUCCAAGUAGGCUGGUCACCUGUACAACGUA
GCCGGUAUUUUUUUUUUUUUUUUUUUUUUGACCGUCUCAAGGUCCAAGU
UAGUCUGCCUAUAAAGGUGCGGAUCCACAGCUGAUGAAAGACUUGUGCG
GUACGGUUAAUCUCCCCUUUUUUUUUUUUUUUUUUUUUAGUAAAUGCGU
CUACUGAAUCCAGCGAUGAUGCUGGCCCAGAUCUUCGACCACAAGUGCA
UAUAGUAGUCAUCGAGGGUCGCCUUUUUUUUUUUUUUUUUUUUUUUGGC
CCAGUUCUGAGACUUCGCUAGAGACUACAGUUACAGCUGCAGUAGUAAC
CACUGCGGCUAUUGCAGGAAAUCCCGUUCAGGUUUUUUUUUUUUUUUUU
UUUUCCGCUCACUAUGAUUAAGAACCAGGUGGAGUGUCACUGCUCUCGA
GGUCUCACGAGAGCGCUCGAUACAGUCCUUGGAAGAAUCUUUUUUUUUU
UUUUUUUUUUUUGUGCGACGAUCACAGAGAACUUCUAUUCAUGCAGGUC
UGCUCUAGAACGAACUGACCUGACGCCUGAACUUAUGAGCGUGCGUAUU
UUUUUUUUUUUUUUUUUUUUUCCUCCCAACAAAUGUCGAUCAAUAGCUG
GGCUGUUGGAGACGCGUCAGCAAAUGCCGUGGCUCCAUAGGACGUGUAG
ACUUCUAUUUUUUUUUUUUUUUUUUUUUCCCGGGACCACAAAUAAUAUU
CUUGCUUGGUUGGGCGCAAGGGCCCCGUAUCAGGUCAUAAACGGGUACA
UGUUGCACAGGCUCCUUUUUUUUUUUUUUUUUUUUUUUCGCUGAGUUAU
UCCGGUCUCAAAAGACGGCAGACGUCAGUCGACAACACGGUCUAAAGCA
GUGCUACAAUCUGCCGUGUUCGUGUUUUUUUUUUUUUUUUUUUUGUGAA
CCUACACGGCGUGCACUGUAGUUCGCAAUUCAUAGGGUACCGGCUCAGA
GUUAUGCCUUGGUUGAAAACUGCCCAGCAUACUUUUUUUUUUUUUUUUU
UUUCAUAUUCCCAUGCUAAGCAAGGGAUGCCGCGAGUCAUGUUAAGCUU
GAAUU

or from a sequence having at least 60%, 70%, 80%, 90%, or even 95% sequence identity with any of these sequences.

Furthermore, in the polymeric carrier cargo complex, the cationic component of the polymeric carrier as defined herein and the nucleic acid cargo are preferably provided in an nitrogen/phosphate ratio (N/P-ratio) in the range of 0.1-20, preferably 0.1-5, more preferably 0.1-1, most preferably 0.5-0.9. It is particularly preferred that the N/P-ratio is at least 0.1, 0.2, 0.3, 0.4, 0.5, 0.75, 1, 1.5 or 2. Preferably, the N/P—ratio lies within a range of about 0.1, 0.3, 0.4, 0.5, 0.75, 1.0, 1.5 or 2 to 20, preferably in a range of about 0.2 (0.5 or 0.75 or 1.0) to 12, and even more preferably in an N/P-ratio of about 0.4 (0.75 or 1.0) to 10. Preferably, the N/P ratio lies in a ratio between 0.1 and 0.9. In this context, the NIP ratio is a measure of the ionic charge of the cationic (side chain) component of the polymeric carrier or of the polymeric carrier as such. In particular, if the cationic properties of the cationic component are generated by nitrogens (of the amino acid side chains), the N/P ratio expresses the ratio of basic nitrogen atoms to phosphate residues in the nucleotide backbone, considering that (side chain) nitrogen atoms in the cationic component of the polymeric carrier contribute to positive charges and phosphate of the phosphate backbone of the nucleic acid contribute to the negative charge. The N/P-ratio is defined as the nitrogen/phosphate ratio (N/P-ratio) of the entire polymeric carrier cargo complex. This is typically illustrative for the content/amount of cationic components, in the polymeric carrier and characteristic for the content/amount of nucleic acids bound or complexed in the polymeric carrier cargo complex. It may be calculated on the basis that, for example, 1 μg RNA typically contains about 3 nmol phosphate residues, provided that RNA exhibits a statistical distribution of bases. Additionally, 1 nmol peptide typically contains about × nmol nitrogen residues, dependent on the molecular weight and the number of its (cationic) amino acids.

In this context it is preferable that in the polymeric carrier cargo complex, the cationic component of the polymeric carrier as defined herein and the nucleic acid cargo are provided in an N/P-ratio of at least about 1 or, preferably, of a range of about 1 to 20 for in vitro transfection purposes.

If the expression of an encoded protein or the transcription of an encoded nucleic acid e.g. an mRNA or siRNA of the nucleic acid cargo is intended for therapeutical purposes (in vivo application) an N/P ratio of at least 0.1 (0.2, 0.3, 0.4, 0.5, 0.6), preferably of a range of about 0.1 (0.2, 0.3, 0.4., 0.5, or 0.6) to 1.5 is preferred. Even more preferred is an N/P ratio range of 0.2 to 0.9 or an NIP ratio range of 0.5 to 0.9. In the case that the polymeric carrier cargo complex is used for (in vivo) immunostimulation e.g. as an immunostimulating agent or adjuvant (for the purpose to induce an innate immune response), an N/P ratio of about 0.1 to 20 is preferred, more particular an N/P ratio of 0.1 to 5 or 0.1 to 1.5.

In the specific case that the induction of IFN-α is intended using the polymeric cargo complex as an (in vivo) immunostimulating agent or adjuvant an N/P ratio of at least 0.1 (0.2, 0.3, 0.4, 0.5, or 0.6) or an N/P ratio range of 0.1 to 1 is preferred or more preferred is an NIP ratio range of 0.2 to 0.9 or an N/P ratio range of 0.5 to 0.9. Otherwise if the induction of TNFα is intended using the polymeric cargo complex as an (in vivo) immunostimulating agent or adjuvant an N/P ratio of 1 to 20 is particularly preferred.

The N/P ratio significantly influences the surface charge of the resulting polymeric carrier cargo complex. Thus it is preferable that the resulting polymeric carrier cargo complex is positively charged for in vitro transfection purposes and negatively or neutrally charged for in vivo transfection purposes, especially if the expression of an encoded protein or the transcription of an encoded nucleic acid of the nucleic acid cargo is intended. The surface charge of the resulting polymeric carrier cargo complex can be indicated as Zetapotential which may be measured by Doppler electrophoresis method using a Zetasizer Nano (Malvern Instruments, Malvern, UK).

It may be preferred that the charge of complex of the polymeric carrier and the cargo nucleic acid is negative, preferably the zetapotential of the complex is negative, i.e. below 0 mV, in particular below −4 mV. A negative charge of the complex generally leads to a preferred uptake into CD19⁺ cells, whereas positively charged complexes (which is the result of a N/P ratio higher than 1) are preferably taken up by CD3⁺ cells (e.g. T cells). Therefore, the negatively charged complexes are preferably suited for adjuvant purposes because they can target particularly antigen-presenting cells, which are the most important cells for initiating an adaptive immune response. Furthermore, these negatively charged complexes preferably induce the anti-viral cytokine IFNalpha and consequently a Th1-shifted immune response.

Therefore, these negatively charged complexes are particularly appropriate for the prophylactic or therapeutic treatment of diseases which is dependent on the induction of a Th1-shifted immune response (e.g. tumour or cancer diseases or infectious diseases like RSV infections) and for the use as adjuvant for protein or peptide antigens which mainly induce a Th2-shifted immune response.

The molar ratio of the nucleic acid molecule used as a cargo in the polymeric carrier cargo complex (“first nucleic acid molecule”) to the nucleic acid molecule encoding the antigen, which is part of the first immunogenic component of the inventive composition, administered in combination with the polymeric carrier cargo complex, is preferably in the range from 0.01 to 100, more preferably in the range from 0.1 to 10, even more preferably in the range from 0.5 to 2, most preferably about 1.

In a further preferred embodiment of the inventive composition the second adjuvant component of the inventive composition comprises at least one emulsion or surfactant-based compound as delivery system compound. The emulsion or surfactant-based compound is preferably an oil-in-water compound, more preferably a squalene-based compound, and/or a water-in-oil compound, more preferably a mineral oil-based compound or a squalene-based compound, and/or a block copolymer surfactant compound and/or a tenside-based compound. The emulsion or surfactant-based compound may be administered as single adjuvant compound, or, especially preferred, in combination with further adjuvant compounds, especially in combination with a vitamin compound. For example the emulsion or surfactant-based compound may be formed by non-ionic surfactant vesicles (NISV), or VSA-3 adjuvant, SAF (Syntex adjuvant formulation) or SAF-1 (threonyl-MDP in an emulsion vehicle). The emulsion or surfactant-based compound may be an oil-in-water emulsion, especially a mineral oil-based compound or a squalene-based compound, preferably a nano-emulsification of 2 components comprising Sorbitan trioleate (0.5% w/v) in squalene oil (5% v/v) and Tween 80 (0.5% w/v) e.g. in sodium citrate buffer (10 mM, pH 6.5) (AddaVax®), or a mixture of Squalene plus Tween 80 plus Span 85 (MF59®), or AS02 (MF59® plus MPL (monophosphoryl lipid A) plus QS-21), or AS03 (Squalene plus Tween 80 plus α-tocopherol), or AF03 (Squalene plus Montane 80 (emulsifier) (=Ceterareth-12 plus Span 80) plus Eumulgin B1 PH (emulsifier)), or a nanoemulsion, or RIBI (bacterial and mycobacterial cell wall components), e.g. Ribi529, or Ribilike adjuvant system (MPL, TMD, CWS). It may be a water-in-oil emulsion, e.g. Murametide (N2-[N-(N-Acetylmuramoyl)-L-alanyl]-D-glutamine methyl ester). Moreover the emulsion or surfactant-based compound may be a mineral oil-based compound, e.g. incomplete Freund's adjuvant (IFA), or complete Freund's adjuvant (CFA), or Specol (Marcol 52 (mineral oil, paraffins, and cycloparaffins, chain length 13-22 C atoms) plus Span 85 plus Tween 85). Moreover the emulsion or surfactant-based compound may be a squalene-based compound, e.g. squalene, or squalene plus squalane (Montanide® ISA51, Montanide® ISA720), or SPT (squalane (5%), Tween 80 (0.2%), Pluronic L121 (1.25%)), or Squalane 1 (Spinacane; Robane®; 2,6,10,15,19,23-hexamethyltetracosane and 2,6,10,15,19,23-hexamethyl-2,6,10,14,18,22-tetracosahexane), or Squalene 2 (Spinacene; Supraene; 2,6,10,15,19,23-hexamethyl-2,6,10,14,18,22 tetracosahexane), or TiterMax Gold Adjuvant. Moreover the emulsion or surfactant-based compound may be a block copolymer surfactant, e.g. pluronics, especially Pluronic L121 (Poloxamer 401). Moreover the emulsion or surfactant-based compound may be a tenside-based compound, e.g. Polysorbate 80 (Tween 80), or SPT (squalane (5%), Tween 80 (0.2%), and Pluronic L121 (1.25%)). Also combinations of the different compounds may be preferred.

In a further preferred embodiment of the inventive composition the second adjuvant component of the inventive composition comprises at least one nucleotide-based or nucleoside-based compound as immune potentiator compound. The nucleotide-based or nucleoside-based compound may be administered as single adjuvant compound, or, especially preferred, in combination with further adjuvant compounds, especially in combination with a vitamin compound. The nucleotide-based or nucleoside-based compound may be a cyclic dinucleotide compound, more preferably a cyclic guanosine monophosphate-adenosine monophosphate compound, e.g. 3′3′-cGAMP or 2′2′-cGAMP, or a cyclic diadenylate monophosphate compound, or a cyclic diguanylate monophosphate (c-di-GMP) compound. c-di-GMP may act as an agonist for cytosolic sensors of cyclic dinucleotides (CDNs) like STING. Moreover the nucleotide-based or nucleoside-based compound may be a cytosine-phosphoguanosine (CpG) dinucleotide motif compound, more preferably a oligodeoxynucleotide containing unmethylated CpG motifs compound (CpG-ODN), e.g. 1018 ISS or CpG 7909 or CpG 1018, or an oligonucleotides containing unmethylated CpG motifs compound, e.g. AS15 (MPL plus CpG plus QS-21 plus liposome). Moreover the nucleotide-based or nucleoside-based compound may be a double-stranded nucleic acid compound, more preferably a double-stranded RNA (dsRNA) compound, e.g. synthetic dsRNA, especially polyionisinic:polycytidylic acid (Poly(I:C)—which may act as an agonist for Toll-like receptor 3), or Hiltonol (polyICLC—poly-IC with poly-lysine), or poly-adenylic acid-poly-uridylic acid complex (Poly rA:Poly rU), or 5′ppp-dsRNA (which may act as an agonist for RIG-I-like receptors), or viral dsRNA. Moreover the nucleotide-based or nucleoside-based compound may be a double-stranded DNA (dsDNA) compound, e.g. IC31 (KLKL(5)KLK peptide vehicle plus ODN1a), or pCMVmCAT1 (plasmid expressing Friend murine leukemia virus receptor). dsDNA may act as an agonist for cytosolic DNA sensors (CDS). Moreover the nucleotide-based or nucleoside-based compound may be a single-stranded nucleic acid compound, more preferably a single-stranded RNA (ssRNA), e.g. guanosine-rich ssRNA, or uridine-rich ssRNA, or polymeric carrier cargo complex formed by peptide CR₁₂C plus isRNA or peptide CR₁₂ plus isRNA (RNAdjuvant). Moreover the nucleotide-based or nucleoside-based compound may be a guanosine analogue compound, e.g. Loxoribine (7-allyl-8-oxoguanosine). Another example is IMOxine™ (oligonucelotide based adjuvant). Also combinations of the different compounds may be preferred. Preferably the second adjuvant component of the inventive composition comprises at least the polymeric carrier cargo complex as described above.

According to a preferred embodiment, the second adjuvant component of the inventive combination or composition, preferably the immune potentiator and/or delivery system compound comprised therein, comprises a cyclic dinucleotide or a xanthenone derivative. The cyclic dinucleotide or the xanthenone derivative as described herein preferably binds to and activates the cellular STING (stimulator of interferon genes) receptor or another cytosolic sensor of cyclic dinucleotides. More preferably, the cyclic dinucleotide or the xanthenone derivative as described herein is selected from the group consisting of a cyclic guanosine monophosphate-adenosine monophosphate compound (cGAMP), a cyclic dimeric adenosine monophosphate compound (c-di-AMP), a cyclic dimeric guanosine monophosphate compound (c-di-GMP), a cyclic dimeric inosine monophosphate compound (c-di-IMP), a cyclic dimeric uridine monophosphate compound (c-di-UMP), or a xanthenone derivative, such as DMXAA (also known as Vadimezan or ASA404), or a derivative of any of these compounds. Even more preferably, the cyclic dinucleotide or the xanthenone derivative as described herein is selected from the group consisting of 3′3′-cGAMP, 2′3′-cGAMP, 2′3′-cGAM(PS)₂ (Rp/Sp), 2′2′-cGAMP, 3′5′-c-di-AMP, 2′3′-c-di-AMP, 2′3′-c-di-AM(PS)₂ (Rp,Rp), 3′5′-c-di-GMP, c-di-GMP fluorinated at 2′ position (c-di[2′FdGMP]), c-di-IMP, c-di-UMP, and DMXAA.

In certain embodiments, the second adjuvant component of the inventive combination or composition, preferably the immune potentiator and/or delivery system compound comprised therein, comprises a cyclic dinucleotide or a xanthenone derivative, wherein the cyclic dinucleotide or the xanthenone derivative is selected from the group consisting of 8-(2-Aminoethylthio)-cyclic diadenosine monophosphate (8-AET-c-diAMP); 8-(2-Aminoethylthio)-cyclic diadenosine monophosphate, immobilized on agarose gel (8-AET-c-diAMP-Agarose); 8-(2-Aminoethylthio)-cyclic diguanosine monophosphate (8-AET-c-diGMP); 8-(2-Aminoethylthio)-cyclic diguanosine monophosphate, immobilized on agarose gel (8-AET-c-diGMP-Agarose); 2′-O-(6-Aminohexylcarbamoyl)-cyclic diadenosine monophosphate (2′-AHC-c-diAMP); 2′-O-(6-Aminohexylcarbamoyl)-cyclic diadenosine monophosphate, immobilized on agarose (2′-AHC-c-diAMP-Agarose); 2′-O-(6-Aminohexylcarbamoyl)-cyclic diguanosine monophosphate (2′-AHC-c-diGMP); 2′-O-(6-Aminohexylcarbamoyl)-cyclic diguanosine monophosphate, immobilized on agarose (2′-AHC-c-diGMP-Agarose); 8-(2-[Biotinyl]aminoethylthio)-cyclic diadenosine monophosphate, (8-[Biotin]-AET-c-diAMP); 8-(2-[Biotinyl]aminoethylthio)-cyclic diguanosine monophosphate (8-[Biotin]-AET-c-diGMP); 2′-O-(6-[Biotinyl]aminohexylcarbamoyl)-cyclic diadenosine monophosphate (2′-[Biotin]-AHC-c-diAMP); 2′-O-(6-[Biotinyl]aminohexylcarbamoyl)-cyclic diguanosine monophosphate (2′-[Biotin]-AHC-c-diGMP); 8-Bromo-cyclic diguanosine monophosphate (8-Br-c-diGMP); 8-Chloro-cyclic diadenosine monophosphate (8-Cl-c-diAMP); cyclic (3′-O-(6-aminohexylcarbamoyl)guanosine-(2′->5′)-monophosphate-adenosine-(3′->5′)-monophosphate) (c[3′-AHC-G(2′,5′)pA(3′,5′)p]); cyclic (3′-O-(6-[biotinyl]aminohexylcarbamoyl)guanosine-(2′->5′)-monophosphate-adenosine-(3′->5′)-monophosphate) (c[3′-[Biotin]-AHC-G(2′,5′)pA(3′,5′)p]); cyclic (3′-O-(6-[fluoresceinyl]aminohexylcarbamoyl)guanosine-(2′->5′)-monophosphate-adenosine-(3′->5′)-monophosphate) (c[3′-Fluo-AHC-G(2′,5′)pA(3′,5′)p]); cyclic (8-(2-aminoethylthio)guanosine-(2′->5′)-monophosphate-adenosine-(3′->5′)-monophosphate) (c[8-AET-G(2′,5′)pA(3′,5′)p]); cyclic (8-(2-aminoethylthio)guanosine-(2′->5′)-monophosphate-adenosine-(3′->5′)-monophosphate), immobilized on agarose gel (c[8-AET-G(2′,5′)pA(3′,5′)p]-Agarose); cyclic (8-(2-[biotinyl]aminoethylthio)-guanosine-(2′->5′)-monophosphate-adenosine-(3′->5′)-monophosphate) (c[8-[Biotin]-AET-G(2′,5′)pA(3′,5′)p]); cyclic (8-(2-[fluoresceinyl]aminoethylthio)-guanosine-(2′->5′)-monophosphate-adenosine-(3′->5′)-monophosphate) (c[8-Fluo-AET-G(2′,5′)pA(3′,5′)p]/8-Fluo-AET-cGAMP(2′-5′)); cyclic (8-bromoguanosine-(2′->5′)-monophosphate-adenosine-(3′->5′)-monophosphate) (c[8-Br-G(2′,5′)pA(3′,5′)p]); cyclic (adenosine monophosphate-8-(2-aminoethylthio)guanosine monophosphate) (c-(Ap-8-AET-Gp)/8-AET-cGAMP); cyclic (adenosine monophosphate-8-(2-aminoethylthio)guanosine monophosphate), immobilized on agarose gel (c-(Ap-8-AET-Gp)-Agarose); cyclic (adenosine monophosphate-8-(2-[biotinyl]aminoethylthio)guanosine monophosphate) (c-(Ap-8-[Biotin]-AET-Gp)/8-[Biotin]-AET-cGAMP); cyclic (adenosine monophosphate-8-(2-[fluoresceinyl]aminoethylthio)guanosine monophosphate) (c-(Ap-8-Fluo-AET-Gp)/8-Fluo-AET-cGAMP); cyclic (adenosine monophosphate-8-bromoguanosine monophosphate) (c-(Ap-8-Br-Gp)/8-Br-cGAMP); cyclic (adenosine monophosphate-guanosine monophosphate) (c-(ApGp)/cGAMP/3′3′-cGAMP/cyclic GMP-AMP/cyclic-AMP-GMP/c[G(3′,5′)pA(3′,5′)p]); cyclic (adenosine monophosphate-inosine monophosphate) (c-(ApIp)); cyclic (adenosine-(2′->5′)-monophosphate-adenosine-(3′->5′)-monophosphate) (c[A(2′,5′)pA(3′,5′)p]/2′3′-c-diAMP/2′,5′-3′,5′-c-diAMP); cyclic (adenosine-(2′->5′)-monophosphate-guanosine-(2′->5′)-monophosphate) (c[A(2′,5′)pG(2′,5′)p]/2′2′-cGAMP/2′,5′-2′,5′-cGAMP); cyclic (guanosine-(2′->5′)-monophosphate-2′-deoxyadenosine-(3′->5′)-monophosphate) (c[G(2′,5′)p-2′-dA(3′,5′)p]); cyclic (guanosine-(2′->5′)-monophosphate-2′-O-(6-aminohexylcarbamoyl)adenosine-(3′->5′)-monophosphate) (c[G(2′,5′)p-2′-AHC-A(3′,5′)p]); cyclic (guanosine-(2′->5′)-monophosphate-2′-O-(6-[biotinyl]aminohexylcarbamoyl)adenosine-(3′->5′)-monophosphate) (c[G(2′,5′)p-2′-[Biotin]-AHC-A(3′,5′)p]); cyclic (guanosine-(2′->5′)-monophosphate-2′-O-(6-[fluoresceinyl]aminohexylcarbamoyl)adenosine-(3′->5′)-monophosphate) (c[G(2′,5′)p-2′-Fluo-AHC-A(3′,5′)p]); cyclic (guanosine-(2′->5′)-monophosphate-adenosine-(3′->5′)-monophosphate) (c[G(2′,5′)pA(3′,5′)p]/cGAMP(2′-5′)/2′3′-cGAMP/2′,5′-3′,5′-cGAMP); cyclic (guanosine-(2′->5′)-monophosphate-guanosine-(3′->5′)-monophosphate) (c[G(2′,5′)pG(3′,5′)p]/2′3′-c-diGMP/2′,5′-3′,5′-c-diGMP); cyclic (guanosine-(2′->5′)-monophosphorothioate-adenosine-(3′->5′)-monophosphorothioate) (c[G(2′,5′)pS-A(3′,5′)pS]/2′3′-cGAMPSS/2′3′-cGsAsMP); set of 2 isomer; cyclic diadenosine monophosphate (c-diAMP/c-di-AMP/cyclic bis (3′->5′) diadenylic acid); cyclic diadenosine monophosphorodithioate, Rp-/Sp-isomers (Rp,Sp-c-diAMPSS); cyclic diadenosine monophosphorodithioate, Rp-isomers (Rp,Rp-c-diAMPSS); cyclic diadenosine monophosphorothioate, Rp-isomer (Rp-c-diAMPS); cyclic diadenosine monophosphorothioate, Sp-isomer (Sp-c-diAMPS); cyclic diadenosine-(2′->5′)-monophosphate/cyclic bis (2′->5′) diadenylic acid (c[A(2′,5′)pA(2′,5′)p]/2′2′-c-diAMP/2′,5′-2′,5′-c-diAMP/c-diAMP(2′-5′)); cyclic diguanosine monophosphate (c-diGMP/c-di-GMP)/cyclic bis (3′->5′) diguanylic acid/cyclic diguanylate; cyclic diguanosine monophosphorodithioate, Rp-/Sp-isomers (Rp,Sp-c-diGMPSS); cyclic diguanosine monophosphorodithioate, Rp-isomers (Rp,Rp-c-diGMPSS); cyclic diguanosine monophosphorothioate, Rp-isomer (Rp-c-diGMPS); cyclic diguanosine monophosphorothioate, Sp-isomer (Sp-c-diGMPS); cyclic diguanosine-(2′->5′)-monophosphate/cyclic bis (2′->5′) diguanylic acid (c[G(2′,5′)pG(2′,5′)p]/2′2′-c-diGMP/2′,5′-2′,5′-c-diGMP/c-diGMP(2′-5′)); cyclic diinosine monophosphate (c-diIMP); 2′-Deoxy-2″-O-methyl-cyclic diadenosine monophosphate (c-di-2′-d-2″-O-Me-AMP/c-(2′-dAp-2′-O-Me-Ap)); 2′-Deoxy-cyclic diadenosine monophosphate (c-di-2′-dAMP/c-(2′-dApAp)); 2′-Deoxy-cyclic diguanosine monophosphate (c-di-2′-dGMP/c-(2′-dGpGp)/c-dG-GMP); Determination and quantification of c-diAMP and c-diGMP in biological systems; 2′, 2″-O-(Di-6-[biotinyl]aminohexylcarbamoyl)-cyclic diadenosine monophosphate (2′,2″-Di-[Biotin]-AHC-c-diAMP); 2′-, 2″-O-(Di-6-[biotinyl]aminohexylcarbamoyl)-cyclic diguanosine monophosphate (2′,2″-Di-[Biotin]-AHC-c-diGMP); 2′-, 2″-O-(Di-methyl)-cyclic diadenosine monophosphate (2′,2″-Di-O-Me-c-diAMP); 2′-, 2″-O-(Di-methyl)-cyclic diguanosine monophosphate (2′,2″-Di-O-Me-c-diGMP); 2′-, 2″-O-(Di-N′-methylanthraniloyl)-cyclic diadenosine monophosphate (Di-MANT-c-diAMP); 2′-, 2″-O-(Di-N′-methylanthraniloyl)-cyclic diguanosine monophosphate (Di-MANT-c-diGMP); 2′-, 2″-O-(Di-[6-aminohexylcarbamoyl])-cyclic diadenosine monophosphate (2′,2″-Di-AHC-c-diAMP); 2′-, 2″-O-(Di-[6-aminohexylcarbamoyl])-cyclic diguanosine monophosphate (2′,2″-Di-AHC-c-diGMP); 8-, 8′-Dibromo-cyclic diguanosine monophosphate (8,8′-Di-Br-c-diGMP); 8-, 8′-Dichloro-cyclic diadenosine monophosphate (8,8′-Di-Cl-c-diAMP); 2′-, 2″-Dideoxy-2′-, 2″-difluoro-cyclic diadenosine monophosphate (2′,2″-Di-F-c-didAMP); 2′-, 2″-Dideoxy-cyclic diadenosine monophosphate (2′,2″-Di-c-didAMP); 2′-, 2″-Dideoxy-cyclic diguanosine monophosphate (2′,2″-Di-c-didGMP); 2′-O-(6-[DY-547]-aminohexylcarbamoyl)-cyclic diguanosine monophosphate (2′-[DY-547]-AHC-c-diGMP/cdGMPDY547); 2′-O-(6-[Fluoresceinyl]aminohexylcarbamoyl)-cyclic diadenosine monophosphate (2′-Fluo-AHC-c-diAMP); 2′-O-(6-[Fluoresceinyl]aminohexylcarbamoyl)-cyclic diguanosine monophosphate (2′-Fluo-AHC-c-diGMP); 2′4)-Methyl-cyclic diadenosine monophosphate (2′-O-Me-c-diAMP/c-(2′-O-Me-ApAp)); 2′-O-Methyl-cyclic diguanosine monophosphate (2′-O-Me-c-diGMP/c-(2′-O-Me-GpGp)); 2′-O-(N′-Methylanthraniloyl)-cyclic diadenosine monophosphate (MANT-c-diAMP); 2′-O-(N′-Methylanthraniloyl)-cyclic diguanosine monophosphate (MANT-c-diGMP); 5′-Phosphoadenylyl-(3′->5′)-adenosine (pApA); and 5′-Phosphoguanylyl-(3′->5′)-guanosine (pGpG).

In a further preferred embodiment of the inventive composition the second adjuvant component of the inventive composition comprises at least one protein-based or peptide-based compound as immune potentiator compound. The protein-based or peptide-based compound may be administered as single adjuvant compound, or, especially preferred, in combination with further adjuvant compounds, especially in combination with a vitamin compound. Preferably the protein-based or peptide-based compound is selected from the following list comprising: CCR5 peptides, pRANTES (CCL5), Trp-Lys-Tyr-Met-Val-Met immunostimulatory peptide, IC31 (KLKL(5)KLK peptide vehicle plus ODN1a), Hiltonol (polylCLC—poly-IC with poly-lysine), albumin-heparin microparticles, β-glucan peptide (BGP), proteinoid microspheres (PODDS™), protein cochleates (stable protein phospholipid-calcium precipitates), e.g. BIORAL™, Murametide (N2-[N-(N-Acetylmuramoyl)-L-alanyl]-D-glutamine methyl ester), pCMVmCAT1 (plasmid expressing Friend murine leukemia virus receptor), protamine, and mRNA complexed with protamine (RNActive®). Further examples are antimicrobial peptides, RSV fusion protein, or adjuvants suitable as antagonists like CGRP neuropeptide. Moreover the protein-based or peptide-based compound may be compound based on a complex with cationic and/or oligocationic and/or polycationic component and nucleic acid molecules, preferably a complex with disulfide-crosslinked cationic component with nucleic acid molecules, e.g. peptide CR₁₂C plus isRNA or peptide CR₁₂ plus is RNA (RNAdjuvant®). For further details of this compound it is referred to the description above. Moreover the protein-based or peptide-based compound may be a metalloprotein compound, e.g. Keyhole limpet hemocyanin (KLH). Moreover the protein-based or peptide-based compound may be a heat shock protein (HSP) compound, e.g. HSP70 or Gp96. Moreover the protein-based or peptide-based compound may be a membrane protein compound, e.g. B7-2. Moreover the protein-based or peptide-based compound may be peptidoglycan compound, more preferably a muropeptide or derivative thereof, e.g. muramyl dipeptide (MDP—which may act as an agonist for NOD2 and NOD-like receptor 3), or Murapalmitine (Nac-Mur-L-Thr-D-isoGln-sn-glycerol dipalmitoyl), or Threonyl muramyl dipeptide (TMDP, Termurtide®, [thr1]-MDP, N-acetyl muramyl-L-threonyl-D-isoglutamine), or muramyl tripeptide, or muramyl tripeptide phosphatidylethanolamine (MTP-PE, (N-acetyl-L-alanyl-D-isoglutaminyl-L-alanine-2-(1,2-dipalmitoyl-sn-glycero-3-(hydroxyphosphoryloxy))-ethylamide, monosodium salt), or muramyl tetrapeptide, especially M-TriLYS-D-ASN, or romurtide (synthetic muramyl dipeptide derivative), or adamantylamide dipeptide, or adamantylamide l-alanyl-d-isoglutamine, or SAF (Synthex adjuvant formulation), or SAF-1 (threonyl-MDP in an emulsion vehicle), or Murametide (N2-[N-(N-Acetylmuramoyl)-L-alanyl]-D-glutamine methyl ester). Moreover the protein-based or peptide-based compound may be a bacterial protein-based compound, e.g. flagellin and flagellin fusion proteins. Moreover the protein-based or peptide-based compound may be a high mobility group protein (HMGB) compound, e.g. HMGB1, which may act as an endogenous immunomodulator. Moreover the protein-based or peptide-based compound may be a lipopeptide compound and/or a lipoprotein compound, e.g. P3C or Pam3Cys (tripalmitoyl-S-glyceryl cysteine). Preferably the second adjuvant component of the inventive composition comprises at least the polymeric carrier cargo complex as described above. Also combinations of the different compounds may be preferred.

According to a preferred embodiment, the second adjuvant component of the inventive combination or composition, preferably the immune potentiator and/or delivery system compound comprised therein, comprises a peptidoglycan, preferably as described herein, or a fragment or variant thereof. Preferably, the second adjuvant component of the inventive combination or composition, preferably the immune potentiator and/or delivery system compound comprised therein, comprises a ligand, more preferably an agonist, of a NOD receptor or a NOD-like receptor, such as a NOD1 receptor or a NOD2 receptor, or a fragment or variant thereof. Said NOD ligand or agonist is preferably a peptidglycan or a fragment or variant thereof. The ligand is preferably selected from the group consisting of C12-iE-DAP (acylated derivative of the dipeptide iE-DAP (γ-D-Glu-mDAP)); iE-DAP (γ-D-Glu-mDAP); iE-Lys (γ-D-Glu-Lys); Tri-DAP (L-Ala-γ-D-Glu-mDAP); Tri-Lys (L-Ala-γ-D-Glu-Lys); CL429 (Pam2C-conjugated murabutide); L18-MDP (muramyldipeptide with a C18 fatty acid chain); MDP (muramyldipeptide (L isoform)); M-TriLYS (synthetic muramyl tripeptide); Murabutide (synthetic derivative of muramyldipeptide); N-Glycolyl-MDP (N-glycolylated muramyldipeptide); M-TriDAP (MurNAc-L-Ala-gamma-D-Glu-m DAP-PGN-like molecule); PGN-ECndi; PGN-ECndss; and PGN-SAndi.

More preferably, the second adjuvant component of the inventive combination or composition, preferably the immune potentiator and/or delivery system compound comprised therein, comprises a ligand, preferably an agonist, of a NOD1 receptor, or a fragment or variant of said ligand. Therein, the ligand is preferably selected from the group consisting of C12-iE-DAP (acylated derivative of the dipeptide iE-DAP (γ-D-Glu-mDAP)); iE-DAP (γ-D-Glu-mDAP); iE-Lys (γ-D-Glu-Lys); Tri-DAP (L-Ala-γ-D-Glu-mDAP); and Tri-Lys (L-Ala-γ-D-Glu-Lys). Most preferably the ligand of a NOD1 receptor as used herein is Tri-DAP.

Alternatively, the second adjuvant component of the inventive combination or composition, preferably the immune potentiator and/or delivery system compound comprised therein, comprises a ligand, preferably an agonist, of a NOD2 receptor, or a fragment or variant of said ligand. Therein, the ligand is preferably selected from the group consisting of CL429 (Pam2C-conjugated murabutide); L18-MDP (muramyldipeptide with a C18 fatty acid chain); MDP (muramyldipeptide (L isoform)); M-TriLYS (synthetic muramyl tripeptide); Murabutide (synthetic derivative of muramyldipeptide); and N-Glycolyl-MDP (N-glycolylated muramyldipeptide). Most preferably the ligand of a NOD2 receptor as used herein is Murabutide.

According to a further embodiment, the second adjuvant component of the inventive combination or composition, preferably the immune potentiator and/or delivery system compound comprised therein, comprises a ligand, preferably an agonist, of a NOD1 receptor and of a NOD2 receptor, or a fragment or variant of said ligand. Therein, the ligand is preferably selected from the group consisting of M-TriDAP (MurNAc-L-Ala-gamma-D-Glu-mDAP-PGN-like molecule); PGN-ECndi; PGN-ECndss; and PGN-SAndi.

In a further preferred embodiment of the inventive composition the second adjuvant component of the inventive composition comprises at least one hydrocarbon-based or carbohydrate-based compound as immune potentiator compound. The hydrocarbon-based or carbohydrate-based compound may be administered as single adjuvant compound, or, especially preferred, in combination with further adjuvant compounds, especially in combination with a vitamin compound. For example the hydrocarbon-based or carbohydrate-based compound may be GMDP (N-acetylglucosaminyl-(β1-4)-N-acetylmuramyl-L-alanyl-D-isoglutamine), or p-Hydroxybenzoique acid methyl ester, or BAK (benzalkonium chloride), or Mannose, or LNFPIII/Lewis X (glycan based adjuvant). Moreover the hydrocarbon-based or carbohydrate-based compound may be a polysaccharide-based compound, e.g. β-glucan peptide (BGP—which may act as an agonist for C-type lectin receptors like Dectin-1), or β-glucan e.g. PLEURAN™, or glucans from algae, or dextran, or inulin, or γ-inulin, or delta inulin polysaccharide, or Algammulin. Moreover the hydrocarbon-based or carbohydrate-based compound may be a polyaminosaccharide-based compound, more preferably a Chitin-derived compound, e.g. chitosan. Moreover the hydrocarbon-based or carbohydrate-based compound may be a glycoside-based compound, more preferably a saponin (triterpene glycoside) or derivative thereof, e.g. Quil-A, or QS-21 (e.g. STIMULON™), or AS01 (MPL plus liposome plus QS-21), or AS02 (MF59® plus MPL plus QS-21), or AS15 (MPL plus CpG plus QS-21 plus liposome), or immuno-stimulatory complexes (ISCOMs), or ISCOMATRIX® (cholesterol plus phospholipid plus saponin), or Abisco-100, or Iscoprep 7.0.3.®, or Quadri A saponin, or GPI0100, or GPI anchor, or Matrix M, or POSintro. Moreover the hydrocarbon-based or carbohydrate-based compound may be an imidazoquinoline compound, preferably an Imiquimods compound, e.g. R-837 (Imiquimod—1-(2-methylpropyl)-1H-imidazol[4,5-c]quinoline-4-amine), or R-848 (Resiquimod), or 3M-012, or S-28463 (4-amino-2-ethoxymethyl-alpha, alpha-dimethyl-1H-imidazo[4, 5-c]quinoline-1-ethanol). Moreover the hydrocarbon-based or carbohydrate-based compound may be a glycolide compound, e.g. DL-PGL (polyester poly (DL-lactide-co-glycolide)), or PLG (polyactide coglycolide), or homo- and co-polymers of lactic and glycolic acid (PLGA, PGA, PLA, e.g. in form of microspheres/nanospheres). Moreover the hydrocarbon-based or carbohydrate-based compound may be an amide-based compound, e.g. Bupivacaine ((RS)-1-Butyl-N-(2,6-dimethylphenyl)piperidine-2-carboxamide). Also combinations of the different compounds may be preferred.

In a further preferred embodiment of the inventive composition the second adjuvant component of the inventive composition comprises at least one lipid-based compound as immune potentiator compound. The lipid-based compound may be administered as single adjuvant compound, or, especially preferred, in combination with further adjuvant compounds, especially in combination with a vitamin compound. Preferably the lipid-based compound is selected from the following list comprising: Arlacel A (dianhydromannitol monooleate), Span 85 (Arlacel 85, sorbitan trioleate), DMPC (Dimyristoyl phosphatidy-1-choline), DMPG (Dimyristoyl phosphatidylglycerol), Murapalmitine (Nac-Mur-D-Ala-D-isoGln-sn-glycerol dipalmitoyl), N-acetylglucosaminyl-N-acetyhnuramyl-L-Ala-D-isoGlu-L-Ala-glycerol dipalmitate (DTP-GDP, disaccharide tripeptide glycerol dipalmitoyl, e.g. ImmTher™), Theramide® (N-acetylglucosaminyl-N-acetylinuramyl-L-Ala-D-isoGlu-L-Ala-dipalmitoxy propylamide, DTP-DPP), stearyl tyrosine, ISCOMATRIX® (cholesterol plus phospholipid plus saponin), DDA (dimethy-1-dioctadecylammonium bromide or chloride), Gerbu Adjuvant (mixture of: i) N-Acetylglucosaminyl-(PI-4)-N-acetylmuramyl-L-alanyl-D-glutamine (GMDP), ii) Dimethyl dioctadecylammonium chloride (DDA), iii) Zinc L-proline salt complex (ZnPro-8)) and Vaxfectin™ (cationic lipid-based formulation). Moreover the lipid-based compound may be a glycolipid compound, more preferably a trehalose dimycolate or derivative thereof, e.g. trehalose-6,6′-dimycolate (TDM), or trehalose-6,6′-dibehenate (TDB), or BAY R1005 (N-(2-Deoxy-2-L-leucylamino-β-D-glucopyranosyl)-N-octadecyldodecanoylamide hydroacetate). Moreover the lipid-based compound may be a lipopolysaccharide compound and/or a lipopolysaccharide derivative compound, more preferably bacterial lipopolysaccharide (LPS). Moreover the lipid-based compound may be a Lipid A compound, e.g. monophosphoryl lipid A (MPL—which may act as an agonist for Toll-like receptor 4, e.g. 3-Q-desacyl-4′-monophosphoryl lipid A), or MPL-SE (MPL stable emulsion), or AS04 (MPL puls Alum), or AS01 (MPL plus liposome plus QS-21), or AS02 (MF59® plus MPL plus QS-21), or AS15 (MPL plus CpG plus QS-21 plus liposome), or DETOX (MPL plus mycobacterial cell-wall skeleton), or glucopyranosil lipid A (GLA), or Walter Reed liposomes (liposomes containing lipid A adsorbed to aluminium hydroxid), or RC529 (2-[(R)-3-tetradecanoyloxytetradecanoylamino]ethyl 2-deoxy-4-O-phosphono-3-O-[(R)-3-tetradecanoyoxytetradecanoyl]-2-[(R)-3-tetradecanoyoxytetradecanoylamino]-β-D-glucopyranos idetriethylammonium salt). Moreover the lipid-based compound may be a lipoidal amine compound, e.g. Avridine® (N,N-dioctadecyl-N′,N′-bis(2-hydroxyethyl) propanediamine). Also combinations of the different compounds may be preferred.

According to a preferred embodiment, the second adjuvant component of the inventive combination or composition, preferably the immune potentiator and/or delivery system compound comprised therein, comprises a lipid nanoparticle. The lipid nanoparticle as used herein preferably comprises one or more cationic lipids and a poly(ethyleneglycol)-lipid (PEG-lipid). Therein, the cationic lipid is preferably an ionizable cationic lipid, more preferably an asymmetric ionizable cationic lipid, even more preferably an asymmetric ionizable amino lipid.

According to a preferred embodiment, the second adjuvant component of the inventive combination or composition, preferably the immune potentiator and/or delivery system compound comprised therein, comprises a lipid nanoparticle comprising a cationic lipid selected from the group consisting of DLinDMA; DlinKC2DMA; DLin-MC3-DMA; CLinDMA; S-Octyl CLinDMA; (2S)-1-{7-[(3β)-cholest-5-en-3-yloxy]heptyloxy}-3-[(4Z)-dec-4-en-1-yloxy]N,N-dimethylpropan-2-amine; (2R)-1-{4-[(3β)-cholest-5-en-3-yloxy]butoxy}-3-[(4Z)dec-4-en-1-yloxy]-N,N-dimethylpropan-2-amine; 1-[(2R)-1-{4-[(3β)-cholest-5-en-3-yloxy]butoxy}-3-(octyloxy)propan-2-yl]guanidine; 1-[(2R)-1-{7-[(3β)-cholest-5-en-3-yloxy]heptyloxy}-N, N-dimethyl-3-[(9Z,12Z)-octadeca-9,12-dien-1-yloxy]propan-2-amine; 1-[(2R)-1-{4-[(3β)-cholest-5-en-3-yloxy]butoxy}-N,N-dimethyl-3-[(9Z,12Z)-octadeca-9,12-dien-1-yloxy]propan-2-amine; (2S)-1-({6-[(3β))-cholest-5-en-3-yloxy]hexyl}oxy)-N,N-dimethyl-3-[(9Z)-octadec-9-en-1-yloxy]propan-2-amine; (3β)-3-[6-{[(2S)-3-[(9Z)-octadec-9-en-1-yloxyl]-2-(pyrrolidin-1-yl)propyl]oxy}hexyl)oxy]cholest-5-ene; (2R)-1-{4-[(3β)cholest-5-en-3-yloxy]butoxy}-3-(octyloxy)propan-2-amine; (2R)-1-({8-[(3β)cholest-5-en-3-yloxy]octyl}oxy)-N,N-dimethyl-3-(pentyloxy)propan-2-amine; (2R)-1-({8-[(3β)-cholest-5-en-3-yloxy]octyl}oxy)-3-(heptyloxy)-N, N-dimethyl propan-2-amine; (2R)-1-({8-[(3β)cholest-5-en-3-yloxy]octyl}oxy)-N, N-dimethyl-3-[(2Z)-pent-2-en-1-yloxy]propan-2-amine; (2S)-1-butoxy-3-({8-[(3β)-cholest-5-en-3-yloxy]octyl}oxy)-N,N-dimethylpropan-2-amine; (2S-1-({8-[(3β)-cholest-5-en-3-yloxy]octyl}oxy)-3-[2,2,3,3,4,4,5,5,6,6,7,7,8,8,9,9-hexadecafluorononyl)oxy]-N,N-dimethylpropan-2-amine; 2-amino-2-{[(9Z,12Z)-octadeca-9,12-dien-1-yloxy]methyl}propane-1,3-diol; 2-amino-3-({9-[(3β,8ξ,9ξ,14ξ,17ξ,20ξ)-cholest-5-en-3-yloxy]nonyl}oxy)-2-{[(9Z,12Z)-octadeca-9,12-dien-1-yloxy]methyl}propan-1-ol; 2-amino-3-({6-[(3β,8ξ, 9ξ,14ξ,17ξ,20ξ-cholest-5-en-3-yloxy]hexyl}oxy)-2-{[(9Z)-octadec-9-en-1-yloxy]methyl}propan-1-ol; (20Z,23Z)-N,N-dimethylnonacosa-20,23-dien-10-amine; (17Z,20Z)-N,N-dimethylhexacosa-17,20-dien-9-amine; (16Z,19Z)-N,N-dimethylpentacosa-16,19-dien-8-amine; (13Z,16Z)-N,N-dimethyldocosa-13,16-dien-5-amine; (12Z,15Z)-N,N-dimethylhenicosa-12,15-dien-4-amine; (14Z,17Z)-N,N-dimethyltricosa-14,17-dien-6-amine; (15Z,18Z)-N,N-dimethyltetracosa-15,18-dien-7-amine; (18Z,21Z)-N,N-dimethylheptacosa-18,21-dien-10-amine; (15Z,18Z)-N,N-dimethyltetracosa-15,18-dien-5-amine; (14Z,17Z)-N,N-dimethyltricosa-14,17-dien-4-amine; (19Z,22Z)-N,N-dimethyloctacosa-19,22-dien-9-amine; (18Z,21Z)-N,N-dimethylheptacosa-18,21-dien-8-amine; (17Z,20Z)-N,Ndimethylhexacosa-17,20-dien-7-amine; (16Z,19Z)-N,N-dimethylpentacosa-16,19-dien-6-amine; (22Z,25Z)-N,N-dimethylhentriaconta-22,25-dien-10-amine; (21Z,24Z)-N,Ndimethyltriaconta-21,24-dien-9-amine; (18Z)-N,N-dimethylheptacos-18-en-10-amine; (17Z)N,N-dimethylhexacos-17-en-9-amine; (19Z,22Z)-N,N-dimethyloctacosa-19,22-dien-7-amine; N,N-dimethylheptacosan-10-amine; (20Z,23Z)-N-ethyl-N-methylnonacosa-20,23-dien-10-amine; 1-[(11Z,14Z)-1-nonylicosa-1,1,14-dien-1-yl]pyrrolidine; (20Z)-N,Ndimethylheptacos-20-en-10-amine; (15Z)-N,N-dimethylheptacos-15-en-10-amine; (14Z)-N,N-dimethylnonacos-14-en-10-amine; (17Z)-N,N-dimethylnonacos-17-en-10-amine; (24Z)N,N-dimethyltritriacont-24-en-10-amine; (20Z)-N,N-dimethylnonacos-20-en-10-amine; (22Z)-N,N-dimethylhentriacont-22-en-10-amine; (16Z)-N, N-dimethylpentacos-16-en-8-amine; (12Z,15Z)-N,N-dimethyl-2-nonylhenicosa-12,15-dien-1-amine; (13Z,16Z)-N,N-dimethyl-3-nonyldocosa-13,16-dien-1-amine; N,N-dimethyl-1-[(1S,2R)-2-octylcyclopropyl]heptadecan-8-amine; 1-[(1S,2R)-2-hexylcyclopropyl]-N,N-dimethylnonadecan-10-amine; N, N-dimethyl-1-[(1S,2R)-2-octylcyclopropyl]nonadecan-10-amine; N,N-dimethyl-21-[(1S,2R)-2-octylcyclopropyl]henicosan-10-amine; N,N-dimethyl-1-[(1S,2S)-2-{[(1R,2R)-2-pentylcyclopropyl]methyl}cyclopropyl]nonadecan-10-amine; N,N-dimethyl-1-[(1S,2R)-2-octylcyclopropyl]hexadecan-8-amine; N,N-dimethyl-1-[(1R,2S)-2-undecylcyclopropyl]tetradecan-5-amine; N,N-dimethyl-3-{7-[(1S,2R)-2-octylcyclopropyl]heptyl}dodecan-1-amine; 1-[(1R,2S)-2-heptylcyclopropyl]-N,Ndimethyloctadecan-9-amine; 1-[(1S,2R)-2-decylcyclopropyl]-N,N-dimethyl pentadecan-6-amine; N,N-dimethyl-1-[(1S,2R)-2-octylcyclopropyl]pentadecan-8-amine; and (11E,20Z,23Z)-N,N-dimethylnonacosa-11,20,23-trien-10-amine; or a pharmaceutically acceptable salt thereof, or a stereoisomer of any of the recited compounds or salts, or any combination thereof.

According to a preferred embodiment, the lipid nanoparticle as comprised in the second adjuvant component of the inventive combination or composition, preferably the immune potentiator and/or delivery system compound comprised therein, comprises a lipid, preferably an amino lipid, as described above, distearoylphosphatidylcholine (DSPC), cholesterol and poly(ethyleneglycol) (PEG), preferably polyethyleneglycol)2000-dimyristoylglycerol (PEG2000-DMG). Most preferably, the lipid nanoparticle as used herein comprises a lipid, preferably an amino lipid, as described above, distearoylphosphatidylcholine (DSPC), cholesterol and poly(ethyleneglycol) (PEG), preferably polyethyleneglycol)2000-dimyristoylglycerol (PEG2000-DMG), in a molar ratio of 58:30:10:2.

Further preferred lipid nanoparticles as comprised in the second adjuvant component of the inventive combination or composition, preferably the immune potentiator and/or delivery system compound comprised therein, are disclosed in the international patent application WO 2015/130584 or in Swaminathan et al. 2016 (G. Swaminathan et al.: A novel lipid nanoparticle adjuvant significantly enhances B cell and T cell responses to sub-unit vaccine antigens. Vaccine 34: 110-119; 2016).

In a further preferred embodiment of the inventive composition the second adjuvant component of the inventive composition comprises at least one polymeric compound as immune potentiator compound. The polymeric compound may be administered as single adjuvant compound, or, especially preferred, in combination with further adjuvant compounds, especially in combination with a vitamin compound. For example the polymeric compound may be POLYGEN® Vaccine Adjuvant. Other examples are copolymers like Optivax (CRL1005), L121 or Poloaxmer4010 or biopolymers or polyethylene carbamate derivatives. Moreover the polymeric compound may be an anorganic-organic polymer compound, e.g. polyphosphazene. Moreover the polymeric compound may be a polyacrylic compound, e.g. polymethylmethacrylate (PMMA), or Carbopol 934P. Also combinations of the different compounds may be preferred.

In a further preferred embodiment of the inventive composition the second adjuvant component of the inventive composition comprises at least one cytokine or at least one hormone compound, preferably a chemokine compound and/or an interferon compound and/or tumor necrosis factor (TNF) compound and/or an adhesion molecule compound and/or a steroid compound, or at least one enzyme or at least one cell compound as immune potentiator compound. One or more of these compounds may be administered as single adjuvant compound, or, especially preferred, in combination with further adjuvant compounds, especially in combination with a vitamin compound. The cytokine compound may be e.g. GM-CSF, or Flt-3 ligand, or ligands of human TLR1-10, or ligands of murine TLR1-13, or UC-1V150, or Ampligen™. The interleukin compound may be e.g. IL-1, IL-1β, IL-2, IL-4, IL-6, IL-7, IL-10, IL-12, IL-15, IL-18 etc., or Sclavo peptide (IL-1β 163-171 peptide), or IL-2 in pcDNA3, or IL-2/Ig plasmid, or IL-4 in pcDNA3, or IL-10 plasmid, or hIL-12 (N222L), or IL-12 DNA, or IL-12 plasmid, or IL-12/GM-CSF plasmid, or rAd5-hIL-12N222L, or IL-15 plasmid, or rAd5-IL15. The interferon compound may be e.g. lymphotactin, or RANTES, or defensins. The tumor necrosis factor (TNF) compound may be e.g. TNFα, or CD40 ligand. The adhesion molecule compound may be e.g. ICAM-1, or LAF-3. The steroid compound may be e.g. Dehydroepiandrosterone (DHEA). The enzyme compound may be e.g. Neuraminidase-galactose oxidase (NAGO). The cell compound may be e.g. dendritic cells, or PBMC (peripheral blood mononuclear cells). Also combinations of the different compounds may be preferred.

In a further preferred embodiment of the inventive composition the second adjuvant component of the inventive composition comprises at least one toxin compound as immune potentiator compound. The toxin compound may be administered as single adjuvant compound, or, especially preferred, in combination with further adjuvant compounds, especially in combination with a vitamin compound. Preferably the toxin compound is a viral toxin compound and/or a viral toxin derivative compound. In an especially preferred embodiment the toxin compound is based on bacterial toxins or bacterial toxin derivatives. Preferably the toxin compound is selected from the following list comprising: cholera toxin (CT), cholera holotoxin, mCT-E112K, cholera toxin B subunit (CTB), cholera toxin A1-subunit-Protein A D-fragment fusion protein, CTA1-DD gene fusion protein, chimeric A1 subunit of cholera toxin (CTA1)-DD, E. coli heat-labile enterotoxin (LT), LT(R192G), LTK63, LTK72, LT-R192G, LT B subunit, LT-OA (E. coli labile enterotoxin protoxin), LT 5 oral adjuvant (E. coli labile enterotoxin-protoxin), Bordetella pertussis component Vaccine Adjuvant, Corynebacterium-derived P40, killed Corynebacterium parvum vaccine adjuvant, Diphtheria toxoid, and Tetanus toxoid (TT). Generally, microbe derived adjuvants may be used. Moreover, plant derived adjuvants like Tomatine adjuvant (glycoalkaloid) may be used. Also combinations of the different compounds may be preferred.

In a further preferred embodiment of the inventive composition the second adjuvant component of the inventive composition comprises at least one vehicle compound as delivery system compound. The vehicle compound may be administered as single adjuvant compound, or, especially preferred, in combination with further adjuvant compounds, especially in combination with a vitamin compound. Preferably the vehicle compound is a liposome compound, e.g. cationic liposomal vaccine adjuvant, or Stealth liposomes, or JVRS-100 (cationic liposomal DNA complex), or cytokine-containing liposomes, or immunoliposomes containing antibodies to costimulatory molecules, or DRVs (immunoliposomes prepared from dehydration-rehydration vesicles), or MTP-PE liposomes, or Sendai proteoliposomes, or Sendai containing lipid matrices, or Walter Reed liposomes (liposomes containing lipid A adsorbed to aluminium hydroxid), or CAF01 (liposomes plus DDA plus TDB), or AS01 (MPL plus liposome plus QS-21), or AS15 (MPL plus CpG plus QS-21 plus liposome). Moreover the vehicle compound may be formed by a virosome compound (unilamellar liposomal vehicles incorporating virus derived proteins, such as influenza haemagglutinin), e.g. IRIVs (immunopotentiating reconstituted influenza virosomes), or liposomes of lipids plus hemagglutinin. Moreover the vehicle compound may be formed by a virus-like particle (VLP) compound, e.g. Ty particles (Ty-VLPs). Moreover the vehicle compound may be formed by microparticles and/or nanoparticles, e.g. polymeric microparticles (PLG), or cationic microparticles, or albumin-heparin microparticles, or CRL1005 (block copolymer P1205), or peptomere nanoparticle, or CAP™ (calcium phosphate nanoparticles), or microspheres, or PODDS® (proteinoid microspheres), or nanospheres. Moreover, the vehicle compound may be formed by a protein cochleate compound, especially by stable protein phospholipid-calcium precipitates, e.g. BIORAL™ Also combinations of the different compounds may be preferred.

In a further preferred embodiment of the inventive composition the second adjuvant component of the inventive composition comprises at least one mineral salts compound as delivery system compound. The mineral salts compound may be administered as single adjuvant compound, or, especially preferred, in combination with further adjuvant compounds, especially in combination with a vitamin compound. Preferably the mineral salts compound is an aluminium compound, e.g. aluminium hydroxide, or aluminium phosphate, or Alum (aluminium hydroxide gel, aluminium hydroxide gel suspension), or high protein adsorbency aluminium hydroxide gel (HPA), or low viscosity aluminium hydroxide gel (LV), or DOC (deoxycholic acid sodium salt)/Alum complex, or aluminium phosphate gel, or aluminium potassium sulfate, or aluminium salts, such as Adju-phos, Alhydrogel or Rehydragel, or amorphous aluminium hydroxyphosphate sulfate. Moreover the mineral salts compound may be based on a calcium compound, e.g. calcium phosphate gel. Also combinations of the different compounds may be preferred.

Further examples of mineral salt compounds as used herein include the salts of iron and zirconium. In a preferred embodiment, the mineral salt compound as used herein is a phosphate salt of aluminium, calcium, iron or zirconium.

In a particularly preferred embodiment of the inventive combination or composition, the second adjuvant component of the inventive combination or composition, preferably the immune potentiator and/or delivery system compound comprised therein, comprises at least one aluminium compound or a calcium compound, more preferably an aluminium salt or a calcium salt, even more preferably an aluminium phosphate salt or a calcium phosphate salt.

In a preferred embodiment, the second adjuvant component of the inventive combination or composition, preferably the immune potentiator and/or delivery system compound comprised therein, comprises an aluminium compound selected from the group consisting of aluminium phosphate, aluminium hydroxide, alum or an adjuvant compound based on any of these. The at least one aluminum compound can take any suitable physical form, but is preferably amorphous.

According to a preferred embodiment, the second adjuvant component of the inventive combination or composition, preferably the immune potentiator and/or delivery system compound comprised therein, comprises aluminium phosphate or a compound based thereon.

The term “aluminum phosphate” as used herein typically comprises aluminium phosphate strictu sensu (Al(PO₄)) as well as aluminum hydroxyphosphates. In the context of the present invention, an aluminium phosphate compound may optionally contain a small amount of sulfate (e.g. aluminum hydroxyphosphate sulfate). Aluminium phosphate is preferably obtained by precipitation, and the reaction conditions and concentrations during precipitation influence the degree of substitution of phosphate for hydroxyl in the salt. Hydroxyphosphates preferably have a PO₄/Al molar ratio between 0.3 and 1.2. Hydroxyphosphates may be distinguished from AlPO₄ strictu sensu by the presence of hydroxyl groups. For example, an IR spectrum band at 3164 cm⁻¹ (e.g. when heated to 200° C.) may indicate the presence of hydroxyl groups.

The PO₄/Al³⁺ molar ratio of an aluminum phosphate as used herein is preferably in a range from 0.3 to 1.2, more preferably in a range from 0.8 to 1.2, and even more preferably 0.95±0.1. The aluminum phosphate is preferably amorphous, particularly for hydroxyphosphate salts. For example, an amorphous aluminum hydroxyphosphate with PO₄/Al molar ratio in a range from 0.84 to 0.92. The aluminum phosphate is preferably particulate. Typical diameters of the particles are preferably in the range from 0.5 to 20 μm (e.g. about 5 to 10 μm).

According to a particularly preferred embodiment, the second adjuvant component of the inventive combination or composition, preferably the immune potentiator and/or delivery system compound comprised therein, comprises aluminium hydroxyphosphate, preferably amorphous aluminium hydroxyphosphate. Even more preferably, the second adjuvant component of the inventive combination or composition, preferably the immune potentiator and/or delivery system compound comprised therein, comprises Adju-Phos.

Alternatively, the second adjuvant component of the inventive combination or composition, preferably the immune potentiator and/or delivery system compound comprised therein, comprises aluminium hydroxide or a compound based thereon.

The term “aluminum hydroxide” as used herein comprises aluminium hydroxide strictu sensu (Al(OH)₃) as well as aluminum oxyhydroxide (AlO(OH)). Preferably, the aluminium hydroxide used herein is an aluminium salt, which is preferably at least partially crystalline. Aluminium oxyhydroxide may preferably be distinguished from other aluminium compounds, such as aluminium hydroxide, by infrared (IR) spectroscopy, in particular by the presence of an adsorption band at 1070 cm⁻¹ and a shoulder at 3090 to 3100 cm⁻¹.

According to a particularly preferred embodiment, the second adjuvant component of the inventive combination or composition, preferably the immune potentiator and/or delivery system compound comprised therein, comprises an aluminium hydroxide gel, preferably a sterilized aluminium hydroxide wet gel suspension. Preferably, the second adjuvant component of the inventive combination or composition, preferably the immune potentiator and/or delivery system compound comprised therein, comprises Alhydrogel.

In preferred embodiments of the inventive composition the second adjuvant component of the inventive composition may be selected from any of the classes (1) mineral salts, e.g., aluminium hydroxide and aluminium or calcium phosphate gels; (2) emulsions including: oil emulsions and surfactant based formulations, e.g., microfluidised detergent stabilised oil-in-water emulsion, purified saponin, oil-in-water emulsion, stabilised water-in-oil emulsion; (3) particulate adjuvants, e.g., virosomes (unilamellar liposomal vehicles incorporating viral protein, such as influenza haemagglutinin), structured complex of saponins and lipids, polylactide co-glycolide (PLG); (4) microbial derivatives; (5) endogenous human immunomodulators; and/or (6) inert vehicles, such as gold particles; (7) microorganism derived adjuvants; (8) tensoactive compounds; (9) carbohydrates; or combinations thereof.

In preferred embodiments of the inventive composition the second adjuvant component of the inventive composition may comprise at least one compound selected from the list consisting of: 3′3′-cGAMP, 2′2′-cGAMP, 1018 ISS, CpG 7909, CpG 1018, AS15 (MPL plus CpG plus QS-21 plus liposome), synthetic dsRNA, especially polyionisinic:polycytidylic acid (Poly(I:C)), Hiltonol (polylCLC—poly-IC with poly-lysine), poly-adenylic acid-poly-uridylic acid complex (Poly rA:Poly rU), 5′ppp-dsRNA, viral dsRNA, IC31 (KLKL(5)KLK peptide vehicle plus ODN1a), pCMVmCAT1 (plasmid expressing Friend murine leukemia virus receptor), guanosine-rich ssRNA, uridine-rich ssRNA, CR₁₂C plus isRNA, CR₁₂ plus isRNA, Loxoribine (7-allyl-8-oxoguanosine), IMOxine™, CCR5 peptides, pRANTES (CCL5), Trp-Lys-Tyr-Met-Val-Met immunostimulatory peptide, albumin-heparin microparticles, β-glucan peptide (BGP), PODDS® (proteinoid microspheres), stable protein phospholipid-calcium precipitates, pCMVmCAT1 (plasmid expressing Friend murine leukemia virus receptor), PAMPs (Pathogen-associated molecular patterns), protamine, mRNA complexed with protamine, antimicrobial peptides, RSV fusion protein, CGRP neuropeptide, Keyhole limpet hemocyanin (KLH), HSP70, Gp96, B7-2, muramyl dipeptide (MDP), Murapalmitine (Nac-Mur-L-Thr-D-isoGln-sn-glycerol dipalmitoyl), Threonyl muramyl dipeptide (TMDP; Termurtide®; [thr1]-MDP; N-acetyl muramyl-L-threonyl-D-isoglutamine), muramyl tripeptide, muramyl tripeptide phosphatidylethanolamine (MTP-PE, (N-acetyl-L-alanyl-D-isoglutaminyl-L-alanine-2-(1,2-dipalmitoyl-sn-glycero-3-(hydroxyphosphoryloxy))-ethylamide, monosodium salt), muramyl tetrapeptide, especially M-TriLYS-D-ASN, romurtide (synthetic muramyl dipeptide derivative), adamantylamide dipeptide, adamantylamide l-alanyl-d-isoglutamine, SAF (Syntex adjuvant formulation), SAF-1 (threonyl-MDP in an emulsion vehicle), flagellin and flagellin fusion proteins (flagellin may act as an agonist for Toll-like receptor 5 and NOD-like receptors), HMGB1, P3C, Pam3Cys (tripalmitoyl-S-glyceryl cysteine), GMDP (N-acetylglucosaminyl-(β1-4)-N-acetylmuramyl-L-alanyl-D-isoglutamine), p-Hydroxybenzoique acid methyl ester, BAK (benzalkonium chloride), Mannose, LNFPIII/Lewis X (glycan based adjuvant), β-glucan, glucans from algae, dextran, or inulin, γ-inulin, delta inulin polysaccharide, Algammulin, chitosan, Quil-A, QS-21 (e.g. STIMULON™), AS01 (MPL plus liposome plus QS-21), AS02 (MF59® plus MPL plus QS-21), immuno-stimulatory complexes (ISCOMs), ISCOMATRIX® (cholesterol plus phospholipid plus saponin), Abisco-100, Iscoprep 7.0.3.®, Quadri A saponin, GPI0100, GPI anchor, Matrix M, POSintro, R-837 (Imiquimod—1-(2-methylpropyl)-1H-imidazol[4,5-c]quinoline-4-amine), R-848 (Resiquimod), 3M-012, S-28463 (4-amino-2-ethoxymethyl-alpha, alpha-dimethyl-1H-imidazo[4,5-c]quinoline-1-ethanol), DL-PGL (polyester poly (DL-lactide-co-glycolide)), PLG (polyactide coglycolide), PLGA plus PGA plus PLA (homo- and co-polymers of lactic and glycolic acid, e.g. in form of microspheres/nanospheres), Bupivacaine ((RS)-1-Butyl-N-(2,6-dimethylphenyl)piperidine-2-carboxamide), Arlacel A (dianhydromannitol monooleate), Span 85 (Arlacel 85, sorbitan trioleate), DMPC (Dimyristoyl phosphatidy-1-choline), DMPG (Dimyristoyl phosphatidylglycerol), N-acetylglucosaminyl-N-acetyhnuramyl-L-Ala-D-isoGlu-L-Ala-glycerol dipalmitate (DTP-GDP, disaccharide tripeptide glycerol dipalmitoyl, e.g. ImmTher™), Theramide® (N-acetylglucosaminyl-N-acetylinuramyl-L-Ala-D-isoGlu-L-Ala-dipalmitoxy propylamide; DTP-DPP), stearyl tyrosine, DDA (dimethy-1-dioctadecylammonium bromide or chloride), Gerbu Adjuvant (mixture of: i)N-Acetylglucosaminyl-(P1-4)-N-acetylmuramyl-L-alanyl-D-glutamine (GMDP), ii) Dimethyl dioctadecylammonium chloride (DDA), iii) Zinc L-proline saltcomplex (ZnPro-8)), Vaxfectin™ (cationic lipid-based formulation), trehalose-6,6′-dimycolate (TDM), trehalose-6,6′-dibehenate (TDB), BAY R1005 (N-(2-Deoxy-2-L-leucylamino-β-D-glucopyranosyl)-N-octadecyldodecanoyl-amide hydroacetate), monophosphoryl lipid A (MPL, e.g. 3-Q-desacyl-4′-monophosphoryl lipid A), MPL-SE (MPL stable emulsion), AS04 (MPL puls Alum), DETOX (MPL plus mycobacterial cell-wall skeleton), glucopyranosil lipid A (GLA), RC529 (2-[(R)-3-tetradecanoyloxytetradecanoylamino]ethyl 2-deoxy-4-O-phosphono-3-O-[(R)-3-tetradecanoyoxytetradecanoyl]-2-[(R)-3-tetradecanoyoxytetradecanoylamino]-β-D-glucopyranos idetriethylammonium salt), Avridine® (N, N-dioctadecyl-N′, N′-bis (2-hydroxyethyl) propanediamine), POLYGEN® Vaccine Adjuvant, copolymers like Optivax (CRL1005), L121 or Poloaxmer4010, biopolymers, polyethylene carbamate derivatives, polyphosphazene, polymethylmethacrylate (PMMA), Carbopol 934P, retinoic acid, esp. all-trans retinoic acid (ATRA), retinyl palmitate, retinol ester, retinol, retinal, tretinoin, Retin-A, isotretinoin, alitretinoin, etretinate, acitretin, tazarotene, bexarotene, Adapalene (polyaromatisches retinoid), tocopherol, AS03 (Squalene plus Tween 80 plus α-tocopherol), vitamin D3, Calcitrol (25-dihydroxycholecalciferol), IL-1, IL-113, IL-2, IL-4, IL-6, IL-7, IL-10, IL-12, IL-15, IL-18, Sclavo peptide (IL-1β 163-171 peptide), IL-2 in pcDNA3, IL-2/Ig plasmid, IL-4 in pcDNA3, IL-10 plasmid, hIL-12 (N222L), IL-12 DNA, IL-12 plasmid, IL-12/GM-CSF plasmid, rAd5-hIL-12N222L, IL-15 plasmid, rAd5-IL15, GM-CSF, Flt-3 ligand, ligands of human TLR1-10, ligands of murine TLR1-13, UC-1V150, Ampligen™, lymphotactin, RANTES, defensins, IFN-α, IFN-γ, IFN-γ in pCDNA3, recombinant hIFN-γ, TNFα, CD40 ligand, ICAM-1, LAF-3, Dehydroepiandrosterone (DHEA), Neuraminidase-galactose oxidase (NAGO), dendritic cells, PBMC (peripheral blood mononuclear cells), cholera toxin (CT), cholera holotoxin, mCT-E112K, cholera toxin B subunit (CTB), cholera toxin A1-subunit-ProteinA D-fragment fusion protein, CTA1-DD gene fusion protein, chimeric A1 subunit of cholera toxin (CTA1)-DD, E. coli heat-labile enterotoxin (LT), LT(R192G), LTK63, LTK72, LT-R192G, LT B subunit, LT-OA (E. coli labile enterotoxin protoxin), LT 5 oral adjuvant (E. coli labile enterotoxin-protoxin), Bordetella pertussis component Vaccine Adjuvant, Corynebacterium-derived P40, killed Corynebacterium parvum vaccine adjuvant, Diphtheria toxoid, Tetanus toxoid (TT), microbe derived adjuvants, plant derived adjuvants, Tomatine adjuvant, cationic liposomal vaccine adjuvant, Stealth liposomes, JVRS-100 (cationic liposomal DNA complex), cytokine-containing liposomes, immunoliposomes containing antibodies to costimulatory molecules, DRVs (immunoliposomes prepared from dehydration-rehydration vesicles), MTP-PE liposomes, Sendai proteoliposomes, Sendai containing lipid matrices, Walter Reed liposomes (liposomes containing lipid A adsorbed to aluminium hydroxid), CAF01 (liposomes plus DDA plus TDB), liposomes (lipids plus hemagglutinin), IRIVs (immunopotentiating reconstituted influenza virosomes), virosomes (unilamellar liposomal vehicles incorporating viral protein, such as influenza haemagglutinin), Ty particles (Ty-VLPs), polymeric microparticles (PLG), cationic microparticles, CRL1005 (block copolymer P1205), peptomere nanoparticle, CAP™ (calcium phosphate nanoparticles), microspheres, nanospheres, stable protein phospholipid-calcium precipitates, e.g. BIORAL™, non-ionic surfactant vesicles (NISV), VSA-3 adjuvant, AddaVax®, MF59® (Squalene plus Tween 80 plus Span 85), AF03 (Squalene plus Montane 80 (emulsifier) plus Eumulgin B1 PH (emulsifier)), nanoemulsion, RIBI (bacterial and mycobacterial cell wall components), Ribi529, Ribilike adjuvant system (MPL, TMD, CWS), Murametide (N2-[N-(N-Acetylmuramoyl)-L-alanyl]-D-glutamine methyl ester), incomplete Freund's adjuvant (IFA), complete Freund's adjuvant (CFA), Specol (Marcol 52 (mineral oil, paraffins, and cycloparaffins, chain length 13-22 C atoms) and Span 85 and Tween 85), squalene, Montanide® (squalene and squalane) ISA51, Montanide® ISA720, SPT (squalane (5%), Tween 80 (0.2%), Pluronic L121(1.25%)), Squalane 1 (Spinacane; Robane®; 2,6,10,15,19,23-hexamethyltetracosane and 2,6,10,15,19,23-hexamethyl-2,6,10,14,18,22-tetracosahexane), Squalene 2 (Spinacene; Supraene; 2,6,10,15,19,23-hexamethyl-2,6,10,14,18,22 tetracosahexane), TiterMax Gold Adjuvant, pluronics, Pluronic L121 (Poloxamer 401), Polysorbate 80 (Tween 80), aluminium hydroxide, aluminium phosphate, Alum (aluminium hydroxide gel, aluminium hydroxide gel suspension), high protein adsorbency aluminium hydroxide gel (HPA), low viscosity aluminium hydroxide gel (LV), DOC (deoxycholic acid sodium salt)/Alum complex, aluminium phosphate gel, aluminium potassium sulfate, aluminium salts like Adju-phos or Alhydrogel or Rehydragel, amorphous aluminium hydroxyphosphate sulfate, calcium phosphate gel, AF, Provax, and PMM.

Adjuvants for nucleic acid vaccines (DNA) have been disclosed in, for example, Kobiyama, et al., Vaccines, 2013, 1(3), 278-292, the contents of which are incorporated herein by reference in their entirety. Any of the adjuvants disclosed by Kobiyama may be used as the second adjuvant component of the inventive composition.

Other adjuvants which may be utilized as the second adjuvant component of the inventive composition include any of those listed on the web-based vaccine adjuvant database, http://www.violinet.org/vaxjo/ and described in for example Sayers, et al, J. Biomedicine and Biotechnology, volume 2012 (2012), Article ID 831486, 13 pages, the content of which is incorporated herein by reference in its entirety.

Specific adjuvants may include cationic liposome-DNA complex JVRS-100, aluminum hydroxide vaccine adjuvant, aluminum phosphate vaccine adjuvant, aluminum potassium sulfate adjuvant, alhydrogel, ISCOM(s)™, Freund's Complete Adjuvant, Freund's Incomplete Adjuvant, CpG DNA Vaccine Adjuvant, Cholera toxin, Cholera toxin B subunit, Liposomes, Saponin Vaccine Adjuvant, DDA Adjuvant, Squalene-based Adjuvants, Etx B subunit Adjuvant, IL-12 Vaccine Adjuvant, LTK63 Vaccine Mutant Adjuvant, TiterMax Gold Adjuvant, Ribi Vaccine Adjuvant, Montanide ISA 720 Adjuvant, Corynebacterium-denved P40 Vaccine Adjuvant, MPL™ Adjuvant, AS04, AS02, Lipopolysaccharide Vaccine Adjuvant, Muramyl Dipeptide Adjuvant, CRL1005, Killed Corynebacterium parvum Vaccine Adjuvant, Montanide ISA 51, Bordetella pertussis component Vaccine Adjuvant, Cationic Liposomal Vaccine Adjuvant, Adamantylamide Dipeptide Vaccine Adjuvant, Arlacel A, VSA-3 Adjuvant, Aluminum vaccine adjuvant, Polygen Vaccine Adjuvant, ADJUMER™, Algal Glucan, Bay R1005, Theramide®, Stearyl Tyrosine, Specol, Algammulin, AVRIDINE®, Calcium Phosphate Gel, CTA1-DD gene fusion protein, DOC/Alum Complex, Gamma Inulin, Gerbu Adjuvant, GM-CSF, GMDP, Recombinant hIFN-gamma/Interferon-g, Interleukin- [symbol] β, Inter leukin-2, Interleukin-7, Sclavo peptide, Rehydragel LV, Rehydragel HP A, Loxoribine, MF59, MTP-PE Liposomes, Murametide, Murapalmitine, D-Murapalmitine, NAGO, Non-Ionic Surfactant Vesicles, PMMA, Protein Cochleates, QS-21, SPT (Antigen Formulation), nanoemulsion vaccine adjuvant, AS03, Quil-A vaccine adjuvant, RC529 vaccine adjuvant, LTR192G Vaccine Adjuvant, E. coli heat-labile toxin, LT, amorphous aluminum hydroxyphosphate sulfate adjuvant, Calcium phosphate vaccine adjuvant, Montanide Incomplete Seppic Adjuvant, Imiquimod, Resiquimod, AF03, Flagellin, Poly(LC), ISCOMATRIX®, Abisco-100 vaccine adjuvant, Albumin-heparin microparticles vaccine adjuvant, AS-2 vaccine adjuvant, B7-2 vaccine adjuvant, DHEA vaccine adjuvant, Immunoliposomes Containing Antibodies to Costimulatory Molecules, SAF-1, Sendai Proteo liposomes, Sendai-containing Lipid Matrices, Threonyl muramyl dipeptide (TMDP), Ty Particles vaccine adjuvant, Bupivacaine vaccine adjuvant, DL-PGL (Polyester poly (DL-lactide-co-glycolide)) vaccine adjuvant, IL-15 vaccine adjuvant, LTK72 vaccine adjuvant, MPL-SE vaccine adjuvant, non-toxic mutant E112K of Cholera Toxin mCT-EI 12K, and/or Matrix-S.

In especially preferred embodiments of the invention the second adjuvant component comprises two or more different adjuvant components. For example the different adjuvant components are a vitamin compound, especially a vitamin A compound or a vitamin A derivative compound and a polymeric carrier cargo complex as described above.

In another preferred embodiment, the combination of the composition according to the invention comprises a second (adjuvant) component, which comprises a mineral salt adjuvant as described herein, preferably an aluminium salt, more preferably an aluminium phosphate salt, such as Adju-Phos, and another adjuvant compound as described herein, preferably a polymeric carrier cargo complex as described herein.

The nucleic acid molecule of the first immunogenic component of the inventive composition, namely the nucleic acid molecule encoding at least one epitope of at least one antigen, may be any DNA or RNA as defined herein. Preferably, such a coding DNA or RNA may be a single- or a double-stranded DNA or RNA, more preferably a single-stranded DNA or RNA, and/or a circular or linear DNA or RNA, more preferably a linear DNA or RNA. Furthermore such a coding DNA or RNA may be a genomic DNA, a viral RNA or DNA, a replicon, a plasmid DNA or an mRNA. Even more preferably, the coding DNA or RNA may be a (linear) single-stranded DNA or RNA. Most preferably, the nucleic acid molecule according to the present invention may be a linear single-stranded messenger RNA (mRNA). Such an mRNA may occur as a mono-, di-, or even multicistronic RNA, i.e. an RNA which carries the coding sequences of one, two or more proteins or peptides representing at least one epitope of at least one antigen. A monocistronic mRNA may typically be an mRNA, that encodes only one open reading frame. An open reading frame in this context is a sequence of several nucleotide triplets (codons) that can be translated into a peptide or protein. A di- or multicistronic mRNA typically may have two (dicistronic) or more (multicistronic) open reading frames (ORF). Translation of such an mRNA yields two (dicistronic) or more (multicistronic) distinct translation products (provided the ORFs are not identical). For expression in eukaryotes such mRNAs may for example comprise an internal ribosomal entry site (IRES) sequence. That means such coding sequences in di-, or even multicistronic mRNA may be separated by at least one IRES sequence, e.g. as defined herein.

In this context, the terms ‘open reading frame’ or ‘ORF’, ‘coding region’ and ‘coding sequence’ are typically used interchangeably.

According to an especially preferred embodiment of the invention, the mRNA of the inventive composition is modified. Preferably the mRNA is stabilized by modifying and increasing the G (guanosine)/C (cytosine) content of the mRNA of the coding region thereof. Therein, the G/C content of the mRNA of the coding region is increased compared to the G/C content of the coding region of its particular wild type coding sequence, i.e. the unmodified mRNA. However, the encoded amino acid sequence of the mRNA is preferably not modified compared to the encoded amino acid sequence of the particular wild type/unmodified mRNA.

The modification of the G/C-content of the mRNA of the inventive composition is based on the fact that RNA sequences having an increased G (guanosine)/C (cytosine) content are more stable than RNA sequences having an increased A (adenosine)/U (uracil) content. The codons of a coding sequence or a whole RNA might therefore be varied compared to the wild type coding sequence or mRNA, such that they include an increased amount of G/C nucleotides while the translated amino acid sequence is retained. In respect to the fact that several codons code for one and the same amino acid (so-called degeneration of the genetic code), the most favourable codons for the stability can be determined (so-called alternative codon usage). Preferably, the G/C content of the coding region of the mRNA according to the invention is increased by at least 7%, more preferably by at least 15%, particularly preferably by at least 20%, compared to the G/C content of the coding region of the wild type RNA. According to a specific embodiment at least 5%, 10%, 20%, 30%, 40%, 50%, 60%, more preferably at least 70%, even more preferably at least 80% and most preferably at least 90%, 95% or even 100% of the substitutable codons in the region coding for a protein or peptide as defined herein or its fragment or variant thereof or the whole sequence of the wild type mRNA sequence or coding sequence are substituted, thereby increasing the G/C content of said sequence. In this context, it is particularly preferable to increase the G/C content of the mRNA of the inventive composition to the maximum (i.e. 100% of the substitutable codons), in particular in the coding region, compared to the wild type sequence.

The coding region of the mRNA sequence of the inventive composition may occur as a mono-, di-, or even multicistronic mRNA, i.e. an mRNA sequence which carries the coding sequences of one, two or more proteins or peptides which have the function of at least one epitope of at least one antigen. Such coding sequences of the di-, or even multicistronic mRNAs may be separated by at least one internal ribosome entry site (IRES) sequence. Thus, the mRNA according to the invention may further comprise one or more internal ribosome entry site (IRES) sequences or IRES-motifs, which may separate several open reading frames, especially if the mRNA encodes for two or more peptides or proteins (bi- or multicistronic mRNA). For example, the internal ribosome entry site sequence may be derived from EMCV (encephalomyocarditis virus) or from FMDV (Foot and mouth disease virus). Furthermore signal peptides may be used which induce the cleavage of the resulting polypeptide which comprises several proteins or peptides, e.g. a signal peptide sequence derived from F2A peptide from FMDV.

By a further embodiment, the mRNA of the inventive composition preferably comprises at least one of the following structural elements: a 5′- and/or 3′-untranslated region element (UTR element), particularly a 5′-UTR element which comprises or consists of a nucleic acid sequence which is derived from the 5′-UTR of a TOP gene or from a fragment, homolog or a variant thereof, or a 5′- and/or 3′-UTR element which may be derivable from a gene that provides a stable mRNA or from a homolog, fragment or variant thereof; a histone stem-loop structure, preferably a histone stem-loop in its 3′ untranslated region; a 5′-CAP structure; a poly-A tail (poly(A) sequence); or a poly(C) sequence as will be outlined in more detail below.

In a preferred embodiment the mRNA comprises at least one 5′- or 3′-UTR element. In this context an UTR element comprises or consists of a nucleic acid sequence which is derived from the 5′- or 3′-UTR of any naturally occurring gene or which is derived from a fragment, a homolog or a variant of the 5′- or 3′-UTR of a gene. Preferably the 5′- or 3′-UTR element used according to the present invention is heterologous to the coding region of the mRNA sequence of the inventive composition. Even if 5′- or 3′-UTR elements derived from naturally occurring genes are preferred, also synthetically engineered UTR elements may be used in the context of the present invention.

In a particularly preferred embodiment the mRNA sequence comprises at least one 5′-untranslated region element (5′-UTR element) which comprises or consists of a nucleic acid sequence which is derived from the 5′-UTR of a TOP gene or which is derived from a fragment, homolog or variant of the 5′-UTR of a TOP gene, wherein it is particularly preferred that the 5′-UTR element does not comprise a TOP-motif or a 5′-TOP, as defined above.

In some embodiments, the nucleic acid sequence of the 5′-UTR element which is derived from a 5′-UTR of a TOP gene terminates at its 3′-end with a nucleotide located at position 1, 2, 3, 4, 5, 6, 7, 8, 9 or 10 upstream of the start codon (e.g. A(U/T)G) of the gene or mRNA it is derived from. Thus, the 5′-UTR element does not comprise any part of the protein coding region. Thus, preferably, the only protein coding part of mRNA of the inventive composition is provided by the coding region.

The nucleic acid sequence which is derived from the 5′-UTR of a TOP gene is preferably derived from a eukaryotic TOP gene, preferably a plant or animal TOP gene, more preferably a chordate TOP gene, even more preferably a vertebrate TOP gene, most preferably a mammalian TOP gene, such as a human TOP gene.

For example, the 5′-UTR element is preferably selected from 5′-UTR elements comprising or consisting of a nucleic acid sequence which is derived from a nucleic acid sequence selected from the group consisting of SEQ ID Nos. 1-1363, SEQ ID NO. 1395, SEQ ID NO. 1421 and SEQ ID NO. 1422 of the patent application WO2013/143700, whose disclosure is incorporated herein by reference, from the homologs of SEQ ID Nos. 1-1363, SEQ ID NO. 1395, SEQ ID NO. 1421 and SEQ ID NO. 1422 of the patent application WO2013/143700, from a variant thereof, or preferably from a corresponding RNA sequence. The term “homologs of SEQ ID Nos. 1-1363, SEQ ID NO. 1395, SEQ ID NO. 1421 and SEQ ID NO. 1422 of the patent application WO2013/143700” refers to sequences of other species than homo sapiens, which are homologous to the sequences according to SEQ ID Nos. 1-1363, SEQ ID NO. 1395, SEQ ID NO. 1421 and SEQ ID NO. 1422 of the patent application WO2013/143700.

In a preferred embodiment, the 5′-UTR element comprises or consists of a nucleic acid sequence which is derived from a nucleic acid sequence extending from nucleotide position 5 (i.e. the nucleotide that is located at position 5 in the sequence) to the nucleotide position immediately 5′ to the start codon (located at the 3′ end of the sequences), e.g. the nucleotide position immediately 5′ to the ATG sequence, of a nucleic acid sequence selected from SEQ ID Nos. 1-1363, SEQ ID NO. 1395, SEQ ID NO. 1421 and SEQ ID NO. 1422 of the patent application WO2013/143700, from the homologs of SEQ ID Nos. 1-1363, SEQ ID NO. 1395, SEQ ID NO. 1421 and SEQ ID NO. 1422 of the patent application WO2013/143700 from a variant thereof, or a corresponding RNA sequence. It is particularly preferred that the 5′-UTR element is derived from a nucleic acid sequence extending from the nucleotide position immediately 3′ to the 5′-TOP to the nucleotide position immediately 5′ to the start codon (located at the 3′ end of the sequences), e.g. the nucleotide position immediately 5′ to the ATG sequence, of a nucleic acid sequence selected from SEQ ID Nos. 1-1363, SEQ ID NO. 1395, SEQ ID NO. 1421 and SEQ ID NO. 1422 of the patent application WO2013/143700, from the homologs of SEQ ID Nos. 1-1363, SEQ ID NO. 1395, SEQ ID NO. 1421 and SEQ ID NO. 1422 of the patent application WO2013/143700, from a variant thereof, or a corresponding RNA sequence.

In a particularly preferred embodiment, the 5′-UTR element comprises or consists of a nucleic acid sequence which is derived from a 5′-UTR of a TOP gene encoding a ribosomal protein or from a variant of a 5′-UTR of a TOP gene encoding a ribosomal protein. For example, the 5′-UTR element comprises or consists of a nucleic acid sequence which is derived from a 5′-UTR of a nucleic acid sequence according to any of SEQ ID NOs: 67, 170, 193, 244, 259, 554, 650, 675, 700, 721, 913, 1016, 1063, 1120, 1138, and 1284-1360 of the patent application WO2013/143700, a corresponding RNA sequence, a homolog thereof, or a variant thereof as described herein, preferably lacking the 5′-TOP motif. As described above, the sequence extending from position 5 to the nucleotide immediately 5′ to the ATG (which is located at the 3′end of the sequences) corresponds to the 5′-UTR of said sequences.

Preferably, the 5′-UTR element comprises or consists of a nucleic acid sequence which is derived from a 5′-UTR of a TOP gene encoding a ribosomal large protein (RPL) or from a homolog or variant of a 5′-UTR of a TOP gene encoding a ribosomal large protein (RPL). For example, the 5′-UTR element comprises or consists of a nucleic acid sequence which is derived from a 5′-UTR of a nucleic acid sequence according to any of SEQ ID NOs: 67, 259, 1284-1318, 1344, 1346, 1348-1354, 1357, 1358, 1421 and 1422 of the patent application WO2013/143700, a corresponding RNA sequence, a homolog thereof, or a variant thereof as described herein, preferably lacking the 5′-TOP motif.

In a particularly preferred embodiment, the 5′-UTR element comprises or consists of a nucleic acid sequence which is derived from the 5′-UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, or from a variant of the 5′-UTR of a ribosomal protein Large 32 gene, preferably from a vertebrate ribosomal protein Large 32 (L32) gene, more preferably from a mammalian ribosomal protein Large 32 (L32) gene, most preferably from a human ribosomal protein Large 32 (L32) gene, wherein preferably the 5′-UTR element does not comprise the 5′-TOP of said gene.

A preferred sequence for a 5′-UTR element corresponds to SEQ ID No. 1368 of the patent application WO2013/143700.

Accordingly, in a particularly preferred embodiment, the 5′-UTR element comprises or consists of a nucleic acid sequence which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence as mentioned above, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

In some embodiments, the mRNA of the inventive composition comprises a 5′-UTR element which comprises or consists of a nucleic acid sequence which is derived from the 5′-UTR of a vertebrate TOP gene, such as a mammalian, e.g. a human TOP gene, selected from RPSA, RPS2, RPS3, RPS3A, RPS4, RPS5, RPS6, RPS7, RPS8, RPS9, RPS10, RPS11, RPS12, RPS13, RPS14, RPS15, RPS15A, RPS16, RPS17, RPS18, RPS19, RPS20, RPS21, RPS23, RPS24, RPS25, RPS26, RPS27, RPS27A, RPS28, RPS29, RPS30, RPL3, RPL4, RPL5, RPL6, RPL7, RPL7A, RPL8, RPL9, RPL10, RPL10A, RPL11, RPL12, RPL13, RPL13A, RPL14, RPL15, RPL17, RPL18, RPL18A, RPL19, RPL21, RPL22, RPL23, RPL23A, RPL24, RPL26, RPL27, RPL27A, RPL28, RPL29, RPL30, RPL31, RPL32, RPL34, RPL35, RPL35A, RPL36, RPL36A, RPL37, RPL37A, RPL38, RPL39, RPL40, RPL41, RPLP0, RPLP1, RPLP2, RPLP3, RPLP0, RPLP1, RPLP2, EEF1A1, EEF1B2, EEF1D, EEF1G, EEF2, EIF3E, EIF3F, EIF3H, EIF2S3, EIF3C, EIF3K, EIF3EIP, EIF4A2, PABPC1, HNRNPA1, TPT1, TUBB1, UBA52, NPM1, ATP5G2, GNB2L1, NME2, UQCRB, or from a homolog or variant thereof, wherein preferably the 5′-UTR element does not comprise a TOP-motif or the 5′-TOP of said genes, and wherein optionally the 5′-UTR element starts at its 5′-end with a nucleotide located at position 1, 2, 3, 4, 5, 6, 7, 8, 9 or 10 downstream of the 5′ terminal oligopyrimidine tract (TOP) and wherein further optionally the 5′-UTR element which is derived from a 5′-UTR of a TOP gene terminates at its 3′-end with a nucleotide located at position 1, 2, 3, 4, 5, 6, 7, 8, 9 or 10 upstream of the start codon (A(U/T)G) of the gene it is derived from.

In further particularly preferred embodiments, the 5′-UTR element comprises or consists of a nucleic acid sequence which is derived from the 5′-UTR of a ribosomal protein Large 32 gene (RPL32), a ribosomal protein Large 35 gene (RPL35), a ribosomal protein Large 21 gene (RPL21), an ATP synthase, H+ transporting, mitochondrial F1 complex, alpha subunit 1, cardiac muscle (ATP5A1) gene, an hydroxysteroid (17-beta) dehydrogenase 4 gene (HSD17B4), an androgen-induced 1 gene (AIG1), cytochrome c oxidase subunit VIc gene (COX6C), or a N-acylsphingosine amidohydrolase (acid ceramidase) 1 gene (ASAH1) or from a variant thereof, preferably from a vertebrate ribosomal protein Large 32 gene (RPL32), a vertebrate ribosomal protein Large 35 gene (RPL35), a vertebrate ribosomal protein Large 21 gene (RPL21), a vertebrate ATP synthase, H+ transporting, mitochondrial F1 complex, alpha subunit 1, cardiac muscle (ATP5A1) gene, a vertebrate hydroxysteroid (17-beta) dehydrogenase 4 gene (HSD17B4), a vertebrate androgen-induced 1 gene (AIG1), a vertebrate cytochrome c oxidase subunit VIc gene (COX6C), or a vertebrate N-acylsphingosine amidohydrolase (acid ceramidase) 1 gene (ASAH1) or from a variant thereof, more preferably from a mammalian ribosomal protein Large 32 gene (RPL32), a ribosomal protein Large 35 gene (RPL35), a ribosomal protein Large 21 gene (RPL21), a mammalian ATP synthase, H+ transporting, mitochondrial F1 complex, alpha subunit 1, cardiac muscle (ATP5A1) gene, a mammalian hydroxysteroid (17-beta) dehydrogenase 4 gene (HSD17B4), a mammalian androgen-induced 1 gene (AIG1), a mammalian cytochrome c oxidase subunit VIc gene (COX6C), or a mammalian N-acylsphingosine amidohydrolase (acid ceramidase) 1 gene (ASAH1) or from a variant thereof, most preferably from a human ribosomal protein Large 32 gene (RPL32), a human ribosomal protein Large 35 gene (RPL35), a human ribosomal protein Large 21 gene (RPL21), a human ATP synthase, H+ transporting, mitochondrial F1 complex, alpha subunit 1, cardiac muscle (ATP5A1) gene, a human hydroxysteroid (17-beta) dehydrogenase 4 gene (HSD17B4), a human androgen-induced 1 gene (AIG1), a human cytochrome c oxidase subunit VIc gene (COX6C), or a human N-acylsphingosine amidohydrolase (acid ceramidase) 1 gene (ASAH1) or from a variant thereof, wherein preferably the 5′-UTR element does not comprise the 5′-TOP of said gene.

Accordingly, in a particularly preferred embodiment, the 5′-UTR element comprises or consists of a nucleic acid sequence which has an identity of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID No. 1368, or SEQ ID NOs 1412-1420 of the patent application WO2013/143700, or a corresponding RNA sequence, or wherein the at least one 5′-UTR element comprises or consists of a fragment of a nucleic acid sequence which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according to SEQ ID No. 1368, or SEQ ID NOs 1412-1420 of the patent application WO2013/143700, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

Accordingly, in a particularly preferred embodiment, the 5′-UTR element comprises or consists of a nucleic acid sequence which has an identity of at least about 20%, preferably of at least about 40%, preferably of at least about 50%, preferably of at least about 60%, preferably of at least about 70%, more preferably of at least about 80%, more preferably of at least about 90%, even more preferably of at least about 95%, even more preferably of at least about 99% to the nucleic acid sequence according SEQ ID No. 1414 of the patent application WO2013/143700 (5′-UTR of ATP5A1 lacking the 5′ terminal oligopyrimidine tract) or preferably to a corresponding RNA sequence, wherein, preferably, the fragment is as described above, i.e. being a continuous stretch of nucleotides representing at least 20% etc. of the full-length 5′-UTR. Preferably, the fragment exhibits a length of at least about 20 nucleotides or more, preferably of at least about 30 nucleotides or more, more preferably of at least about 40 nucleotides or more. Preferably, the fragment is a functional fragment as described herein.

In a further preferred embodiment, the mRNA of the inventive composition further comprises at least one 3′-UTR element which comprises or consists of a nucleic acid sequence derived from the 3′-UTR of a chordate gene, preferably a vertebrate gene, more preferably a mammalian gene, most preferably a human gene, or from a variant of the 3′-UTR of a chordate gene, preferably a vertebrate gene, more preferably a mammalian gene, most preferably a human gene.

The term ‘3′-UTR element’ refers to a nucleic acid sequence which comprises or consists of a nucleic acid sequence that is derived from a 3′-UTR or from a variant of a 3′-UTR. A 3′-UTR element in the sense of the present invention may represent the 3′-UTR of an mRNA. Thus, in the sense of the present invention, preferably, a 3′-UTR element may be the 3′-UTR of an mRNA, preferably of an artificial mRNA, or it may be the transcription template for a 3′-UTR of an mRNA. Thus, a 3′-UTR element preferably is a nucleic acid sequence which corresponds to the 3′-UTR of an mRNA, preferably to the 3′-UTR of an artificial mRNA, such as an mRNA obtained by transcription of a genetically engineered vector construct. Preferably, the 3′-UTR element fulfils the function of a 3′-UTR or encodes a sequence which fulfils the function of a 3′-UTR.

Preferably, the mRNA comprises a 3′-UTR element which may be derivable from a gene that relates to an mRNA with an enhanced half-life (that provides a stable mRNA), for example a 3′-UTR element as defined and described below.

In a particularly preferred embodiment, the 3′-UTR element comprises or consists of a nucleic acid sequence which is derived from a 3′-UTR of a gene selected from the group consisting of an albumin gene, an α-globin gene, a β-globin gene, a tyrosine hydroxylase gene, a lipoxygenase gene, and a collagen alpha gene, such as a collagen alpha 1(I) gene, or from a variant of a 3′-UTR of a gene selected from the group consisting of an albumin gene, an α-globin gene, a β-globin gene, a tyrosine hydroxylase gene, a lipoxygenase gene, and a collagen alpha gene, such as a collagen alpha 1(I) gene according to SEQ ID No. 1369-1390 of the patent application WO2013/143700 whose disclosure is incorporated herein by reference. In a particularly preferred embodiment, the 3′-UTR element comprises or consists of a nucleic acid sequence which is derived from a 3′-UTR of an albumin gene, preferably a vertebrate albumin gene, more preferably a mammalian albumin gene, most preferably a human albumin gene according SEQ ID No: 1369 of the patent application WO2013/143700. The mRNA sequence may comprise or consist of a nucleic acid sequence which is derived from the 3′-UTR of the human albumin gene according to GenBank Accession number NM_000477.5, or from a fragment or variant thereof.

In this context it is particularly preferred that the mRNA of the inventive composition comprises a 3′-UTR element comprising a corresponding RNA sequence derived from the nucleic acids according to SEQ ID No. 1369-1390 of the patent application WO2013/143700 or a fragment, homolog or variant thereof.

Most preferably the 3′-UTR element comprises the nucleic acid sequence derived from a fragment of the human albumin gene according to SEQ ID No: 1376 of the patent application WO2013/143700.

In another particularly preferred embodiment, the 3′-UTR element comprises or consists of a nucleic acid sequence which is derived from a 3′-UTR of an α-globin or β-globin gene, preferably a vertebrate α- or β-globin gene, more preferably a mammalian α- or 3-globin gene, most preferably a human α- or β-globin gene according to SEQ ID No. 1370 of the patent application WO2013/143700 (3′-UTR of Homo sapiens hemoglobin, alpha 1 (HBA1)), or according to SEQ ID No. 1371 of the patent application WO2013/143700 (3′-UTR of Homo sapiens hemoglobin, alpha 2 (HBA2)), or according to SEQ ID No. 1372 of the patent application WO2013/143700 (3′-UTR of Homo sapiens hemoglobin, beta (HBB)).

For example, the 3′-UTR element may comprise or consist of the center, α-complex-binding portion of the 3′-UTR of an α-globin gene, corresponding to SEQ ID No. 1393 of the patent application WO2013/143700.

In this context it is particularly preferred that the 3′-UTR element of the mRNA of the inventive composition comprises or consists of a corresponding RNA sequence of the nucleic acid sequence according to the above or a homolog, a fragment or variant thereof.

The term ‘a nucleic acid sequence which is derived from the 3′-UTR of a [ . . . ] gene’ preferably refers to a nucleic acid sequence which is based on the 3′-UTR sequence of a [ . . . ] gene or on a part thereof, such as on the 3′-UTR of an albumin gene, an α-globin gene, a β-globin gene, a tyrosine hydroxylase gene, a lipoxygenase gene, or a collagen alpha gene, such as a collagen alpha 1(I) gene, preferably of an albumin gene or on a part thereof. This term includes sequences corresponding to the entire 3′-UTR sequence, i.e. the full length 3′-UTR sequence of a gene, and sequences corresponding to a fragment of the 3′-UTR sequence of a gene, such as an albumin gene, α-globin gene, β-globin gene, tyrosine hydroxylase gene, lipoxygenase gene, or collagen alpha gene, such as a collagen alpha 1(1) gene, preferably of an albumin gene.

The term ‘a nucleic acid sequence which is derived from a variant of the 3’-UTR of a [ . . . ] gene′ preferably refers to a nucleic acid sequence which is based on a variant of the 3′-UTR sequence of a gene, such as on a variant of the 3′-UTR of an albumin gene, an α-globin gene, a β-globin gene, a tyrosine hydroxylase gene, a lipoxygenase gene, or a collagen alpha gene, such as a collagen alpha 1(I) gene, or on a part thereof as described above. This term includes sequences corresponding to the entire sequence of the variant of the 3′-UTR of a gene, i.e. the full length variant 3′-UTR sequence of a gene, and sequences corresponding to a fragment of the variant 3′-UTR sequence of a gene. A fragment in this context preferably consists of a continuous stretch of nucleotides corresponding to a continuous stretch of nucleotides in the full-length variant 3′-UTR, which represents at least 20%, preferably at least 30%, more preferably at least 40%, more preferably at least 50%, even more preferably at least 60%, even more preferably at least 70%, even more preferably at least 80%, and most preferably at least 90% of the full-length variant 3′-UTR. Such a fragment of a variant, in the sense of the present invention, is preferably a functional fragment of a variant as described herein.

Preferably, the at least one 5′-UTR element and the at least one 3′-UTR element act synergistically to increase protein production from the mRNA of the inventive composition as described above.

In a particularly preferred embodiment, the mRNA of the inventive composition comprises a histone stem-loop sequence/structure. Such histone stem-loop sequences are preferably selected from histone stem-loop sequences as disclosed in WO2012/019780, whose disclosure is incorporated herewith by reference.

A histone stem-loop sequence, suitable to be used within the present invention, is preferably selected from at least one of the following formulae (I) or (II):

(stem-loop sequence without stem bordering elements): formula (I)

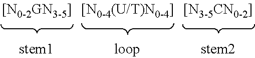

(stem-loop sequence with stem bordering elements): formula (II)

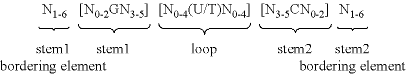

wherein:
stem1 or stem2 bordering elements N₁₋₆ is a consecutive sequence of 1 to 6, preferably of 2 to 6, more preferably of 2 to 5, even more preferably of 3 to 5, most preferably of 4 to 5 or 5 N, wherein each N is independently from another selected from a nucleotide selected from A, U, T, G and C, or a nucleotide analogue thereof;
stem1 [N₀₋₂GN₃₋₅] is reverse complementary or partially reverse complementary with element stem2, and is a consecutive sequence between of 5 to 7 nucleotides;
   wherein N₀₋₂ is a consecutive sequence of 0 to 2, preferably of 0 to 1, more preferably of 1 N, wherein each N is independently from another selected from a nucleotide selected from A, U, T, G and C or a nucleotide analogue thereof;
   wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof, and
   wherein G is guanosine or an analogue thereof, and may be optionally replaced by a cytidine or an analogue thereof, provided that its complementary nucleotide cytidine in stem2 is replaced by guanosine;
loop sequence [N₀₋₄(U/T)N₀₋₄] is located between elements stem1 and stem2, and is a consecutive sequence of 3 to 5 nucleotides, more preferably of 4 nucleotides;
   wherein each N₀₋₄ is independent from another a consecutive sequence of 0 to 4, preferably of 1 to 3, more preferably of 1 to 2 N, wherein each N is independently from another selected from a nucleotide selected from A, U, T, G and C or a nucleotide analogue thereof; and
   wherein U/T represents uridine, or optionally thymidine;
stem2 [N₃₋₅CN₀₋₂] is reverse complementary or partially reverse complementary with element stem1, and is a consecutive sequence between of 5 to 7 nucleotides;
   wherein N₃₋₅ is a consecutive sequence of 3 to 5, preferably of 4 to 5, more preferably of 4 N, wherein each N is independently from another selected from a nucleotide selected from A, U, T, G and C or a nucleotide analogue thereof;
   wherein N₀₋₂ is a consecutive sequence of 0 to 2, preferably of 0 to 1, more preferably of 1 N, wherein each N is independently from another selected from a nucleotide selected from A, U, T, G or C or a nucleotide analogue thereof; and
   wherein C is cytidine or an analogue thereof, and may be optionally replaced by a guanosine or an analogue thereof provided that its complementary nucleoside guanosine in stem1 is replaced by cytidine;
      wherein stem1 and stem2 are capable of base pairing with each other forming a reverse complementary sequence, wherein base pairing may occur between stem1 and stem2, e.g. by Watson-Crick base pairing of nucleotides A and U/T or G and C or by non-Watson-Crick base pairing e.g. wobble base pairing, reverse Watson-Crick base pairing, Hoogsteen base pairing, reverse Hoogsteen base pairing or are capable of base pairing with each other forming a partially reverse complementary sequence, wherein an incomplete base pairing may occur between stem1 and stem2, on the basis that one or more bases in one stem do not have a complementary base in the reverse complementary sequence of the other stem.

According to a further preferred embodiment of the first inventive aspect, the inventive mRNA sequence may comprise at least one histone stem-loop sequence according to at least one of the following specific formulae (Ia) or (IIa):

(stem-loop sequence without stem bordering elements): formula (Ia)

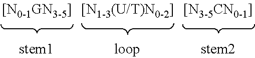

(stem-loop sequence with stem bordering elements): formula (IIa)

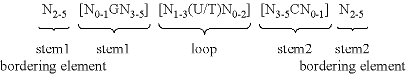

wherein N, C, G, T and U are as defined above.

According to a further more particularly preferred embodiment of the first aspect, the inventive mRNA sequence may comprise at least one histone stem-loop sequence according to at least one of the following specific formulae (Ib) or (IIb):

(stem-loop sequence without stem bordering elements): formula (Ib)

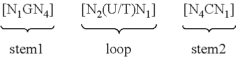

(stem-loop sequence with stem bordering elements): formula (IIb)

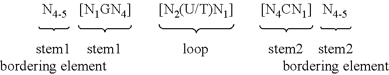

wherein N, C, G, T and U are as defined above.

A particular preferred histone stem-loop sequence is the sequence according to SEQ ID No: 5.

Stem-loop nucleotide sequence
(SEQ ID NO: 5)
CAAAGGCTCTTTTCAGAGCCACCA

More preferably the stem-loop sequence is the corresponding RNA sequence of the nucleic acid sequence according to SEQ ID NO: 6

Stem-loop nucleotide sequence
(SEQ ID NO: 6)
CAAAGGCUCUUUUCAGAGCCACCA

In a particular preferred embodiment, the mRNA of the inventive composition comprises, additionally to the coding region encoding at least one epitope of at least one antigen, a poly(A) sequence, also called poly-A tail, preferably at the 3′ terminus of the mRNA. When present, such a poly(A) sequence comprises a sequence of about 25 to about 400 adenosine nucleotides, preferably a sequence of about 50 to about 400 adenosine nucleotides, more preferably a sequence of about 50 to about 300 adenosine nucleotides, even more preferably a sequence of about 50 to about 250 adenosine nucleotides, most preferably a sequence of about 60 to about 250 adenosine nucleotides. In this context the term “about” refers to a deviation of ±10% of the value(s) it is attached to. This poly(A) sequence is preferably located 3′ of the coding region comprised in the mRNA according to the invention.

According to a further preferred embodiment, the mRNA of the inventive composition can be modified by a sequence of at least 10 cytosines, preferably at least 20 cytosines, more preferably at least 30 cytosines (so-called “poly(C) sequence”). Particularly, the mRNA may contain a poly(C) sequence of typically about 10 to 200 cytosine nucleotides, preferably about 10 to 100 cytosine nucleotides, more preferably about 10 to 70 cytosine nucleotides or even more preferably about 20 to 50 or even 20 to 30 cytosine nucleotides. This poly(C) sequence is preferably located 3′ of the coding region, more preferably 3′ of an optional poly(A) sequence comprised in the mRNA according to the present invention.

For further improvement of the resistance to e.g. in vivo degradation (e.g. by an exo- or endonuclease), the mRNA of the inventive composition is provided as a stabilized nucleic acid, e.g. in the form of a modified nucleic acid. In this context the G/C content is preferably increased as outlined above. According to a further embodiment of the invention it is therefore preferred that the mRNA is further stabilized, preferably by backbone modifications, sugar modifications and/or base modifications. All of these modifications may be introduced into the mRNA without impairing the mRNA's function to be translated in the host cell (cancer cell).

A backbone modification in the context of the present invention is preferably a modification in which phosphates of the backbone of the nucleotides contained in the mRNA are chemically modified, e.g. anionic internucleoside linkage, N3′→P5′ modifications, replacement of non-bridging oxygen atoms by boranes, neutral internucleoside linkage, amide linkage of the nucleosides, methylene(methylimino) linkages, formacetal and thioformacetal linkages, introduction of sulfonyl groups, or the like.

A sugar modification in the context of the present invention is preferably a chemical modification of the sugar of the nucleotides of the mRNA, e.g. methylation of the ribose residue or the like.

Further details about the chemical modification of the RNA, especially the mRNA, will be apparent from the following, wherein the term “RNA modification” as used herein may refer to chemical modifications comprising sugar modifications, backbone modifications as well as base modifications or lipid modifications. In this context, a modified RNA molecule as defined herein may contain nucleotide analogues/modifications, e.g. backbone modifications, sugar modifications or base modifications. A backbone modification in connection with the present invention is a modification, in which phosphates of the backbone of the nucleotides contained in an RNA molecule as defined herein are chemically modified. A sugar modification in connection with the present invention is a chemical modification of the sugar of the nucleotides of the RNA molecule as defined herein. Furthermore, a base modification in connection with the present invention is a chemical modification of the base moiety of the nucleotides of the RNA molecule. In this context, nucleotide analogues or modifications are preferably selected from nucleotide analogues, which are applicable for transcription and/or translation.

The modified nucleosides and nucleotides, which may be incorporated into a modified RNA molecule as described herein, can be modified in the sugar moiety. For example, the 2′ hydroxyl group (OH) can be modified or replaced with a number of different “oxy” or “deoxy” substituents. Examples of “oxy”-2′ hydroxyl group modifications include, but are not limited to, alkoxy or aryloxy (—OR, e.g., R═H, alkyl, cycloalkyl, aryl, aralkyl, heteroaryl or sugar); polyethyleneglycols (PEG), —O(CH₂CH₂O)nCH₂CH₂OR; “locked” nucleic acids (LNA) in which the 2′ hydroxyl is connected, e.g., by a methylene bridge, to the 4′ carbon of the same ribose sugar; and amino groups (—O-amino, wherein the amino group, e.g., NRR, can be alkylamino, dialkylamino, heterocyclyl, arylamino, diarylamino, heteroarylamino, or diheteroaryl amino, ethylene diamine, polyamino) or aminoalkoxy.

“Deoxy” modifications include hydrogen, amino (e.g. NH₂; alkylamino, dialkylamino, heterocyclyl, arylamino, diaryl amino, heteroaryl amino, diheteroaryl amino, or amino acid); or the amino group can be attached to the sugar through a linker, wherein the linker comprises one or more of the atoms C, N, and 0.

The sugar group can also contain one or more carbons that possess the opposite stereochemical configuration than that of the corresponding carbon in ribose. Thus, a modified RNA molecule can include nucleotides containing, for instance, arabinose as the sugar.

The phosphate backbone may further be modified in the modified nucleosides and nucleotides, which may be incorporated into a modified RNA molecule as described herein. The phosphate groups of the backbone can be modified by replacing one or more of the oxygen atoms with a different substituent. Further, the modified nucleosides and nucleotides can include the full replacement of an unmodified phosphate moiety with a modified phosphate as described herein. Examples of modified phosphate groups include, but are not limited to, phosphorothioate, phosphoroselenates, borano phosphates, borano phosphate esters, hydrogen phosphonates, phosphoroamidates, alkyl or aryl phosphonates and phosphotriesters. Phosphorodithioates have both non-linking oxygens replaced by sulfur. The phosphate linker can also be modified by the replacement of a linking oxygen with nitrogen (bridged phosphoroamidates), sulfur (bridged phosphorothioates) and carbon (bridged methylene-phosphonates).

The modified nucleosides and nucleotides, which may be incorporated into a modified RNA molecule as described herein, can further be modified in the nucleobase moiety. Examples of nucleobases found in RNA include, but are not limited to, adenine, guanine, cytosine and uracil. For example, the nucleosides and nucleotides described herein can be chemically modified on the major groove face. In some embodiments, the major groove chemical modifications can include an amino group, a thiol group, an alkyl group, or a halo group.

In particularly preferred embodiments of the present invention, the nucleotide analogues/modifications are selected from base modifications, which are preferably selected from 2-amino-6-chloropurineriboside-5′-triphosphate, 2-Aminopurine-riboside-5′-triphosphate; 2-aminoadenosine-5′-triphosphate, 2′-amino-2′-deoxycytidine-triphosphate, 2-thiocytidine-5′-triphosphate, 2-thiouridine-5′-triphosphate, 2′-Fluorothymidine-5′-triphosphate, 2′-O-Methyl inosine-5′-triphosphate 4-thiouridine-5′-triphosphate, 5-aminoallylcytidine-5′-triphosphate, 5-aminoallyluridine-5′-triphosphate, 5-bromocytidine-5′-triphosphate, 5-bromouridine-5′-triphosphate, 5-Bromo-2′-deoxycytidine-5′-triphosphate, 5-Bromo-2′-deoxyuridine-5′-triphosphate, 5-iodocytidine-5′-triphosphate, 5-Iodo-2′-deoxycytidine-5′-triphosphate, 5-iodouridine-5′-triphosphate, 5-Iodo-2′-deoxyuridine-5′-triphosphate, 5-methylcytidine-5′-triphosphate, 5-methyluridine-5′-triphosphate, 5-Propynyl-2′-deoxycytidine-5′-triphosphate, 5-propynyl-2′-deoxyuridine-5′-triphosphate, 6-azacytidine-5′-triphosphate, 6-azauridine-5′-triphosphate, 6-chloropurineriboside-5′-triphosphate, 7-deazaadenosine-5′-triphosphate, 7-deazaguanosine-5′-triphosphate, 8-azaadenosine-5′-triphosphate, 8-azidoadenosine-5′-triphosphate, benzimidazole-riboside-5′-triphosphate, N1-methyladenosine-5′-triphosphate, N1-methylguanosine-5′-triphosphate, N6-methyladenosine-5′-triphosphate, 06-methylguanosine-5′-triphosphate, pseudouridine-5′-triphosphate, or puromycin-5′-triphosphate, xanthosine-5′-triphosphate. Particular preference is given to nucleotides for base modifications selected from the group of base-modified nucleotides consisting of 5-methylcytidine-5′-triphosphate, 7-deazaguanosine-5′-triphosphate, 5-bromocytidine-5′-triphosphate, and pseudouridine-5′-triphosphate.

In some embodiments, modified nucleosides include pyridin-4-one ribonucleoside, 5-aza-uridine, 2-thio-5-aza-uridine, 2-thiouridine, 4-thio-pseudouridine, 2-thio-pseudouridine, 5-hydroxyuridine, 3-methyluridine, 5-carboxymethyl-uridine, 1-carboxymethyl-pseudouridine, 5-propynyl-uridine, 1-propynyl-pseudouridine, 5-taurinomethyluridine, 1-taurinomethyl-pseudouridine, 5-taurinomethyl-2-thio-uridine, 1-taurinomethyl-4-thio-uridine, 5-methyl-uridine, 1-methyl-pseudouridine, 4-thio-1-methyl-pseudouridine, 2-thio-1-methyl-pseudouridine, 1-methyl-1-deaza-pseudouridine, 2-thio-1-methyl-1-deaza-pseudouridine, dihydrouridine, dihydropseudouridine, 2-thio-dihydrouridine, 2-thio-dihydropseudouridine, 2-methoxyuridine, 2-methoxy-4-thio-uridine, 4-methoxy-pseudouridine, and 4-methoxy-2-thio-pseudouridine.

In some embodiments, modified nucleosides include 5-aza-cytidine, pseudoisocytidine, 3-methyl-cytidine, N4-acetylcytidine, 5-formylcytidine, N4-methylcytidine, 5-hydroxymethylcytidine, 1-methyl-pseudoisocytidine, pyrrolo-cytidine, pyrrolo-pseudoisocytidine, 2-thio-cytidine, 2-thio-5-methyl-cytidine, 4-thio-pseudoisocytidine, 4-thio-1-methyl-pseudoisocytidine, 4-thio-1-methyl-1-deaza-pseudoisocytidine, 1-methyl-1-deaza-pseudoisocytidine, zebularine, 5-aza-zebularine, 5-methyl-zebularine, 5-aza-2-thio-zebularine, 2-thio-zebularine, 2-methoxy-cytidine, 2-methoxy-5-methyl-cytidine, 4-methoxy-pseudoisocytidine, and 4-methoxy-1-methyl-pseudoisocytidine.

In other embodiments, modified nucleosides include 2-aminopurine, 2, 6-diaminopurine, 7-deaza-adenine, 7-deaza-8-aza-adenine, 7-deaza-2-aminopurine, 7-deaza-8-aza-2-aminopurine, 7-deaza-2,6-diaminopurine, 7-deaza-8-aza-2,6-diaminopurine, 1-methyladenosine, N6-methyladenosine, N6-isopentenyladenosine, N6-(cis-hydroxyisopentenyl)adenosine, 2-methylthio-N6-(cis-hydroxyisopentenyl) adenosine, N6-glycinylcarbamoyladenosine, N6-threonylcarbamoyladenosine, 2-methylthio-N6-threonyl carbamoyladenosine, N6,N6-dimethyladenosine, 7-methyladenine, 2-methylthio-adenine, and 2-methoxy-adenine.

In other embodiments, modified nucleosides include inosine, 1-methyl-inosine, wyosine, wybutosine, 7-deaza-guanosine, 7-deaza-8-aza-guanosine, 6-thio-guanosine, 6-thio-7-deaza-guanosine, 6-thio-7-deaza-8-aza-guanosine, 7-methyl-guanosine, 6-thio-7-methyl-guanosine, 7-methylinosine, 6-methoxy-guanosine, 1-methylguanosine, N2-methylguanosine, N2,N2-dimethylguanosine, 8-oxo-guanosine, 7-methyl-8-oxo-guanosine, 1-methyl-6-thio-guanosine, N2-methyl-6-thio-guanosine, and N2,N2-dimethyl-6-thio-guanosine.

In some embodiments, the nucleotide can be modified on the major groove face and can include replacing hydrogen on C-5 of uracil with a methyl group or a halo group.

In specific embodiments, a modified nucleoside is 5′-O-(1-thiophosphate)-Adenosine, 5′-O-(1-thiophosphate)-cytidine, 5′-O-(1-thiophosphate)-guanosine, 5-O-(1-thiophosphate)-uridine or 5′-O-(1-thiophosphate)-pseudouridine.

In further specific embodiments, a modified RNA may comprise nucleoside modifications selected from 6-aza-cytidine, 2-thio-cytidine, α-thio-cytidine, pseudo-iso-cytidine, 5-aminoallyl-uridine, 5-iodo-uridine, N1-methyl-pseudouridine, 5,6-dihydrouridine, α-thio-uridine, 4-thio-uridine, 6-aza-uridine, 5-hydroxy-uridine, deoxy-thymidine, 5-methyl-uridine, pyrrolo-cytidine, inosine, α-thio-guanosine, 6-methyl-guanosine, 5-methyl-cytdine, 8-oxo-guanosine, 7-deaza-guanosine, N1-methyl-adenosine, 2-amino-6-chloro-purine, N6-methyl-2-amino-purine, pseudo-iso-cytidine, 6-chloro-purine, N6-methyl-adenosine, α-thio-adenosine, 8-azido-adenosine, 7-deaza-adenosine.

Further nucleotide analogues are such as those disclosed in WO2013/052523.

According to a further embodiment, a modified RNA molecule as defined herein can contain a lipid modification. Such a lipid-modified RNA molecule typically comprises an RNA molecule as defined herein. Such a lipid-modified RNA molecule as defined herein typically further comprises at least one linker covalently linked with that RNA molecule, and at least one lipid covalently linked with the respective linker. Alternatively, the lipid-modified RNA molecule comprises at least one RNA molecule as defined herein and at least one (bifunctional) lipid covalently linked (without a linker) with that RNA molecule. According to a third alternative, the lipid-modified RNA molecule comprises an RNA molecule as defined herein, at least one linker covalently linked with that RNA molecule, and at least one lipid covalently linked with the respective linker, and also at least one (bifunctional) lipid covalently linked (without a linker) with that RNA molecule. In this context, it is particularly preferred that the lipid modification is present at the terminal ends of a linear RNA sequence.

According to another preferred embodiment of the invention, a modified RNA molecule as defined herein, can be modified by the addition of a so-called “5′ CAP” structure, namely by modification of the 5′-end of a RNA molecule.

A 5′-cap is an entity, typically a modified nucleotide entity, which generally “caps” the 5′-end of a mature mRNA. A 5′-cap may typically be formed by a modified nucleotide, particularly by a derivative of a guanine nucleotide. Preferably, the 5′-cap is linked to the 5′-terminus via a 5′-5′-triphosphate linkage. A 5′-cap may be methylated, e.g. m7GpppN, wherein N is the terminal 5′ nucleotide of the nucleic acid carrying the 5′-cap, typically the 5′-end of an RNA. m7GpppN is the 5′-CAP structure which naturally occurs in mRNA transcribed by polymerase II and is therefore not considered as modification comprised in a modified RNA in this context. Accordingly, a modified RNA of the present invention may comprise a m7GpppN as 5′-CAP, but additionally the modified RNA comprises at least one further modification as defined herein.

Further examples of 5′-cap structures include glyceryl, inverted deoxy abasic residue (moiety), 4′,5′ methylene nucleotide, 1-(beta-D-erythrofuranosyl) nucleotide, 4′-thio nucleotide, carbocyclic nucleotide, 1,5-anhydrohexitol nucleotide, L-nucleotides, alpha-nucleotide, modified base nucleotide, threo-pentofuranosyl nucleotide, acyclic 3′,4′-seco nucleotide, acyclic 3,4-dihydroxybutyl nucleotide, acyclic 3,5 dihydroxypentyl nucleotide, 3′-3′-inverted nucleotide moiety, 3′-3′-inverted abasic moiety, 3′-2′-inverted nucleotide moiety, 3′-2′-inverted abasic moiety, 1,4-butanediol phosphate, 3′-phosphoramidate, hexylphosphate, aminohexyl phosphate, 3′-phosphate, 3′phosphorothioate, phosphorodithioate, or bridging or non-bridging methylphosphonate moiety. These modified 5′-CAP structures are regarded as at least one modification in this context.

Particularly preferred modified 5′-cap structures are CAP1 (methylation of the ribose of the adjacent nucleotide of m7G), CAP2 (methylation of the ribose of the 2nd nucleotide downstream of the m7G), CAP3 (methylation of the ribose of the 3rd nucleotide downstream of the m7G), CAP4 (methylation of the ribose of the 4th nucleotide downstream of the m7G), ARCA (anti-reverse CAP analogue, modified ARCA (e.g. phosphothioate modified ARCA), inosine, N1-methyl-guanosine, 2′-fluoro-guanosine, 7-deaza-guanosine, 8-oxo-guanosine, 2-amino-guanosine, LNA-guanosine, and 2-azido-guanosine.

According to a further preferred embodiment of the invention, the mRNA of the inventive composition is optimized for translation, preferably optimized for translation by replacing codons for less frequent tRNAs of a given amino acid by codons for more frequently occurring tRNAs of the respective amino acid. This is based on the finding that the translation efficiency is also determined by a different frequency in the occurrence of tRNAs in cells. Thus, if so-called “less frequent codons” are present in the inventive mRNA to an increased extent, the corresponding modified RNA is translated to a significantly poorer degree than in the case where codons coding for more frequent tRNAs are present. Preferably, the coding region of the mRNA is modified compared to the corresponding region of the wild type RNA or coding sequence such that at least one codon of the wild type sequence which codes for a tRNA which is relatively rare or less frequent in the cell is exchanged for a codon which codes for a tRNA which is more or most frequent in the cell and carries the same amino acid as the relatively rare or less frequent tRNA. By this modification, the sequences of the mRNA can be modified such that codons for which more frequently occurring tRNAs are available are inserted. In other words, according to the invention, by this modification all codons of the wild type sequence which code for a tRNA which is relatively rare in the cell can in each case be exchanged for a codon which codes for a respective tRNA which is relatively frequent in the cell and which, in each case, carries the same amino acid as the relatively rare tRNA. Furthermore, it is particularly preferable to link the sequential G/C content which is increased, in particular maximized, in the mRNA with the “frequent” codons without modifying the amino acid sequence of the protein encoded by the coding region of the mRNA or of the coding region. This preferred embodiment allows provision of a particularly efficiently translated and stabilized (modified) mRNA.

Substitutions, additions or eliminations of bases are preferably carried out using a DNA matrix for preparation of the nucleic acid molecule by techniques of the well known site directed mutagenesis or with an oligonucleotide ligation. In such a process, for preparation of the at least one RNA as defined herein a corresponding DNA molecule may be transcribed in vitro. This DNA matrix preferably comprises a suitable promoter, e.g. a T7 or SP6 promoter, for in vitro transcription, which is followed by the desired nucleotide sequence for the at least one RNA to be prepared and a termination signal for in vitro transcription. The DNA molecule, which forms the matrix of the at least one RNA of interest, may be prepared by fermentative proliferation and subsequent isolation as part of a plasmid which can be replicated in bacteria. Plasmids which may be mentioned as suitable for the present invention are e.g. the plasmids pT7 Ts (GenBank accession number AB255037.1; Lai et al., Development 1995, 121: 2349 to 2360), pGEM® series, e.g. pGEM®-1 (GenBank accession number X65300.1; from Promega) and pSP64 (GenBank accession number X65327.1); cf. also Mezei and Storts, Purification of PCR Products, in: Griffin and Griffin (ed.), PCR Technology: Current Innovation, CRC Press, Boca Raton, Fla., 2001.

The mRNA may be prepared using any method known in the art, including synthetic methods such as e.g. solid phase synthesis, as well as in vitro methods, such as in vitro transcription reactions.

In summary, in the context of the present invention, an mRNA is typically an RNA, which is composed of several structural elements, e.g. an optional 5′-CAP structure, an optional 5′-UTR region, an upstream positioned ribosomal binding site followed by a coding region, an optional 3′-UTR region, which may be followed by a poly-A tail (and/or a poly-C-tail). An mRNA may occur as a mono-, di-, or even multicistronic RNA, i.e. a RNA which carries the coding sequences of one, two or more proteins or peptides representing at least one epitope of at least one antigen. Such coding sequences in di-, or even multicistronic mRNA may be separated by at least one IRES sequence, e.g. as defined herein.

In a particularly preferred embodiment the mRNA of the inventive composition may be complexed with a cationic component. Cationic compounds being particularly preferred agents in this context include protamine, nucleoline, spermine or spermidine, or other cationic peptides or proteins, such as poly-L-lysine (PLL), poly-arginine, basic polypeptides, cell penetrating peptides (CPPs), including HIV-binding peptides, HIV-1 Tat (HIV), Tat-derived peptides, Penetratin, VP22 derived or analog peptides, HSV VP22 (Herpes simplex), MAP, KALA or protein transduction domains (PTDs), PpT620, proline-rich peptides, arginine-rich peptides, lysine-rich peptides, MPG-peptide(s), Pep-1, L-oligomers, Calcitonin peptide(s), Antennapedia-derived peptides (particularly from Drosophila antennapedia), pAntp, pIsl, FGF, Lactoferrin, Transportan, Buforin-2, Bac715-24, SynB, SynB(1), pVEC, hCT-derived peptides, SAP, or histones. Protamine is particularly preferred. Nevertheless, it is also possible that the mRNA of the inventive composition is naked.

The nucleotide acid molecule of the first immunogenic component of the inventive composition, preferably the mRNA molecule, encodes at least one epitope of at least one antigen. In preferred embodiments of the invention the at least one antigen is selected from the group consisting of an antigen from a pathogen associated with infectious diseases, an antigen associated with allergies, an antigen associated with autoimmune diseases, and an antigen associated with cancer or tumor diseases, or a fragment, variant and/or derivative of said antigen.

Preferably the at least one antigen is derived from a pathogen, preferably a viral, bacterial, fungal or protozoan pathogen, preferably selected from the list consisting of: Rabies virus, Ebolavirus, Marburgvirus, Hepatitis B virus, human Papilloma virus (hPV), Bacillus anthracis, Respiratory syncytial virus (RSV), Herpes simplex virus (HSV), Dengue virus, Rotavirus, Influenza virus, human immunodeficiency virus (HIV), Yellow Fever virus, Mycobacterium tuberculosis, Plasmodium, Staphylococcus aureus, Chlamydia trachomatis, Cytomegalovirus (CMV) and Hepatitis B virus (HBV).

In this context the mRNA of the inventive composition may encode for a protein or a peptide, which comprises at least one epitope of a pathogenic antigen or a fragment, variant or derivative thereof. Such pathogenic antigens are derived from pathogenic organisms, in particular bacterial, viral or protozoological (multicellular) pathogenic organisms, which evoke an immunological reaction by subject, in particular a mammalian subject, more particularly a human. More specifically, pathogenic antigens are preferably surface antigens, e.g. proteins (or fragments of proteins, e.g. the exterior portion of a surface antigen) located at the surface of the virus or the bacterial or protozoological organism.

Pathogenic antigens are peptide or protein antigens preferably derived from a pathogen associated with infectious disease which are preferably selected from antigens derived from the pathogens Acinetobacter baumannii, Anaplasma genus, Anaplasma phagocytophilum, Ancylostoma braziliense, Ancylostoma duodenale, Arcanobacterium haemolyticum, Ascaris lumbricoides, Aspergillus genus, Astroviridae, Babesia genus, Bacillus anthracis, Bacillus cereus, Bartonella henselae, BK virus, Blastocystis hominis, Blastomyces dermatitidis, Bordetella pertussis, Borrelia burgdorferi, Borrelia genus, Borrelia spp, Brucella genus, Brugia malayi, Bunyaviridae family, Burkholderia cepacia and other Burkholderia species, Burkholderia mallei, Burkholderia pseudomallei, Caliciviridae family, Campylobacter genus, Candida albicans, Candida spp, Chlamydia trachomatis, Chlamydophila pneumoniae, Chlamydophila psittaci, CJD prion, Clonorchis sinensis, Clostridium botulinum, Clostridium difficile, Clostridium perfringens, Clostridium perfringens, Clostridium spp, Clostridium tetani, Coccidioides spp, coronaviruses, Corynebacterium diphtheriae, Coxiella bumetii, Crimean-Congo hemorrhagic fever virus, Cryptococcus neoformans, Cryptosporidium genus, Cytomegalovirus (CMV), Dengue viruses (DEN-1, DEN-2, DEN-3 and DEN-4), Dientamoeba fragilis, Ebolavirus (EBOV), Echinococcus genus, Ehrlichia chaffeensis, Ehrlichia ewingii, Ehrlichia genus, Entamoeba histolytica, Enterococcus genus, Enterovirus genus, Enteroviruses, mainly Coxsackie A virus and Enterovirus 71 (EV71), Epidermophyton spp, Epstein-Barr Virus (EBV), Escherichia coli O157:H7, O111 and O104:H4, Fasciola hepatica and Fasciola gigantica, FFI prion, Filarioidea superfamily, Flaviviruses, Francisella tularensis, Fusobacterium genus, Geotrichum candidum, Giardia intestinalis, Gnathostoma spp, GSS prion, Guanarito virus, Haemophilus ducreyi, Haemophilus influenzae, Helicobacter pylori, Henipavirus (Hendra virus Nipah virus), Hepatitis A Virus, Hepatitis B Virus (HBV), Hepatitis C Virus (HCV), Hepatitis D Virus, Hepatitis E Virus, Herpes simplex virus 1 and 2 (HSV-1 and HSV-2), Histoplasma capsulatum, HIV (Human immunodeficiency virus), Hortaea werneckii, Human bocavirus (HBoV), Human herpesvirus 6 (HHV-6) and Human herpesvirus 7 (HHV-7), Human metapneumovirus (hMPV), Human papillomavirus (HPV), Human parainfluenza viruses (HPIV), Japanese encephalitis virus, JC virus, Junin virus, Kingella kingae, Klebsiella granulomatis, Kuru prion, Lassa virus, Legionella pneumophila, Leishmania genus, Leptospira genus, Listeria monocytogenes, Lymphocytic choriomeningitis virus (LCMV), Machupo virus, Malassezia spp, Marburg virus, Measles virus, Metagonimus yokagawai, Microsporidia phylum, Molluscum contagiosum virus (MCV), Mumps virus, Mycobacterium leprae and Mycobacterium lepromatosis, Mycobacterium tuberculosis, Mycobacterium ulcerans, Mycoplasma pneumoniae, Naegleria fowleri, Necator americanus, Neisseria gonorrhoeae, Neisseria meningitidis, Nocardia asteroides, Nocardia spp, Onchocerca volvulus, Orientia tsutsugamushi, Orthomyxoviridae family (Influenza), Paracoccidioldes brasiliensis, Paragonimus spp, Paragonimus westermani, Parvovirus B19, Pasteurella genus, Plasmodium genus, Pneumocystis jirovecii, Poliovirus, Rabies virus, Respiratory syncytial virus (RSV), Rhinovirus, rhinoviruses, Rickettsia akari, Rickettsia genus, Rickettsia prowazekii, Rickettsia rickettsii, Rickettsia typhi, Rift Valley fever virus, Rotavirus, Rubella virus, Sabia virus, Salmonella genus, Sarcoptes scabiei, SARS coronavirus, Schistosoma genus, Shigella genus, Sin Nombre virus, Hantavirus, Sporothrix schenckii, Staphylococcus genus, Staphylococcus genus, Streptococcus agalactiae, Streptococcus pneumoniae, Streptococcus pyogenes, Strongyloides stercoralis, Taenia genus, Taenia solium, Tick-borne encephalitis virus (TBEV), Toxocara canis or Toxocara cati, Toxoplasma gondii, Treponema pallidum, Trichinella spiralis, Trichomonas vaginalis, Trichophyton spp, Trichuris trichiura, Trypanosoma brucei, Trypanosoma cruzi, Ureaplasma urealyticum, Varicella zoster virus (VZV), Varicella zoster virus (VZV), Variola major or Variola minor, vCJD prion, Venezuelan equine encephalitis virus, Vibrio cholerae, West Nile virus, Western equine encephalitis virus, Wuchereria bancrofti, Yellow fever virus, Yersinia enterocolitica, Yersinia pestis, and Yersinia pseudotuberculosis.

Furthermore, the pathogenic antigen (antigen derived from a pathogen associated with infectious disease) may be preferably selected from the following antigens: Outer membrane protein A OmpA, biofilm associated protein Bap, transport protein MucK (Acinetobacter baumannii, Acinetobacter infections)); variable surface glycoprotein VSG, microtubule-associated protein MAPP15, trans-sialidase TSA (Trypanosoma brucei, African sleeping sickness (African trypanosomiasis)); HIV p24 antigen, HIV envelope proteins (Gp120, Gp41, Gp160), polyprotein GAG, negative factor protein Nef, trans-activator of transcription Tat (HIV (Human immunodeficiency virus), AIDS (Acquired immunodeficiency syndrome)); galactose-inhibitable adherence protein GIAP, 29 kDa antigen Eh29, GaI/GaINAc lectin, protein CRT, 125 kDa immunodominant antigen, protein M17, adhesin ADH112, protein STIRP (Entamoeba histolytica, Amoebiasis); Major surface proteins 1-5 (MSP1a, MSP1b, MSP2, MSP3, MSP4, MSP5), type IV secretion system proteins (VirB2, VirB7, VirB11, VirD4) (Anaplasma genus, Anaplasmosis); protective Antigen PA, edema factor EF, lethal factor LF, the S-layer homology proteins SLH (Bacillus anthracis, Anthrax); acranolysin, phospholipase D, collagen-binding protein CbpA (Arcanobacterium haemolyticum, Arcanobacterium haemolyticum infection); nucleocapsid protein NP, glycoprotein precursor GPC, glycoprotein GP1, glycoprotein GP2 (Junin virus, Argentine hemorrhagic fever); chitin-protein layer proteins, 14 kDa surface antigen A14, major sperm protein MSP, MSP polymerization-organizing protein MPOP, MSP fiber protein 2 MFP2, MSP polymerization-activating kinase MPAK, ABA-1-like protein ALB, protein ABA-1, cuticulin CUT-1 (Ascaris lumbricoides, Ascariasis); 41 kDa allergen Asp v13, allergen Asp β, major conidial surface protein rodlet A, protease Pep1p, GPI-anchored protein Gel1p, GPI-anchored protein Crf1p (Aspergillus genus, Aspergillosis); family VP26 protein, VP29 protein (Astroviridae, Astrovirus infection); Rhoptry-associated protein 1 RAP-1, merozoite surface antigens MSA-1, MSA-2 (a1, a2, b, c), 12D3, 1105, 21B4, P29, variant erythrocyte surface antigen VESA1, Apical Membrane Antigen 1 AMA-1 (Babesia genus, Babesiosis); hemolysin, enterotoxin C, PXO1-51, glycolate oxidase, ABC-transporter, penicillin-bingdn protein, zinc transporter family protein, pseudouridine synthase Rsu, plasmid replication protein RepX, oligoendopeptidase F, prophage membrane protein, protein HemK, flagellar antigen H, 28.5-kDa cell surface antigen (Bacillus cereus, Bacillus cereus infection); large T antigen LT, small T antigen, capsid protein VP1, capsid protein VP2 (BK virus, BK virus infection); 29 kDa-protein, caspase-3-like antigens, glycoproteins (Blastocystis hominis, Blastocystis hominis infection); yeast surface adhesin WI-1 (Blastomyces dermatitidis, Blastomycosis); nucleoprotein N, polymerase L, matrix protein Z, glycoprotein GP (Machupo virus, Bolivian hemorrhagic fever); outer surface protein A OspA, outer surface protein OspB, outer surface protein OspC, decorin binding protein A DbpA, decorin binding protein B DbpB, flagellar filament 41 kDa core protein Fla, basic membrane protein A precursor BmpA (Immunodominant antigen P39), outer surface 22 kDa lipoprotein precursor (antigen IPLA7), variable surface lipoprotein vlsE (Borrelia genus, Borrelia infection); Botulinum neurotoxins BoNT/A1, BoNT/A2, BoNT/A3, BoNT/B, BoNT/C, BoNT/D, BoNT/E, BoNT/F, BoNT/G, recombinant botulinum toxin F Hc domain FHc (Clostridium botulinum, Botulism (and Infant botulism)); nucleocapsid, glycoprotein precursor (Sabia virus, Brazilian hemorrhagic fever); copper/Zinc superoxide dismutase SodC, bacterioferritin Bfr, 50S ribosomal protein RpIL, OmpA-like transmembrane domain-containing protein Omp31, immunogenic 39-kDa protein M5 P39, zinc ABC transporter periplasmic zinc-binding protein znuA, periplasmic immunogenic protein Bp26, 30S ribosomal protein S12 RpsL, glyceraldehyde-3-phosphate dehydrogenase Gap, 25 kDa outer-membrane immunogenic protein precursor Omp25, invasion protein B IaIB, trigger factor Tig, molecular chaperone DnaK, putative peptidyl-prolyl cis-trans isomerase SurA, lipoprotein Omp19, outer membrane protein MotY Omp16, conserved outer membrane protein D15, malate dehydrogenase Mdh, component of the Type-IV secretion system (T4SS) VirJ, lipoprotein of unknown function BAB1_0187 (Brucella genus, Brucellosis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia cepacia and other Burkholderia species, Burkholderia infection); mycolyl-transferase Ag85A, heat-shock protein Hsp65, protein TB10.4, 19 kDa antigen, protein PstS3, heat-shock protein Hsp70 (Mycobacterium ulcerans, Buruli ulcer); norovirus major and minor viral capsid proteins VP1 and VP2, genome polyprotein, Sapoviurus capsid protein VP1, protein Vp3, geome polyprotein (Caliciviridae family, Calicivirus infection (Norovirus and Sapovirus)); major outer membrane protein PorA, flagellin FlaA, surface antigen CjaA, fibronectin binding protein CadF, aspartate/glutamate-binding ABC transporter protein Peb1A, protein FspA1, protein FspA2 (Campylobacter genus, Campylobacteriosis); glycolytic enzyme enolase, secreted aspartyl proteinases SAP1-10, glycophosphatidylinositol (GPI)-linked cell wall protein, protein Hyr1, complement receptor 3-related protein CR3-RP, adhesin Als3p, heat shock protein 90 kDa hsp90, cell surface hydrophobicity protein CSH (usually Candida albicans and other Candida species, Candidiasis); 17-kDa antigen, protein P26, trimeric autotransporter adhesins TAAs, Bartonella adhesin A BadA, variably expressed outer-membrane proteins Vomps, protein Pap3, protein HbpA, envelope-associated protease HtrA, protein OMP89, protein GroEL, protein LalB, protein OMP43, dihydrolipoamide succinyltransferase SucB (Bartonella henselae, Cat-scratch disease); amastigote surface protein-2, amastigote-specific surface protein SSP4, cruzipain, trans-sialidase TS, trypomastigote surface glycoprotein TSA-1, complement regulatory protein CRP-10, protein G4, protein G2, paraxonemal rod protein PAR2, paraflagellar rod component Part, mucin-Associated Surface Proteins MPSP (Trypanosoma cruzi, Chagas Disease (American trypanosomiasis)); envelope glycoproteins (gB, gC, gE, gH, gI, gK, gL) (Varicella zoster virus (VZV), Chickenpox); major outer membrane protein MOMP, probable outer membrane protein PMPC, outer membrane complex protein B OmcB, heat shock proteins Hsp60 HSP10, protein IncA, proteins from the type Ill secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 (Chlamydia trachomatis, Chlamydia); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila pneumoniae, Chlamydophila pneumoniae infection); cholera toxin B CTB, toxin coregulated pilin A TcpA, toxin coregulated pilin TcpF, toxin co-regulated pilus biosynthesis ptrotein F TcpF, cholera enterotoxin subunit A, cholera enterotoxin subunit B, Heat-stable enterotoxin ST, mannose-sensitive hemagglutinin MSHA, outer membrane protein U Porin ompU, Poring B protein, polymorphic membrane protein-D (Vibrio cholerae, Cholera); propionyl-CoA carboxylase PCC, 14-3-3 protein, prohibitin, cysteine proteases, glutathione transferases, gelsolin, cathepsin L proteinase CatL, Tegumental Protein 20.8 kDa TP20.8, tegumental protein 31.8 kDa TP31.8, lysophosphatidic acid phosphatase LPAP, (Clonorchis sinensis, Clonorchiasis); surface layer proteins SLPs, glutamate dehydrogenase antigen GDH, toxin A, toxin B, cysteine protease Cwp84, cysteine protease Cwp13, cysteine protease Cwp19, Cell Wall Protein CwpV, flagellar protein FliC, flagellar protein FliD (Clostridium difficile, Clostridium difficile infection); rhinoviruses: capsid proteins VP1, VP2, VP3, VP4; coronaviruses: sprike proteins S, envelope proteins E, membrane proteins M, nucleocapsid proteins N (usually rhinoviruses and coronaviruses, Common cold (Acute viral rhinopharyngitis; Acute coryza)); prion protein Prp (CJD prion, Creutzfeldt-Jakob disease (CJD)); envelope protein Gc, envelope protein Gn, nucleocapsid proteins (Crimean-Congo hemorrhagic fever virus, Crimean-Congo hemorrhagic fever (CCHF)); virulence-associated DEAD-box RNA helicase VAD1, galactoxylomannan-protein GaIXM, glucuronoxylomannan GXM, mannoprotein MP (Cryptococcus neoformans, Cryptococcosis); acidic ribosomal protein P2 CpP2, mucin antigens Muc1, Muc2, Muc3 Muc4, Muc5, Muc6, Muc7, surface adherence protein CP20, surface adherence protein CP23, surface protein CP12, surface protein CP21, surface protein CP40, surface protein CP60, surface protein CP15, surface-associated glycopeptides gp40, surface-associated glycopeptides gp15, oocyst wall protein AB, profilin PRF, apyrase (Cryptosporidium genus, Cryptosporidiosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, adult-specific secreted factor Xa serine protease inhibitor anticoagulant AP, cathepsin D-like aspartic protease ARR-1 (usually Ancylostoma braziliense; multiple other parasites, Cutaneous larva migrans (CLM)); cathepsin L-like proteases, 53125-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia solium, Cysticercosis); pp65 antigen, membrane protein pp15, capsid-proximal tegument protein pp150, protein M45, DNA polymerase UL54, helicase UL105, glycoprotein gM, glycoprotein gN, glcoprotein H, glycoprotein B gB, protein UL83, protein UL94, protein UL99 (Cytomegalovirus (CMV), Cytomegalovirus infection); capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 (Dengue viruses (DEN-1, DEN-2, DEN-3 and DEN-4)-Flaviviruses, Dengue fever); 39 kDa protein (Dientamoeba fragilis, Dientamoebiasis); diphtheria toxin precursor Tox, diphteria toxin DT, pilin-specific sortase SrtA, shaft pilin protein SpaA, tip pilin protein SpaC, minor pilin protein SpaB, surface-associated protein DIP1281 (Corynebacterium diphtheriae, Diphtheria); glycoprotein GP, nucleoprotein NP, minor matrix protein VP24, major matrix protein VP40, transcription activator VP30, polymerase cofactor VP35, RNA polymerase L (Ebolavirus (EBOV), Ebola hemorrhagic fever); prion protein (vCJD prion, Variant Creutzfeldt-Jakob disease (vCJD, nvCJD)); UvrABC system protein B, protein Flp1, protein Flp2, protein Flp3, protein TadA, hemoglobin receptor HgbA, outer membrane protein TdhA, protein CpsRA, regulator CpxR, protein SapA, 18 kDa antigen, outer membrane protein NcaA, protein LspA, protein LspA1, protein LspA2, protein LspB, outer membrane component DsrA, lectin DItA, lipoprotein Hip, major outer membrane protein OMP, outer membrane protein OmpA2 (Haemophilus ducreyi, Chancroid); aspartyl protease 1 Pep1, phospholipase B PLB, alpha-mannosidase 1 AMN1, glucanosyltransferase GEL1, urease URE, peroxisomal matrix protein Pmp1, praline-rich antigen Pra, humal T-cell reative protein TcrP (Coccidioides immitis and Coccidioides posadasii, Coccidioidomycosis); allergen Tri r 2, heat shock protein 60 Hsp60, fungal actin Act, antigen Tri r2, antigen Tri r4, antigen Tri t1, protein IV, glycerol-3-phosphate dehydrogenase Gpd1, osmosensor HwSho1A, osmosensor HwSho1B, histidine kinase HwHhk7B, allergen Mala s 1, allergen Mala s 11, thioredoxin Trx Mala s 13, allergen Mala f, allergen Mala s (usually Trichophyton spp, Epidermophyton spp., Malassezia spp., Hortaea werneckii, Dermatophytosis); protein EG95, protein EG10, protein EG18, protein EgA31, protein EM18, antigen EPC1, antigen B, antigen 5, protein P29, protein 14-3-3, 8-kDa protein, myophilin, heat shock protein 20 HSP20, glycoprotein GP-89, fatty acid binding protein FAPB (Echinococcus genus, Echinococcosis); major surface protein 2 MSP2, major surface protein 4 MSP4, MSP variant SGV1, MSP variant SGV2, outer membrane protein OMP, outer membrande protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia genus, Ehrlichiosis); secreted antigen SagA, sagA-like proteins SalA and SalB, collagen adhesin Scm, surface proteins Fms1 (EbpA(fm), Fms5 (EbpB(fm), Fms9 (EpbC(fm) and Fms10, protein EbpC(fm), 96 kDa immunoprotective glycoprotein G1 (Enterococcus genus, Enterococcus infection); genome polyprotein, polymerase 3D, viral capsid protein VP1, viral capsid protein VP2, viral capsid protein VP3, viral capsid protein VP4, protease 2A, protease 3C (Enterovirus genus, Enterovirus infection); outer membrane proteins OM, 60 kDa outer membrane protein, cell surface antigen OmpA, cell surface antigen OmpB (sca5), 134 kDa outer membrane protein, 31 kDa outer membrane protein, 29.5 kDa outer membrane protein, cell surface protein SCA4, cell surface protein Adr1 (RP827), cell surface protein Adr2 (RP828), cell surface protein SCA1, Invasion protein invA, cell division protein fts, secretion proteins sec Ofamily, virulence proteins virB, tlyA, tlyC, parvulin-like protein Plp, preprotein translocase SecA, 120-kDa surface protein antigen SPA, 138 kD complex antigen, major 100-kD protein (protein I), intracytoplasmic protein D, protective surface protein antigen SPA (Rickettsia prowazekii, Epidemic typhus); Epstein-Barr nuclear antigens (EBNA-1, EBNA-2, EBNA-3A, EBNA-3B, EBNA-3C, EBNA-leader protein (EBNA-LP)), latent membrane proteins (LMP-1, LMP-2A, LMP-2B), early antigen EBV-EA, membrane antigen EBV-MA, viral capsid antigen EBV-VCA, alkaline nuclease EBV-AN, glycoprotein H, glycoprotein gp350, glycoprotein gp110, glycoprotein gp42, glycoprotein gHgL, glycoprotein gB (Epstein-Barr Virus (EBV), Epstein-Barr Virus Infectious Mononucleosis); cpasid protein VP2, capsid protein VP1, major protein NS1 (Parvovirus B19, Erythema infectiosum (Fifth disease)); pp65 antigen, glycoprotein 105, major capsid protein, envelope glycoprotein H, protein U51 (Human herpesvirus 6 (HHV-6) and Human herpesvirus 7 (HHV-7), Exanthem subitum); thioredoxin-glutathione reductase TGR, cathepsins L1 and L2, Kunitz-type protein KTM, leucine aminopeptidase LAP, cysteine proteinase Fas2, saposin-like protein-2 SAP-2, thioredoxin peroxidases TPx, Prx-1, Prx-2, cathepsin I cysteine proteinase CL3, protease cathepsin L CL1, phosphoglycerate kinase PGK, 27-kDa secretory protein, 60 kDa protein HSP35alpha, glutathione transferase GST, 28.5 kDa tegumental antigen 28.5 kDa TA, cathepsin B3 protease CatB3, Type I cystatin stefin-1, cathepsin L5, cathepsin L1g and cathepsin B, fatty acid binding protein FABP, leucine aminopeptidases LAP (Fasciola hepatica and Fasciola gigantica, Fasciolosis); prion protein (FFI prion, Fatal familial insomnia (FFI)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, secreted larval acidic proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Filarioidea superfamily, Filariasis); phospholipase C PLC, heat-labile enterotoxin B, Iota toxin component Ib, protein CPE1281, pyruvate ferredoxin oxidoreductase, elongation factor G EF-G, perfringolysin 0 Pfo, glyceraldehyde-3-phosphate dehydrogenase GapC, Fructose-bisphosphate aldolase Alf2, clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (Clostridium perfringens, Food poisoning by Clostridium perfringens); leukotoxin IktA, adhesion FadA, outer membrane protein RadD, high-molecular weight arginine-binding protein (Fusobacterium genus, Fusobacterium infection); phospholipase C PLC, heat-labile enterotoxin B, Iota toxin component Ib, protein CPE1281, pyruvate ferredoxin oxidoreductase, elongation factor G EF-G, perfringolysin 0 Pfo, glyceraldehyde-3-phosphate dehydrogenase GapC, fructose-bisphosphate aldolase Alf2, clostridium perfringens enterotoxin CPE, alpha toxin AT, alpha toxoid ATd, epsilon-toxoid ETd, protein HP, large cytotoxin TpeL, endo-beta-N-acetylglucosaminidase Naglu, phosphoglyceromutase Pgm (usually Clostridium perfringens; other Clostridium species, Gas gangrene (Clostridial myonecrosis)); lipase A, lipase B, peroxidase Dec1 (Geotrichum candidum, Geotrichosis); prion protein (GSS prion, Gerstmann-Sträussler-Scheinker syndrome (GSS)); cyst wall proteins CWP1, CWP2, CWP3, variant surface protein VSP, VSP1, VSP2, VSP3, VSP4, VSP5, VSP6, 56 kDa antigen, pyruvate ferredoxin oxidoreductase PFOR, alcohol dehydrogenase E ADHE, alpha-giardin, alpha8-giardin, alpha1-guiardin, beta-giardin, cystein proteases, glutathione-S-transferase GST, arginine deiminase ADI, fructose-1,6-bisphosphat aldolase FBA, Giardia trophozoite antigens GTA (GTA1, GTA2), ornithine carboxyl transferase OCT, striated fiber-asseblin-like protein SALP, uridine phosphoryl-like protein UPL, alpha-tubulin, beta-tubulin (Giardia intestinalis, Giardiasis); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein (Burkholderia mallei, Glanders); cyclophilin CyP, 24 kDa third-stage larvae protien GS24, excretion-secretion products ESPs (40, 80, 120 and 208 kDa) (Gnathostoma spinigerum and Gnathostoma hispidum, Gnathostomiasis); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR (Neisseria gonorrhoeae, Gonorrhea); outer membrane protein A OmpA, outer membrane protein C OmpC, outer membrane protein K17 OmpK17 (Klebsiella granulomatis, Granuloma inguinale (Donovanosis)); fibronectin-binding protein Sfb, fibronectin/fibrinogen-binding protein FBP54, fibronectin-binding protein FbaA, M protein type 1 Emm1, M protein type 6 Emm6, immunoglobulin-binding protein 35 Sib35, Surface protein R28 Spr28, superoxide dismutase SOD, C5a peptidase ScpA, antigen I/II Agl/II, adhesin AspA, G-related alpha2-macroglobulin-binding protein GRAB, surface fibrillar protein M5 (Streptococcus pyogenes, Group A streptococcal infection); C protein β antigen, arginine deiminase proteins, adhesin BibA, 105 kDA protein BPS, surface antigens c, surface antigens R, surface antigens X, trypsin-resistant protein R1, trypsin-resistant protein R3, trypsin-resistant protein R4, surface immunogenic protein Sip, surface protein Rib, Leucine-rich repeats protein LrrG, serine-rich repeat protein Srr-2, C protein alpha-antigen Bca, Beta antigen Bag, surface antigen Epsilon, alpha-like protein ALP1, alpha-like protein ALP5 surface antigen delta, alpha-like protein ALP2, alpha-like protein ALP3, alpha-like protein ALP4, Cbeta protein Bac (Streptococcus agalactiae, Group B streptococcal infection); transferrin-binding protein 2 Tbp2, phosphatase P4, outer membrane protein P6, peptidoglycan-associated lipoprotein Pal, protein D, protein E, adherence and penetration protein Hap, outer membrane protein 26 Omp26, outer membrane protein P5 (Fimbrin), outer membrane protein D15, outer membrane protein OmpP2, 5′-nucleotidase NucA, outer membrane protein P1, outer membrane protein P2, outer membrane lipoprotein Pcp, Lipoprotein E, outer membrane protein P4, fuculokinase fucK, [Cu,Zn]-superoxide dismutase SodC, protease HtrA, protein 0145, alpha-galactosylceramide (Haemophilus influenzae, Haemophilus influenzae infection); polymerase 3D, viral capsid protein VP1, viral capsid protein VP2, viral capsid protein VP3, viral capsid protein VP4, protease 2A, protease 3C (Enteroviruses, mainly Coxsackie A virus and Enterovirus 71 (EV71), Hand, foot and mouth disease (HFMD)); RNA polymerase L, protein L, glycoprotein Gn, glycoprotein Gc, nucleocapsid protein S, envelope glycoprotein G1, nucleoprotein NP, protein N, polyprotein M (Sin Nombre virus, Hantavirus, Hantavirus Pulmonary Syndrome (HPS)); heat shock protein HspA, heat shock protein HspB, citrate synthase GltA, protein UreB, heat shock protein Hsp60, neutrophil-activating protein NAP, catalase KatA, vacuolating cytotoxin VacA, urease alpha UreA, urease beta Ureb, protein Cpn10, protein groES, heat shock protein Hsp10, protein MopB, cytotoxicity-associated 10 kDa protein CAG, 36 kDa antigen, beta-lactamase HcpA, Beta-lactamase HcpB (Helicobacter pylori, Helicobacter pylori infection); integral membrane proteins, aggregation-prone proteins, 0-antigen, toxin-antigens Stx2B, toxin-antigen Stx1B, adhesion-antigen fragment Int28, protein EspA, protein EspB, Intimin, protein Tir, protein IntC300, protein Eae (Escherichia coli O157:H7, O111 and O104:H4, Hemolytic-uremic syndrome (HUS)); RNA polymerase L, protein L, glycoprotein Gn, glycoprotein Gc, nucleocapsid protein S, envelope glycoprotein G1, nucleoprotein NP, protein N, polyprotein M (Bunyaviridae family, Hemorrhagic fever with renal syndrome (HFRS)); glycoprotein G, matrix protein M, nucleoprotein N, fusion protein F, polymerase L, protein W, proteinC, phosphoprotein p, non-structural protein V (Henipavirus (Hendra virus Nipah virus), Henipavirus infections); polyprotein, glycoproten Gp2, hepatitis A surface antigen HBAg, protein 2A, virus protein VP1, virus protein VP2, virus protein VP3, virus protein VP4, protein P1B, protein P2A, protein P3AB, protein P3D (Hepatitis A Virus, Hepatitis A); hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 (Hepatitis B Virus (HBV), Hepatitis B); envelope glycoprotein E1 gp32 gp35, envelope glycoprotein E2 NS1 gp68 gp70, capsid protein C, core protein Core, polyprotein, virus protein VP1, virus protein VP2, virus protein VP3, virus protein VP4, antigen G, protein NS3, protein NS5A, (Hepatitis C Virus, Hepatitis C); virus protein VP1, virus protein VP2, virus protein VP3, virus protein VP4, large hepatitis delta antigen, small hepatitis delta antigen (Hepatitis D Virus, Hepatitis D); virus protein VP1, virus protein VP2, virus protein VP3, virus protein VP4, capsid protein E2 (Hepatitis E Virus, Hepatitis E); glycoprotein L UL1, uracil-DNA glycosylase UL2, protein UL3, protein UL4, DNA replication protein UL5, portal protein UL6, virion maturation protein UL7, DNA helicase UL8, replication origin-binding protein UL9, glycoprotein M UL10, protein UL11, alkaline exonuclease UL12, serine-threonine protein kinase UL13, tegument protein UL14, terminase UL15, tegument protein UL16, protein UL17, capsid protein VP23 UL18, major capsid protein VP5 UL19, membrane protein UL20, tegument protein UL21, Glycoprotein H (UL22), Thymidine Kinase UL23, protein UL24, protein UL25, capsid protein P40 (UL26, VP24, VP22A), glycoprotein B (UL27), ICP18.5 protein (UL28), major DNA-binding protein ICP8 (UL29), DNA polymerase UL30, nuclear matrix protein UL31, envelope glycoprotein UL32, protein UL33, inner nuclear membrane protein UL34, capsid protein VP26 (UL35), large tegument protein UL36, capsid assembly protein UL37, VP19C protein (UL38), ribonucleotide reductase (Large subunit) UL39, ribonucleotide reductase (Small subunit) UL40, tegument protein/virion host shutoff VHS protein (UL41), DNA polymerase processivity factor UL42, membrane protein UL43, glycoprotein C (UL44), membrane protein UL45, tegument proteins VP11/12 (UL46), tegument protein VP13/14 (UL47), virion maturation protein VP16 (UL48, Alpha-TIF), envelope protein UL49, dUTP diphosphatase UL50, tegument protein UL51, DNA helicase/primase complex protein UL52, glycoprotein K (UL53), transcriptional regulation protein IE63 (ICP27, UL54), protein UL55, protein UL56, viral replication protein ICP22 (IE68, US1), protein US2, serine/threonine-protein kinase US3, glycoprotein G (US4), glycoprotein J (US5), glycoprotein D (US6), glycoprotein I (US7), glycoprotein E (US8), tegument protein US9, capsid/tegument protein US10, Vmw21 protein (US11), ICP47 protein (IE12, US12), major transcriptional activator ICP4 (IE175, RS1), E3 ubiquitin ligase ICP0 (IE110), latency-related protein 1 LRP1, latency-related protein 2 LRP2, neurovirulence factor RL1 (ICP34.5), latency-associated transcript LAT (Herpes simplex virus 1 and 2 (HSV-1 and HSV-2), Herpes simplex); heat shock protein Hsp60, cell surface protein H1C, dipeptidyl peptidase type IV DppIV, M antigen, 70 kDa protein, 17 kDa histone-like protein (Histoplasma capsulatum, Histoplasmosis); fatty acid and retinol binding protein-1 FAR-1, tissue inhibitor of metalloproteinase TIMP (TMP), cysteine proteinase ACEY-1, cysteine proteinase ACCP-1, surface antigen Ac-16, secreted protein 2 ASP-2, metalloprotease 1 MTP-1, aspartyl protease inhibitor API-1, surface-associated antigen SAA-1, surface-associated antigen SAA-2, adult-specific secreted factor Xa, serine protease inhibitor anticoagulant AP, cathepsin D-like aspartic protease ARR-1, glutathione S-transferase GST, aspartic protease APR-1, acetylcholinesterase AChE (Ancylostoma duodenale and Necator americanus, Hookworm infection); protein NS1, protein NP1, protein VP1, protein VP2, protein VP3 (Human bocavirus (HBoV), Human bocavirus infection); major surface protein 2 MSP2, major surface protein 4 MSP4, MSP variant SGV1, MSP variant SGV2, outer membrane protein OMP, outer membrande protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia ewingii, Human ewingii ehrlichiosis); major surface proteins 1-5 (MSP1a, MSP1b, MSP2, MSP3, MSP4, MSP5), type IV secretion system proteins VirB2, VirB7, VirB11, VirD4 (Anaplasma phagocytophilum, Human granulocytic anaplasmosis (HGA)); protein NS1, small hydrophobic protein NS2, SH protein, fusion protein F, glycoprotein G, matrix protein M, matrix protein M2-1, matrix protein M2-2, phosphoprotein P, nucleoprotein N, polymerase L (Human metapneumovirus (hMPV), Human metapneumovirus infection); major surface protein 2 MSP2, major surface protein 4 MSP4, MSP variant SGV1, MSP variant SGV2, outer membrane protein OMP, outer membrande protein 19 OMP-19, major antigenic protein MAP1, major antigenic protein MAP1-2, major antigenic protein MAP1B, major antigenic protein MAP1-3, Erum2510 coding protein, protein GroEL, protein GroES, 30-kDA major outer membrane proteins, GE 100-kDa protein, GE 130-kDa protein, GE 160-kDa protein (Ehrlichia chaffeensis, Human monocytic ehrlichiosis); replication protein E1, regulatory protein E2, protein E3, protein E4, protein E5, protein E6, protein E7, protein E8, major capsid protein L1, minor capsid protein L2 (Human papillomavirus (HPV), Human papillomavirus (HPV) infection); fusion protein F, hemagglutinin-neuramidase HN, glycoprotein G, matrix protein M, phosphoprotein P, nucleoprotein N, polymerase L (Human parainfluenza viruses (HPIV), Human parainfluenza virus infection); Hemagglutinin (HA), Neuraminidase (NA), Nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein (Orthomyxoviridae family, Influenza virus (flu)); genome polyprotein, protein E, protein M, capsid protein C (Japanese encephalitis virus, Japanese encephalitis); RTX toxin, type IV pili, major pilus subunit PilA, regulatory transcription factors PilS and PilR, protein sigma54, outer membrane proteins (Kingella kingae, Kingella kingae infection); prion protein (Kuru prion, Kuru); nucleoprotein N, polymerase L, matrix protein Z, glycoprotein GP (Lassa virus, Lassa fever); peptidoglycan-associated lipoprotein PAL, 60 kDa chaperonin Cpn60 (groEL, HspB), type IV pilin PilE, outer membrane protein MIP, major outer membrane protein MompS, zinc metalloproteinase MSP (Legionella pneumophila, Legionellosis (Legionnaires' disease, Pontiac fever)); P4 nuclease, protein WD, ribonucleotide reductase M2, surface membrane glycoprotein Pg46, cysteine proteinase CP, glucose-regulated protein 78 GRP-78, stage-specific S antigen-like protein A2, ATPase F1, beta-tubulin, heat shock protein 70 Hsp70, KMP-11, glycoprotein GP63, protein BT1, nucleoside hydrolase NH, cell surface protein B1, ribosomal protein P1-like protein P1, sterol 24-c-methyltransferase SMT, LACK protein, histone H1, SPB1 protein, thiol specific antioxidant TSA, protein antigen STI1, signal peptidase SP, histone H2B, surface antigen PSA-2, cystein proteinase b Cpb (Leishmania genus, Leishmaniasis); major membrane protein I, serine-rich antigen—45 kDa, 10 kDa caperonin GroES, HSP kDa antigen, amino-oxononanoate synthase AONS, protein recombinase A RecA, Acetyl-/propionyl-coenzyme A carboxylase alpha, alanine racemase, 60 kDa chaperonin 2, ESAT-6-like protein EcxB (L-ESAT-6), protein Lsr2, protein ML0276, Heparin-binding hemagglutinin HBHA, heat-shock protein 65 Hsp65, mycP1 or ML0041 coding protein, htrA2 or ML0176 coding protein, htrA4 or ML2659 coding protein, gcp or ML0379 coding protein, clpC or ML0235 coding protein (Mycobacterium leprae and Mycobacterium lepromatosis, Leprosy); outer membrane protein LipL32, membrane protein LIC10258, membrane protein LP30, membrane protein LIC12238, Ompa-like protein Lsa66, surface protein LigA, surface protein LigB, major outer membrane protein OmpL1, outer membrane protein LipL41, protein LigAni, surface protein LcpA, adhesion protein LipL53, outer membrane protein UpL32, surface protein Lsa63, flagellin FlaB1, membran lipoprotein LipL21, membrane protein pL40, leptospiral surface adhesin Lsa27, outer membrane protein OmpL36, outer membrane protein OmpL37, outer membrane protein OmpL47, outer membrane protein OmpL54, acyltransferase LpxA (Leptospira genus, Leptospirosis); listeriolysin O precursor Hly (LLO), invasion-associated protein lap (P60), Listeriolysin regulatory protein PrfA, Zinc metalloproteinase Mpl, Phosphatidylinositol—specific phospholipase C PLC (PlcA, PlcB), O-acetyltransferase Oat, ABC-transporter permease Im.G_1771, adhesion protein LAP, LAP receptor Hsp60, adhesin LapB, haemolysin listeriolysin O LLO, protein ActA, Internalin A InlA, protein InlB (Listeria monocytogenes, Listeriosis); outer surface protein A OspA, outer surface protein OspB, outer surface protein OspC, decorin binding protein A DbpA, decorin binding protein B DbpB, flagellar filament 41 kDa core protein Fla, basic membrane protein A BmpA (Immunodominant antigen P39), outer surface 22 kDa lipoprotein precursor (antigen IPLA7), variable surface lipoprotein vlsE (usually Borrelia burgdorferi and other Borrelia species, Lyme disease (Lyme borreliosis)); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, protein Cox-2 (Wuchereria bancrofti and Brugia malayi, Lymphatic filariasis (Elephantiasis)); glycoprotein GP, matrix protein Z, polymerase L, nucleoprotein N (Lymphocytic choriomeningitis virus (LCMV), Lymphocytic choriomeningitis); thrombospondin-related anonymous protein TRAP, SSP2 Sporozoite surface protein 2, apical membrane antigen 1 AMA1, rhoptry membrane antigen RMA1, acidic basic repeat antigen ABRA, cell-traversal protein PF, protein Pvs25, merozoite surface protein 1 MSP-1, merozoite surface protein 2 MSP-2, ring-infected erythrocyte surface antigen RESALiver stage antigen 3 LSA-3, protein Eba-175, serine repeat antigen 5 SERA-5, circumsporozoite protein CS, merozoite surface protein 3 MSP3, merozoite surface protein 8 MSP8, enolase PF10, hepatocyte erythrocyte protein 17 kDa HEP17, erythrocyte membrane protein 1 EMP1, protein Kbetamerozoite surface protein 4/5 MSP 4/5, heat shock protein Hsp90, glutamate-rich protein GLURP, merozoite surface protein 4 MSP-4, protein STARP, circumsporozoite protein-related antigen precursor CRA (Plasmodium genus, Malaria); nucleoprotein N, membrane-associated protein VP24, minor nucleoprotein VP30, polymerase cofactor VP35, polymerase L, matrix protein VP40, envelope glycoprotein GP (Marburg virus, Marburg hemorrhagic fever (MHF)); protein C, matrix protein M, phosphoprotein P, non-structural protein V, hemagglutinin glycoprotein H, polymerase L, nucleoprotein N, fusion protein F (Measles virus, Measles); members of the ABC transporter family (LoIC, OppA, and PotF), putative lipoprotein releasing system transmembrane protein LoIC/E, flagellin FliC, Burkholderia intracellular motility A BimA, bacterial Elongation factor-Tu EF-Tu, 17 kDa OmpA-like protein, boaA coding protein, boaB coding protein (Burkholderia pseudomallei, Melioidosis (Whitmore's disease)); pilin proteins, minor pilin-associated subunit pilC, major pilin subunit and variants pilE, pilS, phase variation protein porA, Porin B PorB, protein TraD, Neisserial outer membrane antigen H.8, 70 kDa antigen, major outer membrane protein PI, outer membrane proteins PIA and PIB, W antigen, surface protein A NspA, transferrin binding protein TbpA, transferrin binding protein TbpB, PBP2, mtrR coding protein, ponA coding protein, membrane permease FbpBC, FbpABC protein system, LbpAB proteins, outer membrane protein Opa, outer membrane transporter FetA, iron-repressed regulator MpeR, factor H-binding protein fHbp, adhesin NadA, protein NhbA, repressor FarR (Neisseria meningitidis, Meningococcal disease); 66 kDa protein, 22 kDa protein (usually Metagonimus yokagawai, Metagonimiasis); polar tube proteins (34, 75, and 170 kDa in Glugea, 35, 55 and 150 kDa in Encephalitozoon), kinesin-related protein, RNA polymerase II largest subunit, similar of integral membrane protein YIPA, anti-silencing protein 1, heat shock transcription factor HSF, protein kinase, thymidine kinase, NOP-2 like nucleolar protein (Microsporidia phylum, Microsporidiosis); CASP8 and FADD-like apoptosis regulator, Glutathione peroxidase GPX1, RNA helicase NPH-II NPH2, Poly(A) polymerase catalytic subunit PAPL, Major envelope protein P43K, early transcription factor 70 kDa subunit VETFS, early transcription factor 82 kDa subunit VETFL, metalloendopeptidase G1-type, nucleoside triphosphatase I NPH1, replication protein A28-like MC134L, RNA polymease 7 kDa subunit RPO7 (Molluscum contagiosum virus (MCV), Molluscum contagiosum (MC)); matrix protein M, phosphoprotein PN, small hydrophobic protein SH, nucleoprotein N, protein V, fusion glycoprotein F, hemagglutinin-neuraminidase HN, RNA polymerase L (Mumps virus, Mumps); Outer membrane proteins OM, cell surface antigen OmpA, cell surface antigen OmpB (sca5), cell surface protein SCA4, cell surface protein SCA1, intracytoplasmic protein D, crystalline surface layer protein SLP, protective surface protein antigen SPA (Rickettsia typhi, Murine typhus (Endemic typhus)); adhesin P1, adhesion P30, protein p116, protein P40, cytoskeletal protein HMW1, cytoskeletal protein HMW2, cytoskeletal protein HMW3, MPN152 coding protein, MPN426 coding protein, MPN456 coding protein, MPN-500coding protein (Mycoplasma pneumoniae, Mycoplasma pneumonia); NocA, Iron dependent regulatory protein, VapA, VapD, VapF, VapG, caseinolytic protease, filament tip-associated 43-kDa protein, protein P24, protein P61, 15-kDa protein, 56-kDa protein (usually Nocardia asteroides and other Nocardia species, Nocardiosis); venom allergen homolog-like protein VAL-1, abundant larval transcript ALT-1, abundant larval transcript ALT-2, thioredoxin peroxidase TPX, vespid allergen homologue VAH, thiordoxin peroxidase 2 TPX-2, antigenic protein SXP (peptides N, N1, N2, and N3), activation associated protein-1 ASP-1, Thioredoxin TRX, transglutaminase BmTGA, glutathione-S-transferases GST, myosin, vespid allergen homologue VAH, 175 kDa collagenase, glyceraldehyde-3-phosphate dehydrogenase GAPDH, cuticular collagen Col-4, Secreted Larval Acidic Proteins SLAPs, chitinase CHI-1, maltose binding protein MBP, glycolytic enzyme fructose-1,6-bisphosphate aldolase Fba, tropomyosin TMY-1, nematode specific gene product OvB20, onchocystatin CPI-2, Cox-2 (Onchocerca volvulus, Onchocerciasis (River blindness)); 43 kDa secreted glycoprotein, glycoprotein gp0, glycoprotein gp75, antigen Pb27, antigen Pb40, heat shock protein Hsp65, heat shock protein Hsp70, heat shock protein Hsp90, protein P10, triosephosphate isomerase TPI, N-acetyl-glucosamine-binding lectin Paracoccin, 28 kDa protein Pb28 (Paracoccidioides brasiliensis, Paracoccidioidomycosis (South American blastomycosis)); 28-kDa cruzipain-like cystein protease Pw28CCP (usually Paragonimus westermani and other Paragonimus species, Paragonimiasis); outer membrane protein OmpH, outer membrane protein Omp28, protein PM1539, protein PM0355, protein PM1417, repair protein MutL, protein BcbC, prtein PM0305, formate dehydrogenase-N, protein PM0698, protein PM1422, DNA gyrase, lipoprotein PlpE, adhesive protein Cp39, heme aquisition system receptor HasR, 39 kDa capsular protein, iron-regulated OMP IROMP, outer membrane protein OmpA87, fimbrial protein Ptf, fimbrial subunit protein PtfA, transferrin binding protein TbpI, esterase enzyme MesA, Pasteurella multocida toxin PMT, adhesive protein Cp39 (Pasteurella genus, Pasteurellosis); “filamentous hemagglutinin FhaB, adenylate cyclase CyaA, pertussis toxin subunit 4 precursor PtxD, pertactin precursor Prn, toxin subunit 1 PtxA, protein Cpn60, protein brkA, pertussis toxin subunit 2 precursor PtxB, pertussis toxin subunit 3 precursor PtxC, pertussis toxin subunit 5 precursor PtxE, pertactin Prn, protein Fim2, protein Fim3;” (Bordetella pertussis, Pertussis (Whooping cough)); “F1 capsule antigen, virulence-associated V antigen, secreted effector protein LcrV, V antigen, outer membrane protease Pla, secreted effector protein YopD, putative secreted protein-tyrosine phosphatase YopH, needle complex major subunit YscF, protein kinase YopO, putative autotransporter protein YapF, inner membrane ABC-transporter YbtQ (Irp7), putative sugar binding protein YPO0612, heat shock protein 90 HtpG, putative sulfatase protein YdeN, outer-membrane lipoprotein carrier protein LolA, secretion chaperone YerA, putative lipoprotein YPO0420, hemolysin activator protein HpmB, pesticin/yersiniabactin outer membrane receptor Psn, secreted effector protein YopE, secreted effector protein YopF, secreted effector protein YopK, outer membrane protein YopN, outer membrane protein YopM, Coagulase/fibrinolysin precursor Pla;” (Yersinia pestis, Plague); protein PhpA, surface adhesin PsaA, pneumolysin Ply, ATP-dependent protease Clp, lipoate-protein ligase LplA, cell wall surface anchored protein psrP, sortase SrtA, glutamyl-tRNA synthetase GItX, choline binding protein A CbpA, pneumococcal surface protein A PspA, pneumococcal surface protein C PspC, 6-phosphogluconate dehydrogenase Gnd, iron-binding protein PiaA, Murein hydrolase LytB, proteon LytC, protease A1 (Streptococcus pneumoniae, Pneumococcal infection); major surface protein B, kexin-like protease KEX1, protein A12, 55 kDa antigen P55, major surface glycoprotein Msg (Pneumocystis jirovecii, Pneumocystis pneumonia (PCP)); genome polyprotein, polymerase 3D, viral capsid protein VP1, viral capsid protein VP2, viral capsid protein VP3, viral capsid protein VP4, protease 2A, protease 3C (Poliovirus, Poliomyelitis); protein Nfa1, exendin-3, secretory lipase, cathepsin B-like protease, cysteine protease, cathepsin, peroxiredoxin, protein Cry1Ac (usually Naegleria fowleri, Primary amoebic meningoencephalitis (PAM)); agnoprotein, large T antigen, small T antigen, major capsid protein VP1, minor capsid protein Vp2 (JC virus, Progressive multifocal leukoencephalopathy); low calcium response protein E LCrE, chlamydial outer protein N CopN, serine/threonine-protein kinase PknD, acyl-carrier-protein S-malonyltransferase FabD, single-stranded DNA-binding protein Ssb, major outer membrane protein MOMP, outer membrane protein 2 Omp2, polymorphic membrane protein family (Pmp1, Pmp2, Pmp3, Pmp4, Pmp5, Pmp6, Pmp7, Pmp8, Pmp9, Pmp10, Pmp11, Pmp12, Pmp13, Pmp14, Pmp15, Pmp16, Pmp17, Pmp18, Pmp19, Pmp20, Pmp21) (Chlamydophila psittaci, Psittacosis); outer membrane protein P1, heat shock protein B HspB, peptide ABC transporter, GTP-binding protein, protein IcmB, ribonuclease R, phosphatas SixA, protein DsbD, outer membrane protein TolC, DNA-binding protein PhoB, ATPase DotB, heat shock protein B HspB, membrane protein Com1, 28 kDa protein, DNA-3-methyladenine glycosidase I, pouter membrane protein OmpH, outer membrane protein AdaA, glycine cleavage system T-protein (Coxiella burnetii, Q fever); nucleoprotein N, large structural protein L, phophoprotein P, matrix protein M, glycoprotein G (Rabies virus, Rabies); fusionprotein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 (Respiratory syncytial virus (RSV), Respiratory syncytial virus infection); genome polyprotein, polymerase 3D, viral capsid protein VP1, viral capsid protein VP2, viral capsid protein VP3, viral capsid protein VP4, protease 2A, protease 3C (Rhinovirus, Rhinovirus infection); outer membrane proteins OM, cell surface antigen OmpA, cell surface antigen OmpB (sca5), cell surface protein SCA4, cell surface protein SCA1, protein PS120, intracytoplasmic protein D, protective surface protein antigen SPA (Rickettsia genus, Rickettsial infection); outer membrane proteins OM, cell surface antigen OmpA, cell surface antigen OmpB (sca5), cell surface protein SCA4, cell surface protein SCA1, intracytoplasmic protein D (Rickettsia akari, Rickettsialpox); envelope glycoprotein GP, polymerase L, nucleoprotein N, non-structural protein NSS (Rift Valley fever virus, Rift Valley fever (RVF)); outer membrane proteins OM, cell surface antigen OmpA, cell surface antigen OmpB (sca5), cell surface protein SCA4, cell surface protein SCA1, intracytoplasmic protein D (Rickettsia rickettsii, Rocky mountain spotted fever (RMSF)); “non-structural protein 6 NS6, non-structural protein 2 NS2, intermediate capsid protein VP6, inner capsid protein VP2, non-structural protein 3 NS3, RNA-directed RNA polymerase L, protein VP3, non-structural protein 1 NS1, non-structural protein 5 NS5, outer capsid glycoprotein VP7, non-structural glycoprotein 4 NS4, outer capsid protein VP4” (Rotavirus, Rotavirus infection); polyprotein P200, glycoprotein E1, glycoprotein E2, protein NS2, capsid protein C (Rubella virus, Rubella); chaperonin GroEL (MopA), inositol phosphate phosphatase SopB, heat shock protein HslU, chaperone protein DnaJ, protein TviB, protein IroN, flagellin FliC, invasion protein SipC, glycoprotein gp43, outer membrane protein LamB, outer membrane protein PagC, outer membrane protein TolC, outer membrane protein NmpC, outer membrane protein FadL, transport protein SadA, transferase WgaP, effector proteins SifA, SteC, SseL, SseJ and SseF (Salmonella genus, Salmonellosis), protein 14, non-structural protein NS7b, non-structural protein NS8a, protein 9b, protein 3a, nucleoprotein N, non-structural protein NS3b, non-structural protein NS6, protein 7a, non-structural protein NS8b, membrane protein M, envelope small membrane protein EsM, replicase polyprotein 1a, spike glycoprotein S, replicase polyprotein 1 ab; (SARS coronavirus, SARS (Severe Acute Respiratory Syndrome)); serin protease, Atypical Sarcoptes Antigen 1 ASA1, glutathione S-transferases GST, cystein protease, serine protease, apolipoprotein (Sarcoptes scabiei, Scabies); glutathione S-transferases GST, paramyosin, hemoglbinase SM32, major egg antigen, 14 kDa fatty acid-binding protein Sm14, major larval surface antigen P37, 22.6 kDa tegumental antigen, calpain CANP, triphospate isomerase Tim, surface protein 9B, outer capsid protein VP2, 23 kDa integral membrane protein Sm23, Cu/Zn-superoxide dismutase, glycoprotein Gp, myosin (Schistosoma genus, Schistosomiasis (Bilharziosis)); 60 kDa chaperonin, 56 kDa type-specific antigen, pyruvate phosphate dikinase, 4-hydroxybenzoate octaprenyltransferase (Orientia tsutsugamushi, Scrub typhus); dehydrogenase GuaB, invasion protein Spa32, invasin IpaA, invasin IpaB, invasin IpaC, invasin IpaD, invasin IpaH, invasin IpaJ (Shigella genus, Shigellosis (Bacillary dysentery)); protein P53, virion protein US10 homolog, transcriptional regulator IE63, transcriptional transactivator IE62, protease P33, alpha trans-inducing factor 74 kDa protein, deoxyuridine 5′-triphosphate nucleotidohydrolase, transcriptional transactivator IE4, membrane protein UL43 homolog, nuclear phosphoprotein UL3 homolog, nuclear protein UL4 homolog, replication origin-binding protein, membrane protein 2, phosphoprotein 32, protein 57, DNA polymerase processivity factor, portal protein 54, DNA primase, tegument protein UL14 homolog, tegument protein UL21 homolog, tegument protein UL55 homolog, tripartite terminase subunit UL33 homolog, tripartite terminase subunit UL15 homolog, capsid-binding protein 44, virion-packaging protein 43 (Varicella zoster virus (VZV), Shingles (Herpes zoster)); truncated 3-beta hydroxy-5-ene steroid dehydrogenase homolog, virion membrane protein A13, protein A19, protein A31, truncated protein A35 homolog, protein A37.5 homolog, protein A47, protein A49, protein A51, semaphorin-like protein A43, serine proteinase inhibitor 1, serine proteinase inhibitor 2, serine proteinase inhibitor 3, protein A6, protein B15, protein C1, protein C5, protein C6, protein F7, protein F8, protein F9, protein F11, protein F14, protein F15, protein F16 (Variola major or Variola minor, Smallpox (Variola)); adhesin/glycoprotein gp70, proteases (Sporothrix schenckii, Sporotrichosis); heme-iron binding protein IsdB, collagen adhesin Cna, clumping factor A ClfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus, Staphylococcal food poisoning); heme-iron binding protein IsdB, collagen adhesin Cna, clumping factor A ClfA, protein MecA, fibronectin-binding protein A FnbA, enterotoxin type A EntA, enterotoxin type B EntB, enterotoxin type C EntC1, enterotoxin type C EntC2, enterotoxin type D EntD, enterotoxin type E EntE, Toxic shock syndrome toxin-1 TSST-1, Staphylokinase, Penicillin binding protein 2a PBP2a (MecA), secretory antigen SssA (Staphylococcus genus e.g. aureus, Staphylococcal infection); antigen Ss-IR, antigen NIE, strongylastacin, Na+-K+ ATPase Sseat-6, tropomysin SsTmy-1, protein LEC-5, 41 kDa antigen P5, 41-kDa larval protein, 31-kDa larval protein, 28-kDa larval protein (Strongyloides stercoralis, Strongyloidiasis); glycerophosphodiester phosphodiesterase GlpQ (Gpd), outer membrane protein TmpB, protein Tp92, antigen TpF1, repeat protein Tpr, repeat protein F TprF, repeat protein G TprG, repeat protein I TprI, repeat protein J TprJ, repeat protein K TprK, treponemal membrane protein A TmpA, lipoprotein, 15 kDa Tpp15, 47 kDa membrane antigen, miniferritin TpF1, adhesin Tp0751, lipoprotein TP0136, protein TpN17, protein TpN47, outer membrane protein TP0136, outer membrane protein TP0155, outer membrane protein TP0326, outer membrane protein TP0483, outer membrane protein TP0956 (Treponema pallidum, Syphilis); Cathepsin L-like proteases, 53/25-kDa antigen, 8 kDa family members, cysticercus protein with a marginal trypsin-like activity TsAg5, oncosphere protein TSOL18, oncosphere protein TSOL45-1A, lactate dehydrogenase A LDHA, lactate dehydrogenase B LDHB (Taenia genus, Taeniasis); tetanus toxin TetX, tetanus toxin C TTC, 140 kDa S layer protein, flavoprotein beta-subunit CT3, phospholipase (lecithinase), phosphocarrier protein HPr (Clostridium tetani, Tetanus (Lockjaw)); genome polyprotein, protein E, protein M, capsid protein C (Tick-borne encephalitis virus (TBEV), Tick-borne encephalitis); 58-kDa antigen, 68-kDa antigens, Toxocara larvae excretory-secretory antigen TES, 32-kDa glycoprotein, glycoprotein TES-70, glycoprotein GP31, excretory-secretory antigen TcES-57, perienteric fluid antigen Pe, soluble extract antigens Ex, excretory/secretory larval antigens ES, antigen TES-120, polyprotein allergen TBA-1, cathepsin L-like cysteine protease c-cpl-1, 26-kDa protein (Toxocara canis or Toxocara cati, Toxocariasis (Ocular Larva Migrans (OLM) and Visceral Larva Migrans (VLM))); microneme proteins (MIC1, MIC2, MIC3, MIC4, MIC5, MIC6, MIC7, MIC8), rhoptry protein Rop2, rhoptry proteins (Rop1, Rop2, Rop3, Rop4, Rop5, Rop6, Rop7, Rop16, Rjop17), protein SR1, surface antigen P22, major antigen p24, major surface antigen p30, dense granule proteins (GRA1, GRA2, GRA3, GRA4, GRA5, GRA6, GRA7, GRA8, GRA9, GRA10), 28 kDa antigen, surface antigen SAG1, SAG2 related antigen, nucleoside-triphosphatase 1, nucleoside-triphosphatase 2, protein Stt3, HesB-like domain-containing protein, rhomboid-like protease 5, toxomepsin 1 (Toxoplasma gondii, Toxoplasmosis); 43 kDa secreted glycoprotein, 53 kDa secreted glycoprotein, paramyosin, antigen Ts21, antigen Ts87, antigen p46000, TSL-1 antigens, caveolin-1 CAV-1, 49 kDa newborn larva antigen, prosaposin homologue, serine protease, serine proteinase inhibitor, 45-kDa glycoprotein Gp45 (Trichinella spiralis, Trichinellosis); Myb-like transcriptional factors (Myb1, Myb2, Myb3), adhesion protein AP23, adhesion protein AP33, adhesin protein AP33-3, adhesins AP51, adhesin AP65, adhesion protein AP65-1, alpha-actinin, kinesin-associated protein, teneurin, 62 kDa proteinase, subtilisin-like serine protease SUB1, cysteine proteinase gene 3 CP3, alpha-enolase Eno1, cysteine proteinase CP30, heat shock proteins (Hsp70, Hsp60), immunogenic protein P270, (Trichomonas vaginalis, Trichomoniasis); beta-tubulin, 47-kDa protein, secretory leucocyte-like proteinase-1 SLP-1, 50-kDa protein TT50, 17 kDa antigen, 43/47 kDa protein (Trichuris trichiura, Trichuriasis (Whipworm infection)); protein ESAT-6 (EsxA), 10 kDa filtrate antigen EsxB, secreted antigen 85-B FBPB, fibronectin-binding protein A FbpA (Ag85A), serine protease PepA, PPE family protein PPE18, fibronectin-binding protein D FbpD, immunogenic protein MPT64, secreted protein MPT51, catalase-peroxidase-peroxynitritase T KATG, periplasmic phosphate-binding lipoprotein PSTS3 (PBP-3, Phos-1), iron-regulated heparin binding hemagglutinin Hbha, PPE family protein PPE14, PPE family protein PPE68, protein Mtb72F, protein Apa, immunogenic protein MPT63, periplasmic phosphate-binding lipoprotein PSTS1 (PBP-1), molecular chaperone DnaK, cell surface lipoprotein Mpt83, lipoprotein P23, phosphate transport system permease protein pstA, 14 kDa antigen, fibronectin-binding protein C FbpC1, Alanine dehydrogenase TB43, Glutamine synthetase 1, ESX-1 protein, protein CFP10, TB10.4 protein, protein MPT83, protein MTB12, protein MTB8, Rpf-like proteins, protein MTB32, protein MTB39, crystallin, heat-shock protein HSP65, protein PST-S (usually Mycobacterium tuberculosis, Tuberculosis); outer membrane protein FobA, outer membrane protein FobB, intracellular growth locus IglC1, intracellular growth locus IglC2, aminotransferase Wbtl, chaperonin GroEL, 17 kDa major membrane protein TUL4, lipoprotein LpnA, chitinase family 18 protein, isocitrate dehydrogenase, Nif3 family protein, type IV pili glycosylation protein, outer membrane protein tolC, FAD binding family protein, type IV pilin multimeric outer membrane protein, two component sensor protein KdpD, chaperone protein DnaK, protein TolQ (Francisella tularensis, Tularemia); “MB antigen, urease, protein GyrA, protein GyrB, protein ParC, protein ParE, lipid associated membrane proteins LAMP, thymidine kinase TK, phospholipase PL-A1, phospholipase PL-A2, phospholipase PL-C, surface-expressed 96-kDa antigen;” (Ureaplasma urealyticum, Ureaplasma urealyticum infection); non-structural polyprotein, structural polyprotein, capsid protein CP, protein E1, protein E2, protein E3, protease P1, protease P2, protease P3 (Venezuelan equine encephalitis virus, Venezuelan equine encephalitis); glycoprotein GP, matrix protein Z, polymerase L, nucleoprotein N (Guanarito virus, Venezuelan hemorrhagic fever); polyprotein, protein E, protein M, capsid protein C, protease NS3, protein NS1, protein NS2A, protein AS2B, brotein NS4A, protein NS4B, protein NS5 (West Nile virus, West Nile Fever); cpasid protein CP, protein E1, protein E2, protein E3, protease P2 (Western equine encephalitis virus, Western equine encephalitis); genome polyprotein, protein E, protein M, capsid protein C, protease NS3, protein NS1, protein NS2A, protein AS2B, protein NS4A, protein NS4B, protein NS5 (Yellow fever virus, Yellow fever); putative Yop targeting protein YobB, effector protein YopD, effector protein YopE, protein YopH, effector protein YopJ, protein translocation protein YopK, effector protein YopT, protein YpkA, flagellar biosyntheses protein FlhA, peptidase M48, potassium efflux system KefA, transcriptional regulatoer RovA, adhesin Ifp, translocator portein LcrV, protein PcrV, invasin Inv, outer membrane protein OmpF-like porin, adhesin YadA, protein kinase C, phospholipase C1, protein PsaA, mannosyltransferase-like protein WbyK, protein YscU, antigen YPMa (Yersinia pseudotuberculosis, Yersinia pseudotuberculosis infection); effector protein YopB, 60 kDa chaperonin, protein WbcP, tyrosin-protein phosphatase YopH, protein YopQ, enterotoxin, Galactoside permease, reductaase NrdE, protein YasN, Invasin Inv, adhesin YadA, outer membrane porin F OmpF, protein UspA1, protein EibA, protein Hia, cell surface protein AiI, chaperone SycD, protein LcrD, protein LcrG, protein LcrV, protein SycE, protein YopE, regulator protein TyeA, protein YopM, protein YopN, protein YopO, protein YopT, protein YopD, protease ClpP, protein MyfA, protein FilA, and protein PsaA (Yersinia enterocolitica, Yersiniosis). (in brackets is the particular pathogen or the family of pathogens of which the antigen(s) is/are derived and the infectious disease with which the pathogen is associated)

In particularly preferred embodiments the pathogenic antigen is selected from
HIV p24 antigen, HIV envelope proteins (Gp120, Gp41, Gp160), polyprotein GAG, negative factor protein Nef, trans-activator of transcription Tat if the infectious disease is HIV, preferably an infection with Human immunodeficiency virus,
major outer membrane protein MOMP, probable outer membrane protein PMPC, outer membrane complex protein B OmcB, heat shock proteins Hsp60 HSP10, protein IncA, proteins from the type III secretion system, ribonucleotide reductase small chain protein NrdB, plasmid protein Pgp3, chlamydial outer protein N CopN, antigen CT521, antigen CT425, antigen CT043, antigen TC0052, antigen TC0189, antigen TC0582, antigen TC0660, antigen TC0726, antigen TC0816, antigen TC0828 if the infectious disease is an infection with Chlamydia trachomatis,
pp65 antigen, membrane protein pp15, capsid-proximal tegument protein pp150, protein M45, DNA polymerase UL54, helicase UL105, glycoprotein gM, glycoprotein gN, glcoprotein H, glycoprotein B gB, protein UL83, protein UL94, protein UL99 if the infectious disease is Cytomegalovirus infection, preferably an infection with Cytomegalovirus (CMV);
capsid protein C, premembrane protein prM, membrane protein M, envelope protein E (domain I, domain II, domain II), protein NS1, protein NS2A, protein NS2B, protein NS3, protein NS4A, protein 2K, protein NS4B, protein NS5 if the infectious disease is Dengue fever, preferably an infection with Dengue viruses (DEN-1, DEN-2, DEN-3 and DEN-4)-Flaviviruses;
hepatitis B surface antigen HBsAg, Hepatitis B core antigen HbcAg, polymerase, protein Hbx, preS2 middle surface protein, surface protein L, large S protein, virus protein VP1, virus protein VP2, virus protein VP3, virus protein VP4 if the infectious disease is Hepatitis B, preferably an infection with Hepatitis B Virus (HBV);
replication protein E1, regulatory protein E2, protein E3, protein E4, protein E5, protein E6, protein E7, protein E8, major capsid protein L1, minor capsid protein L2 if the infectious disease is Human papillomavirus (HPV) infection, preferably an infection with Human papillomavirus (HPV);
fusion protein F, hemagglutinin-neuramidase HN, glycoprotein G, matrix protein M, phosphoprotein P, nucleoprotein N, polymerase L if the infectious disease is Human parainfluenza virus infection, preferably an infection with Human parainfluenza viruses (HPIV);
Hemagglutinin (HA), Neuraminidase (NA), Nucleoprotein (NP), M1 protein, M2 protein, NS1 protein, NS2 protein (NEP protein: nuclear export protein), PA protein, PB1 protein (polymerase basic 1 protein), PB1-F2 protein and PB2 protein (Orthomyxoviridae family, Influenza virus (flu));
nucleoprotein N, large structural protein L, phophoprotein P, matrix protein M, glycoprotein G if the infectious disease is Rabies, preferably an infection with Rabies virus;
fusionprotein F, nucleoprotein N, matrix protein M, matrix protein M2-1, matrix protein M2-2, phophoprotein P, small hydrophobic protein SH, major surface glycoprotein G, polymerase L, non-structural protein 1 NS1, non-structural protein 2 NS2 if the infectious disease is Respiratory syncytial virus infection, preferably an infection with Respiratory syncytial virus (RSV);
secretory antigen SssA (Staphylococcus genus, Staphylococcal food poisoning); secretory antigen SssA (Staphylococcus genus e.g. aureus, Staphylococcal infection); molecular chaperone DnaK, cell surface lipoprotein Mpt83, lipoprotein P23, phosphate transport system permease protein pstA, 14 kDa antigen, fibronectin-binding protein C FbpC1, Alanine dehydrogenase TB43, Glutamine synthetase 1, ESX-1 protein, protein CFP10, TB10.4 protein, protein MPT83, protein MTB12, protein MTB8, Rpf-like proteins, protein MTB32, protein MTB39, crystallin, heat-shock protein HSP65, protein PST-S if the infectious disease is Tuberculosis, preferably an infection with Mycobacterium tuberculosis;
genome polyprotein, protein E, protein M, capsid protein C, protease NS3, protein NS1, protein NS2A, protein AS2B, protein NS4A, protein NS4B, protein NS5 if the infectious disease is Yellow fever, preferably an infection with Yellow fever virus.

According to a preferred embodiment, the at least one first (immunogenic) component of the combination or composition according to the invention comprises at least one nucleic acid encoding at least one epitope, antigenic peptide or protein derived from a protein of an influenza virus or a fragment or variant thereof, wherein the influenza virus is preferably selected from an influenza A, B or C virus, more preferably an influenza A virus. Preferably, the at least one epitope, antigenic peptide or protein is derived from hemagglutinin (HA), neuraminidase (NA), nucleoprotein (NP), matrix protein 1 (M1), matrix protein 2 (M2), non-structural protein 1 (NS1), non-structural protein 2 (NS2), nuclear export protein (NEP), polymerase acidic protein (PA), polymerase basic protein PB1, PB1-F2, or polymerase basic protein 2 (PB2) of an influenza virus or a fragment or variant thereof.

In a particularly preferred embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID Nos. 30044-43876, 60087-73919, 90130-103962, 120173-134005, 150216-164048, or 180259-194091, as described in international patent application PCT/EP2016/060112, or a fragment or variant of any of these RNA sequences.

In another embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID Nos. 56083-58208, 86126-88251, 116169-118294, 146212-148337, 176255-178380, or 206298-208423 as described in international patent application PCT/EP2016/060112, or a fragment or variant of any of these RNA sequences.

Alternatively, the at least one first (immunogenic) component of the combination or composition according to the invention may comprise at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID Nos. 43877-56082, 73920-86125, 103963-116168, 134006-146211, 164049-176254, or 194092-206297 as described in international patent application PCT/EP2016/060112, or a fragment or variant of any of these RNA sequences.

According to a further embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID Nos. 58209-60086, 88252-90129, 118295-120172, 148338-150215, 178381-180258, or 208424-210301 as described in international patent application PCT/EP2016/060112, or a fragment or variant of any of these RNA sequences.

Most preferably, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID Nos. 210323-210326, 210328-210332, 210334-210339, 210341-210345, 210347-210350, 210352-210355, 210357-210360, 210362-210365, 210367-210370, 210372-210376, 210378-210382, 210384-210387, 210389-210392, 210394-210398, or 210401-210402 as described in international patent application PCT/EP2016/060112, or a fragment or variant of any of these RNA sequences.

According to a preferred embodiment, the at least one first (immunogenic) component of the combination or composition according to the invention comprises at least one nucleic acid encoding at least one epitope, antigenic peptide or protein derived from a protein of an Noro virus or a fragment or variant thereof, wherein the Noro virus is preferably selected from the group consisting of genogroup I Norovirus, genogroup II Norovirus, genogroup III Norovirus, genogroup IV Norovirus, and genogroup V Norovirus; more preferably from a Norovirus selected from the group consisting of a GI.1 to GI.17 Norovirus, GII.1 to GII.24 Norovirus, GIII.1 to GIII.4 Norovirus, GIV.1 to GIV.4 Norovirus and GV.1 to GV.4 Norovirus; even more preferably from a Norovirus selected from the group consisting of GI.1 Norovirus and GII.4 Norovirus, even more preferably from a GII.4 Norovirus, still more preferably from a GII.4 CIN-1 Norovirus or a GII.4 Sydney Norovirus or a GII.4 Sydney 2012 Norovirus. Preferably, the at least one epitope, antigenic peptide or protein is derived from capsid protein VP1 or capsid protein VP2 of a Norovirus as defined herein, or a fragment or variant thereof.

In a preferred embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NO: 2582 to SEQ ID NO: 20686 as described in international patent application PCT/EP2016/060115, or a fragment or variant of any of these RNA sequences.

According to a particularly preferred embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs:5163-7743, 15487-18067, 18068-20648, 7744-10324, 10325-12905, or 12906-15486 as described in international patent application PCT/EP2016/060115, or a fragment or variant of any of these RNA sequences.

In another embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 20670 to 20682 as described in international patent application PCT/EP2016/060115, or a fragment or variant of any of these RNA sequences.

According to a preferred embodiment, the at least one first (immunogenic) component of the combination or composition according to the invention comprises at least one nucleic acid encoding at least one epitope, antigenic peptide or protein derived from a protein of a Rhinovirus or a fragment or variant thereof, wherein the Rhinovirus is preferably selected from the group consisting of human rhinovirus A, human rhinovirus B and human rhinovirus C. Preferably, the at least one epitope, antigenic peptide or protein is derived from a Rhinovirus capsid protein or a Rhinovirus non-structural protein of a Rhinovirus as defined herein, or a fragment or variant thereof. More preferably, the at least one epitope, antigenic peptide or protein is derived from a Rhinovirus capsid protein, wherein the Rhinovirus capsid protein is preferably selected from the group consisting of Rhinovirus capsid protein VP0, Rhinovirus capsid protein P1, Rhinovirus capsid protein VP1, Rhinovirus capsid protein VP2, Rhinovirus capsid protein VP3 and Rhinovirus capsid protein VP4, or a fragment or variant of any of these proteins. Even more preferably, the at least one epitope, antigenic peptide or protein is derived from a Rhinovirus non-structural protein, wherein the Rhinovirus non-structural protein is preferably selected from the group consisting of Rhinovirus protease 2A, Rhinovirus protein 2B, Rhinovirus protein 2C, Rhinovirus protein P2, Rhinovirus protein 3A, Rhinovirus viral priming protein VPg (3B), Rhinovirus protein 3AB, Rhinovirus protease 3C, Rhinovirus RNA dependent RNA polymerase RDRP (3D), Rhinovirus protein 3CD, or a fragment or variant of any of these.

In a preferred embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 2764-22104, 22127-22129, 22131-22134, 22136-22138, 22140-22143, 22145-22148, 22150-22153, 22155-22158, 22160-22163 or 22165-22166 as described in international patent application PCT/EP2016/060114, or a fragment or variant of any of these RNA sequences.

According to a particularly preferred embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 22127-22129, 22131-22134, 22136-22138, 22140-22143, 22145-22148, 22150-22153, 22155-22158, 22160-22163 or 22165-22166 as described in international patent application PCT/EP2016/060114, or a fragment or variant of any of these RNA sequences.

Alternatively, the at least one first (immunogenic) component of the combination or composition according to the invention may comprise at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 5527-8289, 16579-19341 or 19342-22104 as described in international patent application PCT/EP2016/060114, or a fragment or variant of any of these RNA sequences.

According to a further preferred embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 8290-11052 as described in international patent application PCT/EP2016/060114, or a fragment or variant of any of these RNA sequences.

In another embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 11053-13815 as described in international patent application PCT/EP2016/060114, or a fragment or variant of any of these RNA sequences.

According to a further embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 13816-16578 as described in international patent application PCT/EP2016/060114, or a fragment or variant of any of these RNA sequences.

In a preferred embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 22128, 22133, 22137, 22142, 22147, 22152, 22157 or 22162 as described in international patent application PCT/EP2016/060114, or a fragment or variant of any of these RNA sequences.

According to a particularly preferred embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 22129, 22134, 22138, 22143, 22148, 22153, 22158 or 22163 as described in international patent application PCT/EP2016/060114, or a fragment or variant of any of these RNA sequences.

In a further preferred embodiment of the invention the nucleic acid molecule, preferably the mRNA, of the first immunogenic component of the inventive composition encodes at least one epitope of a protein or peptide which is a tumor antigen or a fragment, variant or derivative thereof. It is most preferably understood that a protein or peptide acting as tumor antigen according to the invention is derived from mammals, in particular humans, in particular from mammalian tumors, and does not qualify as selection, marker or reporter protein. In particular, such tumor antigens are derived from mammalian, in particular from human tumors. These tumor antigenic proteins or peptides are understood to be antigenic, as they are meant to treat the subject by triggering the subject's immune response such that the subject's immune system is enabled to combat the subject's tumor cells by TH1 and/or TH2 immune responses. Accordingly, such antigenic tumor proteins are typically mammalian, in particular human proteins characterizing the subject's cancer type.

Tumor antigens are preferably located on the surface of the (tumor) cell characterizing a mammalian, in particular human tumor (in e.g. systemic or solid tumor diseases). Tumor antigens may also be selected from proteins, which are overexpressed in tumor cells compared to a normal cell. Furthermore, tumor antigens also includes antigens expressed in cells which are (were) not themselves (or originally not themselves) degenerated but are associated with the supposed tumor. Antigens which are connected with tumor-supplying vessels or (re)formation thereof, in particular those antigens which are associated with neovascularization, e.g. growth factors, such as VEGF, bFGF etc., are also included herein. Antigens connected with a tumor furthermore include antigens from cells or tissues, typically embedding the tumor. Further, some substances (usually proteins or peptides) are expressed in patients suffering (knowingly or not-knowingly) from a cancer disease and they occur in increased concentrations in the body fluids of said patients. These substances are also referred to as “tumor antigens”, however they are not antigens in the stringent meaning of an immune response inducing substance. The class of tumor antigens can be divided further into tumor-specific antigens (TSAs) and tumor-associated-antigens (TAAs). TSAs can only be presented by tumor cells and never by normal “healthy” cells. They typically result from a tumor specific mutation. TAAs, which are more common, are usually presented by both tumor and healthy cells. These antigens are recognized and the antigen-presenting cell can be destroyed by cytotoxic T cells. Additionally, tumor antigens can also occur on the surface of the tumor in the form of, e.g., a mutated receptor. In this case, they can be recognized by antibodies.

Further, tumor associated antigens may be classified as tissue-specific antigens, also called melanocyte-specific antigens, cancer-testis antigens and tumor-specific antigens. Cancer-testis antigens are typically understood to be peptides or proteins of germ-line associated genes which may be activated in a wide variety of tumors. Human cancer-testis antigens may be further subdivided into antigens which are encoded on the X chromosome, so-called CT-X antigens, and those antigens which are not encoded on the X chromosome, the so-called non-X CT antigens. Cancer-testis antigens which are encoded on the X-chromosome comprises, for example, the family of melanoma antigen genes, the so-called MAGE-family. The genes of the MAGE-family may be characterised by a shared MAGE homology domain (MHD). Each of these antigens, i.e. melanocyte-specific antigens, cancer-testis antigens and tumor-specific antigens, may elicit autologous cellular and humoral immune response. Accordingly, the tumor antigen encoded by the inventive nucleic acid sequence is preferably a melanocyte-specific antigen, a cancer-testis antigen or a tumor-specific antigen, preferably it may be a CT-X antigen, a non-X CT-antigens, a binding partner for a CT-X antigen or a binding partner for a non-X CT-antigen or a fragment, variant or derivative of said tumor antigen.

Particular preferred tumor antigens are selected from the list consisting of 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, calreticulin, CAMEL, CASP-8/m, cathepsin B, cathepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R171, HLA-A11/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1R, IL-13Ra2, IL-2R, IL-5, immature laminin receptor, kallikrein-2, kallikrein-4, Ki67, KIAA0205, KIAA0205/m, KK-LC-1, K-Ras/m, LAGS-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MC1R, M-CSF, ME1/m, mesothelin, MG50/PXDN, MMP11, MN/CA IX-antigen, MRP-3, MUC-1, MUC-2, MUM-1/m, MUM-2/m, MUM-3/m, myosin class I/m, NA88-A, N-acetylglucosaminyltransferase-V, Neo-PAP, Neo-PAP/m, NFYC/m, NGEP, NMP22, NPM/ALK, N-Ras/m, NSE, NY-ESO-B, NY-ESO-1, OA1, OFA-iLRP, OGT, OGT/m, OS-9, OS-9/m, osteocalcin, osteopontin, p15, p190 minor bcr-abl, p53, p53/m, PAGE-4, PAI-1, PAI-2, PAP, PART-1, PATE, PDEF, Pim-1-Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD168, RU1, RU2, S-100, SAGE, SART-1, SART-2, SART-3, SCC, SIRT2/m, Sp17, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGFbeta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, and WT1. Such tumor antigens preferably may be selected from the group consisting of p53, CA125, EGFR, Her2/neu, hTERT, PAP, MAGE-A1, MAGE-A3, Mesothelin, MUC-1, GP100, MART-1, Tyrosinase, PSA, PSCA, PSMA, STEAP-1, VEGF, VEGFR1, VEGFR2, Ras, CEA or WT1, and more preferably from PAP, MAGE-A3, WT1, and MUC-1. Such tumor antigens preferably may be selected from the group consisting of MAGE-A1 (e.g. MAGE-A1 according to accession number M77481), MAGE-A2, MAGE-A3, MAGE-A6 (e.g. MAGE-A6 according to accession number NM_005363), MAGE-C1, MAGE-C2, melan-A (e.g. melan-A according to accession number NM_005511), GP100 (e.g. GP100 according to accession number M77348), tyrosinase (e.g. tyrosinase according to accession number NM_000372), surviving (e.g. survivin according to accession number AF077350), CEA (e.g. CEA according to accession number NM_004363), Her-2/neu (e.g. Her-2/neu according to accession number M11730), WT1 (e.g. WT1 according to accession number NM_000378), PRAME (e.g. PRAME according to accession number NM_006115), EGFRI (epidermal growth factor receptor 1) (e.g. EGFRI (epidermal growth factor receptor 1) according to accession number AF288738), MUC1, mucin-1 (e.g. mucin-1 according to accession number NM_002456), SEC61G (e.g. SEC61G according to accession number NM_014302), hTERT (e.g. hTERT accession number NM_198253), 5T4 (e.g. 5T4 according to accession number NM_006670), TRP-2 (e.g. TRP-2 according to accession number NM_001922), STEAP1, PCA, PSA, PSMA, etc.

Furthermore tumor antigens also may encompass idiotypic antigens associated with a cancer or tumor disease, particularly lymphoma or a lymphoma associated disease, wherein said idiotypic antigen is an immunoglobulin idiotype of a lymphoid blood cell or a T cell receptor idiotype of a lymphoid blood cell.

Tumor antigenic proteins for the treatment of cancer or tumor diseases, are typically proteins of mammalian origin, preferably of human origin. Their selection for treatment of the subject depends on the tumor type to be treated and the expression profile of the individual tumor. A human suffering from prostate cancer, is e.g. preferably treated by a tumor antigen, which is typically expressed (or overexpressed) in prostate carcinoma or specifically overexpressed in the subject to be treated, e.g. any of PSMA, PSCA, and/or PSA.

Preferably, the encoded tumor antigen is no reporter protein (e.g. Luciferase, Green Fluorescent Protein (GFP), Enhanced Green Fluorescent Protein (EGFP), β-Galactosidase) and no marker or selection protein (e.g. alpha-Globin, Galactokinase and Xanthine:guanine phosphoribosyl transferase (GPT)). Preferably, the nucleic acid molecule of the invention does not contain a (bacterial) antibiotics resistance gene, in particular not a neo gene sequence (Neomycin resistance gene) or CAT gene sequence (chloramphenicol acetyl transferase, chloramphenicol resistance gene).

In particularly preferred embodiments of the inventive composition the tumor antigen is a melanocyte-specific antigen, a cancer-testis antigen or a tumor-specific antigen, preferably a CT-X antigen, a non-X CT-antigen, a binding partner for a CT-X antigen or a binding partner for a non-X CT-antigen or a tumor-specific antigen, more preferably a CT-X antigen, a binding partner for a non-X CT-antigen or a tumor-specific antigen or a fragment, variant or derivative of said tumor antigen.

Preferably the tumor antigen is selected from the list of: 5T4, 707-AP, 9D7, AFP, AlbZIP HPG1, alpha-5-beta-1-integrin, alpha-5-beta-6-integrin, alpha-actinin-4/m, alpha-methylacyl-coenzyme A racemase, ART-4, ARTC1/m, B7H4, BAGE-1, BCL-2, bcr/abl, beta-catenin/m, BING-4, BRCA1/m, BRCA2/m, CA 15-3/CA 27-29, CA 19-9, CA72-4, CA125, calreticulin, CAMEL, CASP-8/m, cathepsin B, cathepsin L, CD19, CD20, CD22, CD25, CDE30, CD33, CD4, CD52, CD55, CD56, CD80, CDC27/m, CDK4/m, CDKN2A/m, CEA, CLCA2, CML28, CML66, COA-1/m, coactosin-like protein, collage XXIII, COX-2, CT-9/BRD6, Cten, cyclin B1, cyclin D1, cyp-B, CYPB1, DAM-10, DAM-6, DEK-CAN, EFTUD2/m, EGFR, ELF2/m, EMMPRIN, EpCam, EphA2, EphA3, ErbB3, ETV6-AML1, EZH2, FGF-5, FN, Frau-1, G250, GAGE-1, GAGE-2, GAGE-3, GAGE-4, GAGE-5, GAGE-6, GAGE7b, GAGE-8, GDEP, GnT-V, gp100, GPC3, GPNMB/m, HAGE, HAST-2, hepsin, Her2/neu, HERV-K-MEL, HLA-A*0201-R171, HLA-A11/m, HLA-A2/m, HNE, homeobox NKX3.1, HOM-TES-14/SCP-1, HOM-TES-85, HPV-E6, HPV-E7, HSP70-2M, HST-2, hTERT, iCE, IGF-1R, IL-13Ra2, IL-2R, IL-5, immature laminin receptor, kallikrein-2, kallikrein-4, Ki67, KIAA0205, KIAA0205/m, KK-LC-1, K-Ras/m, LAGE-A1, LDLR-FUT, MAGE-A1, MAGE-A2, MAGE-A3, MAGE-A4, MAGE-A6, MAGE-A9, MAGE-A10, MAGE-A12, MAGE-B1, MAGE-B2, MAGE-B3, MAGE-B4, MAGE-B5, MAGE-B6, MAGE-B10, MAGE-B16, MAGE-B17, MAGE-C1, MAGE-C2, MAGE-C3, MAGE-D1, MAGE-D2, MAGE-D4, MAGE-E1, MAGE-E2, MAGE-F1, MAGE-H1, MAGEL2, mammaglobin A, MART-1/melan-A, MART-2, MART-2/m, matrix protein 22, MC1R, M-CSF, ME1/m, mesothelin, MG50/PXDN, MMP11, MN/CA IX-antigen, MRP-3, MUC-1, MUC-2, MUM-1/m, MUM-2/m, MUM-3/m, myosin class I/m, NA88-A, N-acetylglucosaminyltransferase-V, Neo-PAP, Neo-PAP/m, NFYC/m, NGEP, NMP22, NPM/ALK, N-Ras/m, NSE, NY-ESO-B, OA1, OFA-iLRP, OGT, OGT/m, OS-9, OS-9/m, osteocalcin, osteopontin, p15, p190 minor bcr-abl, p53, p53/m, PAGE-4, PAI-1, PAI-2, PAP, PART-1, PATE, PDEF, Pim-1-Kinase, Pin-1, Pml/PARalpha, POTE, PRAME, PRDX5/m, prostein, proteinase-3, PSA, PSCA, PSGR, PSM, PSMA, PTPRK/m, RAGE-1, RBAF600/m, RHAMM/CD168, RU1, RU2, S-100, SAGE, SART-1, SART-2, SART-3, SCC, SIRT2/m, Sp17, SSX-1, SSX-2/HOM-MEL-40, SSX-4, STAMP-1, STEAP-1, survivin, survivin-2B, SYT-SSX-1, SYT-SSX-2, TA-90, TAG-72, TARP, TEL-AML1, TGFbeta, TGFbetaRII, TGM-4, TPI/m, TRAG-3, TRG, TRP-1, TRP-2/6b, TRP/INT2, TRP-p8, tyrosinase, UPA, VEGFR1, VEGFR-2/FLK-1, WT1 and a immunoglobulin idiotype of a lymphoid blood cell or a T cell receptor idiotype of a lymphoid blood cell, or a fragment, variant or derivative of said tumor antigen; preferably survivin or a homologue thereof, an antigen from the MAGE-family or a binding partner thereof or a fragment, variant or derivative of said tumor antigen.

More preferably the tumor antigen is selected from the following list: p53, CA125, EGFR, Her2/neu, hTERT, PAP, MAGE-A1, MAGE-A3, MAGE-C1, MAGE-C2, Mesothelin, MUC-1, NY-ESO-1, GP100, MART-1, Tyrosinase, PSA, PSCA, PSMA, VEGF, VEGFR1, VEGFR2, Ras, CEA, Survivin, 5T4, STEAP and WT1.

According to a preferred embodiment, the at least one first (immunogenic) component of the combination or composition according to the invention comprises at least one nucleic acid encoding at least one epitope, antigenic peptide or protein derived from a tumor antigen as described herein, or a fragment or variant thereof.

Preferably, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 2, 3, 4, 5, 6, 7, 9, 10, 11, 12, 13, 14, 16, 17, 18, 19, 20, 21, 23, 24, 25, 26, 27, 28, 30, 31, 32, 33, 34, 35, 37, 38, 39, 40, 41, 42, 44, 45, 46, 47, 48, 49, 51, 52, 53, 54, 55, 56, 58, 59, 60, 61, 62, 63, 65, 66, 67, 68, 69, 70, 72, 73, 74, 75, 76, 77, 79, 80, 81, 82, 83, 84, 86, 87, 88, 89, 90, 91, 93, 94, 95, 96, 97, 98, 100, 101, 102, 103, 104, 105, 107, 108, 109, 110, 111, 112, 114, 115, 116, 117, 118, 119, 121, 122, 123, 124, 125, 126, 128, 129, 130, 131, 132, 133, 135, 136, 137, 138, 139, 140, 142, 143, 144, 145, 146, 147, 149, 150, 151, 152, 153, 154, 156, 157, 158, 159, 160, 161, 163, 164, 165, 166, 167, 168, 170, 171, 172, 173, 174, 175, 177, 178, 179, 180, 181, 182, 184, 185, 186, 187, 188, 189, 191, 192, 193, 194, 195, 196, 198, 199, 200, 201, 202, 203, 205, 206, 207, 208, 209, 210, 212, 213, 214, 215, 216, 217, 219, 220, 221, 222, 223, 224, 226, 227, 228, 229, 230, 231, 233, 234, 235, 236, 237, 238, 240, 241, 242, 243, 244, 245, 247, 248, 249, 250, 251, 252, 254, 255, 256, 257, 258, 259, 261, 262, 263, 264, 265, 266, 268, 269, 270, 271, 272, 273, 275, 276, 277, 278, 279, 280, 282, 283, 284, 285, 286, 287, 289, 290, 291, 292, 293, 294, 296, 297, 298, 299, 300, 301, 303, 304, 305, 306, 307, 308, 310, 311, 312, 313, 314, 315, 317, 318, 319, 320, 321, 322, 324, 325, 326, 327, 328, 329, 331, 332, 333, 334, 335, 336, 338, 339, 340, 341, 342, 343, 345, 346, 347, 348, 349, 350, 352, 353, 354, 355, 356, 357, 359, 360, 361, 362, 363, 364, 366, 367, 368, 369, 370, 371, 373, 374, 375, 376, 377, 378, 380, 381, 382, 383, 384, 385, 387, 388, 389, 390, 391, 392, 394, 395, 396, 397, 398, 399, 401, 402, 403, 404, 405, 406, 408, 409, 410, 411, 412, 413, 415, 416, 417, 418, 419, 420, 422, 423, 424, 425, 426, 427, 429, 430, 431, 432, 433, 434, 436, 437, 438, 439, 440, 441, 443, 444, 445, 446, 447, 448, 450, 451, 452, 453, 454, 455, 457, 458, 459, 460, 461, 462, 464, 465, 466, 467, 468, 469, 471, 472, 473, 474, 475, 476, 478, 479, 480, 481, 482, 483, 485, 486, 487, 488, 489, 490, 492, 493, 494, 495, 496, 497, 499, 500, 501, 502, 503, 504, 506, 507, 508, 509, 510, 511, 513, 514, 515, 516, 517, 518, 520, 521, 522, 523, 524, 525, 527, 528, 529, 530, 531, 532, 534, 535, 536, 537, 538, 539, 541, 542, 543, 544, 545, 546, 548, 549, 550, 551, 552, 553, 555, 556, 557, 558, 559, 560, 562, 563, 564, 565, 566, 567, 569, 570, 571, 572, 573, 574, 576, 577, 578, 579, 580, 581, 583, 584, 585, 586, 587, 588, 590, 591, 592, 593, 594, 595, 597, 598, 599, 600, 601, 602, 604, 605, 606, 607, 608, 609, 611, 612, 613, 614, 615, 616, 618, 619, 620, 621, 622, 623, 625, 626, 627, 628, 629, 630, 632, 633, 634, 635, 636, 637, 639, 640, 641, 642, 643, 644, 646, 647, 648, 649, 650, 651, 653, 654, 655, 656, 657, 658, 660, 661, 662, 663, 664, 665, 667, 668, 669, 670, 671, 672, 674, 675, 676, 677, 678, 679, 681, 682, 683, 684, 685, 686, 688, 689, 690, 691, 692, 693, 695, 696, 697, 698, 699, 700, 702, 703, 704, 705, 706, 707, 709, 710, 711, 712, 713, 714, 716, 717, 718, 719, 720, 721, 723, 724, 725, 726, 727, 728, 730, 731, 732, 733, 734, 735, 737, 738, 739, 740, 741, 742, 744, 745, 746, 747, 748, 749, 751, 752, 753, 754, 755, 756, 758, 759, 760, 761, 762, 763, 765, 766, 767, 768, 769, 770, 772, 773, 774, 775, 776, 777, 779, 780, 781, 782, 783, 784, 786, 787, 788, 789, 790, 791, 793, 794, 795, 796, 797, 798, 800, 801, 802, 803, 804, 805, 807, 808, 809, 810, 811, 812, 814, 815, 816, 817, 818, 819, 821, 822, 823, 824, 825, 826, 828, 829, 830, 831, 832, 833, 835, 836, 837, 838, 839, 840, 842, 843, 844, 845, 846, 847, 849, 850, 851, 852, 853, 854, 856, 857, 858, 859, 860, 861, 863, 864, 865, 866, 867, 868, 870, 871, 872, 873, 874, 875, 877, 878, 879, 880, 881, 882, 884, 885, 886, 887, 888, 889, 891, 892, 893, 894, 895, 896, 898, 899, 900, 901, 902, 903, 905, 906, 907, 908, 909, 910, 912, 913, 914, 915, 916, 917, 919, 920, 921, 922, 923, 924, 926, 927, 928, 929, 930, 931, 933, 934, 935, 936, 937, 938, 940, 941, 942, 943, 944, 945, 947, 948, 949, 950, 951, 952, 954, 955, 956, 957, 958, 959, 961, 962, 963, 964, 965, 966, 968, 969, 970, 971, 972, 973, 975, 976, 977, 978, 979, 980, 982, 983, 984, 985, 986, 987, 989, 990, 991, 992, 993, 994, 996, 997, 998, 999, 1000, 1001, 1003, 1004, 1005, 1006, 1007, 1008, 1010, 1011, 1012, 1013, 1014, 1015, 1017, 1018, 1019, 1020, 1021, 1022, 1024, 1025, 1026, 1027, 1028, 1029, 1031, 1032, 1033, 1034, 1035, 1036, 1038, 1039, 1040, 1041, 1042, 1043, 1045, 1046, 1047, 1048, 1049, 1050, 1052, 1053, 1054, 1055, 1056, 1057, 1059, 1060, 1061, 1062, 1063, 1064, 1066, 1067, 1068, 1069, 1070, 1071, 1073, 1074, 1075, 1076, 1077, 1078, 1080, 1081, 1082, 1083, 1084, 1085, 1087, 1088, 1089, 1090, 1091, 1092, 1094, 1095, 1096, 1097, 1098, 1099, 1101, 1102, 1103, 1104, 1105, 1106, 1108, 1109, 1110, 1111, 1112, 1113, 1115, 1116, 1117, 1118, 1119, 1120, 1122, 1123, 1124, 1125, 1126, 1127, 1129, 1130, 1131, 1132, 1133, 1134, 1136, 1137, 1138, 1139, 1140, 1141, 1143, 1144, 1145, 1146, 1147, 1148, 1150, 1151, 1152, 1153, 1154, 1155, 1157, 1158, 1159, 1160, 1161, 1162, 1164, 1165, 1166, 1167, 1168, 1169, 1171, 1172, 1173, 1174, 1175, 1176, 1178, 1179, 1180, 1181, 1182, 1183, 1185, 1186, 1187, 1188, 1189, 1190, 1192, 1193, 1194, 1195, 1196, 1197, 1199, 1200, 1201, 1202, 1203, 1204, 1206, 1207, 1208, 1209, 1210, 1211, 1213, 1214, 1215, 1216, 1217, 1218, 1220, 1221, 1222, 1223, 1224, 1225, 1227, 1228, 1229, 1230, 1231, 1232, 1234, 1235, 1236, 1237, 1238, 1239, 1241, 1242, 1243, 1244, 1245, 1246, 1248, 1249, 1250, 1251, 1252, 1253, 1255, 1256, 1257, 1258, 1259, 1260, 1262, 1263, 1264, 1265, 1266, 1267, 1269, 1270, 1271, 1272, 1273, 1274, 1276, 1277, 1278, 1279, 1280, 1281, 1283, 1284, 1285, 1286, 1287, 1288, 1290, 1291, 1292, 1293, 1294, 1295, 1297, 1298, 1299, 1300, 1301, 1302, 1304, 1305, 1306, 1307, 1308, 1309, 1311, 1312, 1313, 1314, 1315, 1316, 1318, 1319, 1320, 1321, 1322, 1323, 1325, 1326, 1327, 1328, 1329, 1330, 1332, 1333, 1334, 1335, 1336, 1337, 1339, 1340, 1341, 1342, 1343, 1344, 1346, 1347, 1348, 1349, 1350, 1351, 1353, 1354, 1355, 1356, 1357, 1358, 1360, 1361, 1362, 1363, 1364, 1365, 1367, 1368, 1369, 1370, 1371, 1372, 1374, 1375, 1376, 1377, 1378, 1379, 1381, 1382, 1383, 1384, 1385, 1386, 1388, 1389, 1390, 1391, 1392, 1393, 1395, 1396, 1397, 1398, 1399, 1400, 1402, 1403, 1404, 1405, 1406, 1407, 1409, 1410, 1411, 1412, 1413, 1414, 1416, 1417, 1418, 1419, 1420, 1421, 1423, 1424, 1425, 1426, 1427, 1428, 1430, 1431, 1432, 1433, 1434, 1435, 1437, 1438, 1439, 1440, 1441, 1442, 1444, 1445, 1446, 1447, 1448, 1449, 1451, 1452, 1453, 1454, 1455, 1456, 1458, 1459, 1460, 1461, 1462, 1463, 1465, 1466, 1467, 1468, 1469, 1470, 1472, 1473, 1474, 1475, 1476, 1477, 1479, 1480, 1481, 1482, 1483, 1484, 1486, 1487, 1488, 1489, 1490, 1491, 1493, 1494, 1495, 1496, 1497, 1498, 1500, 1501, 1502, 1503, 1504, 1505, 1507, 1508, 1509, 1510, 1511, 1512, 1514, 1515, 1516, 1517, 1518, 1519, 1521, 1522, 1523, 1524, 1525, 1526, 1528, 1529, 1530, 1531, 1532, 1533, 1535, 1536, 1537, 1538, 1539, 1540, 1542, 1543, 1544, 1545, 1546, 1547, 1549, 1550, 1551, 1552, 1553, 1554, 1556, 1557, 1558, 1559, 1560, 1561, 1563, 1564, 1565, 1566, 1567, 1568, 1570, 1571, 1572, 1573, 1574, 1575, 1577, 1578, 1579, 1580, 1581, 1582, 1584, 1585, 1586, 1587, 1588, 1589, 1591, 1592, 1593, 1594, 1595, 1596, 1598, 1599, 1600, 1601, 1602, 1603, 1605, 1606, 1607, 1608, 1609, 1610, 1612, 1613, 1614, 1615, 1616, 1617, 1619, 1620, 1621, 1622, 1623, 1624, 1626, 1627, 1628, 1629, 1630, 1631, 1633, 1634, 1635, 1636, 1637, 1638, 1640, 1641, 1642, 1643, 1644, 1645, 1647, 1648, 1649, 1650, 1651, 1652, 1654, 1655, 1656, 1657, 1658, 1659, 1661, 1662, 1663, 1664, 1665, 1666, 1668, 1669, 1670, 1671, 1672, 1673, 1675, 1676, 1677, 1678, 1679, 1680, 1682, 1683, 1684, 1685, 1686, 1687, 1689, 1690, 1691, 1692, 1693, 1694, 1696, 1697, 1698, 1699, 1700, 1701, 1703, 1704, 1705, 1706, 1707, 1708, 1710, 1711, 1712, 1713, 1714, 1715, 1717, 1718, 1719, 1720, 1721, 1722, 1724, 1725, 1726, 1727, 1728, 1729, 1731, 1732, 1733, 1734, 1735, 1736, 1738, 1739, 1740, 1741, 1742, 1743, 1745, 1746, 1747, 1748, 1749, 1750, 1752, 1753, 1754, 1755, 1756, 1757, 1759, 1760, 1761, 1762, 1763, 1764, 1766, 1767, 1768, 1769, 1770, 1771, 1773, 1774, 1775, 1776, 1777, 1778, 1780, 1781, 1782, 1783, 1784, 1785, 1787, 1788, 1789, 1790, 1791, 1792, 1794, 1795, 1796, 1797, 1798, 1799, 1801, 1802, 1803, 1804, 1805, 1806, 1808, 1809, 1810, 1811, 1812, 1813, 1815, 1816, 1817, 1818, 1819, 1820, 1822, 1823, 1824, 1825, 1826, 1827, 1829, 1830, 1831, 1832, 1833, 1834, 1836, 1837, 1838, 1839, 1840, 1841, 1843, 1844, 1845, 1846, 1847, 1848, 1850, 1851, 1852, 1853, 1854, 1855, 1857, 1858, 1859, 1860, 1861, 1862, 1864, 1865, 1866, 1867, 1868, 1869, 1871, 1872, 1873, 1874, 1875, 1876, 1878, 1879, 1880, 1881, 1882, 1883, 1885, 1886, 1887, 1888, 1889, 1890, 1892, 1893, 1894, 1895, 1896, 1897, 1899, 1900, 1901, 1902, 1903, 1904, 1906, 1907, 1908, 1909, 1910, 1911, 1913, 1914, 1915, 1916, 1917, 1918, 1920, 1921, 1922, 1923, 1924, 1925, 1927, 1928, 1929, 1930, 1931, 1932, 1934, 1935, 1936, 1937, 1938, 1939, 1941, 1942, 1943, 1944, 1945, 1946, 1948, 1949, 1950, 1951, 1952, 1953, 1955, 1956, 1957, 1958, 1959, 1960, 1962, 1963, 1964, 1965, 1966, 1967, 1969, 1970, 1971, 1972, 1973, 1974, 1976, 1977, 1978, 1979, 1980, 1981, 1983, 1984, 1985, 1986, 1987, 1988, 1990, 1991, 1992, 1993, 1994, 1995, 1997, 1998, 1999, 2000, 2001, 2002, 2004, 2005, 2006, 2007, 2008, 2009, 2011, 2012, 2013, 2014, 2015, 2016, 2018, 2019, 2020, 2021, 2022, 2023, 2025, 2026, 2027, 2028, 2029, 2030, 2032, 2033, 2034, 2035, 2036, 2037, 2039, 2040, 2041, 2042, 2043, 2044, 2046, 2047, 2048, 2049, 2050, 2051, 2053, 2054, 2055, 2056, 2057, 2058, 2060, 2061, 2062, 2063, 2064, 2065, 2067, 2068, 2069, 2070, 2071, 2072, 2074, 2075, 2076, 2077, 2078, 2079, 2081, 2082, 2083, 2084, 2085, 2086, 2088, 2089, 2090, 2091, 2092, 2093, 2095, 2096, 2097, 2098, 2099, 2100, 2102, 2103, 2104, 2105, 2106, 2107, 2109, 2110, 2111, 2112, 2113, 2114, 2116, 2117, 2118, 2119, 2120, 2121, 2123, 2124, 2125, 2126, 2127, 2128, 2130, 2131, 2132, 2133, 2134, 2135, 2137, 2138, 2139, 2140, 2141, 2142, 2144, 2145, 2146, 2147, 2148, 2149, 2151, 2152, 2153, 2154, 2155, 2156, 2158, 2159, 2160, 2161, 2162, 2163, 2165, 2166, 2167, 2168, 2169, 2170, 2172, 2173, 2174, 2175, 2176, 2177, 2179, 2180, 2181, 2182, 2183, 2184, 2186, 2187, 2188, 2189, 2190, 2191, 2193, 2194, 2195, 2196, 2197, 2198, 2200, 2201, 2202, 2203, 2204, 2205, 2207, 2208, 2209, 2210, 2211, 2212, 2214, 2215, 2216, 2217, 2218, 2219, 2221, 2222, 2223, 2224, 2225, 2226, 2228, 2229, 2230, 2231, 2232, 2233, 2235, 2236, 2237, 2238, 2239, 2240, 2242, 2243, 2244, 2245, 2246, 2247, 2249, 2250, 2251, 2252, 2253, 2254, 2256, 2257, 2258, 2259, 2260, 2261, 2263, 2264, 2265, 2266, 2267, 2268, 2270, 2271, 2272, 2273, 2274, 2275, 2277, 2278, 2279, 2280, 2281, 2282, 2284, 2285, 2286, 2287, 2288, 2289, 2291, 2292, 2293, 2294, 2295, 2296, 2298, 2299, 2300, 2301, 2302, 2303, 2305, 2306, 2307, 2308, 2309, 2310, 2312, 2313, 2314, 2315, 2316, 2317, 2319, 2320, 2321, 2322, 2323, 2324, 2326, 2327, 2328, 2329, 2330, 2331, 2333, 2334, 2335, 2336, 2337, 2338, 2340, 2341, 2342, 2343, 2344, 2345, 2347, 2348, 2349, 2350, 2351, 2352, 2354, 2355, 2356, 2357, 2358, 2359, 2361, 2362, 2363, 2364, 2365, 2366, 2368, 2369, 2370, 2371, 2372, 2373, 2375, 2376, 2377, 2378, 2379, 2380, 2382, 2383, 2384, 2385, 2386, 2387, 2389, 2390, 2391, 2392, 2393, 2394, 2396, 2397, 2398, 2399, 2400, 2401, 2403, 2404, 2405, 2406, 2407, 2408, 2410, 2411, 2412, 2413, 2414, 2415, 2417, 2418, 2419, 2420, 2421, 2422, 2424, 2425, 2426, 2427, 2428, 2429, 2431, 2432, 2433, 2434, 2435, 2436, 2438, 2439, 2440, 2441, 2442, 2443, 2445, 2446, 2447, 2448, 2449, 2450, 2452, 2453, 2454, 2455, 2456, 2457, 2459, 2460, 2461, 2462, 2463, 2464, 2466, 2467, 2468, 2469, 2470, 2471, 2473, 2474, 2475, 2476, 2477, 2478, 2480, 2481, 2482, 2483, 2484, 2485, 2487, 2488, 2489, 2490, 2491, 2492, 2494, 2495, 2496, 2497, 2498, 2499, 2501, 2502, 2503, 2504, 2505, 2506, 2508, 2509, 2510, 2511, 2512, 2513, 2515, 2516, 2517, 2518, 2519, 2520, 2522, 2523, 2524, 2525, 2526, 2527, 2529, 2530, 2531, 2532, 2533, 2534, 2536, 2537, 2538, 2539, 2540, 2541, 2543, 2544, 2545, 2546, 2547, 2548, 2550, 2551, 2552, 2553, 2554, 2555, 2557, 2558, 2559, 2560, 2561, 2562, 2564, 2565, 2566, 2567, 2568, 2569, 2571, 2572, 2573, 2574, 2575, 2576, 2578, 2579, 2580, 2581, 2582, 2583, 2585, 2586, 2587, 2588, 2589, 2590, 2592, 2593, 2594, 2595, 2596, 2597, 2599, 2600, 2601, 2602, 2603, 2604, 2606, 2607, 2608, 2609, 2610, 2611, 2613, 2614, 2615, 2616, 2617, 2618, 2620, 2621, 2622, 2623, 2624, 2625, 2627, 2628, 2629, 2630, 2631, 2632, 2634, 2635, 2636, 2637, 2638, 2639, 2641, 2642, 2643, 2644, 2645, 2646, 2648, 2649, 2650, 2651, 2652, 2653, 2655, 2656, 2657, 2658, 2659, 2660, 2662, 2663, 2664, 2665, 2666, 2667, 2669, 2670, 2671, 2672, 2673, 2674, 2676, 2677, 2678, 2679, 2680, 2681, 2683, 2684, 2685, 2686, 2687, 2688, 2690, 2691, 2692, 2693, 2694, 2695, 2697, 2698, 2699, 2700, 2701, 2702, 2704, 2705, 2706, 2707, 2708, 2709, 2711, 2712, 2713, 2714, 2715, 2716, 2718, 2719, 2720, 2721, 2722, 2723, 2725, 2726, 2727, 2728, 2729, 2730, 2732, 2733, 2734, 2735, 2736, 2737, 2739, 2740, 2741, 2742, 2743, 2744, 2746, 2747, 2748, 2749, 2750, 2751, 2753, 2754, 2755, 2756, 2757, 2758, 2760, 2761, 2762, 2763, 2764, 2765, 2767, 2768, 2769, 2770, 2771, 2772, 2774, 2775, 2776, 2777, 2778, 2779, 2781, 2782, 2783, 2784, 2785, 2786, 2788, 2789, 2790, 2791, 2792, 2793, 2795, 2796, 2797, 2798, 2799, 2800, 2802, 2803, 2804, 2805, 2806, 2807, 2809, 2810, 2811, 2812, 2813, 2814, 2816, 2817, 2818, 2819, 2820, 2821, 2823, 2824, 2825, 2826, 2827, 2828, 2830, 2831, 2832, 2833, 2834, 2835, 2837, 2838, 2839, 2840, 2841, 2842, 2844, 2845, 2846, 2847, 2848, 2849, 2851, 2852, 2853, 2854, 2855, 2856, 2858, 2859, 2860, 2861, 2862, 2863, 2865, 2866, 2867, 2868, 2869, 2870, 2872, 2873, 2874, 2875, 2876, 2877, 2879, 2880, 2881, 2882, 2883, 2884, 2886, 2887, 2888, 2889, 2890, 2891, 2893, 2894, 2895, 2896, 2897, 2898, 2900, 2901, 2902, 2903, 2904, 2905, 2907, 2908, 2909, 2910, 2911, 2912, 2914, 2915, 2916, 2917, 2918, 2919, 2921, 2922, 2923, 2924, 2925, 2926, 2928, 2929, 2930, 2931, 2932, 2933, 2935, 2936, 2937, 2938, 2939, 2940, 2942, 2943, 2944, 2945, 2946, 2947, 2949, 2950, 2951, 2952, 2953, 2954, 2956, 2957, 2958, 2959, 2960, 2961, 2963, 2964, 2965, 2966, 2967, 2968, 2970, 2971, 2972, 2973, 2974, 2975, 2977, 2978, 2979, 2980, 2981, 2982, 2984, 2985, 2986, 2987, 2988, 2989, 2991, 2992, 2993, 2994, 2995, 2996, 2998, 2999, 3000, 3001, 3002, 3003, 3005, 3006, 3007, 3008, 3009, 3010, 3012, 3013, 3014, 3015, 3016, 3017, 3019, 3020, 3021, 3022, 3023, 3024, 3026, 3027, 3028, 3029, 3030, 3031, 3033, 3034, 3035, 3036, 3037, 3038, 3040, 3041, 3042, 3043, 3044, 3045, 3047, 3048, 3049, 3050, 3051, 3052, 3054, 3055, 3056, 3057, 3058, 3059, 3061, 3062, 3063, 3064, 3065, 3066, 3068, 3069, 3070, 3071, 3072, 3073, 3075, 3076, 3077, 3078, 3079, 3080, 3082, 3083, 3084, 3085, 3086, 3087, 3089, 3090, 3091, 3092, 3093, 3094, 3096, 3097, 3098, 3099, 3100, 3101, 3103, 3104, 3105, 3106, 3107, 3108, 3110, 3111, 3112, 3113, 3114, 3115, 3117, 3118, 3119, 3120, 3121, 3122, 3124, 3125, 3126, 3127, 3128, 3129, 3131, 3132, 3133, 3134, 3135, 3136, 3138, 3139, 3140, 3141, 3142, 3143, 3145, 3146, 3147, 3148, 3149, 3150, 3152, 3153, 3154, 3155, 3156, 3157, 3159, 3160, 3161, 3162, 3163, 3164, 3166, 3167, 3168, 3169, 3170, 3171, 3173, 3174, 3175, 3176, 3177, 3178, 3180, 3181, 3182, 3183, 3184, 3185, 3187, 3188, 3189, 3190, 3191, 3192, 3194, 3195, 3196, 3197, 3198, 3199, 3201, 3202, 3203, 3204, 3205, 3206, 3208, 3209, 3210, 3211, 3212, 3213, 3215, 3216, 3217, 3218, 3219, 3220, 3222, 3223, 3224, 3225, 3226, 3227, 3229, 3230, 3231, 3232, 3233, 3234, 3236, 3237, 3238, 3239, 3240, 3241, 3243, 3244, 3245, 3246, 3247, 3248, 3250, 3251, 3252, 3253, 3254, 3255, 3257, 3258, 3259, 3260, 3261, 3262, 3264, 3265, 3266, 3267, 3268, 3269, 3271, 3272, 3273, 3274, 3275, 3276, 3278, 3279, 3280, 3281, 3282, 3283, 3285, 3286, 3287, 3288, 3289, 3290, 3292, 3293, 3294, 3295, 3296, 3297, 3299, 3300, 3301, 3302, 3303, 3304, 3306, 3307, 3308, 3309, 3310, 3311, 3313, 3314, 3315, 3316, 3317, 3318, 3320, 3321, 3322, 3323, 3324, 3325, 3327, 3328, 3329, 3330, 3331, 3332, 3334, 3335, 3336, 3337, 3338, 3339, 3341, 3342, 3343, 3344, 3345, 3346, 3348, 3349, 3350, 3351, 3352, 3353, 3355, 3356, 3357, 3358, 3359, 3360, 3362, 3363, 3364, 3365, 3366, 3367, 3369, 3370, 3371, 3372, 3373, 3374, 3376, 3377, 3378, 3379, 3380, 3381, 3383, 3384, 3385, 3386, 3387, 3388, 3390, 3391, 3392, 3393, 3394, 3395, 3397, 3398, 3399, 3400, 3401, 3402, 3404, 3405, 3406, 3407, 3408, 3409, 3411, 3412, 3413, 3414, 3415, 3416, 3418, 3419, 3420, 3421, 3422, 3423, 3425, 3426, 3427, 3428, 3429, 3430, 3432, 3433, 3434, 3435, 3436, 3437, 3439, 3440, 3441, 3442, 3443, 3444, 3446, 3447, 3448, 3449, 3450, 3451, 3453, 3454, 3455, 3456, 3457, 3458, 3460, 3461, 3462, 3463, 3464, 3465, 3467, 3468, 3469, 3470, 3471, 3472, 3474, 3475, 3476, 3477, 3478, 3479, 3481, 3482, 3483, 3484, 3485, 3486, 3488, 3489, 3490, 3491, 3492, 3493, 3495, 3496, 3497, 3498, 3499, 3500, 3502, 3503, 3504, 3505, 3506, 3507, 3509, 3510, 3511, 3512, 3513, 3514, 3516, 3517, 3518, 3519, 3520, 3521, 3523, 3524, 3525, 3526, 3527 or 3528 as described in international patent application PCT/EP2016/059111, or a fragment or variant of any of these RNA sequences.

According to a particularly preferred embodiment, the at least one first (immunogenic) component of the combination or composition according to the invention comprises at least one RNA sequence selected from RNA sequences being identical or at least 50%, 60%, 70%, 80%, 85%, 86%, 87%, 88%, 89%, 90%, 91%, 92%, 93%, 94%, 95%, 96%, 97%, 98%, or 99%, preferably at least 80%, identical to the RNA sequences according to any one of SEQ ID NOs: 3, 4, 5, 6, 7, 10, 11, 12, 13, 14, 17, 18, 19, 20, 21, 24, 25, 26, 27, 28, 31, 32, 33, 34, 35, 38, 39, 40, 41, 42, 45, 46, 47, 48, 49, 52, 53, 54, 55, 56, 59, 60, 61, 62, 63, 66, 67, 68, 69, 70, 73, 74, 75, 76, 77, 80, 81, 82, 83, 84, 87, 88, 89, 90, 91, 94, 95, 96, 97, 98, 101, 102, 103, 104, 105, 108, 109, 110, 111, 112, 115, 116, 117, 118, 119, 122, 123, 124, 125, 126, 129, 130, 131, 132, 133, 136, 137, 138, 139, 140, 143, 144, 145, 146, 147, 150, 151, 152, 153, 154, 157, 158, 159, 160, 161, 164, 165, 166, 167, 168, 171, 172, 173, 174, 175, 178, 179, 180, 181, 182, 185, 186, 187, 188, 189, 192, 193, 194, 195, 196, 199, 200, 201, 202, 203, 206, 207, 208, 209, 210, 213, 214, 215, 216, 217, 220, 221, 222, 223, 224, 227, 228, 229, 230, 231, 234, 235, 236, 237, 238, 241, 242, 243, 244, 245, 248, 249, 250, 251, 252, 255, 256, 257, 258, 259, 262, 263, 264, 265, 266, 269, 270, 271, 272, 273, 276, 277, 278, 279, 280, 283, 284, 285, 286, 287, 290, 291, 292, 293, 294, 297, 298, 299, 300, 301, 304, 305, 306, 307, 308, 311, 312, 313, 314, 315, 318, 319, 320, 321, 322, 325, 326, 327, 328, 329, 332, 333, 334, 335, 336, 339, 340, 341, 342, 343, 346, 347, 348, 349, 350, 353, 354, 355, 356, 357, 360, 361, 362, 363, 364, 367, 368, 369, 370, 371, 374, 375, 376, 377, 378, 381, 382, 383, 384, 385, 388, 389, 390, 391, 392, 395, 396, 397, 398, 399, 402, 403, 404, 405, 406, 409, 410, 411, 412, 413, 416, 417, 418, 419, 420, 423, 424, 425, 426, 427, 430, 431, 432, 433, 434, 437, 438, 439, 440, 441, 444, 445, 446, 447, 448, 451, 452, 453, 454, 455, 458, 459, 460, 461, 462, 465, 466, 467, 468, 469, 472, 473, 474, 475, 476, 479, 480, 481, 482, 483, 486, 487, 488, 489, 490, 493, 494, 495, 496, 497, 500, 501, 502, 503, 504, 507, 508, 509, 510, 511, 514, 515, 516, 517, 518, 521, 522, 523, 524, 525, 528, 529, 530, 531, 532, 535, 536, 537, 538, 539, 542, 543, 544, 545, 546, 549, 550, 551, 552, 553, 556, 557, 558, 559, 560, 563, 564, 565, 566, 567, 570, 571, 572, 573, 574, 577, 578, 579, 580, 581, 584, 585, 586, 587, 588, 591, 592, 593, 594, 595, 598, 599, 600, 601, 602, 605, 606, 607, 608, 609, 612, 613, 614, 615, 616, 619, 620, 621, 622, 623, 626, 627, 628, 629, 630, 633, 634, 635, 636, 637, 640, 641, 642, 643, 644, 647, 648, 649, 650, 651, 654, 655, 656, 657, 658, 661, 662, 663, 664, 665, 668, 669, 670, 671, 672, 675, 676, 677, 678, 679, 682, 683, 684, 685, 686, 689, 690, 691, 692, 693, 696, 697, 698, 699, 700, 703, 704, 705, 706, 707, 710, 711, 712, 713, 714, 717, 718, 719, 720, 721, 724, 725, 726, 727, 728, 731, 732, 733, 734, 735, 738, 739, 740, 741, 742, 745, 746, 747, 748, 749, 752, 753, 754, 755, 756, 759, 760, 761, 762, 763, 766, 767, 768, 769, 770, 773, 774, 775, 776, 777, 780, 781, 782, 783, 784, 787, 788, 789, 790, 791, 794, 795, 796, 797, 798, 801, 802, 803, 804, 805, 808, 809, 810, 811, 812, 815, 816, 817, 818, 819, 822, 823, 824, 825, 826, 829, 830, 831, 832, 833, 836, 837, 838, 839, 840, 843, 844, 845, 846, 847, 850, 851, 852, 853, 854, 857, 858, 859, 860, 861, 864, 865, 866, 867, 868, 871, 872, 873, 874, 875, 878, 879, 880, 881, 882, 885, 886, 887, 888, 889, 892, 893, 894, 895, 896, 899, 900, 901, 902, 903, 906, 907, 908, 909, 910, 913, 914, 915, 916, 917, 920, 921, 922, 923, 924, 927, 928, 929, 930, 931, 934, 935, 936, 937, 938, 941, 942, 943, 944, 945, 948, 949, 950, 951, 952, 955, 956, 957, 958, 959, 962, 963, 964, 965, 966, 969, 970, 971, 972, 973, 976, 977, 978, 979, 980, 983, 984, 985, 986, 987, 990, 991, 992, 993, 994, 997, 998, 999, 1000, 1001, 1004, 1005, 1006, 1007, 1008, 1011, 1012, 1013, 1014, 1015, 1018, 1019, 1020, 1021, 1022, 1025, 1026, 1027, 1028, 1029, 1032, 1033, 1034, 1035, 1036, 1039, 1040, 1041, 1042, 1043, 1046, 1047, 1048, 1049, 1050, 1053, 1054, 1055, 1056, 1057, 1060, 1061, 1062, 1063, 1064, 1067, 1068, 1069, 1070, 1071, 1074, 1075, 1076, 1077, 1078, 1081, 1082, 1083, 1084, 1085, 1088, 1089, 1090, 1091, 1092, 1095, 1096, 1097, 1098, 1099, 1102, 1103, 1104, 1105, 1106, 1109, 1110, 1111, 1112, 1113, 1116, 1117, 1118, 1119, 1120, 1123, 1124, 1125, 1126, 1127, 1130, 1131, 1132, 1133, 1134, 1137, 1138, 1139, 1140, 1141, 1144, 1145, 1146, 1147, 1148, 1151, 1152, 1153, 1154, 1155, 1158, 1159, 1160, 1161, 1162, 1165, 1166, 1167, 1168, 1169, 1172, 1173, 1174, 1175, 1176, 1179, 1180, 1181, 1182, 1183, 1186, 1187, 1188, 1189, 1190, 1193, 1194, 1195, 1196, 1197, 1200, 1201, 1202, 1203, 1204, 1207, 1208, 1209, 1210, 1211, 1214, 1215, 1216, 1217, 1218, 1221, 1222, 1223, 1224, 1225, 1228, 1229, 1230, 1231, 1232, 1235, 1236, 1237, 1238, 1239, 1242, 1243, 1244, 1245, 1246, 1249, 1250, 1251, 1252, 1253, 1256, 1257, 1258, 1259, 1260, 1263, 1264, 1265, 1266, 1267, 1270, 1271, 1272, 1273, 1274, 1277, 1278, 1279, 1280, 1281, 1284, 1285, 1286, 1287, 1288, 1291, 1292, 1293, 1294, 1295, 1298, 1299, 1300, 1301, 1302, 1305, 1306, 1307, 1308, 1309, 1312, 1313, 1314, 1315, 1316, 1319, 1320, 1321, 1322, 1323, 1326, 1327, 1328, 1329, 1330, 1333, 1334, 1335, 1336, 1337, 1340, 1341, 1342, 1343, 1344, 1347, 1348, 1349, 1350, 1351, 1354, 1355, 1356, 1357, 1358, 1361, 1362, 1363, 1364, 1365, 1368, 1369, 1370, 1371, 1372, 1375, 1376, 1377, 1378, 1379, 1382, 1383, 1384, 1385, 1386, 1389, 1390, 1391, 1392, 1393, 1396, 1397, 1398, 1399, 1400, 1403, 1404, 1405, 1406, 1407, 1410, 1411, 1412, 1413, 1414, 1417, 1418, 1419, 1420, 1421, 1424, 1425, 1426, 1427, 1428, 1431, 1432, 1433, 1434, 1435, 1438, 1439, 1440, 1441, 1442, 1445, 1446, 1447, 1448, 1449, 1452, 1453, 1454, 1455, 1456, 1459, 1460, 1461, 1462, 1463, 1466, 1467, 1468, 1469, 1470, 1473, 1474, 1475, 1476, 1477, 1480, 1481, 1482, 1483, 1484, 1487, 1488, 1489, 1490, 1491, 1494, 1495, 1496, 1497, 1498, 1501, 1502, 1503, 1504, 1505, 1508, 1509, 1510, 1511, 1512, 1515, 1516, 1517, 1518, 1519, 1522, 1523, 1524, 1525, 1526, 1529, 1530, 1531, 1532, 1533, 1536, 1537, 1538, 1539, 1540, 1543, 1544, 1545, 1546, 1547, 1550, 1551, 1552, 1553, 1554, 1557, 1558, 1559, 1560, 1561, 1564, 1565, 1566, 1567, 1568, 1571, 1572, 1573, 1574, 1575, 1578, 1579, 1580, 1581, 1582, 1585, 1586, 1587, 1588, 1589, 1592, 1593, 1594, 1595, 1596, 1599, 1600, 1601, 1602, 1603, 1606, 1607, 1608, 1609, 1610, 1613, 1614, 1615, 1616, 1617, 1620, 1621, 1622, 1623, 1624, 1627, 1628, 1629, 1630, 1631, 1634, 1635, 1636, 1637, 1638, 1641, 1642, 1643, 1644, 1645, 1648, 1649, 1650, 1651, 1652, 1655, 1656, 1657, 1658, 1659, 1662, 1663, 1664, 1665, 1666, 1669, 1670, 1671, 1672, 1673, 1676, 1677, 1678, 1679, 1680, 1683, 1684, 1685, 1686, 1687, 1690, 1691, 1692, 1693, 1694, 1697, 1698, 1699, 1700, 1701, 1704, 1705, 1706, 1707, 1708, 1711, 1712, 1713, 1714, 1715, 1718, 1719, 1720, 1721, 1722, 1725, 1726, 1727, 1728, 1729, 1732, 1733, 1734, 1735, 1736, 1739, 1740, 1741, 1742, 1743, 1746, 1747, 1748, 1749, 1750, 1753, 1754, 1755, 1756, 1757, 1760, 1761, 1762, 1763, 1764, 1767, 1768, 1769, 1770, 1771, 1774, 1775, 1776, 1777, 1778, 1781, 1782, 1783, 1784, 1785, 1788, 1789, 1790, 1791, 1792, 1795, 1796, 1797, 1798, 1799, 1802, 1803, 1804, 1805, 1806, 1809, 1810, 1811, 1812, 1813, 1816, 1817, 1818, 1819, 1820, 1823, 1824, 1825, 1826, 1827, 1830, 1831, 1832, 1833, 1834, 1837, 1838, 1839, 1840, 1841, 1844, 1845, 1846, 1847, 1848, 1851, 1852, 1853, 1854, 1855, 1858, 1859, 1860, 1861, 1862, 1865, 1866, 1867, 1868, 1869, 1872, 1873, 1874, 1875, 1876, 1879, 1880, 1881, 1882, 1883, 1886, 1887, 1888, 1889, 1890, 1893, 1894, 1895, 1896, 1897, 1900, 1901, 1902, 1903, 1904, 1907, 1908, 1909, 1910, 1911, 1914, 1915, 1916, 1917, 1918, 1921, 1922, 1923, 1924, 1925, 1928, 1929, 1930, 1931, 1932, 1935, 1936, 1937, 1938, 1939, 1942, 1943, 1944, 1945, 1946, 1949, 1950, 1951, 1952, 1953, 1956, 1957, 1958, 1959, 1960, 1963, 1964, 1965, 1966, 1967, 1970, 1971, 1972, 1973, 1974, 1977, 1978, 1979, 1980, 1981, 1984, 1985, 1986, 1987, 1988, 1991, 1992, 1993, 1994, 1995, 1998, 1999, 2000, 2001, 2002, 2005, 2006, 2007, 2008, 2009, 2012, 2013, 2014, 2015, 2016, 2019, 2020, 2021, 2022, 2023, 2026, 2027, 2028, 2029, 2030, 2033, 2034, 2035, 2036, 2037, 2040, 2041, 2042, 2043, 2044, 2047, 2048, 2049, 2050, 2051, 2054, 2055, 2056, 2057, 2058, 2061, 2062, 2063, 2064, 2065, 2068, 2069, 2070, 2071, 2072, 2075, 2076, 2077, 2078, 2079, 2082, 2083, 2084, 2085, 2086, 2089, 2090, 2091, 2092, 2093, 2096, 2097, 2098, 2099, 2100, 2103, 2104, 2105, 2106, 2107, 2110, 2111, 2112, 2113, 2114, 2117, 2118, 2119, 2120, 2121, 2124, 2125, 2126, 2127, 2128, 2131, 2132, 2133, 2134, 2135, 2138, 2139, 2140, 2141, 2142, 2145, 2146, 2147, 2148, 2149, 2152, 2153, 2154, 2155, 2156, 2159, 2160, 2161, 2162, 2163, 2166, 2167, 2168, 2169, 2170, 2173, 2174, 2175, 2176, 2177, 2180, 2181, 2182, 2183, 2184, 2187, 2188, 2189, 2190, 2191, 2194, 2195, 2196, 2197, 2198, 2201, 2202, 2203, 2204, 2205, 2208, 2209, 2210, 2211, 2212, 2215, 2216, 2217, 2218, 2219, 2222, 2223, 2224, 2225, 2226, 2229, 2230, 2231, 2232, 2233, 2236, 2237, 2238, 2239, 2240, 2243, 2244, 2245, 2246, 2247, 2250, 2251, 2252, 2253, 2254, 2257, 2258, 2259, 2260, 2261, 2264, 2265, 2266, 2267, 2268, 2271, 2272, 2273, 2274, 2275, 2278, 2279, 2280, 2281, 2282, 2285, 2286, 2287, 2288, 2289, 2292, 2293, 2294, 2295, 2296, 2299, 2300, 2301, 2302, 2303, 2306, 2307, 2308, 2309, 2310, 2313, 2314, 2315, 2316, 2317, 2320, 2321, 2322, 2323, 2324, 2327, 2328, 2329, 2330, 2331, 2334, 2335, 2336, 2337, 2338, 2341, 2342, 2343, 2344, 2345, 2348, 2349, 2350, 2351, 2352, 2355, 2356, 2357, 2358, 2359, 2362, 2363, 2364, 2365, 2366, 2369, 2370, 2371, 2372, 2373, 2376, 2377, 2378, 2379, 2380, 2383, 2384, 2385, 2386, 2387, 2390, 2391, 2392, 2393, 2394, 2397, 2398, 2399, 2400, 2401, 2404, 2405, 2406, 2407, 2408, 2411, 2412, 2413, 2414, 2415, 2418, 2419, 2420, 2421, 2422, 2425, 2426, 2427, 2428, 2429, 2432, 2433, 2434, 2435, 2436, 2439, 2440, 2441, 2442, 2443, 2446, 2447, 2448, 2449, 2450, 2453, 2454, 2455, 2456, 2457, 2460, 2461, 2462, 2463, 2464, 2467, 2468, 2469, 2470, 2471, 2474, 2475, 2476, 2477, 2478, 2481, 2482, 2483, 2484, 2485, 2488, 2489, 2490, 2491, 2492, 2495, 2496, 2497, 2498, 2499, 2502, 2503, 2504, 2505, 2506, 2509, 2510, 2511, 2512, 2513, 2516, 2517, 2518, 2519, 2520, 2523, 2524, 2525, 2526, 2527, 2530, 2531, 2532, 2533, 2534, 2537, 2538, 2539, 2540, 2541, 2544, 2545, 2546, 2547, 2548, 2551, 2552, 2553, 2554, 2555, 2558, 2559, 2560, 2561, 2562, 2565, 2566, 2567, 2568, 2569, 2572, 2573, 2574, 2575, 2576, 2579, 2580, 2581, 2582, 2583, 2586, 2587, 2588, 2589, 2590, 2593, 2594, 2595, 2596, 2597, 2600, 2601, 2602, 2603, 2604, 2607, 2608, 2609, 2610, 2611, 2614, 2615, 2616, 2617, 2618, 2621, 2622, 2623, 2624, 2625, 2628, 2629, 2630, 2631, 2632, 2635, 2636, 2637, 2638, 2639, 2642, 2643, 2644, 2645, 2646, 2649, 2650, 2651, 2652, 2653, 2656, 2657, 2658, 2659, 2660, 2663, 2664, 2665, 2666, 2667, 2670, 2671, 2672, 2673, 2674, 2677, 2678, 2679, 2680, 2681, 2684, 2685, 2686, 2687, 2688, 2691, 2692, 2693, 2694, 2695, 2698, 2699, 2700, 2701, 2702, 2705, 2706, 2707, 2708, 2709, 2712, 2713, 2714, 2715, 2716, 2719, 2720, 2721, 2722, 2723, 2726, 2727, 2728, 2729, 2730, 2733, 2734, 2735, 2736, 2737, 2740, 2741, 2742, 2743, 2744, 2747, 2748, 2749, 2750, 2751, 2754, 2755, 2756, 2757, 2758, 2761, 2762, 2763, 2764, 2765, 2768, 2769, 2770, 2771, 2772, 2775, 2776, 2777, 2778, 2779, 2782, 2783, 2784, 2785, 2786, 2789, 2790, 2791, 2792, 2793, 2796, 2797, 2798, 2799, 2800, 2803, 2804, 2805, 2806, 2807, 2810, 2811, 2812, 2813, 2814, 2817, 2818, 2819, 2820, 2821, 2824, 2825, 2826, 2827, 2828, 2831, 2832, 2833, 2834, 2835, 2838, 2839, 2840, 2841, 2842, 2845, 2846, 2847, 2848, 2849, 2852, 2853, 2854, 2855, 2856, 2859, 2860, 2861, 2862, 2863, 2866, 2867, 2868, 2869, 2870, 2873, 2874, 2875, 2876, 2877, 2880, 2881, 2882, 2883, 2884, 2887, 2888, 2889, 2890, 2891, 2894, 2895, 2896, 2897, 2898, 2901, 2902, 2903, 2904, 2905, 2908, 2909, 2910, 2911, 2912, 2915, 2916, 2917, 2918, 2919, 2922, 2923, 2924, 2925, 2926, 2929, 2930, 2931, 2932, 2933, 2936, 2937, 2938, 2939, 2940, 2943, 2944, 2945, 2946, 2947, 2950, 2951, 2952, 2953, 2954, 2957, 2958, 2959, 2960, 2961, 2964, 2965, 2966, 2967, 2968, 2971, 2972, 2973, 2974, 2975, 2978, 2979, 2980, 2981, 2982, 2985, 2986, 2987, 2988, 2989, 2992, 2993, 2994, 2995, 2996, 2999, 3000, 3001, 3002, 3003, 3006, 3007, 3008, 3009, 3010, 3013, 3014, 3015, 3016, 3017, 3020, 3021, 3022, 3023, 3024, 3027, 3028, 3029, 3030, 3031, 3034, 3035, 3036, 3037, 3038, 3041, 3042, 3043, 3044, 3045, 3048, 3049, 3050, 3051, 3052, 3055, 3056, 3057, 3058, 3059, 3062, 3063, 3064, 3065, 3066, 3069, 3070, 3071, 3072, 3073, 3076, 3077, 3078, 3079, 3080, 3083, 3084, 3085, 3086, 3087, 3090, 3091, 3092, 3093, 3094, 3097, 3098, 3099, 3100, 3101, 3104, 3105, 3106, 3107, 3108, 3111, 3112, 3113, 3114, 3115, 3118, 3119, 3120, 3121, 3122, 3125, 3126, 3127, 3128, 3129, 3132, 3133, 3134, 3135, 3136, 3139, 3140, 3141, 3142, 3143, 3146, 3147, 3148, 3149, 3150, 3153, 3154, 3155, 3156, 3157, 3160, 3161, 3162, 3163, 3164, 3167, 3168, 3169, 3170, 3171, 3174, 3175, 3176, 3177, 3178, 3181, 3182, 3183, 3184, 3185, 3188, 3189, 3190, 3191, 3192, 3195, 3196, 3197, 3198, 3199, 3202, 3203, 3204, 3205, 3206, 3209, 3210, 3211, 3212, 3213, 3216, 3217, 3218, 3219, 3220, 3223, 3224, 3225, 3226, 3227, 3230, 3231, 3232, 3233, 3234, 3237, 3238, 3239, 3240, 3241, 3244, 3245, 3246, 3247, 3248, 3251, 3252, 3253, 3254, 3255, 3258, 3259, 3260, 3261, 3262, 3265, 3266, 3267, 3268, 3269, 3272, 3273, 3274, 3275, 3276, 3279, 3280, 3281, 3282, 3283, 3286, 3287, 3288, 3289, 3290, 3293, 3294, 3295, 3296, 3297, 3300, 3301, 3302, 3303, 3304, 3307, 3308, 3309, 3310, 3311, 3314, 3315, 3316, 3317, 3318, 3321, 3322, 3323, 3324, 3325, 3328, 3329, 3330, 3331, 3332, 3335, 3336, 3337, 3338, 3339, 3342, 3343, 3344, 3345, 3346, 3349, 3350, 3351, 3352, 3353, 3356, 3357, 3358, 3359, 3360, 3363, 3364, 3365, 3366, 3367, 3370, 3371, 3372, 3373, 3374, 3377, 3378, 3379, 3380, 3381, 3384, 3385, 3386, 3387, 3388, 3391, 3392, 3393, 3394, 3395, 3398, 3399, 3400, 3401, 3402, 3405, 3406, 3407, 3408, 3409, 3412, 3413, 3414, 3415, 3416, 3419, 3420, 3421, 3422, 3423, 3426, 3427, 3428, 3429, 3430, 3433, 3434, 3435, 3436, 3437, 3440, 3441, 3442, 3443, 3444, 3447, 3448, 3449, 3450, 3451, 3454, 3455, 3456, 3457, 3458, 3461, 3462, 3463, 3464, 3465, 3468, 3469, 3470, 3471, 3472, 3475, 3476, 3477, 3478, 3479, 3482, 3483, 3484, 3485, 3486, 3489, 3490, 3491, 3492, 3493, 3496, 3497, 3498, 3499, 3500, 3503, 3504, 3505, 3506, 3507, 3510, 3511, 3512, 3513, 3514, 3517, 3518, 3519, 3520, 3521, 3524, 3525, 3526, 3527 or 3528 as described in international patent application PCT/EP2016/059111, or a fragment or variant of any of these RNA sequences.

In a further preferred embodiment of the inventive composition the at least one antigen is associated with allergy or allergic disease and preferably is derived from a source selected from the list consisting of: grass pollen, tree pollen, flower pollen, herb pollen, dust mite, mold, animals, food, and insect venom.

In a further preferred embodiment of the inventive composition the at least one antigen is associated with an autoimmune disease and preferably is selected from the list consisting of:
myelin basic protein (MBP), proteolipid protein (PLP), and myelin oligodendrocyte glycoprotein (MOG), in each case associated with multiple sclerosis (MS);
CD44, preproinsulin, proinsulin, insulin, glutamic acid decaroxylase (GAD65), tyrosine phosphatase-like insulinoma antigen 2 (IA2), zinc transporter (ZnT8), and heat shock protein 60 (HSP60), in each case associated with diabetes Typ I;
interphotoreceptor retinoid-binding protein (IRBP) associated with autoimmune uveitis;
acetylcholine receptor AchR, and insulin-like growth factor-1 receptor (IGF-1R), in each case associated with Myasthenia gravis;
M-protein from beta-hemolytic streptocci (pseudo-autoantigen) associated with Rheumatic Fever;
Macrophage migration inhibitory factor associated with Arthritis;
Ro/La RNP complex, alpha- and beta-fodrin, islet cell autoantigen, poly(ADP)ribose polymerase (PARP), NuMA, NOR-90, Ro60 autoantigen, and p27 antigen, in each case associated with Sjögren's syndrome;
Ro60 autoantigen, low-density lipoproteins, Sm antigens of the U-1 small nuclear ribonucleoprotein complex (B/B′, D1, D2, D3, E, F, G), and RNP ribonucleoproteins, in each case associated with lupus erythematosus;
oxLDL, beta(2)GPI, HSP60/65, and oxLDL/beta(2)GPI, in each case associated with atherosclerosis;
cardiac beta(1)-adrenergic receptor associated with idiopathic dilated cardiomyopathy (DCM);
histidyl-tRNA synthetase (HisRS) associated with myositis;
topoisomerase I associated with scleroderma;
IL-17; and
heat shock proteins.

In an especially preferred embodiment of the inventive composition the immunogenic component comprises at least one nucleic acid molecule, in particular an mRNA sequence, encoding at least one epitope of at least one antigen of Influenza virus, preferably Influenza A virus, wherein the antigen is preferably Hemagglutinin (HA), preferably according to SEQ ID NO. 1 (see also FIG. 1). The second adjuvant component may comprise e.g. an emulsion, preferably an oil-in-water emulsion, more preferably a squalene-based compound, most preferably MF59®, or another adjuvant compound as describe above. In a further preferred embodiment the second adjuvant component may comprise a vitamin A compound or a vitamin A derivative compound, preferably all-trans retinoic acid (ATRA) or retinyl palmitate. It is particularly preferred that the second adjuvant component for the HA antigen comprises at least two adjuvant components, in particular a vitamin A compound or a vitamin A derivative compound, preferably all-trans retinoic acid (ATRA) or retinyl palmitate, and a polymeric carrier cargo complex as described above.

In another preferred embodiment of the inventive composition the immunogenic component comprises at least one nucleic acid molecule, in particular an mRNA sequence, encoding at least one epitope of at least one antigen of Rabies virus, wherein the antigen is preferably glycoprotein G (RAV-G), preferably according to SEQ ID NO. 3 or 4 (see also FIG. 9 or FIG. 10). Preferably the mRNA sequence is at least partly complexed with protamine. Moreover the adjuvant component of this composition preferably comprises an emulsion, preferably an oil-in-water emulsion, more preferably a squalene-based compound, most preferably MF59®.

According to certain embodiments, the present invention provides a combination or a composition comprising

at least a first (immunogenic) component and at least a second (adjuvant) component,
wherein the first (immunogenic) component comprises at least one nucleic acid molecule, preferably an mRNA, encoding at least one epitope of at least one antigen, wherein the at least one antigen is selected from the group consisting of an antigen from a pathogen associated with infectious diseases, an antigen associated with allergies, an antigen associated with autoimmune diseases, and an antigen associated with cancer or tumor diseases, or a fragment, variant and/or derivative of said antigen; and
wherein the second (adjuvant) component comprises at least one immune potentiator compound and/or at least one delivery system compound, wherein the second (adjuvant) component, preferably the immune potentiator compound or the delivery system compound, is a mineral salt adjuvant as described herein, preferably selected from aluminium salts and calcium salts, more preferably selected from aluminium phosphate salts and calcium phosphate salts, most preferably an aluminium phosphate salt, such as Adju-Phos.

In a preferred embodiment, the present invention relates to a combination or a composition comprising

at least a first (immunogenic) component and at least a second (adjuvant) component,
wherein the first (immunogenic) component comprises at least one nucleic acid molecule, preferably an mRNA, encoding at least one epitope of at least one antigen associated with cancer or tumor diseases, preferably as described herein, or a fragment, variant and/or derivative of said antigen; and
wherein the second (adjuvant) component comprises at least one immune potentiator compound and/or at least one delivery system compound, wherein the second (adjuvant) component, preferably the immune potentiator compound or the delivery system compound, is a mineral salt adjuvant as described herein, preferably selected from aluminium salts and calcium salts, more preferably selected from aluminium phosphate salts and calcium phosphate salts, most preferably an aluminium phosphate salt, such as Adju-Phos.

According to a particularly preferred embodiment, the present invention provides a combination or a composition comprising

at least a first (immunogenic) component and at least a second (adjuvant) component,
wherein the first (immunogenic) component comprises at least one nucleic acid molecule, preferably an mRNA, encoding at least one epitope of at least one antigen from a pathogen associated with infectious diseases, preferably as described herein, or a fragment, variant and/or derivative of said antigen; and wherein the second (adjuvant) component comprises at least one immune potentiator compound and/or at least one delivery system compound, wherein the second (adjuvant) component, preferably the immune potentiator compound or the delivery system compound, is a mineral salt adjuvant as described herein, preferably selected from aluminium salts and calcium salts, more preferably selected from aluminium phosphate salts and calcium phosphate salts, most preferably an aluminium phosphate salt, such as Adju-Phos.

In this context, it is particularly preferred that the at least one nucleic acid molecule, preferably an mRNA, encodes at least one epitope of at least one antigen from Influenza virus, Nora virus or Rhinovirus, wherein the at least one nucleic acid molecule comprises one of the nucleic acid sequences specified herein in that context.

One further additive, which may be contained in the inventive composition, may be an anti-bacterial agent. In this context, any anti-bacterial agents known to one of skill in the art may be used in combination with the components of the inventive composition as defined herein. Non-limiting examples of anti-bacterial agents include Amikacin, Amoxicillin, Amoxicillin-clavulanic acid, Amphothericin-B, Ampicillin, Ampicllin-sulbactam, Apramycin, Azithromycin, Aztreonam, Bacitracin, Benzylpenicillin, Caspofungin, Cefaclor, Cefadroxil, Cefalexin, Cefalothin, Cefazolin, Cefdinir, Cefepime, Cefixime, Cefmenoxime, Cefoperazone, Cefoperazone-sulbactam, Cefotaxime, Cefoxitin, Cefbirome, Cefpodoxime, Cefpodoxime-clavulanic acid, Cefpodoxime-sulbactam, Cefbrozil, Cefquinome, Ceftazidime, Ceftibutin, Ceftiofur, Ceftobiprole, Ceftriaxon, Cefuroxime, Chloramphenicole, Florfenicole, Ciprofloxacin, Clarithromycin, Clinafloxacin, Clindamycin, Cloxacillin, Colistin, Cotrimoxazol (Trimthoprim/sulphamethoxazole), Dalbavancin, Dalfopristin/Quinopristin, Daptomycin, Dibekacin, Dicloxacillin, Doripenem, Doxycycline, Enrofloxacin, Ertapenem, Erythromycin, Flucloxacillin, Fluconazol, Flucytosin, Fosfomycin, Fusidic acid, Garenoxacin, Gatifloxacin, Gemifloxacin, Gentamicin, Imipenem, Itraconazole, Kanamycin, Ketoconazole, Levofloxacin, Lincomycin, Linezolid, Loracarbef, Mecillnam (amdinocillin), Meropenem, Metronidazole, Meziocillin, Mezlocillin-sulbactam, Minocycline, Moxifloxacin, Mupirocin, Nalidixic acid, Neomycin, Netilmicin, Nitrofurantoin, Norfloxacin, Ofloxacin, Oxacillin, Pefloxacin, Penicillin V, Piperacillin, Piperacillin-sulbactam, Piperacillin-tazobactam, Rifampicin, Roxythromycin, Sparfloxacin, Spectinomycin, Spiramycin, Streptomycin, Sulbactam, Sulfamethoxazole, Teicoplanin, Telavancin, Telithromycin, Temocillin, Tetracyklin, Ticarcillin, Ticarcillin-clavulanic acid, Tigecycline, Tobramycin, Trimethoprim, Trovafloxacin, Tylosin, Vancomycin, Virginiamycin, and Voriconazole.

Another additive, which may be contained in the inventive composition, may be an anti-viral agents, preferably, but are not limited to, nucleoside analogs (e.g., zidovudine, acyclovir, gangcyclovir, vidarabine, idoxuridine, trifluridine, and ribavirin), foscarnet, amantadine, peramivir, rimantadine, saquinavir, indinavir, ritonavir, alpha-interferons and other interferons, AZT, t-705, zanamivir (Relenza®), and oseltamivir (Tamiflu®). Other anti-viral agents include influenza virus vaccines, e.g., Fluarix® (Glaxo SmithKline), FluMist® (Medlmmune Vaccines), Fluvirin® (Chiron Corporation), Flulaval® (GlaxoSmithKline), Muria® (CSL Biotherapies Inc.), Agriflu® (Novartis) or Fluzone® (Aventis Pasteur).

The two components of the inventive composition, namely the first immunogenic component and the second adjuvant component providing at least one adjuvant component, may be administered as one formulation or the two or more components are provided as separate formulations which may be administered separately.

Moreover the invention relates to a pharmaceutical composition which comprises the composition as defined above in combination with a pharmaceutically acceptable carrier and/or vehicle. In this context a pharmaceutically acceptable carrier or vehicle is an agent which typically may be used within a pharmaceutical composition or vaccine for facilitating administering of the components of the pharmaceutical composition or vaccine to an individual. A pharmaceutically acceptable carrier or vehicle typically includes a liquid or non-liquid material, which is mixed with the first and/or second component of the inventive composition. If the components of the inventive composition are provided in liquid form, the carrier will typically be pyrogen-free water, isotonic saline or buffered aqueous solutions, e.g phosphate, citrate etc. buffered solutions. Ringer or Ringer-Lactate solution is particularly preferred as a liquid basis. At least one of the components of the inventive composition may be prepared for sustained and/or delayed release.

The inventive pharmaceutical composition may be administered in various ways, e.g. the pharmaceutical composition may be prepared for subcutaneous or intramuscular or intradermal or intratumoral injection. Moreover it may be advantageous to administer the two components of the inventive composition in different ways. In an especially preferred embodiment the pharmaceutical composition and especially the second adjuvant component of the composition is prepared for topical or transdermal administration, wherein preferably the pharmaceutical composition is prepared in the form of a transdermal patch and/or the composition comprises enhancers for transdermal delivery. Preferably, especially the adjuvant component, e.g. the vitamin A compound (e.g. ATRA) or the vitamin A derivative compound, is prepared for topical or transdermal administration. For example vitamin A compound or the vitamin A derivative compound-containing creams or lotions or gels may be used for administration. Moreover, vitamin A compound or the vitamin A derivative compound-loaded microneedle patches, which may be solid, hollow or dissolving, may be used for administration. Dermal or transdermal patches are particularly advantageous for slow release of the adjuvant component.

Generally the inventive pharmaceutical composition may be administered orally, parenterally, by inhalation spray, topically, rectally, nasally, buccally, vaginally or via an implanted reservoir. The term parenteral as used herein includes subcutaneous, intravenous, intramuscular, intraarticular, intranodal, intrasynovial, intrasternal, intrathecal, intrahepatic, intralesional, intracranial, transdermal, intradermal, intrapulmonal, intraperitoneal, intracardial, intraarterial, and sublingual injection or infusion techniques.

Preferably, the inventive pharmaceutical composition may be administered by parenteral injection, more preferably by subcutaneous, intravenous, intramuscular, intraarticular, intranodal, intrasynovial, intrasternal, intrathecal, intrahepatic, intralesional, intracranial, transdermal, intradermal, intrapulmonal, intraperitoneal, intracardial, intraarterial, and sublingual injection or via infusion techniques. Particularly preferred is intradermal and intramuscular injection. In one particularly preferred embodiment, the pharmaceutical composition is administered intramuscularly.

Methods for intramuscular administration are known in the art. Typically, a liquid is injected into a skeletal muscle (such as M. gluteus, M. deltoideus or M. vastus lateralis) using, for example, a syringe or a needle-free injection system, such as a jet injection system. Jet injection refers to a needle-free injection method, wherein a fluid comprising the inventive composition and, optionally, further suitable excipients is forced through an orifice, thus generating an ultra-fine liquid stream of high pressure that is capable of penetrating mammalian skin. In principle, the liquid stream forms a hole in the skin, through which the liquid stream is pushed into the target tissue.

Sterile injectable forms of the inventive pharmaceutical compositions may be aqueous or oleaginous suspension. These suspensions may be formulated according to techniques known in the art using suitable dispersing or wetting agents and suspending agents. The sterile injectable preparation may also be a sterile injectable solution or suspension in a non-toxic parenterally-acceptable diluent or solvent, for example as a solution in 1,3-butanediol. Among the acceptable vehicles and solvents that may be employed are water, Ringer's solution and isotonic sodium chloride solution. In addition, sterile, fixed oils are conventionally employed as a solvent or suspending medium. For this purpose, any bland fixed oil may be employed including synthetic mono- or di-glycerides. Fatty acids, such as oleic acid and its glyceride derivatives are useful in the preparation of injectables, as are natural pharmaceutically-acceptable oils, such as olive oil or castor oil, especially in their polyoxyethylated versions. These oil solutions or suspensions may also contain a long-chain alcohol diluent or dispersant, such as carboxymethyl cellulose or similar dispersing agents that are commonly used in the formulation of pharmaceutically acceptable dosage forms including emulsions and suspensions. Other commonly used surfactants, such as Tweens, Spans and other emulsifying agents or bioavailability enhancers which are commonly used in the manufacture of pharmaceutically acceptable solid, liquid, or other dosage forms may also be used for the purposes of formulation of the inventive pharmaceutical composition.

Moreover the inventive pharmaceutical composition as defined herein may also be administered orally in any orally acceptable dosage form including, but not limited to, capsules, tablets, aqueous suspensions or solutions. In the case of tablets for oral use, carriers commonly used include lactose and corn starch. Lubricating agents, such as magnesium stearate, are also typically added. For oral administration in a capsule form, useful diluents include lactose and dried cornstarch. When aqueous suspensions are required for oral use, the active ingredients are combined with emulsifying and suspending agents. If desired, certain sweetening, flavoring or coloring agents may also be added.

In a particularly preferred embodiment the inventive pharmaceutical composition and especially the adjuvant component is administered topically. For topical applications, the inventive pharmaceutical composition may be formulated in a suitable ointment, containing one or both components of the inventive composition suspended or dissolved in one or more carriers. Carriers for topical administration include, but are not limited to, mineral oil, liquid petrolatum, white petrolatum, propylene glycol, polyoxyethylene, polyoxypropylene compound, emulsifying wax and water. Alternatively, the inventive pharmaceutical composition can be formulated in a suitable lotion or cream. In the context of the present invention, suitable carriers include, but are not limited to, mineral oil, sorbitan monostearate, polysorbate 60, cetyl esters wax, cetearyl alcohol, 2-octyldodecanol, benzyl alcohol and water. In especially preferred embodiments of the inventive composition enhancers for transdermal or topical administration may be added to the composition, particularly substances which enhance skin permeability.

The inventive pharmaceutical composition typically comprises a “safe and effective amount” of the components of the inventive pharmaceutical composition. As used herein, a “safe and effective amount” means an amount of the components of the inventive composition as such that is sufficient to significantly induce a positive modification of a disease or disorder as defined herein. At the same time, however, a “safe and effective amount” is small enough to avoid serious side-effects and to permit a sensible relationship between advantage and risk. The determination of these limits typically lies within the scope of sensible medical judgment. A “safe and effective amount” of the components of the inventive pharmaceutical composition will furthermore vary in connection with the particular condition to be treated and also with the age and physical condition of the patient to be treated, the body weight, general health, sex, diet, time of administration, rate of excretion, drug combination, the severity of the condition, the duration of the treatment, the nature of the accompanying therapy, of the particular pharmaceutically acceptable carrier used, and similar factors, within the knowledge and experience of the accompanying doctor. The inventive pharmaceutical composition may be used for human and also for veterinary medical purposes, preferably for human medical purposes, as a pharmaceutical composition in general or preferably as a vaccine or immunostimulating agent.

However, one or more compatible solid or liquid fillers or diluents or encapsulating compounds, which are suitable for administration to a patient to be treated, may be used as well for the pharmaceutical composition according to the invention. The term “compatible” as used here means that these constituents of the inventive pharmaceutical composition are capable of being mixed with the components of the inventive pharmaceutical composition in such a manner that no interaction occurs which would substantially reduce the pharmaceutical effectiveness of the pharmaceutical composition under typical use conditions.

Furthermore the inventive pharmaceutical composition may comprise at least one additional pharmaceutically active component. A pharmaceutically active component in this connection is a compound that has a therapeutic effect to heal, ameliorate or prevent a particular indication or disease. Such compounds include, without implying any limitation, peptides or proteins, preferably as defined herein, nucleic acids, preferably as defined herein, (therapeutically active) low molecular weight organic or inorganic compounds (molecular weight less than 5000, preferably less than 1000), sugars, antigens or antibodies, preferably as defined herein, therapeutic agents already known in the prior art, antigenic cells, antigenic cellular fragments, cellular fractions, cell wall components (e.g. polysaccharides), modified, attenuated or de-activated (e.g. chemically or by irradiation) pathogens (virus, bacteria etc.), etc.

According to another particularly preferred aspect, the inventive composition may be provided or used as a vaccine. Typically, such a vaccine is as defined above for pharmaceutical compositions. In the specific context of the inventive vaccine, the choice of a pharmaceutically acceptable carrier is determined in principle by the manner in which the inventive vaccine is administered. Routes for local administration in general include, for example, topical administration routes but also intradermal, transdermal, subcutaneous, or intramuscular injections or intralesional, intracranial, intrapulmonal, intracardial, and sublingual injections, depending on the disease to be treated. Inventive vaccines are therefore preferably formulated in liquid (or sometimes in solid) form. Preferably, the inventive vaccine may be administered by conventional needle injection or needle-free jet injection. In a preferred embodiment the inventive vaccine may be administered by jet injection as defined herein, preferably intramuscularly or intradermally, more preferably intradermally. Particular approaches, methods and features of the administration of an mRNA comprising composition which may be incorporated as certain further embodiments of the present invention are disclosed in WO2015/024667, the description of which is incorporated herein by reference.

In a preferred embodiment the inventive vaccine may be administered by topical or transdermal routes.

Moreover the invention relates to a kit, preferably a kit of parts, comprising the composition as defined above, or the pharmaceutical composition as defined above, or the vaccine as defined above, and optionally a liquid vehicle for solubilising and optionally technical instructions with information on the administration and dosage of the composition or the pharmaceutical composition or the vaccine. Preferably the nucleic acid molecule component of the composition encoding at least one epitope of at least one antigen is provided in lyophilized form as a separate part. Preferably the kit contains as a part Ringer-Lactate solution.

Moreover the invention relates to a use of the composition as defined above, or the pharmaceutical composition as defined above, or the vaccine as defined above, or the kit as defined above as a medicament.

Moreover the invention relates to a use of the composition as defined above, or the pharmaceutical composition as defined above, or the vaccine as defined above, or the kit as defined above in the treatment or prophylaxis of an infectious disease or an allergy or an autoimmune disease or a cancer or tumor disease.

Moreover the invention relates to a use of the composition as defined above, or the pharmaceutical composition as defined above, or the vaccine as defined above, or the kit as defined above for preparation of a medicament for treatment or prophylaxis of an infectious disease or an allergy or an autoimmune disease or a cancer or tumor disease.

Moreover the invention relates to a nucleic acid molecule encoding at least one epitope of at least one antigen as defined above for use in treatment or prophylaxis of an infectious disease or an allergy or an autoimmune disease or a cancer or tumor disease in combination with an adjuvant component as defined above.

Moreover the invention relates to an adjuvant component as defined above for use in treatment or prophylaxis of an infectious disease or an allergy or an autoimmune disease or a cancer or tumor disease in combination with an immunogenic component as defined above.

Moreover the invention relates to a method of treatment or prophylaxis comprising administering to a subject in need thereof a therapeutically effective amount of an immunogenic component comprising at least one nucleic acid molecule as defined above in combination with an adjuvant component as defined above. The administration of the immunogenic component and of the adjuvant component may be varied in various ways. The administration of the immunogenic component and the adjuvant component may occur either simultaneously or timely staggered, either at the same site of administration or at different sites of administration. The immunogenic component and the adjuvant component may be administered at the same time in one formulation or may be administered at (about) the same time in different formulations, at the same site or at different sites, by the same application route or by different application routes. In another embodiment the immunogenic component and the adjuvant component may be administered separated in time (in a time-staggered manner), i.e. sequentially, and/or are administered at different administration sites. This means that the immunogenic component may be administrated e.g. prior, concurrent or subsequent to the adjuvant component, or vice versa. Alternatively or additionally, the immunogenic component and the adjuvant component may be administered at different administration sites, or at the same administration site, preferably, when administered in a time staggered manner. According to a particularly preferred embodiment, the immunogenic component is to be administered first and the adjuvant component is to be administered subsequent to the RNA respectively the immunogenic component. This procedure ensures that the immune cells such as antigen-presenting cells and T cells have already encountered the antigen before the immune system is stimulated by the adjuvant component, even though a concurrent administration or an administration, wherein the adjuvant component is to be administered prior to the immunogenic component, may lead to the same or at least comparable results. It may be particularly preferred to administer the immunogenic component and the adjuvant component at the same time and the same administration site.

In summary, it is possible to administer the immunogenic component and the adjuvant component at the same site of administration. Moreover it is possible to administer the immunogenic component and the adjuvant component at the same time. Moreover it is possible to provide the immunogenic component and the adjuvant component in one formulation or in separate formulations. It may be advantageous to administer the immunogenic component and the adjuvant component timely staggered, wherein, for example, the immunogenic component is to be administered first and the adjuvant component is to be administered second. It is especially preferred to administer the immunogenic component and the adjuvant component at different sites of administration, preferably by different application routes, wherein generally it is possible to administer the immunogenic component and/or the adjuvant component for example by subcutaneous or intramuscular or intradermal or intratumoral injection, preferably by intramuscular injection. An injection may be carried out by using conventional needle injection or jet injection, preferably by using jet injection.

It is particularly preferred to administer the immunogenic component and/or the adjuvant component by topical or transdermal administration, for example by iontophoresis or by non-cavitational ultrasound or by cavitational ultrasound or by electroporation of by microneedles or by thermal ablation or by microdermabrasion. In this context it is referred to the article of Prausnitz M. R. and Langer R. (Prausnitz M. R. and Langer R. (2008), Nat Biotechnol November 26(11): 1261-1268) generally describing methods for transdermal drug delivery, which may be used for the inventive composition. Advantageously patches with microneedles may be used, especially for slow release. Moreover, in especially preferred embodiments, creams, lotions or gels containing the immunogenic component and/or the adjuvant component may be used.

The above described method of treatment or prophylaxis is preferably provided for treatment or prophylaxis of an infectious disease or an allergy or an autoimmune disease or a cancer or tumor disease.

Preferred embodiments of the present invention are furthermore characterized by the following items:
1. A composition comprising at least a first immunogenic component and at least a second adjuvant component,
   wherein the first immunogenic component comprises at least one nucleic acid molecule encoding at least one epitope of at least one antigen, and
   wherein the second adjuvant component comprises at least one immune potentiator compound and/or at least one delivery system compound.
2. The composition of item 1, wherein the second adjuvant component comprises at least one vitamin compound.
3. The composition of item 2, wherein the vitamin compound is a vitamin A compound and/or vitamin A derivative compound, preferably a retinoid compound.
4. The composition of item 3, wherein the vitamin compound is selected from the list consisting of: retinoic acid, preferably all-trans retinoic acid (ATRA), retinol palmitate, retinol ester, retinol, retinal, tretinoin, Retin-A, isotretinoin, alitretinoin, etretinate, acitretin, tazarotene, bexarotene and Adapalene.
5. The composition of item 2, wherein the vitamin compound is a vitamin E compound and/or a vitamin C compound and/or a vitamin D compound, preferably selected from the list consisting of: tocopherol, mixture of Squalene plus Tween 80 plus α-tocopherol, vitamin D3, and 25-dihydroxycholecalciferol.
6. The composition of one of items 2 to 5, wherein the second adjuvant component provides a further adjuvant component.
7. The composition of item 6 wherein the further adjuvant component comprises a polymeric carrier cargo complex comprising as a carrier a complex of at least one cationic and/or oligocationic and/or polycationic component and as a cargo at least one nucleic acid molecule.
8. The composition of item 7, wherein the cationic and/or oligocationic and/or polycationic component comprises at least one disulfide-crosslinked cationic component.
9. The composition of item 7 or item 8, wherein the cationic and/or oligocationic and/or polycationic component comprises cationic peptides, wherein the cationic peptides are selected from peptides according to formula (I)

(Arg)ₗ;(Lys)ₘ;(His)ₙ;(Orn)ₒ;(Xaa)ₓ,
wherein
   l+m+n+o+x=3-100, and
   l, m, n or o=independently of each other is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, 81-90 and 91-100, provided that the overall content of Arg, Lys, His and Orn represents at least 10% of all amino acids of the cationic peptide; and Xaa is any amino acid selected from native (=naturally occurring) or non-native amino acids except of Arg, Lys, His or Orn; and
   x=any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, 81-90, provided, that the overall content of Xaa does not exceed 90% of all amino acids of the cationic peptide,
   or are selected from peptides according to subformula (Ia)

{(Arg)ₗ;(Lys)ₘ;(His)ₙ;(Orn)ₒ;(Xaa′)ₓ(Cys)_{y}}
or from peptides according to subformula (Ib)

Cys₁{(Arg)ₗ;(Lys)ₘ;(His)ₙ;(Orn)ₒ;(Xaa)ₓ}Cys₂
wherein (Arg)ₗ; (Lys)ₘ; (His)ₙ; (Orn)ₒ; and x are as defined above; Xaa′ is any amino acid selected from native (=naturally occurring) or non-native amino acids except of Arg, Lys, His, Orn; or Cys and y is any number selected from 0, 1, 2, 3, 4, 5, 6, 7, 8, 9, 10, 11, 12, 13, 14, 15, 16, 17, 18, 19, 20, 21-30, 31-40, 41-50, 51-60, 61-70, 71-80, and 81-90, provided that the overall content of Arg (Arginine), Lys (Lysine), His (Histidine) and Orn (Ornithine) represents at least 10% of all amino acids of the oligopeptide and wherein Cys₁ and Cys₂ are Cysteines proximal to, or terminal to (Arg)ₗ;(Lys)ₘ;(His)ₙ;(Orn)ₒ;(Xaa)ₓ
10. The composition of item 8 or item 9, wherein the disulfide-bonds are formed by cysteine residues contained in the cationic peptides.
11. The composition according to item 10, wherein the cysteine residue is located proximal to the terminal ends of the cationic peptides, preferably at the terminal ends of the cationic peptides.
12. The composition of one of items 7 to 11, wherein the cationic and/or oligocationic and/or polycationic component comprises an arginine-rich peptide, preferably the peptide CysArg₁₂Cys according to SEQ ID NO. 7 and/or the peptide CysArg₁₂ according to SEQ ID NO. 8.
13. The composition of one of items 7 to 12, wherein the nucleic acid molecule is an RNA molecule, preferably a guanosine-rich and uracil-rich RNA molecule.
14. The composition of one of items 7 to 13, wherein the cargo nucleic acid molecule is an immunostimulatory nucleic acid molecule, preferably an immunostimulatory RNA molecule (isRNA), more preferably a non-coding immunostimulatory nucleic acid molecule, more preferably a nucleic acid molecule according to SEQ ID NO. 2.
15. The composition of one of items from 7 to 14, wherein the cationic and/or oligocationic and/or polycationic component of the polymeric carrier and the cargo nucleic acid molecule comprised in said polymeric carrier cargo complex are provided in a N/P ratio in the range of 0.1-20, or in the range of 0.1-5, or in the range of 0.1-1, or in the range of 0.5-0.9.
16. The composition of one of the preceding items, wherein the second adjuvant component comprises at least one emulsion or surfactant-based compound, preferably an oil-in-water compound, more preferably a squalene-based compound, and/or a water-in-oil compound, more preferably a mineral oil-based compound or a squalene-based compound, and/or a block copolymer surfactant compound and/or a tenside-based compound.
17. The composition of item 16, wherein the at least one emulsion or surfactant-based compound is selected from the list consisting of: non-ionic surfactant vesicles (NISV), VSA-3 adjuvant, SAF, SAF-1 (threonyl-MDP in an emulsion vehicle), nano-emulsification of 2 components comprising Sorbitan trioleate (0.5% w/v) in squalene oil (5% v/v) and Tween 80 (0.5% w/v) in sodium citrate buffer (10 mM, pH 6.5), mixture of Squalene plus Tween 80 plus Span 85, AS02 (Squalene plus Tween 80 plus Span 85 plus MPL (monophosphoryl lipid A) plus QS-21), AS03 (Squalene plus Tween 80 plus α-tocopherol), AF03 (Squalene plus Montane 80 (emulsifier) plus Eumulgin B1 PH (emulsifier)), nanoemulsion, RIBI (bacterial and mycobacterial cell wall components), Ribi529, Ribilike adjuvant system (MPL, TMD, CWS), Murametide (N2-[N-(N-Acetylmuramoyl)-L-alanyl]-D-glutamine methyl ester), incomplete Freund's adjuvant (IFA), complete Freund's adjuvant (CFA), Specol (Marco) 52 (mineral oil, paraffins, and cycloparaffins, chain length 13-22 C atoms) plus Span 85 plus Tween 85), squalene, squalene plus squalane, SPT (squalane (5%), Tween 80 (0.2%), Pluronic L121 (1.25%)), Squalane 1 (2,6,10,15,19,23-hexamethyltetracosane, 2,6,10,15,19,23-hexamethyl-2,6,10,14,18,22-tetracosahexane), Squalene 2 (Spinacene; Supraene; 2,6,10,15,19, 23-hexamethyl-2,6,10,14,18,22 tetracosahexane), TiterMax Gold Adjuvant, pluronics, Pluronic L121 (Poloxamer 401) and Polysorbate 80 (Tween 80).
18. The composition of one of the preceding items, wherein the second adjuvant component comprises at least one nucleotide-based or nucleoside-based compound, preferably a cyclic dinucleotide compound, more preferably a cyclic guanosine monophosphate-adenosine monophosphate compound or a cyclic diadenylate monophosphate compound or a cyclic diguanylate monophosphate compound, and/or a cytosine-phosphoguanosine (CpG) dinucleotide motif compound, more preferably an oligodeoxynucleotide containing unmethylated CpG motifs compound or an oligonucleotide containing unmethylated CpG motifs compound, and/or a double-stranded nucleic acid compound, more preferably a double-stranded RNA (dsRNA) compound or a double-stranded DNA (dsDNA) compound, and/or a single-stranded nucleic acid compound, more preferably a single-stranded RNA (ssRNA), and/or a guanosine analogue compound.
19. The composition of one of the preceding items, wherein the second adjuvant component comprises at least one protein-based or peptide-based compound, preferably a metalloprotein compound and/or a heat shock protein compound and/or a membrane protein compound and/or a peptidoglycan compound, more preferably a muropeptide or derivative thereof, and/or a bacterial protein-based compound and/or a high mobility group protein compound and/or a lipopeptide compound and/or a lipoprotein compound.
20. The composition of one of the preceding items, wherein the second adjuvant component comprises at least one hydrocarbon-based or carbohydrate-based compound, preferably a polysaccharide-based compound and/or a polyaminosaccharide-based compound, more preferably a chitin-derived compound, and/or a glycoside-based compound, more preferably a saponin or derivative thereof, and/or an imidazoquinoline compound and/or a glycolide compound and/or an amide-based compound.
21. The composition of one of the preceding items, wherein the second adjuvant component comprises at least one lipid-based compound, preferably a glycolipid compound, more preferably a trehalose dimycolate or derivative thereof, and/or a lipopolysaccharide compound and/or a lipopolysaccharide derivative compound, more preferably bacterial lipopolysaccharide (LPS) or a lipid A compound, and/or a lipoidal amine compound.
22. The composition of one of the preceding items, wherein the second adjuvant component comprises at least one polymeric compound, preferably an anorganic-organic polymer compound and/or a polyacrylic compound.
23. The composition of one of the preceding items, wherein the second adjuvant component comprises at least one cytokine or at least one hormone compound, preferably a chemokine compound and/or an interferon compound and/or tumor necrosis factor (TNF) compound and/or an adhesion molecule compound and/or a steroid compound, or at least one enzyme compound or at least one cell compound.
24. The composition of one of the preceding items, wherein the second adjuvant component comprises at least one toxin compound, preferably a viral toxin compound and/or a viral toxin derivative compound and/or a bacterial toxin compound and/or a bacterial toxin derivative compound.
25. The composition of one of the preceding items, wherein the second adjuvant component comprises at least one vehicle compound, preferably a liposome compound and/or a virosome compound and/or a virus-like particle compound and/or a microparticle compound and/or a nanoparticle compound and/or a protein cochleate compound.
26. The composition of one of the preceding items, wherein the second adjuvant component comprises at least one mineral salts compound, preferably an aluminium compound and/or a calcium compound.
27. The composition of one of the preceding items, wherein the second adjuvant component comprises at least one compound selected from the list consisting of: 3′3′-cGAMP, 2′2′-cGAMP, 1018 ISS, CpG 7909, CpG 1018, AS15 (MPL plus CpG plus QS-21 plus liposome), synthetic dsRNA, especially polyionisinic:polycytidylic acid (Poly(I:C)), Hiltonol (polylCLC—poly-IC with poly-lysine), poly-adenylic acid-poly-uridylic acid complex (Poly rA:Poly rU), 5′ppp-dsRNA, viral dsRNA, IC31 (KLKL(5)KLK peptide vehicle plus ODN1a), pCMVmCAT1 (plasmid expressing Friend murine leukemia virus receptor), guanosine-rich ssRNA, uridine-rich ssRNA, polymeric carrier cargo complex formed by peptide CR₁₂C and isRNA or peptide CR₁₂ and isRNA, Loxoribine (7-allyl-8-oxoguanosine), CCR5 peptides, pRANTES (CCLS), Trp-Lys-Tyr-Met-Val-Met immunostimulatory peptide, albumin-heparin microparticles, β-glucan peptide (BGP), proteinoid microspheres, stable protein phospholipid-calcium precipitates, pCMVmCAT1 (plasmid expressing Friend murine leukemia virus receptor), PAMPs (Pathogen-associated molecular patterns), protamine, antimicrobial peptides, RSV fusion protein, CGRP neuropeptide, Keyhole limpet hemocyanin (KLH), HSP70, Gp96, B7-2, muramyl dipeptide (MDP), Murapalmitine (Nac-Mur-L-Thr-D-isoGl n-sn-glycerol dipalmitoyl), Threonyl muramyl dipeptide (TMDP, [thr1]-MDP, N-acetyl muramyl-L-threonyl-D-isoglutamine), muramyl tripeptide, muramyl tripeptide phosphatidylethanolamine (MTP-PE, (N-acetyl-L-alanyl-D-isoglutaminyl-L-alanine-2-(1,2-dipalmitoyl-sn-glycero-3-(hydroxyphosphoryloxy))-ethylamide, monosodium salt), muramyl tetrapeptide, especially M-TriLYS-D-ASN, romurtide (synthetic muramyl dipeptide derivative), adamantylamide dipeptide, adamantylamide l-alanyl-d-isoglutamine, SAF (Syntex adjuvant formulation), SAF-1 (threonyl-MDP in an emulsion vehicle), flagellin and flagellin fusion proteins, HMGB1, P3C, Pam3Cys (tripalmitoyl-S-glyceryl cysteine), GMDP (N-acetylglucosaminyl-(β1-4)-N-acetylmuramyl-L-alanyl-D-isoglutamine), p-Hydroxybenzoique acid methyl ester, BAK (benzalkonium chloride), Mannose, LNFPIII/Lewis X, β-glucan, glucans from algae, dextran, inulin, γ-inulin, delta inulin polysaccharide, Algammulin, chitosan, Quil-A, QS-21, AS01 (MPL plus liposome plus QS-21), AS02 (Squalene plus Tween 80 plus Span 85 plus MPL (monophosphoryl lipid A) plus QS-21), immuno-stimulatory complexes (ISCOMs), cholesterol plus phospholipid plus saponin, Abisco-100, Iscoprep 7.0.3.®, Quadri A saponin, GPI0100, GPI anchor, Matrix M, POSintro, R-837 (Imiquimod), R-848 (Resiquimod), 3M-012, S-28463 (4-amino-2-ethoxymethyl-alpha, alpha-dimethyl-1H-imidazo[4,5-c]quinoline-1-ethanol), DL-PGL (polyester poly (DL-lactide-co-glycolide)), PLG (polyactide coglycolide), PLGA plus PGA plus PLA (homo- and co-polymers of lactic and glycolic acid), Bupivacaine ((RS)-1-Butyl-N-(2,6-dimethylphenyl)piperidine-2-carboxamide), Arlacel A (dianhydromannitol monooleate), Span 85 (Arlacel 85, sorbitan trioleate), DMPC (Dimyristoyl phosphatidy-1-choline), DMPG (Dimyristoyl phosphatidylglycerol), N-acetylglucosaminyl-N-acetyhnuramyl-L-Ala-D-isoGlu-L-Ala-glycerol dipalmitate (DTP-GDP, disaccharide tripeptide glycerol dipalmitoyl), N-acetylglucosaminyl-N-acetylinuramyl-L-Ala-D-isoGlu-L-Ala-dipalmitoxy propylamide (DTP-DPP), stearyl tyrosine, DDA (dimethy-1-dioctadecylammonium bromide or chloride), Gerbu Adjuvant (mixture of: i) N-Acetylglucosaminyl-(PI-4)-N-acetylmuramyl-L-alanyl-D-glutamine (GMDP), ii) Dimethyl dioctadecylammonium chloride (DDA), iii) Zinc L-proline saltcomplex (ZnPro-8)), trehalose-6,6′-dimycolate (TDM), trehalose-6,6′-dibehenate (TDB), BAY R1005 (N-(2-Deoxy-2-L-leucylamino-β-D-glucopyranosyl)-N-octadecyldodecanoyl-amide hydroacetate), monophosphoryl lipid A (MPL), MPL-SE (MPL stable emulsion), AS04 (MPL puls Alum), DETOX (MPL plus mycobacterial cell-wall skeleton), glucopyranosil lipid A (GLA), RC529 (2-[(R)-3-tetradecanoyloxytetradecanoylamino]ethyl 2-deoxy-4-O-phosphono-3-O-[(R)-3-tetradecanoyoxytetradecanoyl]-2-[(R)-3-tetradecanoyoxytetradecanoylamino]-13-D-glucopyranos idetriethylammonium salt), N,N-dioctadecyl-N′,N′-bis(2-hydroxyethyl) propanediamine, POLYGEN® Vaccine Adjuvant, copolymers like Optivax (CRL1005), L121 or Poloaxmer4010, biopolymers, polyethylene carbamate derivatives, polyphosphazene, polymethylmethacrylate (PMMA), Carbopol 934P, retinoic acid, esp. all-trans retinoic acid (ATRA), retinyl palmitate, retinol ester, retinol, retinal, tretinoin, Retin-A, isotretinoin, alitretinoin, etretinate, acitretin, tazarotene, bexarotene, Adapalene (polyaromatisches retinoid), tocopherol, AS03 (Squalene plus Tween 80 plus α-tocopherol), vitamin D3, Calcitrol (25-dihydroxycholecalciferol), IL-1, IL-1β, IL-2, IL-4, IL-6, IL-7, IL-10, IL-12, IL-15, IL-18, Sciavo peptide (IL-1β163-171 peptide), IL-2 in pcDNA3, IL-2/Ig plasmid, IL-4 in pcDNA3, IL-10 plasmid, hIL-12 (N222L), IL-12 DNA, IL-12 plasmid, IL-12/GM-CSF plasmid, rAd5-hIL-12N222L, IL-15 plasmid, rAd5-IL15, GM-CSF, Flt-3 ligand, ligands of human TLR1-10, ligands of murine TLR1-13, UC-1V150, Ampligen™, lymphotactin, RANTES, defensins, IFN-α, IFN-γ, IFN-γ in pCDNA3, recombinant hIFN-γ, TNFα, CD40 ligand, ICAM-1, LAF-3, Dehydroepiandrosterone (DHEA), Neuraminidase-galactose oxidase (NAGO), dendritic cells, PBMC (peripheral blood mononuclear cells), cholera toxin (CT), cholera holotoxin, mCT-E112K, cholera toxin B subunit (CTB), cholera toxin A1-subunit-ProteinA D-fragment fusion protein, CTA1-DD gene fusion protein, chimeric A1 subunit of cholera toxin (CTA1)-DD, E. coli heat-labile enterotoxin (LT), LT(R192G), LTK63, LTK72, LT-R192G, LT B subunit, LT-OA (E. coli labile enterotoxin protoxin), LT 5 oral adjuvant (E. coli labile enterotoxin-protoxin), Bordetella pertussis component Vaccine Adjuvant, Corynebacterium-derived P40, killed Corynebacterium parvum vaccine adjuvant, Diphtheria toxoid, Tetanus toxoid (TT), microbe derived adjuvants, plant derived adjuvants, Tomatine adjuvant, cationic liposomal vaccine adjuvant, Stealth liposomes, JVRS-100 (cationic liposomal DNA complex), cytokine-containing liposomes, immunoliposomes containing antibodies to costimulatory molecules, DRVs (immunoliposomes prepared from dehydration-rehydration vesicles), MTP-PE liposomes, Sendai proteoliposomes, Sendai containing lipid matrices, Walter Reed liposomes (liposomes containing lipid A adsorbed to aluminium hydroxid), CAF01 (liposomes plus DDA plus TDB), liposomes (lipids plus hemagglutinin), IRIVs (immunopotentiating reconstituted influenza virosomes), virosomes (unilamellar liposomal vehicles incorporating viral protein, such as influenza haemagglutinin), Ty particles (Ty-VLPs), polymeric microparticles (PLG), cationic microparticles, CRL1005 (block copolymer P1205), peptomere nanoparticle, calcium phosphate nanoparticles, microspheres, nanospheres, stable protein phospholipid-calcium precipitates (cochleates), non-ionic surfactant vesicles (NISV), VSA-3 adjuvant, nano-emulsification of 2 components comprising Sorbitan trioleate (0.5% w/v) in squalene oil (5% v/v) and Tween 80 (0.5% w/v), Squalene plus Tween 80 plus Span 85, AF03 (Squalene plus Montane 80 (emulsifier) plus Eumulgin B1 PH (emulsifier)), nanoemulsion, RIBI (bacterial and mycobacterial cell wall components), Ribi529, Ribilike adjuvant system (MPL, TMD, CWS), Murametide (N2-[N-(N-Acetylmuramoyl)-L-alanyl]-D-glutamine methyl ester), incomplete Freund's adjuvant (IFA), complete Freund's adjuvant (CFA), Specol (Marcol 52 (mineral oil, paraffins, and cycloparaffins, chain length 13-22 C atoms) and Span 85 and Tween 85), squalene plus squalane ISA51, squalene plus squalane ISA720, SPT (squalane (5%), Tween 80 (0.2%), Pluronic L121 (1.25%)), Squalane 1 (Spinacane; 2,6,10,15,19,23-hexamethyltetracosane, 2,6,10,15,19,23-hexamethyl-2,6,10,14,18,22-tetracosahexane), Squalene 2 (Spinacene; Supraene; 2,6,10,15,19,23-hexamethyl-2,6,10,14,18,22 tetracosahexane), TiterMax Gold Adjuvant, pluronics, Pluronic L121 (Poloxamer 401), Polysorbate 80 (Tween 80), aluminium hydroxide, aluminium phosphate, Alum (aluminium hydroxide gel, aluminium hydroxide gel suspension), high protein adsorbency aluminium hydroxide gel (HPA), low viscosity aluminium hydroxide gel (LV), DOC (deoxycholic acid sodium salt)/Alum complex, aluminium phosphate gel, aluminium potassium sulfate, aluminium salts like Adju-phos or Alhydrogel or Rehydragel, amorphous aluminium hydroxyphosphate sulfate, calcium phosphate gel, AF, Provax, and PMM.
28. The composition of one of the preceding items, wherein the at least one nucleic acid molecule of the immunogenic component is an RNA molecule, preferably an mRNA molecule.
29. The composition of item 28, wherein the G/C content of the coding region of the mRNA molecule is increased compared with the G/C content of the coding region of the wild type mRNA, and wherein the coded amino acid sequence of said GC-enriched mRNA is preferably not being modified compared with the coded amino acid sequence of the wild type mRNA.
30. The composition of item 28 or item 29, wherein the mRNA molecule comprises additionally a 5′-UTR element and/or a 3′-UTR element and/or additionally at least one histone stem-loop and/or additionally a 5′-CAP structure and/or a poly(A) sequence and/or a poly(C) sequence.
31. The composition of one of items 28 to 30, wherein the mRNA molecule is naked and/or complexed with a cationic component, preferably with protamine.
32. The composition of one of the preceding items, wherein said at least one antigen is selected from the group consisting of an antigen from a pathogen associated with infectious diseases, an antigen associated with allergies, an antigen associated with autoimmune diseases, and an antigen associated with cancer or tumor diseases, or a fragment, variant and/or derivative of said antigen.
33. The composition of one of the preceding items, wherein said at least one antigen is derived from a pathogen, preferably a viral, bacterial, fungal or protozoan pathogen, preferably selected from the list consisting of: Rabies virus, Ebolavirus, Marburgvirus, Hepatitis B virus, human Papilloma virus (hPV), Bacillus anthracis, Respiratory syncytial virus (RSV), Herpes simplex virus (HSV), Dengue virus, Rotavirus, Influenza virus, human immunodeficiency virus (HIV), Yellow Fever virus, Mycobacterium tuberculosis, Plasmodium, Staphylococcus aureus, Chlamydia trachomatis, Cytomegalovirus (CMV) and Hepatitis B virus (HBV).
34. The composition of one of the preceding items, wherein said at least one antigen is a tumor antigen, preferably a melanocyte-specific antigen, a cancer-testis antigen or a tumor-specific antigen, preferably a CT-X antigen, a non-X CT-antigen, a binding partner for a CT-X antigen or a binding partner for a non-X CT-antigen or a fragment, variant or derivative of said tumor antigen.
35. The composition of one of the preceding items, wherein said at least one antigen is associated with allergy or allergic disease and preferably is derived from a source selected from the list consisting of: grass pollen, tree pollen, flower pollen, herb pollen, dust mite, mold, animals, food, and insect venom.
36. The composition of item 33, wherein the immunogenic component comprises at least one nucleic acid molecule encoding at least one epitope of at least one antigen of Influenza virus, preferably Influenza A virus, wherein the antigen is preferably the Hemagglutinin (HA), preferably according to SEQ ID NO. 1.
37. The composition of item 36, wherein the second adjuvant component comprises at least two adjuvant components, preferably a vitamin A compound or a vitamin A derivative compound, more preferably all-trans retinoic acid (ATRA) or retinyl palmitate, and a polymeric carrier cargo complex according to one of items 7 to 15.
38. The composition of item 33, wherein the immunogenic component comprises at least one nucleic acid molecule encoding at least one epitope of at least one antigen of Rabies virus, wherein the antigen is preferably the glycoprotein G (RAV-G), preferably according to SEQ ID NO. 3.
39. The composition of item 38, wherein the nucleic acid molecule is at least partly complexed with protamine and wherein preferably the adjuvant component comprises an emulsion, preferably an oil-in-water emulsion, more preferably a squalene-based compound, most preferably a mixture of Squalene plus Tween 80 plus Span 85.
40. The composition of one of the preceding items, wherein the immunogenic component and the adjuvant component are provided as separate formulations or are provided as one formulation.
41. A pharmaceutical composition comprising the composition as defined according to any one of items 1 to 40 and a pharmaceutically acceptable carrier and/or vehicle.
42. The pharmaceutical composition of item 41, wherein the pharmaceutical composition is prepared for subcutaneous or intramuscular or intradermal or intratumoral injection.
43. The pharmaceutical composition of item 42, wherein the pharmaceutical composition is prepared for topical or transdermal administration, wherein preferably the pharmaceutical composition is prepared in the form of a transdermal patch and/or the composition comprises enhancers for transdermal delivery.
44. A vaccine, comprising the composition as defined according to any one of items 1 to 40 or the pharmaceutical composition as defined according to any one of items 41 to 43.
45. A kit, preferably a kit of parts, comprising the composition according to any one of items 1 to 40, or the pharmaceutical composition according to any one of items 41 to 43, or the vaccine according to item 44, and optionally a liquid vehicle for solubilising and optionally technical instructions with information on the administration and dosage of the composition or the pharmaceutical composition or the vaccine.
46. The composition according to any one of items 1 to 40, or the pharmaceutical composition according to any one of items 41 to 43, or the vaccine according to item 44, or the kit according to item 45 for use as a medicament.
47. The composition according to any one of items 1 to 40, or the pharmaceutical composition according to any one of items 41 to 43, or the vaccine according to item 44, or the kit according to item 45 for use in the treatment or prophylaxis of an infectious disease or an allergy or an autoimmune disease or a cancer or tumor disease.
48. Use of the composition as defined according to any one of items 1 to 40, or the pharmaceutical composition according to any one of items 41 to 43, or the vaccine according to item 44, or the kit according to item 45 for preparation of a medicament for treatment or prophylaxis of an infectious disease or an allergy or an autoimmune disease or a cancer or tumor disease.
49. A nucleic acid molecule encoding at least one epitope of at least one antigen as defined in any one of items 1 to 40 for use in treatment or prophylaxis of an infectious disease or an allergy or an autoimmune disease or a cancer or tumor disease in combination with an adjuvant component as defined in any one of items 1 to 40.
50. An adjuvant component as defined in any one of items 1 to 40 for use in treatment or prophylaxis of an infectious disease or an allergy or an autoimmune disease or a cancer or tumor disease in combination with an immunogenic component as defined in any one of items 1 to 40.
51. A method of treatment or prophylaxis comprising administering to a subject in need thereof a therapeutically effective amount of an immunogenic component comprising at least one nucleic acid molecule as defined in any one of items 1 to 40 in combination with an adjuvant component as defined in any one of items 1 to 40.
52. The method of treatment or prophylaxis of item 51, wherein the immunogenic component and the adjuvant component are to be administered at different sites of administration, preferably by different application routes.
53. The method of treatment or prophylaxis of item 51 or item 52, wherein the immunogenic component and/or the adjuvant component are administered by subcutaneous or intramuscular or intradermal or intratumoral injection.
54. The method of treatment or prophylaxis of one of items 51 to 53, wherein the immunogenic component and/or the adjuvant component are administered by topical or transdermal administration, preferably by iontophoresis or by non-cavitational ultrasound or by cavitational ultrasound or by electroporation of by microneedles or by thermal ablation or by microdermabrasion.
55. The method of treatment or prophylaxis of one of items 51 to 54, wherein the method is provided for treatment or prophylaxis of an infectious disease or an allergy or an autoimmune disease or a cancer or tumor disease.

In the present invention, if not otherwise indicated, different features of alternatives and embodiments may be combined with each other, where suitable. Furthermore, the term “comprising” shall not be narrowly construed as being limited to “consisting of” only, if not specifically mentioned. Rather, in the context of the present invention, “consisting of” is an embodiment specifically contemplated by the inventors to fall under the scope of “comprising”, wherever “comprising” is used herein.

All publications, patents and patent applications cited in this specification are herein incorporated by reference as if each individual publication or patent application were specifically and individually indicated to be incorporated by reference.

Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

The examples and figures shown in the following are merely illustrative and shall describe the present invention in a further way. These figures and examples shall not be construed to limit the present invention thereto.

### BRIEF DESCRIPTION OF FIGURES

FIG. 1: G/C-enriched mRNA sequence R2564 coding for the hemagglutinin (HA) protein of influenza A virus (A/Netherlands/602/2009 (H1N1)), corresponding to SEQ ID NO: 1.

FIG. 2: RNA sequence of the non-coding immunostimulatory GU-rich RNA R2025, corresponding to SEQ ID NO: 2.

FIG. 3: shows HA-specific IgG1 titers in sera of immunized mice at day 14 after booster vaccination. The experiment was performed as described in Example 2 and HA-specific antibody IgG1 titers were determined by ELISA. The intramuscular vaccination with a combination of the HA-mRNA (R2564) and M-TriLYS-D-ASN, Beta-glucan peptide, HMGB1 or AddaVAx as an adjuvant induces higher antibody titers against the HA protein compared to vaccination with the HA-mRNA (R2564) alone.

FIG. 4: shows HA-specific IgG2a titers in sera of immunized mice at day 14 after booster vaccination. The experiment was performed as described in Example 2 and HA-specific antibody IgG2a titers were determined by ELISA. The intramuscular vaccination with a combination of the HA-mRNA (R2564) and M-TriLYS-D-ASN, Beta-glucan peptide, HMGB1 or AddaVAx as an adjuvant induces higher antibody titers against the HA protein compared to vaccination with the HA-mRNA (R2564) alone.

FIG. 5: shows that the intramuscular vaccination with a combination of the HA-mRNA (R2564) and M-TriLYS-D-ASN, Beta-glucan peptide, HMGB1 or AddaVax as an adjuvant induces higher neutralizing antibody titers against the HA protein at day 14 after booster vaccination compared to vaccination with the HA-mRNA (R2564) alone.
Balb/c mice (n=6 or 8 per group) were vaccinated intramuscularly on days 0 and 21 with 40 μg HA-mRNA (R2564) either alone or in combination with M-TriLYS-D-ASN, Beta-glucan peptide, HMGB1 or AddaVAx as an adjuvant. Buffer treated mice served as negative controls. Induction of functional humoral responses was analyzed on day 35 by determining the hemagglutination inhibition (HI) antibody titer, which is generally used as a surrogate marker of protection and a HI titer of 1:40 or greater is considered to confer protection. The experiment was performed as described in Example 2.
As can be seen, all mice vaccinated together with an adjuvant developed HI-titers≥1:40. In contrast, only 5 out of 8 mice vaccinated with HA-mRNA alone showed HI-titers ≥1:40. Each dot represents an individual animal and the horizontal lines represent median values.

FIG. 6: shows that the intramuscular vaccination with a combination of the HA-mRNA (R2564) and M-TriLYS-D-ASN, Beta-glucan peptide, HMGB1 or AddaVax as an adjuvant leads to significant increase in the number of multifunctional CD8+ T cells at day 42 after booster vaccination.
Balb/c mice (n=6 or 8 per group) were vaccinated intramuscularly on days 0 and 21 with 40 μg HA-mRNA either alone or in combination with M-TriLYS-D-ASN, Beta-glucan peptide, HMGB1 or AddaVAx as an adjuvant. Buffer treated mice served as negative controls. Induction of IFNγ/TNF double-positive multifunctional CD8+ T cells in the spleen was analyzed 42 days after boost vaccination by intracellular cytokine staining as described in Example 2.
As can be seen in FIG. 6, vaccination with the combination led to a significant increase in the number of multifunctional CD8+ T cells compared to mice vaccinated with HA-mRNA alone.

FIG. 7: shows that the intramuscular vaccination with a combination of the HA-mRNA (R2564) and the polymeric cargo complex (R2391, RNAdjuvant) as an adjuvant (see Example 1) and the additionals.c. injections of retinoic acid (ATRA, all-trans retinoic acid) leads to significant increase in HA-specific IgA titers in sera of immunized mice at day 14 after booster vaccination as described in Example 2 (Statistics calculated by Unpaired t test).

FIG. 8: shows that the intramuscular vaccination with a combination of the HA-mRNA (R2564) and the polymeric cargo complex (R2391, RNAdjuvant) as an adjuvant (see Example 1) and the s.c. injections of retinoic acid (ATRA, all-trans retinoic acid) leads to significant increase in HA-specific IgA titers in intestinal lavage of immunized mice at day 42 after booster vaccination as described in Example 2 (Statistics calculated by Unpaired t test).

FIG. 9: G/C-enriched mRNA sequence R2403 coding for the Rabies virus glycoprotein G (RAV-G) corresponding to SEQ ID NO: 3.

FIG. 10: G/C-enriched mRNA sequence R2507 coding for the Rabies virus glycoprotein G (RAV-G) corresponding to SEQ ID NO: 4

FIG. 11: shows that the intramuscular vaccination with a combination of the HA-mRNA (R2564) and c-di-GMP or Adju-Phos as an adjuvant induces higher neutralizing antibody titers against the HA protein at day 14 after booster vaccination compared to vaccination with the HA-mRNA (R2564) alone. This is in contrast to the combination with Alhydrogel, which abrogates the immune response. Mice were immunized as described in Example 4.

FIG. 12: shows that the intramuscular vaccination with a combination of the HA-mRNA (R2564) and c-di-GMP or Adju-Phos as an adjuvant induces higher frequencies of HA-specific IFN-γ+/TNF+ CD4+ T cells at day 14 after booster vaccination compared to vaccination with the HA-mRNA (R2564) alone. This is in contrast to the combination with Alhydrogel, which abrogates the immune response. Mice were immunized as described in Example 4.

FIG. 13: shows that the intramuscular vaccination with a combination of the protamine-formulated HA-mRNA (R2630) and c-di-GMP or Adju-Phos as an adjuvant induces higher neutralizing antibody titers against the HA protein at day 14 after booster vaccination compared to vaccination with the protamine-formulated HA-mRNA (R2630) alone. This is in contrast to the combination with Alhydrogel, which abrogates the immune response. Mice were immunized as described in Example 5.

FIG. 14: shows that the intramuscular vaccination with a combination of the protamine-formulated HA-mRNA (R2630) and c-di-GMP or Adju-Phos as an adjuvant induces higher frequencies of HA-specific IFN-γ+/TNF+CD4+ T cells at day 14 after booster vaccination compared to vaccination with the protamine-formulated HA-mRNA (R2630) alone. This is in contrast to the combination with Alhydrogel, which abrogates the immune response. Mice were immunized as described in Example 5.

FIG. 15: shows that the intramuscular vaccination with a combination of the HA-mRNA (R2564) and AddaVax, c-di-GMP or Adju-Phos as an adjuvant induces higher neutralizing antibody titers against the HA protein at day 14 after booster vaccination compared to vaccination with the HA-mRNA (R2564) alone. Mice were immunized as described in Example 6.

FIG. 16: shows that the intramuscular vaccination with a combination of the HA-mRNA (R2564) and AddaVax, c-di-GMP or Adju-Phos as an adjuvant induces higher frequencies of HA-specific IFN-γ+/TNF+CD4+ T cells at day 14 after booster vaccination compared to vaccination with the HA-mRNA (R2564) alone. Mice were immunized as described in Example 6.

FIG. 17: shows that the intramuscular vaccination with a combination of the RAV-G-mRNA (R1803) and AddaVax as an adjuvant induces higher rabies-specific virus neutralization titers (VNTs) at day 14 after booster vaccination compared to vaccination with the RAV-G-mRNA (R1803) alone. Mice were immunized as described in Example 7.

FIG. 18: shows that the intramuscular vaccination with a combination of the Protamine-formulated RAV-G mRNA (R2403) and AddaVax as an adjuvant induces higher rabies-specific virus neutralization titers (VNTs) at day 14 after booster vaccination compared to vaccination with the Protamine-formulated RAV-G mRNA (R2403) alone. Mice were immunized as described in Example 8.

### EXAMPLES

### Example 1: Preparation of the RNA

1. Preparation of DNA and mRNA Constructs

For the present example a DNA sequence encoding the hemagglutinin (HA) protein of influenza A virus (A/Netherlands/602/2009(H1N1)) was prepared and used for subsequent in vitro transcription reactions.

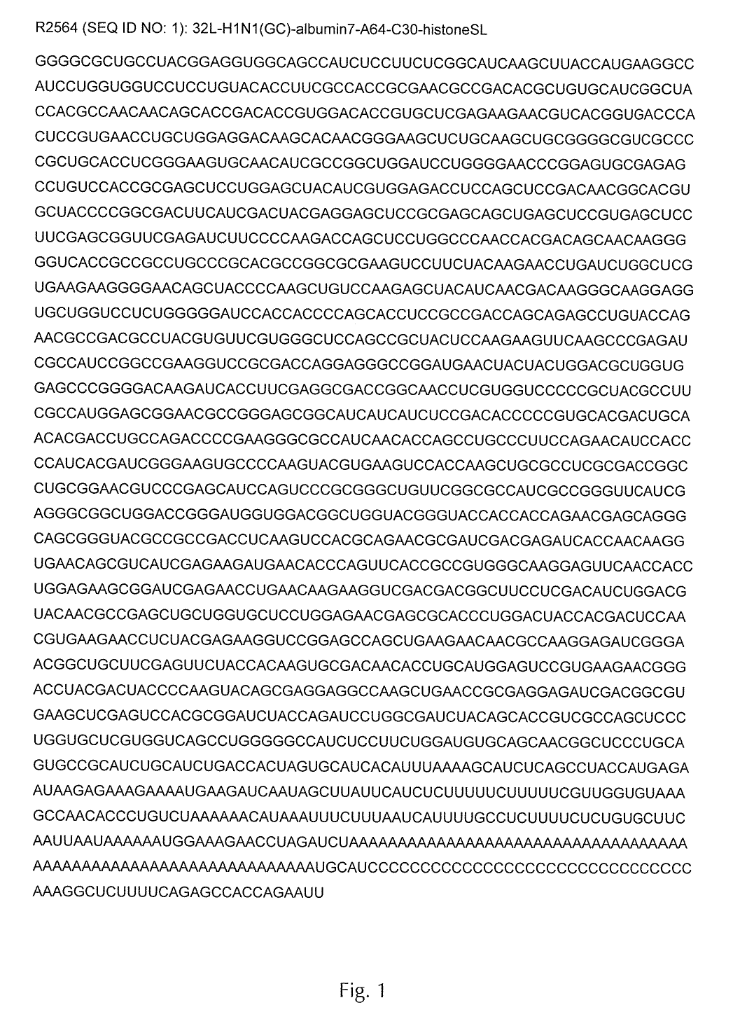

According to a first preparation, the DNA sequence coding for the above mentioned mRNA was prepared. The construct R2564 was prepared by introducing a 5′-TOP-UTR derived from the ribosomal protein 32L, modifying the wild type coding sequence by introducing a GC-optimized sequence for stabilization, followed by a stabilizing sequence derived from the albumin-3′-UTR, a stretch of 64 adenosines (poly(A)-sequence), a stretch of 30 cytosines (poly(C)-sequence), and a histone stem loop according. In SEQ ID NO: 1 (see FIG. 1) the sequence of the corresponding mRNA is shown.

For further examples DNA sequences, encoding glycoprotein G (RAV-G) of the Pasteur vaccine strain were prepared and used for subsequent in vitro transcription. The corresponding mRNA sequences RAV-G(GC)-muag-A64-C30-histoneSL (R2403 or R1803) and 32L-RAV-G(GC)-albumin7-A64-C30-histoneSL (R2507) are shown in FIGS. 9 and 10 according to SEQ. ID NO. 3 and 4.

### 2. Preparation of DNA and Non-Coding Immunostimulatory RNA Constructs

For the present examples a DNA sequence encoding the non-coding immunostimulatory RNA (isRNA) R2025 was prepared and used for subsequent in vitro transcription reactions.

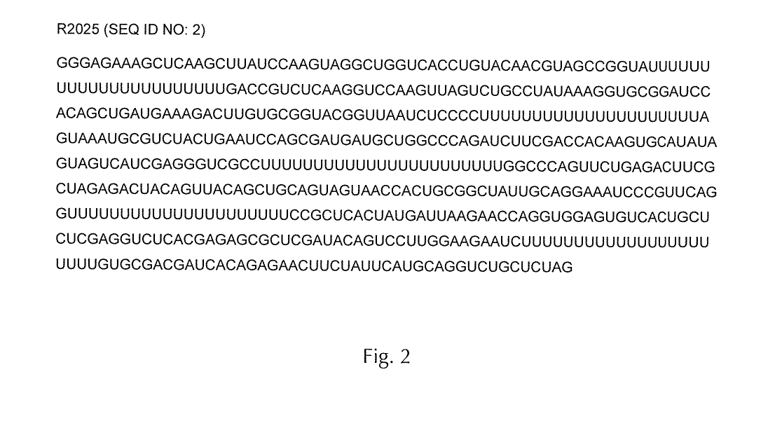

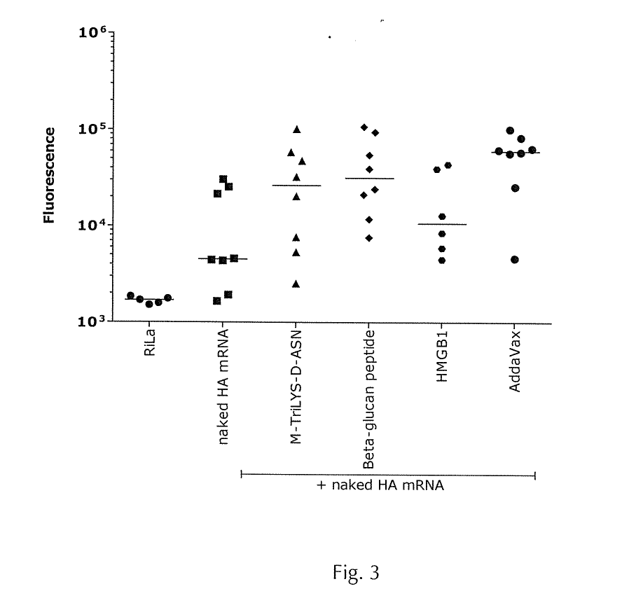

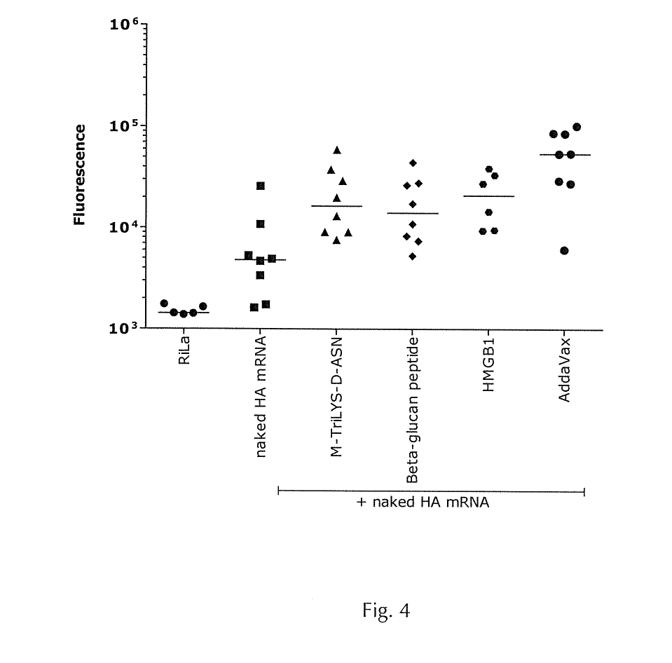

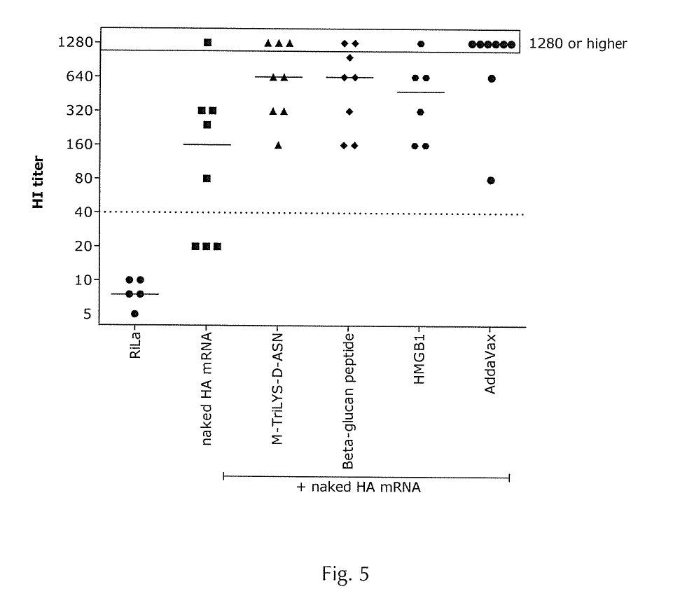

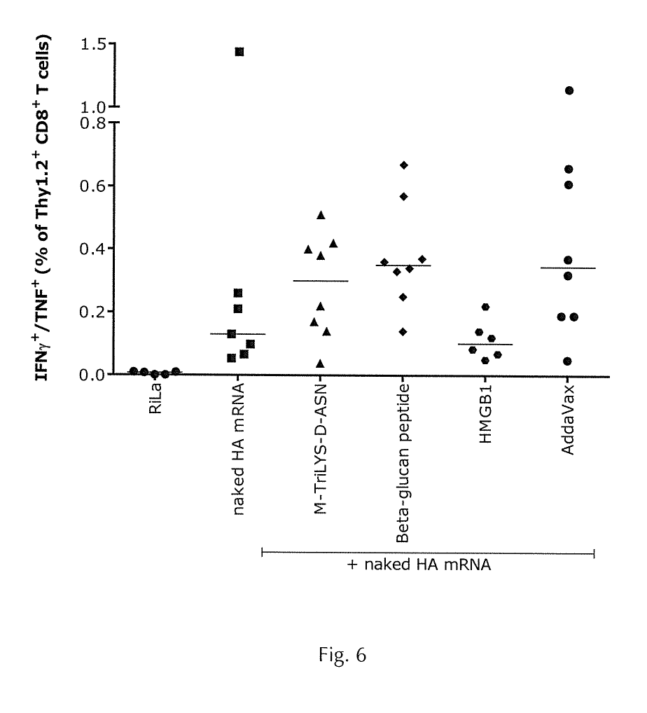

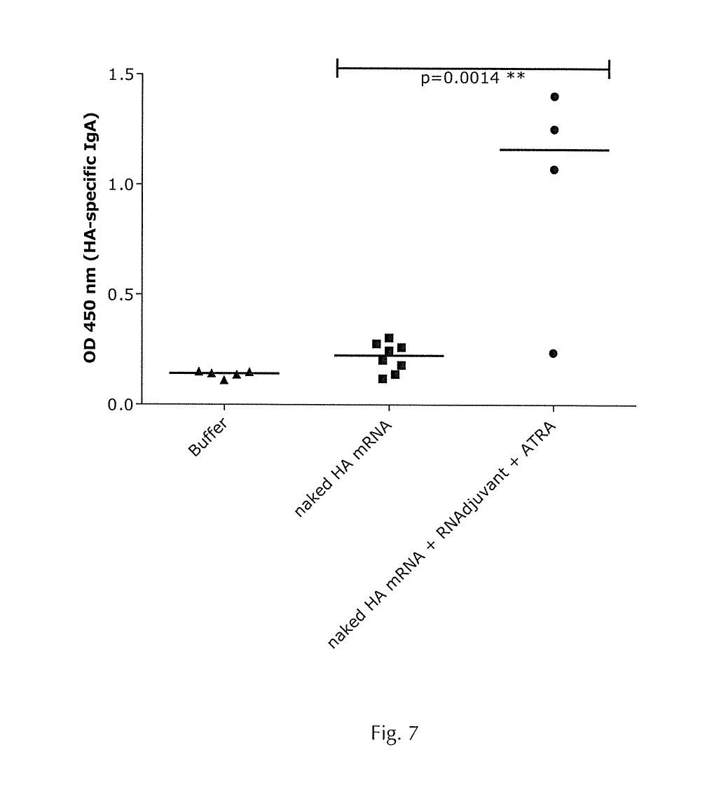

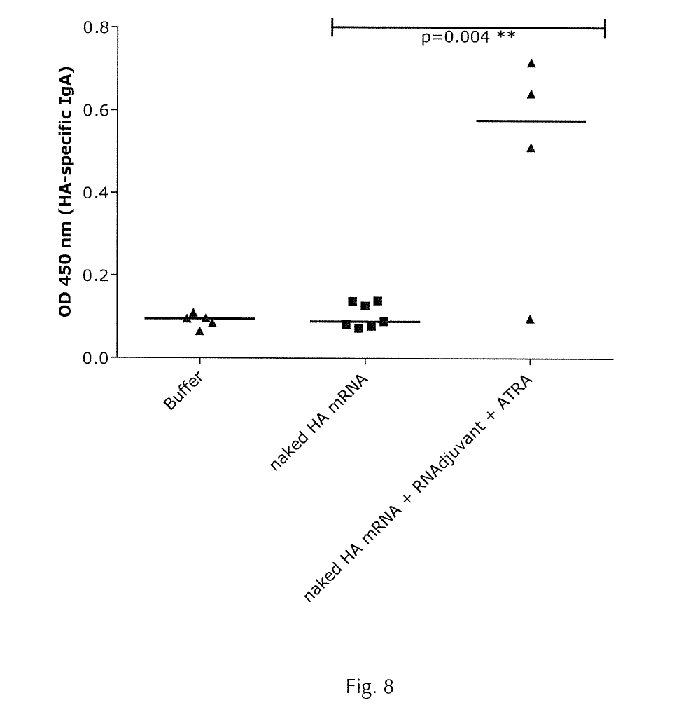

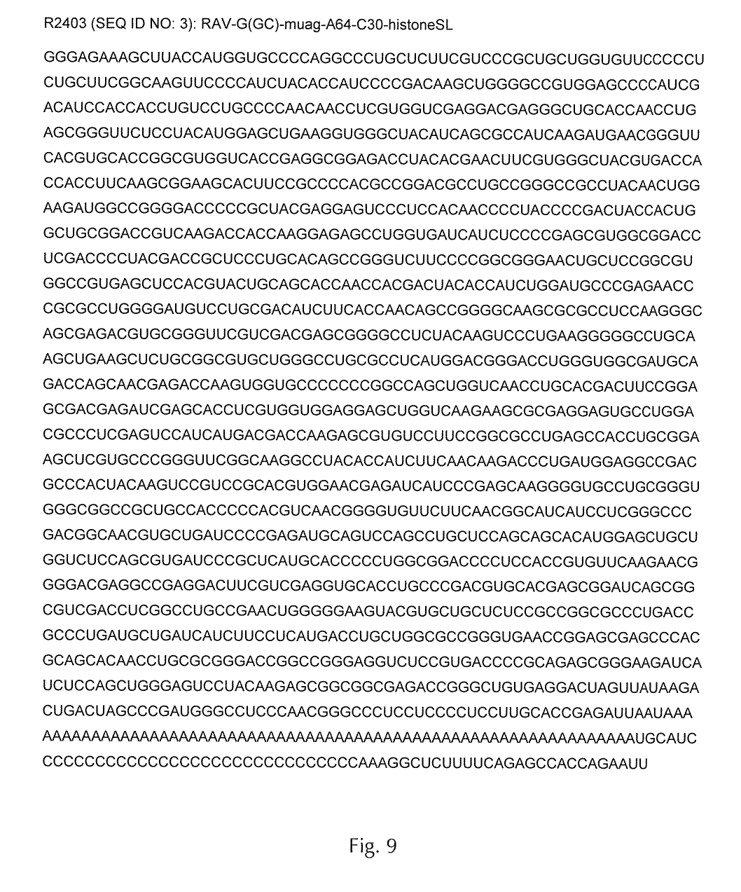

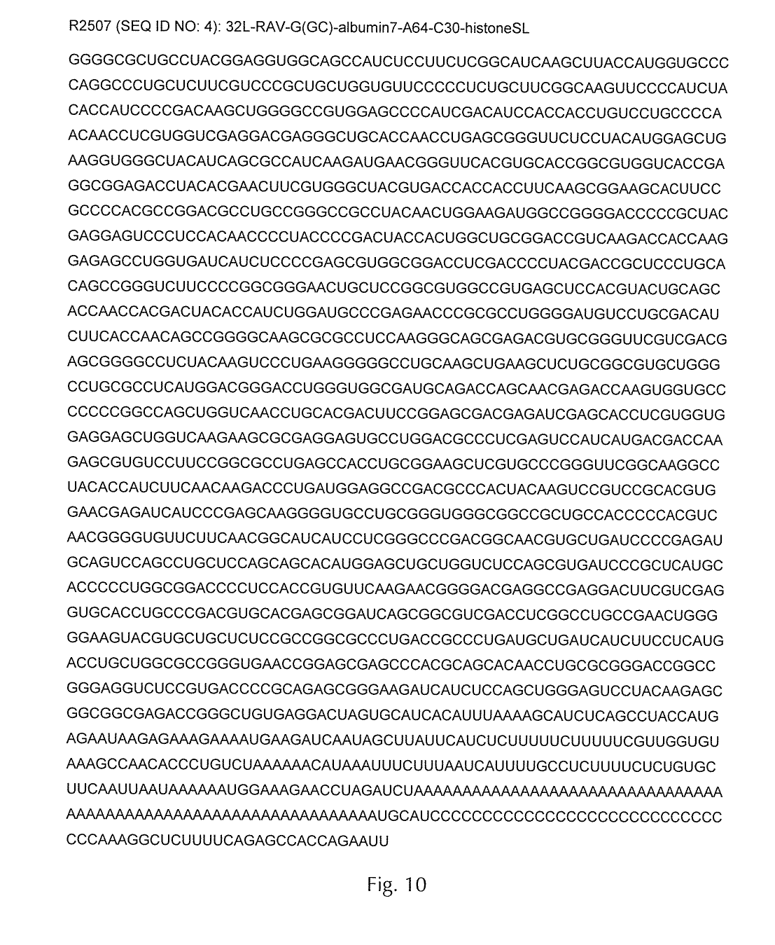

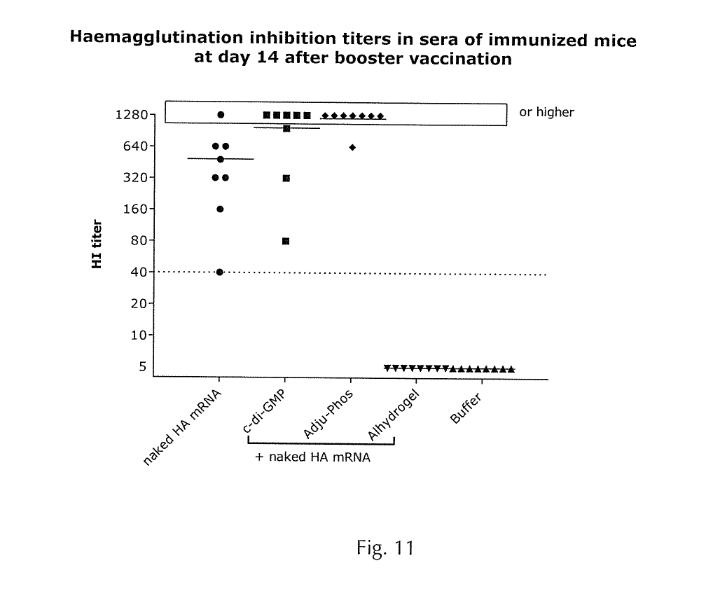

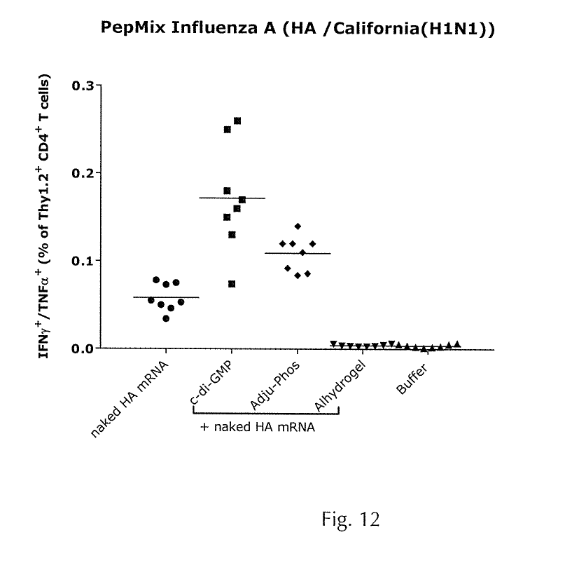

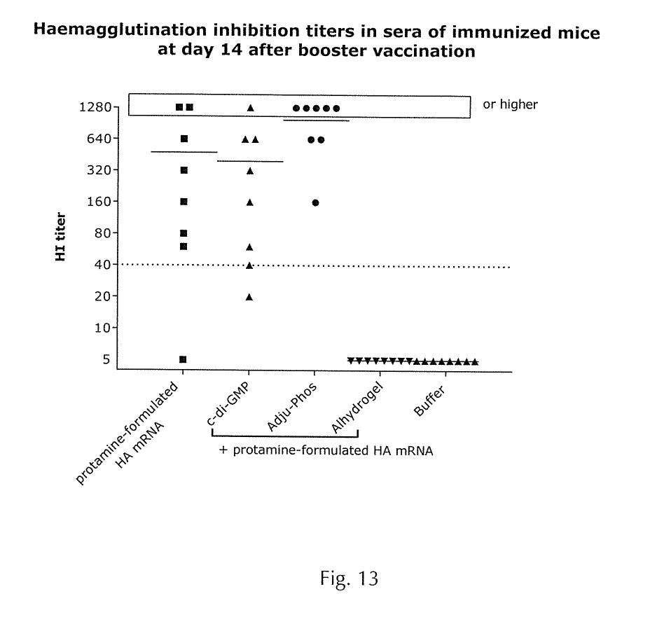

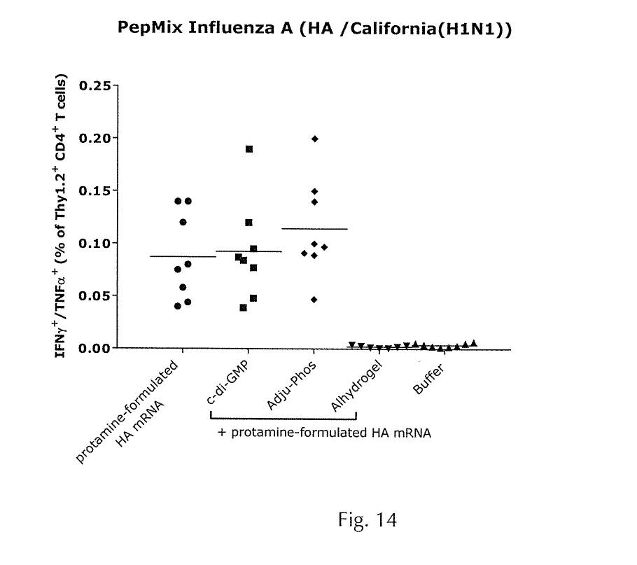

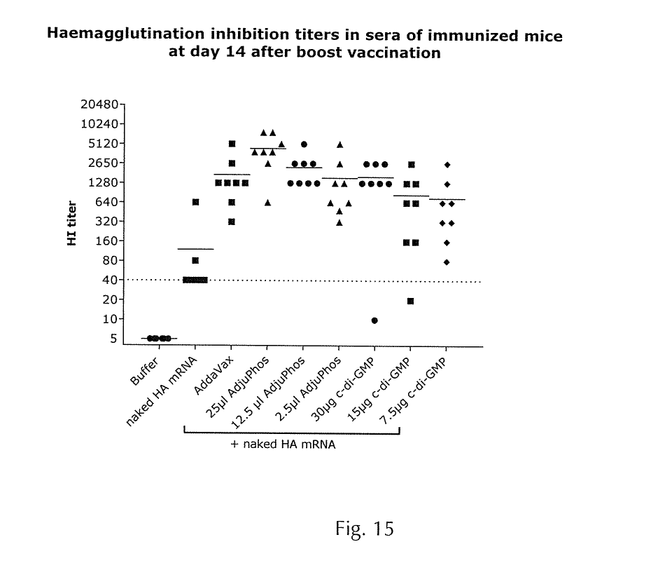

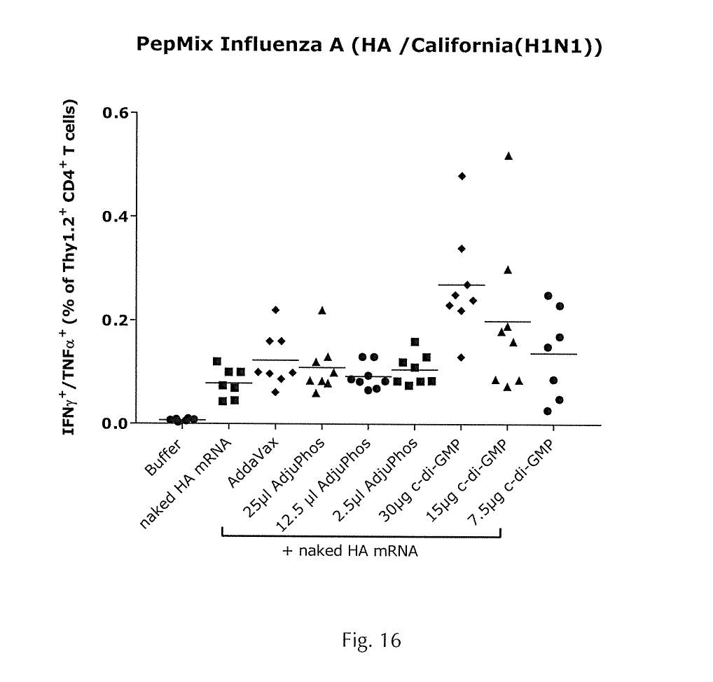

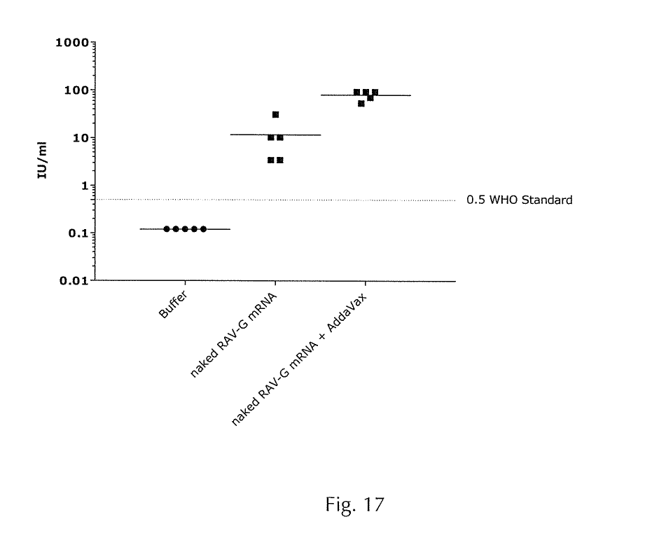

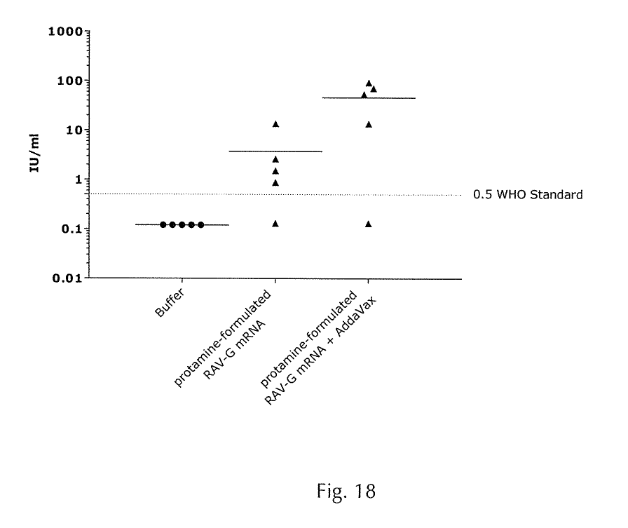

According to a first preparation, the DNA sequence coding for the above mentioned RNA was prepared. In SEQ ID NO: 2 (see FIG. 2) the sequence of the corresponding RNA is shown.

TABLE 1
RNA constructs

| RNA | Description | FIG. | SEQ ID NO. |
|---|---|---|---|
| R2564, | Influenza HA encoding mRNA | 1 | SEQ ID NO. 1 |
| P2630 | | | |
| R2025 | Non-coding immunostimulatory RNA | 2 | SEQ ID NO. 2 |
| R2403, | Rabies G Protein encoding mRNA | 9 | SEQ ID NO. 3 |
| R1803 | | | |
| R2507 | Rabies G Protein encoding mRNA | 10 | SEQ ID NO. 4 |

### 3. In Vitro Transcription

The respective DNA plasmids prepared according to paragraph 1 were transcribed in vitro using T7 polymerase. The in vitro transcription of influenza HA encoding R2564 and of rabies G-Protein encoding R2403, R1803, and R2507 was performed in the presence of a CAP analog (m⁷GpppG). The isRNA R2025 was prepared without CAP analog. Subsequently the RNA was purified using PureMessenger® (CureVac, Tubingen, Germany; WO2008/077592A1).

### 4. Preparation of the Polymeric Cargo Complex (RNAdjuvant)

Cationic peptide as cationic component of the polymeric carrier:

CR₁₂C:
(SEQ ID NO: 7)
Cys-Arg-Arg-Arg-Arg-Arg-Arg-Arg-Arg-Arg-Arg-Arg-
Arg-Cys
(Cys-Arg₁₂-Cys)

For synthesis of polymeric carrier cargo complexes the RNA sequence R2025 as defined in section 2 above was mixed with the cationic CR₁₂C peptide component as defined above. Therefore, the defined amount of the RNA was mixed with the respective cationic component in mass ratios as indicated, thereby forming a complex. If polymerizing cationic components were used according to the present invention polymerization of the cationic components took place simultaneously to complexation of the nucleic acid cargo. Afterwards the resulting solution was adjusted with water to a final volume of 50 μl and incubated for 30 minutes at room temperature. Further details are described in WO2012013326.

The mass ratio of peptide:RNA was 1:3,7. The polymeric carrier cargo complex is formed by the disulfide-crosslinked cationic peptide CR₁₂C as carrier and the immunostimulatory R2025 as nucleic acid cargo. This polymeric carrier cargo complex R2025/CR₁₂C (designated R2391) was used as adjuvant in the following examples.

### 5. Preparation of the Vaccine

The naked mRNA R2564 or the mRNA R1803 were administered in Ringer's Lactate solution.

For protamine-complexation, the mRNA R2564 or the mRNA R2403 was complexed with protamine in a mass ratio of 2:1. After incubation the same amount of naked mRNA R2564 or R2403 was added to the nanoparticles. This vaccine formulation is referred to as R2630 or R1830 RNActive®.

The adjuvants were dissolved in water to the following concentrations: 1 mg/ml M-TriLYS-D-ASN, 3 mg/ml Beta-glucan peptide (BGP), 0.5 mg/mlmouse HMGB1 and 0.2 mg/ml 5′ppp-dsRNA.

The oil-in-water adjuvant AddaVax was mixed with mRNA solution at a 0.25:1, a 0.75:1 or a 1:1 ratio.

The lyophilized polymeric carrier cargo complex R2391 was dissolved in Ringer's Lactate solution to a final concentration of 2 μg/μl.

The co-formulations of naked mRNA R2564 or naked mRNA R1803 and the additional adjuvants M-TriLYS-D-ASN, Beta-glucan peptide, HMGB1, 5′ppp-dsRNA, AddaVax®, c-di-GMP, Adju-Phos®, Alhydrogel® or R2391 were generated by mixing both components directly before administration.

The co-formulations of R2630 RNActive® or R2403 RNActive® and the additional adjuvants M-TriLYS-D-ASN, Beta-glucan peptide, HMGB1, 5′ppp-dsRNA, AddaVax®, c-di-GMP, Adju-Phos® Alhydrogel® or R2391 were generated by mixing both components directly before administration.

### 6. Preparation of the ATRA and the Retinylpalmitate (PP) Working Solution

ATRA was dissolved in PEG400 to a final concentration of 2 g/l.

Retinylpalmitate was dissolved in sunflower oil to a final concentration of 7.8 μl/ml.

### Example 2: Induction of a Humoral and Cellular Immune Response after Intramuscular Vaccination of Mice with Naked Influenza HA-Encoding mRNA

### Immunization

On day zero, BALB/c mice, 8 week old, were injected intramuscularly (i.m.) into both M. tibialis with the influenza HA-encoding mRNA (R2564) alone or in combination with 10 μg M-TriLYS-D-ASN, 50 μg Beta-glucan peptide, 3 μg HMGB1, or AddaVax (RNA solution:AddaVax=1:0.75) as an adjuvant as shown in Table 2. Mice injected with Ringer Lactate (RiLa) buffer served as controls. All animals received boost injections on day 21. Blood samples were collected on day 21, day 35, day 49 and day 63. Spleens and intestinal lavages were collected on day 63.

One group of mice received i.m. the influenza HA-encoding mRNA (R2564) in combination with the polymeric carrier cargo complex (R2391, “RNAdjuvant”) and additionally s.c. injections of retinoic acid (ATRA, all-trans retinoic acid) on day 0, day 2, day 4, day 7, day 21, day 23, day 25 and day 28.

HA-specific IgG1, IgG2a and IgA titers were measured by ELISA or Haemagglutination Inhibition Assay. HA-specific T cells were measured by intracellular cytokine staining.

TABLE 2
Animal groups

| | | | | | | Polymeric | |
|---|---|---|---|---|---|---|---|
| | | | | | | carrier cargo | |
| | Strain | No. | Route | RiLa | HA RNA | complex | Additional |
| Group | sex | mice | volume | buffer | R2564 | R2391 | adjuvant |
| 1 | BALB/c | 6 | i.m. | 2 × 25 μl | — | — | — |
| | Female | | 2 × 25 μl | | | | |
| 2 | BALB/c | 8 | i.m. | | 2 × 20 μg | — | — |
| | Female | | 2 × 25 μl | | | | |
| 3 | BALB/c | 8 | i.m. | | 2 × 20 μg | — | 10 μg M-TriLYS-D- |
| | Female | | 2 × 25 μl | | | | ASN |
| 4 | BALB/c | 8 | i.m. | | 2 × 20 μg | — | 50 μg Beta-glucan |
| | Female | | 2 × 25 μl | | | | peptide |
| 5 | BALB/c | 6 | i.m. | | 2 × 20 μg | — | 3 μg HMGB1 |
| | Female | | 2 × 25 μl | | | | |
| 6 | BALB/c | 8 | i.m. | | 2 × 20 μg | — | AddaVax (RNA |
| | Female | | 2 × 25 μl | | | | solution:AddaVax = |
| | | | | | | | 1:0.75) |
| 7 | BALB/c | 8 | i.m. | | 2 × 20 μg | 2 × 20 μg | 200 μg retinoic acid |
| | Female | | 2 × 25 μl | | | | (ATRA) |

### Example 3: Induction of a Humoral and Cellular Immune Response after Intramuscular Vaccination of Mice with Naked or Protamine-Formulated Influenza HA-Encoding mRNA

### Immunization

On day zero, BALB/c mice were injected intramuscularly (i.m.) into both M. tibialis with the naked influenza HA-encoding mRNA (R2564) or the protamine-formulated influenza HA-encoding mRNA (R2630 RNActive®) alone or in combination with M-TriLYS-D-ASN, Beta-glucan peptide, 5′ppp-dsRNA, or AddaVax as an adjuvant as shown in Table 3. Mice injected with Ringer Lactate (RiLa) buffer served as controls. All animals received boost injections on day 21. Blood samples were collected on day 21 and day 28. Spleens and intestinal lavages were collected on day 28.

One group of mice received i.m. the naked influenza HA-encoding mRNA (R2564) or the protamine-formulated influenza HA-encoding mRNA (R2630 RNActive®) and additionally s.c. injections of retinyl palmitat (RP) on day 0, day 3, day 6, day 21 and day 24.

HA-specific IgG1, IgG2a and IgA titers were measured by ELISA or Haemagglutination Inhibition Assay. HA-specific T cells were measured by intracellular cytokine staining.

TABLE 3
Animal groups

| | | | | | | Protamine- |
|---|---|---|---|---|---|---|
| | | | | | | formulated |
| | Strain | No. | Route | Additional | HA mRNA | HA mRNA |
| Group | sex | mice | volume | adjuvant | R2564 | R2630 |
| 1 | BALB/c | 6 | i.m. | — | — | — |
| | Female | | 2 × 25 μl | | | |
| 2 | BALB/c | 6 | i.m. | — | 2 × 20 μg | — |
| | Female | | 2 × 25 μl | | | |
| 3 | BALB/c | 6 | i.m. | — | — | 2 × 20 μg |
| | Female | | 2 × 25 μl | | | |
| 4 | BALB/c | 7 | i.m. | 10 μg M-TriLYS- | 2 × 20 μg | — |
| | Female | | 2 × 25 μl | D-ASN | | |
| 5 | BALB/c | 7 | i.m. | 50 μg Beta- | 2 × 20 μg | — |
| | Female | | 2 × 25 μl | glucan peptide | | |
| 6 | BALB/c | 7 | i.m. | AddaVax | 2 × 20 μg | — |
| | Female | | 2 × 25 μl | 1:1 v/v | | |
| 7 | BALB/c | 6 | i.m. | 8 μg 5′ppp-dsRNA | 2 × 20 μg | — |
| | Female | | 2 × 25 μl | | | |
| 8 | BALB/c | 6 | | 360 μg | 2 × 20 μg | — |
| | Female | | | retinylpalmitate | | |
| 9 | BALB/c | 7 | | 10 μg M-TriLYS | — | 2 × 20 μg |
| | Female | | | D-ASN | | |
| 10 | BALB/c | 7 | | 50 μg Beta- | — | 2 × 20 μg |
| | Female | | | glucan peptide | | |
| 11 | BALB/c | 7 | | AddaVax | — | 2 × 20 μg |
| | Female | | | 1:1 v/v | | |
| 12 | BALB/c | 6 | | 360 μg retinyl | — | 2 × 20 μg |
| | Female | | | palmitate | | |

### Example 4: Induction of a Humoral and Cellular Immune Response after Intramuscular Vaccination of Mice with Naked Influenza HA-Encoding mRNA

### Immunization

On day zero, BALB/c mice were injected intramuscularly (i.m.) into both M. tibialis with the naked influenza HA-encoding mRNA (R2564) alone or in combination with c-di-GMP, Adju-Phos®or Alhydrogel® as an adjuvant as shown in Table 4. Mice injected with Ringer Lactate (RiLa) buffer served as controls. All animals received boost injections on day 21. Blood samples were collected on day 21 and day 35. Spleens were collected on day 35.

HA-specific IgG1 and IgG2a titers were measured by ELISA or Haemagglutination Inhibition Assay. HA-specific T cells were measured by intracellular cytokine staining.

TABLE 4
Animal groups

| | Strain | No. | Route | Additional | HA mRNA |
|---|---|---|---|---|---|
| Group | sex | mice | volume | adjuvant | R2564 |
| 1 | BALB/c | 8 | i.m. | — | 2x 20 μg |
| | Female | | 2x 25 μl | | |
| 2 | BALB/c | 8 | i.m. | 7.5 μg | 2x 20 μg |
| | Female | | 2x 25 μl | c-di-GMP | |
| 3 | BALB/c | 8 | i.m. | 25 μl Adju- | 2x 20 μg |
| | Female | | 2x 25 μl | Phos ® | |
| 4 | BALB/c | 8 | i.m. | 25 μl | 2x 20 μg |
| | Female | | 2x 25 μl | Alhydrogel ® | |
| 5 | BALB/c | 8 | i.m. | — | — |
| | Female | | 2x 25 μl | | |

### Example 5: Induction of a Humoral and Cellular Immune Response after Intramuscular Vaccination of Mice with Protamine-Formulated Influenza HA-Encoding mRNA

### Immunization

On day zero, BALB/c mice were injected intramuscularly (i.m.) into both M. tibialis with the protamine-formulated influenza HA-encoding mRNA (R2630 RNActive®) alone or in combination with c-di-GMP, Adju-Phos® or Alhydrogel® as an adjuvant as shown in Table 5. Mice injected with Ringer Lactate (RiLa) buffer served as controls. All animals received boost injections on day 21. Blood samples were collected on day 21 and day 35. Spleens were collected on day 35.

HA-specific IgG1 and IgG2a titers were measured by ELISA or Haemagglutination Inhibition Assay. HA-specific T cells were measured by intracellular cytokine staining.

TABLE 5
Animal groups

| | | | | | Protamine- |
|---|---|---|---|---|---|
| | | | | | formulated |
| | Strain | No. | Route | Additional | HA mRNA |
| Group | sex | mice | volume | adjuvant | R2630 |
| 1 | BALB/c | 8 | i.m. | — | 2x 20 μg |
| | Female | | 2x 25 μl | | |
| 2 | BALB/c | 8 | i.m. | 7.5 μg | 2x 20 μg |
| | Female | | 2x 25 μl | c-di-GMP | |
| 3 | BALB/c | 8 | i.m. | 25 μl Adju- | 2x 20 μg |
| | Female | | 2x 25 μl | Phos ® | |
| 4 | BALB/c | 8 | i.m. | 25 μl | 2x 20 μg |
| | Female | | 2x 25 μl | Alhydrogel ® | |
| 5 | BALB/c | 8 | i.m. | — | — |
| | Female | | 2x 25 μl | | |

### Example 6: Induction of a Humoral and Cellular Immune Response after Intramuscular Vaccination of Mice with Naked Influenza HA-Encoding mRNA

### Immunization

On day zero, BALB/c mice were injected intramuscularly (i.m.) into both M. tibialis with the naked influenza HA-encoding mRNA (R2564) alone or in combination with AddaVax® or different amounts of c-di-GMP or Adju-Phos® as an adjuvant as shown in Table 6. Mice injected with Ringer Lactate (RiLa) buffer served as controls. All animals received boost injections on day 21. Blood samples were collected on day 21 and day 35. Spleens were collected on day 35.

HA-specific IgG1 and IgG2a titers were measured by ELISA or Haemagglutination Inhibition Assay. HA-specific T cells were measured by intracellular cytokine staining.

TABLE 6
Animal groups

| | Strain | No. | Route | Additional | HA mRNA |
|---|---|---|---|---|---|
| Group | sex | mice | volume | adjuvant | R2564 |
| 1 | BALB/c | 8 | i.m. | — | 2x 20 μg |
| | Female | | 2x 25 μl | | |
| 3 | BALB/c | 8 | i.m. | 10 μl | 2x 20 μg |
| | Female | | 2x 25 μl | AddaVax ® | |
| 4 | BALB/c | 8 | i.m. | 25 μl | 2x 20 μg |
| | Female | | 2x 25 μl | Adju-Phos ® | |
| 5 | BALB/c | 8 | i.m. | 12.5 μl | 2x 20 μg |
| | Female | | 2x 25 μl | Adju-Phos ® | |
| 6 | BALB/c | 8 | i.m. | 2.5 μl | 2x 20 μg |
| | Female | | 2x 25 μl | Adju-Phos ® | |
| 7 | BALB/c | 8 | i.m. | 30 μg | 2x 20 μg |
| | Female | | 2x 25 μl | c-di-GMP | |
| 8 | BALB/c | 8 | i.m. | 15 μg | 2x 20 μg |
| | Female | | 2x 25 μl | c-di-GMP | |
| 9 | BALB/c | 8 | i.m. | 7.5 μg | 2x 20 μg |
| | Female | | 2x 25 μl | c-di-GMP | |
| 10 | BALB/c | 8 | i.m. | — | 2x 20 μg |
| | Female | | 2x 25 μl | | |

### Example 7: Induction of a Humoral and Cellular Immune Response after Intramuscular Vaccination of Mice with Naked Rabies RAV-G Encoding mRNA

### Immunization

On day zero, BALB/c mice were injected intramuscularly (i.m.) into both M. tibialis with the naked rabies RAV-G-encoding mRNA (R1803) alone or in combination with AddaVax® as an adjuvant as shown in Table 7. Mice injected with Ringer Lactate (RiLa) buffer served as controls. All animals received boost injections on day 21. Blood samples were collected on day 21 and day 35. Spleens were collected on day 35.

RAV-G-specific antibody titers were measured by Virus neutralization titers (VNT). RAV-G-specific T cells were measured by ELISPOT analysis.

TABLE 7
Animal groups

| | Strain | No. | Route | Additional | RAV-G mRNA |
|---|---|---|---|---|---|
| Group | sex | mice | volume | adjuvant | R1803 |
| 1 | BALB/c | 8 | i.m. | — | 2x 20 μg |
| | Female | | 2x 25 μl | | |
| 2 | BALB/c | 8 | i.m. | 10 μl | 2x 20 μg |
| | Female | | 2x 25 μl | AddaVax ® | |
| 3 | BALB/c | 8 | i.m. | — | 2x 20 μg |
| | Female | | 2x 25 μl | | |

### Example 8: Induction of a Humoral and Cellular Immune Response after Intramuscular Vaccination of Mice with Protamine-Formulated Rabies RAV-G Encoding mRNA

### Immunization

On day zero, BALB/c mice were injected intramuscularly (i.m.) into both M. tibialis with the protamine-formulated rabies RAV-G-encoding mRNA (R2403 RNActive®) alone or in combination with AddaVax® as an adjuvant as shown in Table 8. Mice injected with Ringer Lactate (RiLa) buffer served as controls. All animals received boost injections on day 21. Blood samples were collected on day 21 and day 35. Spleens were collected on day 35.

RAV-G-specific antibody titers were measured by Virus neutralization titers (VNT). RAV-G-specific T cells were measured by ELISPOT analysis

TABLE 8
Animal groups

| | | | | | Protamine- |
|---|---|---|---|---|---|
| | | | | | formulated |
| | Strain | No. | Route | Additional | RAV-G mRNA |
| Group | sex | mice | volume | adjuvant | R2403 |
| 1 | BALB/c | 8 | i.m. | — | 2x 20 μg |
| | Female | | 2x 25 μl | | |
| 2 | BALB/c | 8 | i.m. | 10 μl | 2x 20 μg |
| | Female | | 2x 25 μl | AddaVax ® | |
| 3 | BALB/c | 8 | i.m. | — | — |
| | Female | | 2x 25 μl | | |

### Protocols

### Determination of HA Specific Antibodies by ELISA

ELISA plates are coated with 1 μg/mlInfluenza Antigen A/California/7/2009. Coated plates are incubated using given serum dilutions and binding of specific antibodies to the HA protein was detected using rat monoclonal anti-mouse IgG1 or IgG2a, developed with Amplex or goat polyclonal anti-mouse IgA, developed with TMB.

### Hemagglutination Inhibition Assay

For hemagglutination inhibition (HI) assay mouse sera were heat inactivated (56° C., 30 min), incubated with kaolin, and pre-adsorbed to chicken red blood cells (CRBC) (both Labor Dr. Merck &Kollegen, Ochsenhausen, Germany). For the HI assay, 50 μl of 2-fold dilutions of pre-treated sera were incubated for 45 minutes with 4 hemagglutination units (HAU) of inactivated A/California/5 7/2009 (NIBSC, Potters Bar, UK) and 50 μl 0.5% CRBC were added.

### Virus Neutralization Titers (VNT)

Virus neutralization titers against RAV-G were measured using a Fluorescent Antibody Virus Neutralization (FAVN) according to WHO guidelines

### Intracellular Cytokine Staining

Splenocytes from vaccinated and control mice were isolated according to a standard protocol. Briefly, isolated spleens were ground through a cell strainer and washed in PBS/1% FBS followed by red blood cell lysis. After an extensive washing step with PBS/1% FBS splenocytes were seeded into 96-well plates (2×10⁶ cells/well). Cells were stimulated with PepMix Influenza A (HA/California(H1N1), 0.5 μg/peptide/ml, JPT Peptide Technologies GmbH) and 2.5 μg/ml of an anti-CD28 antibody (BD Biosciences) for 6 hours at 37° C. in the presence of the mixture of GolgiPlug™/GolgiStop™ (Protein transport inhibitors containing Brefeldin A and Monensin, respectively; BD Biosciences). Cells incubated with medium were used as controls. After stimulation cells were washed, incubated with. Aqua Dye to distinguish live/dead cells (Invitrogen, Life Technologies), surface stained with CD4-V450 (1:200, BD Biosciences), CD8 PE-Cy7(1:200), Thy1.2 FITC (1:300) and FcgR block (1:100), fixed and permeabilized using the Cytofix/Cytoperm reagent (BD Biosciences), and stained intracellularly with IL2-PerCP-Cy5.5 (1:100), TNFα-PE (1:100) and IFNγ-APC (1:100) (eBioscience). Cells were collected using a Canto II flow cytometer (Beckton Dickinson). Flow cytometry data were analysed using FlowJo software (Tree Star, Inc.).

### Enzyme-Linked Immunosorbent Spot Assay (ELISPOT)

Splenocytes from vaccinated and control mice were isolated as described above and stimulated with a PepMix (JPT Peptide Technologies GmbH), which contains overlapping peptides of RAV-G (1 μg/ml). Secreted IFNγ was detected using a standard ELISPOT protocol and measured using a plate reader (Immunospot Analyzer, CTL Analyzers LLC).

1. A composition comprising at least a first immunogenic component and at least a second adjuvant component,
wherein the first immunogenic component comprises at least one nucleic acid molecule encoding at least one epitope of at least one antigen, and
wherein the second adjuvant component comprises at least one immune potentiator compound or at least one delivery system compound.

2. The composition of claim 1, wherein the second adjuvant component comprises at least one mineral salt compound.

3. The composition of claim 2, wherein the mineral salt compound is selected from the group consisting of an aluminum salt, a calcium salt, an iron salt and a zirconium salt.

4. The composition of claim 3, wherein the mineral salt compound is selected from the group consisting of an aluminum phosphate salt, a calcium phosphate salt, an iron phosphate salt and a zirconium phosphate salt.

5. (canceled)

6. The composition of claim 1, wherein the second adjuvant component comprises at least one emulsion or surfactant-based compound.

7. (canceled)

8. The composition of claim 1, wherein the second adjuvant component comprises at least one nucleotide-based or nucleoside-based compound.

9. The composition of claim 1, wherein the second adjuvant component comprises at least one protein-based or peptide-based compound.

10. The composition of claim 1, wherein the second adjuvant component comprises at least one hydrocarbon-based or carbohydrate-based compound.

11. The composition of claim 1, wherein the second adjuvant component comprises at least one lipid-based compound.

12. The composition of claim 1, wherein the second adjuvant component comprises at least one polymeric compound.

13. The composition of claim 1, wherein the second adjuvant component comprises at least one cytokine or at least one hormone compound.

14. The composition of claim 1, wherein the second adjuvant component comprises at least one toxin compound.

15. The composition of claim 1, wherein the second adjuvant component comprises at least one vehicle compound, preferably a liposome compound and/or a virosome compound and/or a virus-like particle compound and/or a microparticle compound and/or a nanoparticle compound and/or a protein cochleate compound.

16. The composition of claim 1, wherein the second adjuvant component comprises a polymeric carrier cargo complex comprising as a carrier a complex of at least one cationic or oligocationic or polycationic component and as a cargo at least one nucleic acid molecule.

17-18. (canceled)

19. The composition of claim 1, wherein the at least one nucleic acid molecule of the immunogenic component is an mRNA molecule.

20. The composition of claim 19, wherein the G/C content of the coding region of the mRNA molecule is increased compared with the G/C content of the coding region of the wild type mRNA.

21. The composition of claim 19, wherein the mRNA molecule comprises additionally a 5′-UTR element, 3′-UTR element, at least one histone stem-loop, a 5′-CAP structure, poly(A) sequence, or a poly(C) sequence.

22. The composition of claim 19, wherein the mRNA molecule is complexed with a cationic component.

23. The composition of claim 1, wherein said at least one antigen is selected from the group consisting of an antigen from a pathogen associated with infectious diseases, an antigen associated with allergies, an antigen associated with autoimmune diseases, and an antigen associated with cancer or tumor diseases, or a fragment, variant and/or derivative of said antigen.

24-40. (canceled)

41. A method of treatment or prophylaxis comprising administering to a subject in need thereof a therapeutically effective amount of the composition of claim 1.

42-45. (canceled)


SEQUENCE LISTING (10 sequences)

SEQ ID NO: 1 - RNA, 2083 nt, Influenza virus
ggggcgcugccuacggagguggcagccaucuccuucucggcaucaagcuuaccaugaaggccauccuggugguccuccuguacaccuucgccaccgcgaacgccgacacgcugugcaucggcuaccacgccaacaacagcaccgacaccguggacaccgugcucgagaagaacgucacggugacccacuccgugaaccugcuggaggacaagcacaacgggaagcucugcaagcugcggggcgucgccccgcugcaccucgggaagugcaacaucgccggcuggauccuggggaacccggagugcgagagccuguccaccgcgagcuccuggagcuacaucguggagaccuccagcuccgacaacggcacgugcuaccccggcgacuucaucgacuacgaggagcuccgcgagcagcugagcuccgugagcuccuucgagcgguucgagaucuuccccaagaccagcuccuggcccaaccacgacagcaacaagggggucaccgccgccugcccgcacgccggcgcgaaguccuucuacaagaaccugaucuggcucgugaagaaggggaacagcuaccccaagcuguccaagagcuacaucaacgacaagggcaaggaggugcugguccucugggggauccaccaccccagcaccuccgccgaccagcagagccuguaccagaacgccgacgccuacguguucgugggcuccagccgcuacuccaagaaguucaagcccgagaucgccauccggccgaagguccgcgaccaggagggccggaugaacuacuacuggacgcugguggagcccggggacaagaucaccuucgaggcgaccggcaaccucguggucccccgcuacgccuucgccauggagcggaacgccgggagcggcaucaucaucuccgacacccccgugcacgacugcaacacgaccugccagaccccgaagggcgccaucaacaccagccugcccuuccagaacauccaccccaucacgaucgggaagugccccaaguacgugaaguccaccaagcugcgccucgcgaccggccugcggaacgucccgagcauccagucccgcgggcuguucggcgccaucgccggguucaucgagggcggcuggaccgggaugguggacggcugguacggguaccaccaccagaacgagcagggcagcggguacgccgccgaccucaaguccacgcagaacgcgaucgacgagaucaccaacaaggugaacagcgucaucgagaagaugaacacccaguucaccgccgugggcaaggaguucaaccaccuggagaagcggaucgagaaccugaacaagaaggucgacgacggcuuccucgacaucuggacguacaacgccgagcugcuggugcuccuggagaacgagcgcacccuggacuaccacgacuccaacgugaagaaccucuacgagaagguccggagccagcugaagaacaacgccaaggagaucgggaacggcugcuucgaguucuaccacaagugcgacaacaccugcauggaguccgugaagaacgggaccuacgacuaccccaaguacagcgaggaggccaagcugaaccgcgaggagaucgacggcgugaagcucgaguccacgcggaucuaccagauccuggcgaucuacagcaccgucgccagcucccuggugcucguggucagccugggggccaucuccuucuggaugugcagcaacggcucccugcagugccgcaucugcaucugaccacuagugcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccuagaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 2 - RNA, 547 nt, Artificial Sequence
  R2025 (RNAdjuvant Sequence)
gggagaaagcucaagcuuauccaaguaggcuggucaccuguacaacguagccgguauuuuuuuuuuuuuuuuuuuuuugaccgucucaagguccaaguuagucugccuauaaaggugcggauccacagcugaugaaagacuugugcgguacgguuaaucuccccuuuuuuuuuuuuuuuuuuuuuaguaaaugcgucuacugaauccagcgaugaugcuggcccagaucuucgaccacaagugcauauaguagucaucgagggucgccuuuuuuuuuuuuuuuuuuuuuuuggcccaguucugagacuucgcuagagacuacaguuacagcugcaguaguaaccacugcggcuauugcaggaaaucccguucagguuuuuuuuuuuuuuuuuuuuuccgcucacuaugauuaagaaccagguggagugucacugcucucgaggucucacgagagcgcucgauacaguccuuggaagaaucuuuuuuuuuuuuuuuuuuuuuugugcgacgaucacagagaacuucuauucaugcaggucugcucuag

SEQ ID NO: 3 - RNA, 1792 nt, Artificial Sequence
  R2403 RAV-G(GC)-muag-A64-C30-histoneSL
gggagaaagcuuaccauggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuaguuauaagacugacuagcccgaugggccucccaacgggcccuccuccccuccuugcaccgagauuaauaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 4 - RNA, 1957 nt, Artificial Sequence
  R2507 32L-RAV-G(GC)-albumin7-A64-C30-histoneSL
ggggcgcugccuacggagguggcagccaucuccuucucggcaucaagcuuaccauggugccccaggcccugcucuucgucccgcugcugguguucccccucugcuucggcaaguuccccaucuacaccauccccgacaagcuggggccguggagccccaucgacauccaccaccuguccugccccaacaaccucguggucgaggacgagggcugcaccaaccugagcggguucuccuacauggagcugaaggugggcuacaucagcgccaucaagaugaacggguucacgugcaccggcguggucaccgaggcggagaccuacacgaacuucgugggcuacgugaccaccaccuucaagcggaagcacuuccgccccacgccggacgccugccgggccgccuacaacuggaagauggccggggacccccgcuacgaggagucccuccacaaccccuaccccgacuaccacuggcugcggaccgucaagaccaccaaggagagccuggugaucaucuccccgagcguggcggaccucgaccccuacgaccgcucccugcacagccgggucuuccccggcgggaacugcuccggcguggccgugagcuccacguacugcagcaccaaccacgacuacaccaucuggaugcccgagaacccgcgccuggggauguccugcgacaucuucaccaacagccggggcaagcgcgccuccaagggcagcgagacgugcggguucgucgacgagcggggccucuacaagucccugaagggggccugcaagcugaagcucugcggcgugcugggccugcgccucauggacgggaccuggguggcgaugcagaccagcaacgagaccaaguggugcccccccggccagcuggucaaccugcacgacuuccggagcgacgagaucgagcaccucgugguggaggagcuggucaagaagcgcgaggagugccuggacgcccucgaguccaucaugacgaccaagagcguguccuuccggcgccugagccaccugcggaagcucgugcccggguucggcaaggccuacaccaucuucaacaagacccugauggaggccgacgcccacuacaaguccguccgcacguggaacgagaucaucccgagcaaggggugccugcgggugggcggccgcugccacccccacgucaacgggguguucuucaacggcaucauccucgggcccgacggcaacgugcugauccccgagaugcaguccagccugcuccagcagcacauggagcugcuggucuccagcgugaucccgcucaugcacccccuggcggaccccuccaccguguucaagaacggggacgaggccgaggacuucgucgaggugcaccugcccgacgugcacgagcggaucagcggcgucgaccucggccugccgaacugggggaaguacgugcugcucuccgccggcgcccugaccgcccugaugcugaucaucuuccucaugaccugcuggcgccgggugaaccggagcgagcccacgcagcacaaccugcgcgggaccggccgggaggucuccgugaccccgcagagcgggaagaucaucuccagcugggaguccuacaagagcggcggcgagaccgggcugugaggacuagugcaucacauuuaaaagcaucucagccuaccaugagaauaagagaaagaaaaugaagaucaauagcuuauucaucucuuuuucuuuuucguugguguaaagccaacacccugucuaaaaaacauaaauuucuuuaaucauuuugccucuuuucucugugcuucaauuaauaaaaaauggaaagaaccuagaucuaaaaaaaaaaaaaaaaaaaaaaaaaaaaaaaaaaaaaaaaaaaaaaaaaaaaaaaaaaaaaaaaugcaucccccccccccccccccccccccccccccccaaaggcucuuuucagagccaccagaauu

SEQ ID NO: 5 - DNA, 24 bp, Artificial Sequence
  DNA Stem-loop nucleotide sequence
caaaggctcttttcagagccacca

SEQ ID NO: 6 - RNA, 24 nt, Artificial Sequence
  RNA Stem-loop nucleotide sequence
caaaggcucuuuucagagccacca

SEQ ID NO: 7 - protein, 14 aa, Artificial Sequence
  Cys-Arg12-Cys
CRRRRRRRRRRRRC

SEQ ID NO: 8 - protein, 13 aa, Artificial Sequence
  Cys-Arg12
CRRRRRRRRRRRR

SEQ ID NO: 9 - RNA, 546 nt, Artificial Sequence
  isRNA
gggagaaagcucaagcuuauccaaguaggcuggucaccuguacaacguagccgguauuuuuuuuuuuuuuuuuuuuuugaccgucucaagguccaaguuagucugccuauaaaggugcggauccacagcugaugaaagacuugugcgguacgguuaaucuccccuuuuuuuuuuuuuuuuuuuuuaguaaaugcgucuacugaauccagcgaugaugcuggcccagaucuucgaccacaagugcauauaguagucaucgagggucgccuuuuuuuuuuuuuuuuuuuuuuuggcccaguucugagacuucgcuagagacuacaguuacagcugcaguaguaaccacugcggcuauugcaggaaaucccguucagguuuuuuuuuuuuuuuuuuuuuccgcucacuaugauuaagaaccagguggagugucacugcucucgaggucucacgagagcgcucgauacaguccuuggaagaaucuuuuuuuuuuuuuuuuuuuuuugugcgacgaucacagagaacuucuauucaugcaggucugcucua

SEQ ID NO: 10 - RNA, 1083 nt, Artificial Sequence
  isRNA
gggagaaagcucaagcuuauccaaguaggcuggucaccuguacaacguagccgguauuuuuuuuuuuuuuuuuuuuuugaccgucucaagguccaaguuagucugccuauaaaggugcggauccacagcugaugaaagacuugugcgguacgguuaaucuccccuuuuuuuuuuuuuuuuuuuuuaguaaaugcgucuacugaauccagcgaugaugcuggcccagaucuucgaccacaagugcauauaguagucaucgagggucgccuuuuuuuuuuuuuuuuuuuuuuuggcccaguucugagacuucgcuagagacuacaguuacagcugcaguaguaaccacugcggcuauugcaggaaaucccguucagguuuuuuuuuuuuuuuuuuuuuccgcucacuaugauuaagaaccagguggagugucacugcucucgaggucucacgagagcgcucgauacaguccuuggaagaaucuuuuuuuuuuuuuuuuuuuuuugugcgacgaucacagagaacuucuauucaugcaggucugcucuagaacgaacugaccugacgccugaacuuaugagcgugcguauuuuuuuuuuuuuuuuuuuuuuuccucccaacaaaugucgaucaauagcugggcuguuggagacgcgucagcaaaugccguggcuccauaggacguguagacuucuauuuuuuuuuuuuuuuuuuuuucccgggaccacaaauaauauucuugcuugguugggcgcaagggccccguaucaggucauaaacggguacauguugcacaggcuccuuuuuuuuuuuuuuuuuuuuuuucgcugaguuauuccggucucaaaagacggcagacgucagucgacaacacggucuaaagcagugcuacaaucugccguguucguguuuuuuuuuuuuuuuuuuuugugaaccuacacggcgugcacuguaguucgcaauucauaggguaccggcucagaguuaugccuugguugaaaacugcccagcauacuuuuuuuuuuuuuuuuuuuucauauucccaugcuaagcaagggaugccgcgagucauguuaagcuugaauu